SEQUENCE LISTING (2 sequences)

SEQ ID NO: 1 - protein, 93 aa, Homo sapiens
  MISC_FEATURE: Swiss-Prot Q16627
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 2 - protein, 109 aa, Homo sapiens
  MISC_FEATURE: C-C motif chemokine 14 isoform 2
MKISVAAIPFFLLITIALGTKTESSSQTGGKPKVVKIQLKLVGGPYHPSECCFTYTTYKIPRQRIMDYYETNSQCSKPGIVFITKRGHSVCTNPSDKWVQDYIKDMKEN

(19) United States
(12) Patent Application Publication
(10) Pub. No.: US 2022/0236285 A1
(43) Pub. Date: Jul. 28, 2022

(54) METHODS FOR EVALUATION AND TREATMENT OF RENAL INJURY BASED ON C-C MOTIF CHEMOKINE LIGAND 14 MEASUREMENT

(71) Applicant: ASTUTE MEDICAL, INC., SAN DIEGO, CA (US)

(72) Inventors: JAMES PATRICK KAMPF, SAN DIEGO, CA (US); THOMAS KWAN, SAN DIEGO, CA (US); PAUL MCPHERSON, ENCINITAS, CA (US)

(73) Assignee: ASTUTE MEDICAL, INC., SAN DIEGO, CA (US)

(21) Appl. No.: 17/613,766

(22) PCT Filed: May 22, 2020
(86) PCT No.: PCT/US2020/034400

§ 371 Date: Nov. 23, 2021

Related U.S. Application Data
(60) Provisional application No. 62/852,961, filed on May 24, 2019.

(51) Int. Cl. G01N 33/68

(52) U.S. Cl. CPC G01N 33/6863; G01N 2800/347

(57) ABSTRACT

The present invention relates to methods and compositions for monitoring, diagnosis, prognosis, and determination of treatment regimens in subjects suffering from or suspected of having a renal injury. In particular, disclosed herein are methods, compositions, and kits for detecting C-C motif chemokine 14 in combination with creatinine, urine output, and/or cystatin C for predicting the likelihood of persistent acute kidney injury as well as methods for appropriately treating a subject based on his or her assigned likelihood.

### CROSS REFERENCED APPLICATIONS

This application claims the benefit of U.S. Provisional Application No. 62/852,961 filed on May 24, 2019, which is herein incorporated by reference in its entirety.

### SEQUENCE LISTING

This application contains a sequence listing which has been submitted in ASCII format via EFS-Web and is hereby incorporated by reference in its entirety. Said ASCII copy, created on May 21, 2020, is named 01962_PCT_Sequence_Listing_ST25.txt and is 2,396 bytes in size.

### BACKGROUND

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., McGraw Hill, New York, pages 1741-1830. Renal disease and/or injury may be acute or chronic. Acute and chronic kidney disease are described as follows (from Current Medical Diagnosis & Treatment 2008, 47^{th} Ed, McGraw Hill, New York, pages 785-815): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates more than 10% of hospital admissions, 4-15% of cardiopulmonary bypass surgeries, and approaching up to two-thirds of intensive care admissions with survival related, not only to the severity, but also to the duration of renal dysfunction. (Hoste E A, Bagshaw S M, Bellomo R, Cely C M, Colman R, Cruz D N, et al. Intensive Care Med 2015; 41(8):1411-23; Mehta S, Chauhan K, Patel A, Patel S, Pinotti R, Nadkarni G N, et al., BMC Nephrology. 2018; 19(1):91). ARF is a major global cause of both morbidity and mortality. It is estimated that at least half of ARF cases resolve within 72 hours. Cases of ARF that resolve within 72 hours tend to have markedly better outcomes compared to cases which persist for at least 72 hours, especially for cases of severe ARF. Oliguria lasting at least 72 hours has been identified as a criterion for initiation RRT (Gaudry S, Hajage D, Schortgen F, Martin-Lefevre L, Pons B, Boulet E, et al. The New England Journal of Medicine. 2016; 375(2):122-33). Recent evidence suggests that two-thirds of patients with AKI resolve their renal dysfunction within 3-7 days whereas those who persist have dramatically reduced survival over the following year. (Kellum J A, Sileanu F E, Bihorac A, Hoste E A, Chawla L S. Am J Respir Crit Care Med. 2017; 195(6):784-91) Persistence of AKI at one week or more, termed acute kidney disease (AKD), is of grave importance in that it increases an individual's risk of developing chronic kidney disease and the consequences thereof. This link to chronic kidney disease (CKD) has been established over the last decade and specific recommendations for the management of patients with AKD have been proposed in order to try and influence this transition. (Chawla L S, Bellomo R, Bihorac A, Goldstein S L, Siew E D, Bagshaw S M, et al. Nat Rev Nephrol. 2017; 13(4):241-57.; Chawla L S, Eggers P W, Star R A, Kimmel P L. The New England Journal of Medicine 2014; 371(1):58-66.) It follows that early identification of individuals at risk of AKD would enable appropriate delivery of these proposed interventions, but also may identify individuals where newer therapies to attenuate AKI could be targeted.

Not only is persistence of AKI relevant to longer term outcomes, but clinical decision-making is also critically affected by physician expectations surrounding renal recovery and the decision of when to initiate renal replacement therapy (RRT). Currently this is almost totally dependent on clinical expectations as to the likelihood of recovery with no commercially available diagnostics to aid this decision process. As such, significant controversy exists around the timing of RRT with studies showing that some patients can benefit from the earlier initiation of RRT, while other studies demonstrate that some individuals receive RRT who may not require such treatment as they will recover renal function soon. (Bagshaw S M, Lamontagne F, Joannidis M, Wald R. Critical care 2016; 20(1):245; Forni L G, Joannidis M. Nat Rev Nephrol 2019; 15(1):5-6.) It follows that early and reliable identification of those who will recover renal function may enable treatment to be stratified and avoid the incumbent risks of extracorporeal therapy.

These challenges underscore the need for better methods to detect and assess AKI, particularly in the early and subclinical stages, but also in later stages when recovery and repair of the kidney can occur. Furthermore, there is a need to better identify patients who are at risk of having an AKI.

### SUMMARY

Methods and compositions for evaluating renal function in a subject are provided. As described herein, measurement of C-C motif chemokine 14 (CCL14) can be used for diagnosis, prognosis, risk stratification, staging, monitoring, categorizing and determination of further diagnosis and treatment regimens in subjects suffering or at risk of suffering from an injury to renal function, reduced renal function, acute renal failure (also called acute kidney injury), and/or persistent acute kidney injury.

In various aspects C-C motif chemokine 14 is used, individually or in panels comprising a plurality of kidney injury markers, for evaluating renal status in a subject. These panels may include, for example, C-C motif chemokine 14 in addition to one or more of cystatin C, creatinine (e.g., or plasma levels), and/or urine output. In some aspects, one or more of these kidney injury markers are combined into a single composite value for evaluating renal status in a subject. Measurements of these markers may be combined, for example, by multiplication, division, and/or logistic regression. These methods may comprise performing an assay configured to detect C-C motif chemokine 14 in a body fluid sample, such as urine, obtained from the subject. These methods may comprise performing an assay configured to detect cystatin C in a body fluid sample, such as plasma, obtained from the subject. These assay methods may comprise performing an assay configured to detect creatinine (e.g., serum or plasma levels). These assay methods may comprise performing methods to measure urine output. The urine output may be weight-adjusted. The assay results, for example a composite of a measured concentration of C-C motif chemokine 14 and one or more measurements of cystatin C, creatinine (e.g., serum or plasma levels), and/or urine output, may be correlated to a likelihood of persistent acute kidney injury. The subject may be experiencing current acute kidney injury. “Current acute kidney injury” as used herein may refer to characteristics classifying the subject as being at RIFLE stage I or F, and for example, RIFLE F, or KDIGO stage 2 or 3, for example 3, unless specified otherwise.

These assay results may be used for risk stratification (that is, to identify subjects at risk for a future injury to renal function, for future progression to reduced renal function, for future progression to ARF, for future improvement in renal function, etc.); for diagnosis of existing disease (that is, to identify subjects who have suffered an injury to renal function, who have progressed to reduced renal function, who have progressed to ARF, etc.); for monitoring for deterioration or improvement of renal function; and for predicting a future medical outcome, such as improved or worsening renal function, a decreased or increased mortality risk, a decreased or increased risk that a subject will require renal replacement therapy (i.e., hemodialysis, peritoneal dialysis, hemofiltration, and/or renal transplantation), a decreased or increased risk that a subject will recover from an injury to renal function, a decreased or increased risk that a subject will recover from ARF, a decreased or increased risk that a subject will progress to end stage renal disease, a decreased or increased risk that a subject will progress to chronic renal failure, a decreased or increased risk that a subject will suffer rejection of a transplanted kidney, etc.

Disclosed herein are methods for evaluating renal status in a subject. These methods comprise performing an assay method that is configured to detect kidney injury markers of the present invention in a body fluid sample obtained from the subject. The assay result(s), for example a measured concentration or level of C-C motif chemokine 14 in combination with one or more of cystatin C, creatinine (e.g., serum or plasma), and/or urine output is/are then correlated to the renal status of the subject. This correlation to renal status may include correlating the assay result(s) to one or more of risk stratification, diagnosis, prognosis, staging, classifying and monitoring of the subject as described herein. Thus, kidney injury markers disclosed herein may be used for the evaluation of renal injury.

In certain aspects, the methods for evaluating renal status described herein are methods for risk stratification of the subject; that is, assigning a likelihood of one or more future changes in renal status to the subject. In these aspects, the assay result(s) is/are correlated to one or more such future changes. The following are preferred risk stratification aspects.

In preferred risk stratification aspects, these methods comprise determining a subject's risk for a future injury to renal function, and the assay result(s), for example a measured concentration or level of C-C motif chemokine 14 in combination with one or more markers selected from the group consisting of cystatin C, creatinine (e.g., serum or plasma), and urine output, is/are correlated to a likelihood of such a future injury to renal function. For example, the measured concentration(s) may each be compared to a threshold value. For a “positive going” kidney injury marker, an increased likelihood of suffering a future injury to renal function is assigned to the subject when the measured concentration is above the threshold, relative to a likelihood assigned when the measured concentration is below the threshold. For a “negative going” kidney injury marker, an increased likelihood of suffering a future injury to renal function is assigned to the subject when the measured concentration is below the threshold, relative to a likelihood assigned when the measured concentration is above the threshold.

In other preferred risk stratification aspects, these methods comprise determining a subject's risk for future reduced renal function, and the assay result(s), for example a measured concentration or level of C-C motif chemokine 14 in combination with one or more markers selected from the group consisting of cystatin C, creatinine (e.g., serum or plasma), and urine output, is/are correlated to a likelihood of such reduced renal function. For example, the measured concentrations may each be compared to a threshold value. For a “positive going” kidney injury marker, an increased likelihood of suffering a future reduced renal function is assigned to the subject when the measured concentration is above the threshold, relative to a likelihood assigned when the measured concentration is below the threshold. For a “negative going” kidney injury marker, an increased likelihood of future reduced renal function is assigned to the subject when the measured concentration is below the threshold, relative to a likelihood assigned when the measured concentration is above the threshold.

In still other preferred risk stratification aspects, these methods comprise determining a subject's likelihood for a future improvement in renal function, and the assay result(s), for example a measured concentration or level of C-C motif chemokine 14 in combination with one or more markers selected from the group consisting of cystatin C, creatinine (e.g., serum or plasma), and urine output, is/are correlated to a likelihood of such a future improvement in renal function. For example, the measured concentration(s) may each be compared to a threshold value. For a “positive going” kidney injury marker, an increased likelihood of a future improvement in renal function is assigned to the subject when the measured concentration is below the threshold, relative to a likelihood assigned when the measured concentration is above the threshold. For a “negative going” kidney injury marker, an increased likelihood of a future improvement in renal function is assigned to the subject when the measured concentration is above the threshold, relative to a likelihood assigned when the measured concentration is below the threshold.

In yet other preferred risk stratification aspects, these methods comprise determining a subject's risk for progression to ARF, and the result(s), for example a measured concentration or level of C-C motif chemokine 14 in combination with one or more markers selected from the group consisting of cystatin C, creatinine (e.g., serum or plasma), and urine output. For example, the measured concentration(s) may each be compared to a threshold value. For a “positive going” kidney injury marker, an increased likelihood of progression to ARF is assigned to the subject when the measured concentration is above the threshold, relative to a likelihood assigned when the measured concentration is below the threshold. For a “negative going” kidney injury marker, an increased likelihood of progression to ARF is assigned to the subject when the measured concentration is below the threshold, relative to a likelihood assigned when the measured concentration is above the threshold.

And in other preferred risk stratification aspects, these methods comprise determining a subject's outcome risk, and the assay result(s), for example a measured concentration or level of C-C motif chemokine 14 in combination with one or more markers selected from the group consisting of cystatin C, creatinine (e.g., serum or plasma), and urine output, is/are correlated to a likelihood of the occurrence of a clinical outcome related to a renal injury suffered by the subject. For example, the measured concentration(s) may each be compared to a threshold value. For a “positive going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is above the threshold, relative to a likelihood assigned when the measured concentration is below the threshold. For a “negative going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is below the threshold, relative to a likelihood assigned when the measured concentration is above the threshold.

In such risk stratification aspects, preferably the likelihood or risk assigned is that an event of interest is more or less likely to occur within 180 days of the time at which the body fluid sample is obtained from the subject. In particularly preferred aspects, the likelihood or risk assigned relates to an event of interest occurring within a shorter time period such as 18 months, 120 days, 90 days, 60 days, 45 days, 30 days, 21 days, 14 days, 7 days, 5 days, 96 hours, 72 hours, 48 hours, 36 hours, 24 hours, 12 hours, or less. A risk at 0 hours of the time at which the body fluid sample is obtained from the subject is equivalent to diagnosis of a current condition.

In preferred risk stratification aspects, the subject is selected for risk stratification based on the pre-existence in the subject of one or more known risk factors for prerenal, intrinsic renal, or postrenal ARF. For example, a subject undergoing or having undergone major vascular surgery, coronary artery bypass, or other cardiac surgery; a subject having pre-existing congestive heart failure, preeclampsia, eclampsia, diabetes mellitus, hypertension, coronary artery disease, proteinuria, renal insufficiency, glomerular filtration below the normal range, cirrhosis, serum creatinine above the normal range, or sepsis; or a subject exposed to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin are all preferred subjects for monitoring risks according to the methods described herein. This list is not meant to be limiting. By “pre-existence” in this context is meant that the risk factor exists at the time the body fluid sample is obtained from the subject. In particularly preferred aspects, a subject is chosen for risk stratification based on an existing diagnosis of injury to renal function, reduced renal function, or ARF.

In other aspects, the methods for evaluating renal status described herein are methods for diagnosing a renal injury in the subject; that is, assessing whether or not a subject has suffered from an injury to renal function, reduced renal function, or ARF. In these aspects, the assay result(s) is/are correlated to the occurrence or nonoccurrence of a change in renal status. The following are preferred diagnostic aspects.

In preferred diagnostic aspects, these methods comprise diagnosing the occurrence or nonoccurrence of an injury to renal function, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of such an injury. For example, each of the measured concentration(s) may be compared to a threshold value. For a positive going marker, an increased likelihood of the occurrence of an injury to renal function is assigned to the subject when the measured concentration is above the threshold (relative to the likelihood assigned when the measured concentration is below the threshold); alternatively, when the measured concentration is below the threshold, an increased likelihood of the nonoccurrence of an injury to renal function may be assigned to the subject (relative to the likelihood assigned when the measured concentration is above the threshold). For a negative going marker, an increased likelihood of the occurrence of an injury to renal function is assigned to the subject when the measured concentration is below the threshold (relative to the likelihood assigned when the measured concentration is above the threshold); alternatively, when the measured concentration is above the threshold, an increased likelihood of the nonoccurrence of an injury to renal function may be assigned to the subject (relative to the likelihood assigned when the measured concentration is below the threshold).

In other preferred diagnostic aspects, these methods comprise diagnosing the occurrence or nonoccurrence of reduced renal function, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of an injury causing reduced renal function. For example, each of the measured concentration(s) may be compared to a threshold value. For a positive going marker, an increased likelihood of the occurrence of an injury causing reduced renal function is assigned to the subject when the measured concentration is above the threshold (relative to the likelihood assigned when the measured concentration is below the threshold); alternatively, when the measured concentration is below the threshold, an increased likelihood of the nonoccurrence of an injury causing reduced renal function may be assigned to the subject (relative to the likelihood assigned when the measured concentration is above the threshold). For a negative going marker, an increased likelihood of the occurrence of an injury causing reduced renal function is assigned to the subject when the measured concentration is below the threshold (relative to the likelihood assigned when the measured concentration is above the threshold); alternatively, when the measured concentration is above the threshold, an increased likelihood of the nonoccurrence of an injury causing reduced renal function may be assigned to the subject (relative to the likelihood assigned when the measured concentration is below the threshold).

In yet other preferred diagnostic aspects, these methods comprise diagnosing the occurrence or nonoccurrence of ARF, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of an injury causing ARF example, each of the measured concentration(s) may be compared to a threshold value. For a positive going marker, an increased likelihood of the occurrence of ARF is assigned to the subject when the measured concentration is above the threshold (relative to the likelihood assigned when the measured concentration is below the threshold); alternatively, when the measured concentration is below the threshold, an increased likelihood of the nonoccurrence of ARF may be assigned to the subject (relative to the likelihood assigned when the measured concentration is above the threshold). For a negative going marker, an increased likelihood of the occurrence of ARF is assigned to the subject when the measured concentration is below the threshold (relative to the likelihood assigned when the measured concentration is above the threshold); alternatively, when the measured concentration is above the threshold, an increased likelihood of the nonoccurrence of ARF may be assigned to the subject (relative to the likelihood assigned when the measured concentration is below the threshold).

In still other preferred diagnostic aspects, these methods comprise diagnosing a subject as being in need of renal replacement therapy, and the assay result(s), for example a measured concentration or level of C-C motif chemokine 14 in combination with one or more markers selected from the group consisting of cystatin C, creatinine (e.g., serum or plasma), and urine output, is/are correlated to a need for renal replacement therapy. For example, each of the measured concentration(s) may be compared to a threshold value. For a positive going marker, an increased likelihood of the occurrence of an injury creating a need for renal replacement therapy is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal replacement therapy may be assigned to the subject (relative to the likelihood assigned when the measured concentration is above the threshold). For a negative going marker, an increased likelihood of the occurrence of an injury creating a need for renal replacement therapy is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal replacement therapy may be assigned to the subject (relative to the likelihood assigned when the measured concentration is below the threshold).

In still other preferred diagnostic aspects, these methods comprise diagnosing a subject as being in need of renal transplantation, and the assay result(s), for example a measured concentration or level of C-C motif chemokine 14 in combination with one or more markers selected from the group consisting of cystatin C, creatinine (e.g., serum or plasma), and urine output, is/are correlated to a need for renal transplantation. For example, each of the measured concentration(s) may be compared to a threshold value. For a positive going marker, an increased likelihood of the occurrence of an injury creating a need for renal transplantation is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal transplantation may be assigned to the subject (relative to the likelihood assigned when the measured concentration is above the threshold). For a negative going marker, an increased likelihood of the occurrence of an injury creating a need for renal transplantation is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal transplantation may be assigned to the subject (relative to the likelihood assigned when the measured concentration is below the threshold).

In still other aspects, the methods for evaluating renal status described herein are methods for monitoring a renal injury in the subject; that is, assessing whether or not renal function is improving or worsening in a subject who has suffered from an injury to renal function, reduced renal function, or ARF. In these aspects, the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of a change in renal status. The following are preferred monitoring aspects.

In preferred monitoring aspects, these methods comprise monitoring renal status in a subject suffering from an injury to renal function, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other preferred monitoring aspects, these methods comprise monitoring renal status in a subject suffering from reduced renal function, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In yet other preferred monitoring aspects, these methods comprise monitoring renal status in a subject suffering from acute renal failure, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other additional preferred monitoring aspects, these methods comprise monitoring renal status in a subject at risk of an injury to renal function due to the pre-existence of one or more known risk factors for prerenal, intrinsic renal, or postrenal ARF, and the assay result(s), for example a measured concentration or level of C-C motif chemokine 14 in combination with one or more markers selected from the group consisting of cystatin C, creatinine (e.g., serum or plasma), and urine output, is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In yet other preferred monitoring aspects, these methods comprise monitoring renal status in a subject having, or at risk of, an injury to renal function for future persistence of acute kidney injury. “Future persistence” as used herein refers to an existing acute renal injury that will continue for a period selected from the group consisting of 21 days, 14 days, 7 days, 5 days, 96 hours, 72 hours, 48 hours, 36 hours, 24 hours, and 12 hours. In certain aspects the subject has an acute kidney injury at the time the sample is obtained. This is not meant to imply that the subject must have an acute kidney injury at the time the sample is obtained, but rather that the subject, upon onset of an acute kidney injury, suffers from an acute kidney injury that will persist. In various aspects, the assay result(s), for example a measured concentration or level of C-C motif chemokine 14 in combination with one or more markers selected from the group consisting of cystatin C, creatinine (e.g., serum or plasma), and urine output, is/are correlated to the future persistence of the acute kidney injury in the subject. For example, the measured concentration(s) may be compared to a threshold value. For a positive going marker, when the measured concentration is above the threshold, a future persistence of acute kidney injury may be assigned to the subject; alternatively, when the measured concentration is below the threshold, a future improvement of renal function may be assigned to the subject. For a negative going marker, when the measured concentration is below the threshold, a future persistence of acute kidney injury may be assigned to the subject; alternatively, when the measured concentration is above the threshold, a future improvement of renal function may be assigned to the subject.

In still other aspects, the methods for evaluating renal status described herein are methods for classifying a renal injury in the subject; that is, determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE or KDIGO stage. In these aspects, the assay result(s), for example a measured concentration or level of C-C motif chemokine 14 in combination with one or more markers selected from the group consisting of cystatin C, creatinine (e.g., serum or plasma), and urine output, is/are correlated to a particular class and/or subclass. The following are preferred classification aspects.

In preferred classification aspects, these methods comprise determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE stage, and the assay result(s), for example a measured concentration or level of C-C motif chemokine 14 in combination with one or more markers selected from the group consisting of cystatin C, creatinine (e.g., serum or plasma), and urine output is/are correlated to the injury classification for the subject. For example, the measured concentration may be compared to a threshold value, and when the measured concentration is above the threshold, a particular classification is assigned; alternatively, when the measured concentration is below the threshold, a different classification may be assigned to the subject.

A variety of methods may be used by the skilled artisan to arrive at a desired threshold value for use in these methods. For example, the threshold value may be determined from a population of normal subjects by selecting a concentration representing the 75th, 85th, 90th, 95th, or 99th percentile of a kidney injury marker measured in such normal subjects. Alternatively, the threshold value may be determined from a “diseased” population of subjects, e.g., those suffering from an injury or having a predisposition for an injury (e.g., progression to ARF or some other clinical outcome such as death, dialysis, renal transplantation, etc.), by selecting a concentration representing the 75th, 85th, 90th, 95th, or 99th percentile of a kidney injury marker measured in such subjects. In another alternative, the threshold value may be determined from a prior measurement of a kidney injury marker in the same subject; that is, a temporal change in the level of a kidney injury marker in the subject may be used to assign risk to the subject.

The foregoing discussion is not meant to imply, however, that the kidney injury markers disclosed herein must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result which is determined by combining individual markers may be treated as if it is itself a marker; that is, a threshold may be determined for the composite result as described herein for individual markers, and the composite result for an individual patient compared to this threshold.

The ability of a particular test to distinguish two populations can be established using ROC analysis. For example, ROC curves established from a “first” subpopulation which is predisposed to one or more future changes in renal status, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably 0.75, even more preferably at least 0.8, still even more preferably at least 0.9, and most preferably at least 0.95.

In certain aspects, the measured concentration of one or more kidney injury markers, or a composite of such markers, may be treated as continuous variables. For example, any particular concentration can be converted into a corresponding probability of a future reduction in renal function for the subject, the occurrence of an injury, a classification, etc. In yet another alternative, a threshold that can provide an acceptable level of specificity and sensitivity in separating a population of subjects into “bins” such as a “first” subpopulation (e.g., which is predisposed to one or more future changes in renal status, the occurrence of an injury, a classification, etc.) and a “second” subpopulation which is not so predisposed. A threshold value is selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;

a specificity of greater than 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

a sensitivity of greater than 0.5, preferably at least about 0.6, more preferably at least about 0.7, still more preferably at least about 0.8, even more preferably at least about 0.9 and most preferably at least about 0.95, with a corresponding specificity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

at least about 75% sensitivity, combined with at least about 75% specificity;

a positive likelihood ratio (calculated as sensitivity/(1-specificity)) of greater than 1, at least about 2, more preferably at least about 3, still more preferably at least about 5, and most preferably at least about 10; or

a negative likelihood ratio (calculated as (1-sensitivity)/specificity) of less than 1, less than or equal to about 0.5, more preferably less than or equal to about 0.3, and most preferably less than or equal to about 0.1.

The term “about” in the context of any of the above measurements refers to +/−5% of a given measurement.

Multiple thresholds may also be used to assess renal status in a subject. For example, a “first” subpopulation which is predisposed to one or more future changes in renal status, the occurrence of an injury, a classification, etc., and a “second” subpopulation which is not so predisposed can be combined into a single group. This group is then subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer one or more future changes in renal status in comparison to someone in the first tertile. The third tertile is also assigned an odds ratio that is relative to that first tertile.

In certain aspects, the assay method is an immunoassay. Antibodies for use in such assays will specifically bind a full length kidney injury marker of interest, and may also bind one or more polypeptides that are “related” thereto, as that term is defined hereinafter. Numerous immunoassay formats are known to those of skill in the art. Preferred body fluid samples are selected from the group consisting of urine, blood (including whole blood, serum, and plasma), saliva, and tears.

The foregoing method steps should not be interpreted to mean that the kidney injury marker assay result(s) is/are used in isolation in the methods described herein. Rather, additional variables or other clinical indicia may be included in the methods described herein. For example, a risk stratification, diagnostic, classification, monitoring, etc. method may combine the assay result(s) with one or more variables measured for the subject selected from the group consisting of demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a glomerular filtration rate, an estimated glomerular filtration rate, a urine production rate, a serum or plasma creatinine concentration, a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatinine ratio, a renal failure index calculated as urine sodium/(urine creatinine/plasma creatinine), a serum or plasma neutrophil gelatinase (NGAL) concentration, a urine NGAL concentration, a serum or plasma cystatin C concentration, a serum or plasma cardiac troponin concentration, a serum or plasma BNP concentration, a serum or plasma NTproBNP concentration, and a serum or plasma proBNP concentration. Other measures of renal function which may be combined with one or more kidney injury marker assay result(s) are described hereinafter and in Harrison's Principles of Internal Medicine, 17th Ed., McGraw Hill, New York, pages 1741-1830, and Current Medical Diagnosis & Treatment 2008, 47th Ed, McGraw Hill, New York, pages 785-815, each of which are hereby incorporated by reference in their entirety.

When more than one marker is measured, the individual markers may be measured in samples obtained at the same time, or may be determined from samples obtained at different (e.g., an earlier or later) times. The individual markers may also be measured on the same or different body fluid samples. For example, one kidney injury marker may be measured in a serum or plasma sample and another kidney injury marker may be measured in a urine sample. In addition, assignment of a likelihood may combine an individual kidney injury marker assay result with temporal changes in one or more additional variables.

In various related aspects, the present invention also relates to devices and kits for performing the methods described herein. Suitable kits comprise reagents sufficient for performing an assay for at least one of the described kidney injury markers, together with instructions for performing the described threshold comparisons.

In certain aspects, reagents for performing such assays are provided in an assay device, and such assay devices may be included in such a kit. Preferred reagents can comprise one or more solid phase antibodies, the solid phase antibody comprising antibody that detects the intended biomarker target(s) bound to a solid support. In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter.

Detectable labels may include molecules that are themselves detectable (e.g., fluorescent moieties, electrochemical labels, ecl (electrochemical luminescence) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenyl arsenate, ssDNA, dsDNA, etc.).

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc.) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

In some aspects, subjects may be selected for evaluation based on a measured value of one or more AKI biomarkers which indicate an increased risk of having an acute kidney injury, such as acute kidney injury that meets the definition RIFLE I or F or KDIGO Stage 2 or 3. Such biomarkers or indicia may include, but are not limited to, Insulin-like growth factor-binding protein 7, Metalloproteinase inhibitor 2, Neutrophil gelatinase-associated lipocalin, Neutrophil gelatinase-associated lipocalin, Cystatin-C, Interleukin-18, Hepatitis A virus cellular receptor 1, Glutathione S-transferase P, Fatty acid-binding protein, liver, creatinine, urine output, or combinations thereof.

In certain aspects, the level of a marker may be used as a “rule out” for persistent AKI. In these aspects, the measured level of the marker can be compared to a threshold selected from a population study to separate the population into a first subpopulation below the threshold that is at a reduced likelihood of persistent AKI relative to a second subpopulation above the threshold. Such a threshold can, for example, provide a negative predictive value in the first subpopulation of at least 0.6, more preferably at least 0.7, still more preferably at least 0.75, yet more preferably at least 0.8, and most preferably at least 0.9. One or more markers, including any of the markers disclosed herein, may be used to “rule out” a subject prior to and/or after assessing the likelihood of persistent acute kidney injury based on other markers or composite of markers, which may include the same or different markers used to rule a subject out.

In certain aspects, the level of a marker may be used as a “rule in” for persistent AKI. In these aspects, the measured level of the marker can be compared to a threshold selected from a population study to separate the population into a first subpopulation above the threshold that is at an increased likelihood of persistent AKI relative to a second subpopulation below the threshold. Such a threshold can, for example, provide a positive predictive value in the first subpopulation of at least 0.6, more preferably at least 0.7, still more preferably at least 0.75, yet more preferably at least 0.8, and most preferably at least 0.9. One or more markers, including any of the markers disclosed herein, may be used to “rule in” a subject prior to and/or after assessing the likelihood of persistent acute kidney injury based on other markers or composite of markers, which may include the same or different markers used to rule a subject in. In some aspects, different markers may be used to rule out or rule in a subject.

In certain aspects, a subject that is “ruled out” and/or that is assessed to have a relatively low likelihood of persistent acute kidney injury is assigned to a treatment path for the subject's existing AKI that is “conservative,” meaning it does not include renal replacement therapy (RRT). Likewise, in certain aspects, a subject that is “ruled in” and/or that is assessed to have a relatively high likelihood of persistent acute kidney injury is assigned to a treatment path for the subject's existing AKI that comprises administering renal replacement therapy.

In one aspect of the disclosure, a method for diagnosing an occurrence or nonoccurrence of renal injury, reduced renal function, or acute kidney injury (AKI) in a subject is disclosed. The method comprises performing an analyte binding assay configured to detect C-C motif chemokine 14 in a first body fluid sample obtained from the subject. The method further comprises determining a measured value of one or more kidney injury markers. In some embodiments, the one or more kidney injury markers is selected from the group consisting of: a creatinine concentration in the first body fluid sample or in a second body fluid sample obtained from the subject, a cystatin C concentration in the first or the second body fluid sample, a calculated glomerular filtration rate, and a urine output.

In one aspect of the disclosure, a method for risk stratification of a subject is disclosed. The risk stratification comprises assigning a likelihood of the subject having persistent acute kidney injury. The method comprises performing an analyte binding assay configured to detect C-C motif chemokine 14 in a first body fluid sample obtained from the subject. The method further comprises determining a measured value of one or more kidney injury markers. In some embodiments, the one or more kidney injury markers is selected from the group consisting of: a creatinine concentration in the first body fluid sample or in a second body fluid sample obtained from the subject, a cystatin C concentration in the first or the second body fluid sample, a calculated glomerular filtration rate, and a urine output.

In one aspect of the disclosure, a method for monitoring a renal injury in a subject for future persistence of acute kidney injury is disclosed. The risk stratification comprises assigning a likelihood of the subject having persistent acute kidney injury. The method comprises performing an analyte binding assay configured to detect C-C motif chemokine 14 in a first body fluid sample obtained from the subject. The method further comprises determining a measured value of one or more kidney injury markers. In some embodiments, the one or more kidney injury markers is selected from the group consisting of: a creatinine concentration in the first body fluid sample or in a second body fluid sample obtained from the subject, a cystatin C concentration in the first or the second body fluid sample, a calculated glomerular filtration rate, and a urine output.

In some embodiments of the above methods, the method further comprises measuring a volume of urine output, a urine flow rate, a blood creatinine level, or a urine creatinine level within 7 days after the sample is obtained. In some embodiments, the method further comprises calculating a glomerular filtration rate.

In some embodiments of the above methods, the assay result and the measured value of one or more kidney injury markers are combined into a single value to provide a composite assay result.

In some embodiments of the above methods, the method further comprises correlating the assay result together with the measured value to a diagnosis of the renal status of the subject. The diagnosis comprises an occurrence or nonoccurrence of renal injury, reduced renal function, or acute kidney injury (AKI). Optionally, the assay result and the measured value may be correlated together using a composite assay result, as described above.

In some embodiments, the correlating step may comprise assigning the subject to a predetermined subpopulation of individuals having a known predisposition for renal injury, reduced renal function, or acute kidney injury (AKI). Optionally, the assignment may be made by comparing a composite assay result to a threshold selected in a population study, wherein the threshold separates the population into a first subpopulation above the threshold and a second subpopulation at or below the threshold. The first subpopulation may be at an increased predisposition for having renal injury, reduced renal function, or acute kidney injury (AKI) relative to the second subpopulation.

In some embodiments, the method further comprises treating the subject based on the predetermined subpopulation of individuals to which the subject is assigned. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration.

In some embodiments, the method further comprises treating a subject assigned to the first subpopulation. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration. Optionally, the renal replacement therapy of the subject assigned to the first subpopulation may comprise continuous renal replacement therapy, intermittent hemodialysis, peritoneal dialysis, or renal transplantation.

In some embodiments of the above methods, the method further comprises correlating the assay result together with the measured value to a likelihood of the subject having persistent acute kidney injury (AKI). Optionally, the assay result and the measured value may be correlated together using a composite assay result, as described above.

In some embodiments, the correlating step may comprise assigning the subject to a predetermined subpopulation of individuals having a known predisposition for persistent acute kidney injury (AKI). Optionally, the assignment may be made by comparing a composite assay result to a threshold selected in a population study, wherein the threshold separates the population into a first subpopulation above the threshold and a second subpopulation at or below the threshold. The first subpopulation may be at an increased predisposition for having persistent acute kidney injury (AKI) relative to the second subpopulation.

In some embodiments, the method further comprises treating the subject based on the predetermined subpopulation of individuals to which the subject is assigned. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration.

In some embodiments, the method further comprises treating a subject assigned to the first subpopulation. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration. Optionally, the renal replacement therapy of the subject assigned to the first subpopulation may comprise continuous renal replacement therapy, intermittent hemodialysis, peritoneal dialysis, or renal transplantation.

In some embodiments of the above methods, the method further comprises correlating the assay result together with the measured value to an occurrence or nonoccurrence of a change in renal status in the subject. Optionally, the assay result and the measured value may be correlated together using a composite assay result, as described above.

In some embodiments, the correlating step may comprise assigning the subject to a predetermined subpopulation of individuals having a known predisposition for persistent acute kidney injury (AKI). Optionally, the assignment may be made by comparing a composite assay result to a threshold selected in a population study, wherein the threshold separates the population into a first subpopulation above the threshold and a second subpopulation at or below the threshold. The first subpopulation may be at an increased predisposition for having persistent acute kidney injury (AKI) relative to the second subpopulation.

In some embodiments, the method further comprises treating the subject based on the predetermined subpopulation of individuals to which the subject is assigned. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration.

In some embodiments, the method further comprises treating a subject assigned to the first subpopulation. The treatment may comprise one or more of initiating renal replacement therapy, modifying administration of compounds known to be damaging to the kidney by adjusting the amount or selection of the compound, delaying or avoiding procedures that are known to be damaging to the kidney, or modifying diuretic administration. Optionally, the renal replacement therapy of the subject assigned to the first subpopulation may comprise continuous renal replacement therapy, intermittent hemodialysis, peritoneal dialysis, or renal transplantation.

In some embodiments of the above methods, the subject has RIFLE stage I or F or KDIGO stage 2 or 3 when the first and second body fluid samples are obtained.

In some embodiments of the above methods, the binding reagent is an antibody.

In some embodiments of the above methods, the first body fluid sample is a urine sample.

In some embodiments of the above methods, the second body fluid sample is a blood sample.

In some embodiments of the above methods, the first body fluid sample and the second body fluid sample are the same sample. In other embodiments, the first body fluid sample and the second body fluid sample are different samples.

In some embodiments of the above methods, a composite assay result, as described above, is generated via a function obtained using logistic regression or a linear discriminant analysis. In some embodiments, the single composite value is derived from a logistic regression model or a linear discriminant analysis comprising two or more independent variables selected from the group consisting of CCL14, cystatin C, creatinine, urine output, CCL14×cystatin C, CCL14×cystatin C×creatinine, CCL14×cystatin C/urine output, CCL14×creatinine, CCL14×creatinine/urine output, and CCL14/urine output.

In some embodiments of the above methods, a composite assay result, as described above is selected from the group consisting of CCL14×cystatin C, CCL14×cystatin C×creatinine, CCL14×cystatin C/urine output, CCL14×creatinine, CCL14×creatinine/urine output, CCL14/urine output, and CCL14×cystatin C×creatinine/urine output.

In some embodiments of the above methods, correlating the assay result together with the measured value comprises the use of a decision tree analysis or a random forests analysis.

In some embodiments of the above methods, correlating the assay result together with the measured value comprises the use of a number positive analysis.

In some embodiments of the above methods, the subject has been diagnosed with one or more of congestive heart failure, preeclampsia, eclampsia, diabetes mellitus, hypertension, coronary artery disease, proteinuria, renal insufficiency, glomerular filtration below the normal range, cirrhosis, serum creatinine above the normal range, sepsis, injury to renal function, reduced renal function, and acute kidney injury.

In some embodiments of the above methods, the first and second body fluid samples were obtained within 7 days after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis. In some embodiments, the samples were obtained within 72 hours after the acute medical event. In some embodiments, the samples were obtained within 48 hours after the acute medical event. In some embodiments, the samples were obtained within 24 hours after the acute medical event. In some embodiments, the samples were obtained with 12 hours after the acute medical event.

In some embodiments of the above methods, the first and second body fluid samples were obtained within 7 days after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin. In some embodiments, the samples were obtained within 72 hours after the acute medical event. In some embodiments, the samples were obtained within 48 hours after the acute medical event. In some embodiments, the samples were obtained within 24 hours after the acute medical event. In some embodiments, the samples were obtained with 12 hours after the acute medical event.

In some embodiments of the above methods, the assay is performed by introducing the first body fluid sample obtained from the subject into an assay instrument which contacts all or a portion of the first body fluid sample with a binding reagent. The binding reagent specifically binds to C-C motif chemokine 14 for detection of C-C motif chemokine 14. The assay generates an assay result indicative of binding of C-C motif chemokine 14 to the binding reagent.

In some embodiments of the above methods, the subject has RIFLE stage R or KDIGO stage 1 when the first and second body fluid samples are obtained. In some embodiments, the subject has RIFLE stage I or KDIGO stage 2 when the first and second body fluid samples are obtained. In some embodiments, the subject has RIFLE stage F or KDIGO stage 3 when the first and second body fluid samples are obtained.

### BRIEF DESCRIPTION OF THE DRAWINGS

FIGS. 1A-1D illustrate various examples of decision trees for predicting persistent KDIGO stage 3 acute kidney injury. FIG. 1A depicts a decision tree for “persistent” and “non-persistent” cohorts where persistence starts within 24 hours after sample collection and continues for at least 72 hours. FIG. 1B depicts another decision tree for “persistent” and “non-persistent” cohorts where persistence starts within 48 hours after sample collection and continues for at least 72 hours. FIG. 1C depicts yet another example of a decision tree for “persistent” and “non-persistent” cohorts where persistence starts within 48 hours after sample collection and continues for at least 72 hours. FIG. 1D depicts yet another example of a decision tree for “persistent” and “non-persistent” cohorts where persistence starts within 72 hours after sample collection and continues for at least 72 hours.

FIGS. 2A-2B depict box plots of urinary C-C motif chemokine ligand 14 concentrations (FIG. 2A) and plasma cystatin C concentrations (FIG. 2B). Open boxes indicate marker levels for patients having various acute and chronic conditions (see Table 1) who did not persist at any stage of acute kidney injury. Shaded boxes indicate marker levels for patients that maintained a minimum KDIGO stage of 1, 2, or 3 acute level kidney injury over a persistence period of at least 72 hours beginning within a progression window of 48 hours from enrollment. Box and whiskers show interquartile ranges and total observed ranges (censored by 1.5 times the box range), respectively.

FIG. 3 depicts the cumulative composite percentage of patients beginning renal replacement therapy (RRT) or suffering death over 90 days following enrollment for each as stratified by the three tertiles defined by urinary C-C motif chemokine 14 measurements in enrollment samples. The numbers below the graph indicate the number of patients in each tertile who remain without experiencing RRT or suffering death at the time points indicated above.

### DETAILED DESCRIPTION

Disclosed herein are methods and compositions for classifying and determination of treatment regimens in subjects suffering from acute kidney injury (AKI). In various aspects, a measured concentration of C-C motif chemokine 14 (CCL14) or one or more markers related thereto, and optionally one or more additional kidney injury markers known in the art, are correlated to a likelihood of persistent AKI in the subject, and the correlation may be used to guide therapy.

As used herein, an “injury to renal function” is an abrupt (within 14 days, preferably within 7 days, more preferably within 72 hours, and still more preferably within 48 hours) measurable reduction in a measure of renal function. Such an injury may be identified, for example, by a decrease in glomerular filtration rate (GFR) or estimated GFR, a reduction in urine output, an increase in serum creatinine, an increase in serum cystatin C, a requirement for renal replacement therapy, etc. “Improvement in Renal Function” is an abrupt (within 14 days, preferably within 7 days, more preferably within 72 hours, and still more preferably within 48 hours) measurable increase in a measure of renal function. Preferred methods for measuring and/or estimating GFR are described hereinafter.

As used herein, “reduced renal function” is an abrupt (within 14 days, preferably within 7 days, more preferably within 72 hours, and still more preferably within 48 hours) reduction in kidney function identified by an absolute increase in serum creatinine of greater than or equal to 0.1 mg/dL (≥8.8 μmol/L), a percentage increase in serum creatinine of greater than or equal to 20% (1.2-fold from baseline), or a reduction in urine output (documented oliguria of less than 0.5 ml/kg per hour).

As used herein, “acute renal failure” or “ARF” is an abrupt (within 14 days, preferably within 7 days, more preferably within 72 hours, and still more preferably within 48 hours) reduction in kidney function identified by an absolute increase in serum creatinine of greater than or equal to 0.3 mg/dl (≥26.4 μmol/l), a percentage increase in serum creatinine of greater than or equal to 50% (1.5-fold from baseline), or a reduction in urine output (documented oliguria of less than 0.5 ml/kg per hour for at least 6 hours). This term is synonymous with “acute kidney injury” or “AKI.”

In this regard, the skilled artisan will understand that the signals obtained from an immunoassay are a direct result of complexes formed between one or more antibodies and the target biomolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. Expression of biomarkers may also be determined by means other than immunoassays, including protein measurements (such as dot blots, western blots, chromatographic methods, mass spectrometry, etc.) and nucleic acid measurements (mRNA quantitation). This list is not meant to be limiting.

As used herein, the term “C-C motif chemokine 14” refers to one or more polypeptides present in a biological sample that are derived from the C-C motif chemokine 14 precursor (human sequence: Swiss-Prot Q16627 (SEQ ID NO: 1)):

MKISVAAIPF FLLITIALGT KTESSSRGPY HPSECCFTYT TYKIPRQRIM 50
DYYETNSQCS KPGIVFITKR GHSVCTNPSD KWVQDYIKDM KEN 93

The following domains have been identified in C-C motif chemokine 14:

| Residues | Length | Domain ID |
|---|---|---|
| 1-19 | 19 | Signal peptide |
| 20-93 | 74 | C-C motif chemokine 14 |
| 22-93 | 72 | HCC-1(3-74) |
| 23-93 | 71 | HCC-1(4-74) |
| 28-93 | 66 | HCC-1(9-74) |
| 27 | | R → QTGGKPKVVKIQLKLVG |
| | | in isoform 2 |
| | | (SEQ ID NO: 2) |

As used herein, the term “relating a signal to the presence or amount” of an analyte reflects this understanding. Assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Because an antibody epitope is on the order of 8 amino acids, an immunoassay configured to detect a marker of interest will also detect polypeptides related to the marker sequence, so long as those polypeptides contain the epitope(s) necessary to bind to the antibody or antibodies used in the assay.

The term “related marker” as used herein with regard to a biomarker such as one of the kidney injury markers described herein refers to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

The term “positive going” marker as that term is used herein refer to a marker that is determined to be elevated in subjects suffering from a disease or condition, relative to subjects not suffering from that disease or condition. The term “negative going” marker as that term is used herein refer to a marker that is determined to be reduced in subjects suffering from a disease or condition, relative to subjects not suffering from that disease or condition.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample may be obtained from a subject, or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain aspects, such a sample may be obtained for the purpose of determining the outcome of an ongoing condition or the effect of a treatment regimen on a condition. Preferred body fluid samples include blood (including whole blood, serum, and plasma), cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, for a kidney injury marker of the present invention, optionally together with other clinical characteristics, to arrive at a diagnosis (that is, the occurrence or nonoccurrence) of an acute renal injury or ARF for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient.

ARF may be categorized as prerenal, intrinsic renal, or postrenal in causation. Intrinsic renal disease can be further divided into glomerular, tubular, interstitial, and vascular abnormalities. Major causes of ARF are described in the following table, which is adapted from the Merck Manual, 17^{th} ed., Chapter 222, and which is hereby incorporated by reference in its entirety:

| Type | Risk Factors |
|---|---|
| Prerenal | |
| ECF volume depletion | Excessive diuresis, hemorrhage, GI losses, loss of |
| | intravascular fluid into the extravascular space (due to |
| | ascites, peritonitis, pancreatitis, or burns), loss of skin |
| | and mucus membranes, renal salt- and water-wasting states |
| Low cardiac output | Cardiomyopathy, MI, cardiac tamponade, pulmonary |
| | embolism, pulmonary hypertension, positive-pressure |
| | mechanical ventilation |
| Low systemic vascular | Septic shock, liver failure, antihypertensive drugs |
| resistance | |
| Increased renal vascular | NSAIDs, cyclosporines, tacrolimus, hypercalcemia, |
| resistance | anaphylaxis, anesthetics, renal artery obstruction, renal |
| | vein thrombosis, sepsis, hepatorenal syndrome |
| Decreased efferent | ACE inhibitors or angiotensin II receptor blockers |
| arteriolar tone (leading to | |
| decreased GFR from | |
| reduced glomerular | |
| transcapillary pressure, | |
| especially in patients with | |
| bilateral renal artery | |
| stenosis) | |
| Intrinsic Renal | |
| Acute tubular injury | Ischemia (prolonged or severe prerenal state): surgery, |
| | hemorrhage, arterial or venous obstruction; Toxins: |
| | NSAIDs, cyclosporines, tacrolimus, aminoglycosides, |
| | foscarnet, ethylene glycol, hemoglobin, myoglobin, |
| | ifosfamide, heavy metals, methotrexate, radiopaque |
| | contrast agents, streptozotocin |
| Acute glomerulonephritis | ANCA-associated: Crescentic glomerulonephritis, |
| | polyarteritis nodosa, Wegener's granulomatosis; Anti- |
| | GBM glomerulonephritis: Goodpasture's syndrome; |
| | Immune-complex: Lupus glomerulonephritis, |
| | postinfectious glomerulonephritis, cryoglobulinemic |
| | glomerulonephritis |
| Acute tubulointerstitial | Drug reaction (eg, β-lactams, NSAIDs, sulfonamides, |
| nephritis | ciprofloxacin, thiazide diuretics, furosemide, phenytoin, |
| | allopurinol, pyelonephritis, papillary necrosis |
| Acute vascular | Vasculitis, malignant hypertension, thrombotic |
| nephropathy | microangiopathies, scleroderma, atheroembolism |
| Infiltrative diseases | Lymphoma, sarcoidosis, leukemia |
| Postrenal | |
| Tubular precipitation | Uric acid (tumor lysis), sulfonamides, triamterene, |
| | acyclovir, indinavir, methotrexate, ethylene glycol |
| | ingestion, myeloma protein, myoglobin |
| Ureteral obstruction | Intrinsic: Calculi, clots, sloughed renal tissue, fungus |
| | ball, edema, malignancy, congenital defects; Extrinsic: |
| | Malignancy, retroperitoneal fibrosis, ureteral trauma |
| | during surgery or high impact injury |
| Bladder obstruction | Mechanical: Benign prostatic hyperplasia, prostate |
| | cancer, bladder cancer, urethral strictures, phimosis, |
| | paraphimosis, urethral valves, obstructed indwelling |
| | urinary catheter; Neurogenic: Anticholinergic drugs, |
| | upper or lower motor neuron lesion |

In the case of ischemic ARF, the course of the disease may be divided into four phases. During an initiation phase, which lasts hours to days, reduced perfusion of the kidney is evolving into injury. Glomerular ultrafiltration reduces, the flow of filtrate is reduced due to debris within the tubules, and back leakage of filtrate through injured epithelium occurs. Renal injury can be mediated during this phase by reperfusion of the kidney. Initiation is followed by an extension phase which is characterized by continued ischemic injury and inflammation and may involve endothelial damage and vascular congestion. During the maintenance phase, lasting from 1 to 2 weeks, renal cell injury occurs, and glomerular filtration and urine output reaches a minimum. A recovery phase can follow in which the renal epithelium is repaired and GFR gradually recovers. Despite this, the survival rate of subjects with ARF may be as low as about 60%.

Acute kidney injury caused by radiocontrast agents (also called contrast media) and other nephrotoxins such as cyclosporine, antibiotics including aminoglycosides and anticancer drugs such as cisplatin manifests over a period of days to-about a week. Contrast induced nephropathy (CIN, which is AKI caused by radiocontrast agents) is thought to be caused by intrarenal vasoconstriction (leading to ischemic injury) and from the generation of reactive oxygen species that are directly toxic to renal tubular epithelial cells. CIN classically presents as an acute (onset within 24-48 h) but reversible (peak 3-5 days, resolution within 1 week) rise in blood urea nitrogen and serum creatinine.

A commonly reported criterion for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16: 3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum creatinine rise.

One study (Lassnigg et al, J Am Soc Nephrol 15:1597-1605, 2004) investigated both increases and decreases in serum creatinine. Patients with a mild fall in serum creatinine of −0.1 to −0.3 mg/dL following heart surgery had the lowest mortality rate. Patients with a larger fall in serum creatinine (more than or equal to −0.4 mg/dL) or any increase in serum creatinine had a larger mortality rate. These findings caused the authors to conclude that even very subtle changes in renal function (as detected by small creatinine changes within 48 hours of surgery) seriously effect patient's outcomes. In an effort to reach consensus on a unified classification system for using serum creatinine to define AKI in clinical trials and in clinical practice, Bellomo et al., Crit Care. 8(4):R204-12, 2004, which is hereby incorporated by reference in its entirety for the RIFLE criteria, proposes the following classifications for stratifying AKI patients:

“Risk”: serum creatinine increased 1.5 fold from baseline OR urine production of <0.5 ml/kg body weight/hr for 6 hours;

“Injury”: serum creatinine increased 2.0 fold from baseline OR urine production <0.5 ml/kg/hr for 12 h;

“Failure”: serum creatinine increased 3.0 fold from baseline OR creatinine >355 μmol/l (with a rise of >44) or urine output below 0.3 ml/kg/hr for 24 h or anuria for at least 12 hours;

And Included Two Clinical Outcomes:

“Loss”: persistent need for renal replacement therapy for more than four weeks.
“ESRD”: end stage renal disease—the need for dialysis for more than 3 months.

These criteria are called the RIFLE criteria, which provide a useful clinical tool to classify renal status. As discussed in Kellum, Crit Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies.

More recently, Mehta et al., Crit Care 11:R31 (doi:10.1186.cc5713), 2007, hereby incorporated by reference in its entirety, proposes the following similar classifications for stratifying AKI patients (AKIN), which have been modified from RIFLE:

“Stage I”: increase in serum creatinine of more than or equal to 0.3 mg/dL (≥26.4 μmol/L) or increase to more than or equal to 150% (1.5-fold) from baseline OR urine output less than 0.5 mL/kg per hour for more than 6 hours;

“Stage II”: increase in serum creatinine to more than 200% (>2-fold) from baseline OR urine output less than 0.5 mL/kg per hour for more than 12 hours;

“Stage III”: increase in serum creatinine to more than 300% (>3-fold) from baseline OR serum creatinine ≥354 μmol/L accompanied by an acute increase of at least 44 μmol/L OR urine output less than 0.3 mL/kg per hour for 24 hours or anuria for 12 hours.

Likewise, Kidney Disease: Improving Global Outcomes (KDIGO) Acute Kidney Injury Work Group. KDIGO Clinical Practice Guideline for Acute Kidney Injury, Kidney inter., Suppl. 2012; 2: 1-138, refers to both RIFLE and AKIN, and offers the following AKI staging guidelines:

| Stage | Serum creatinine or | Urine output |
|---|---|---|
| 1 | 1.5-1.9 times baseline | <0.5 ml/kg/h |
| | or ≥0.3 mg/dl (≥26.5 mmol/l) increase | for 6-12 hours |
| 2 | 2.0-2.9 times baseline | <0.5 ml/kg/h |
| | | for ≥12 hours |
| 3 | 3.0 times baseline Or | <0.3 ml/kg/h |
| | Increase in serum creatinine to ≥4.0 mg/dl | for ≥24 hours |
| | (≥353.6 mmol/l) or | or Anuria |
| | Initiation of renal replacement therapy or | for ≥12 hours |
| | In patients <18 years, decrease in eGFR | |
| | to <35 ml/min per 1.73 m2 | |

The CIN Consensus Working Panel (McCollough et al, Rev Cardiovasc Med. 2006; 7(4):177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

These classification systems of AKI generally comprise serum creatinine criteria and urine output criteria for each stage. Wherever specified herein, any stage of AKI may be considered equivalent to (i.e. substituted with) any of the individual criteria that qualifies a subject as being at that particular stage of AKI. In some embodiments, the methods disclosed herein may also be used to correlate to a renal status defined by a particular AKI stage (e.g., the likelihood of reaching a particular AKI stage or the likelihood of persistent AKI at a particular stage), wherein the particular AKI stage can be defined by meeting both a serum creatinine criterion that qualifies the subject for that particular stage and a urine output criterion that qualifies a subject for that particular stage. In some embodiments, the particular AKI stage can be defined by meeting all the criteria (i.e. both of all the serum creatinine criteria and all the urine output criteria). All the methods disclosed herein may define stages of AKI according to any of these embodiments, unless stated otherwise.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease. Because serum creatinine is a marker of filtration rate, it does not differentiate between the causes of AKI (pre-renal, intrinsic renal, post-renal obstruction, atheroembolic, etc.) or the category or location of injury in intrinsic renal disease (for example, tubular, glomerular or interstitial in origin). Urine output is similarly limited. Knowing these things can be of vital importance in managing and treating patients with AKI.

Marker Assays

In general, immunoassays involve contacting a sample containing or suspected of containing a biomarker of interest with at least one antibody that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the latter case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In certain aspects, the present invention provides kits for the analysis of the described kidney injury markers. The kit comprises reagents for the analysis of at least one test sample which comprise at least one antibody that a kidney injury marker. The kit can also include devices and instructions for performing one or more of the diagnostic and/or prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies used in the immunoassays described herein preferably specifically bind to a kidney injury marker of the present invention. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred aspects, Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹²M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Assay Correlations

The term “correlating” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and riskier, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5^{th} percentile of the concentration seen in a normal population. Another method may be to look at serial samples from the same patient, where a prior “baseline” result is used to monitor for temporal changes in a biomarker level.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1−specificity, the ROC graph is sometimes called the sensitivity vs (1−specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.75, even more preferably at least 0.8, still even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

Additional clinical indicia may be combined with the kidney injury marker assay result(s) of the present invention. These include other biomarkers related to renal status. Examples include the following, which recite the common biomarker name, followed by the Swiss-Prot entry number for that biomarker or its parent: Actin (P68133); Adenosine deaminase binding protein (DPP4, P27487); Alpha-1-acid glycoprotein 1 (P02763); Alpha-1-microglobulin (P02760); Albumin (P02768); Angiotensinogenase (Renin, P00797); Annexin A2 (P07355); Beta-glucuronidase (P08236); B-2-microglobulin (P61769); Beta-galactosidase (P16278); BMP-7 (P18075); Brain natriuretic peptide (proBNP, BNP-32, NTproBNP; P16860); Calcium-binding protein Beta (S100-beta, P04271); Carbonic anhydrase 9 (Q16790); Casein Kinase 2 (P68400); Clusterin (P10909); Complement C3 (P01024); Cysteine-rich protein (CYR61, 000622); Cytochrome C (P99999); Epidermal growth factor (EGF, P01133); Endothelin-1 (P05305); Exosomal Fetuin-A (P02765); Fatty acid-binding protein, heart (FABP3, P05413); Fatty acid-binding protein, liver (P07148); Ferritin (light chain, P02792; heavy chain P02794); Fructose-1,6-biphosphatase (P09467); GRO-alpha (CXCL1, (P09341); Growth Hormone (P01241); Hepatocyte growth factor (P14210); Insulin-like growth factor I (P05019); Immunoglobulin G; Immunoglobulin Light Chains (Kappa and Lambda); Interferon gamma (P01308); Lysozyme (P61626); Interleukin-1alpha (P01583); Interleukin-2 (P60568); Interleukin-4 (P05112); Interleukin-9 (P15248); Interleukin-12p40 (P29460); Interleukin-13 (P35225); Interleukin-16 (Q14005); L1 cell adhesion molecule (P32004); Lactate dehydrogenase (P00338); Leucine Aminopeptidase (P28838); Meprin A-alpha subunit (Q16819); Meprin A-beta subunit (Q16820); Midkine (P21741); MIP2-alpha (CXCL2, P19875); MMP-2 (P08253); MMP-9 (P14780); Netrin-1 (095631); Neutral endopeptidase (P08473); Osteopontin (014788); Renal papillary antigen 1 (RPA1); Renal papillary antigen 2 (RPA2); Retinol binding protein (P09455); Ribonuclease; S100 calcium-binding protein A6 (P06703); Serum Amyloid P Component (P02743); Sodium/Hydrogen exchanger isoform (NHE3, P48764); Spermidine/spermine N1-acetyltransferase (P21673); TGF-Beta1 (P01137); Transferrin (P02787); Trefoil factor 3 (TFF3, Q07654); Toll-Like protein 4 (000206); Total protein; Tubulointerstitial nephritis antigen (Q9UJW2); Uromodulin (Tamm-Horsfall protein, P07911).

For purposes of risk stratification, Adiponectin (Q15848); Alkaline phosphatase (P05186); Aminopeptidase N (P15144); CalbindinD28k (P05937); Cystatin C (P01034); 8 subunit of F1FO ATPase (P03928); Gamma-glutamyltransferase (P19440); GSTa (alpha-glutathione-S-transferase, P08263); GSTpi (Glutathione-S-transferase P; GST class-pi; P09211); IGFBP-1 (P08833); IGFBP-2 (P18065); IGFBP-6 (P24592); Integral membrane protein 1 (Itm1, P46977); Interleukin-6 (P05231); Interleukin-8 (P10145); Interleukin-18 (Q14116); IP-10 (10 kDa interferon-gamma-induced protein, P02778); IRPR (IFRD1, 000458); Isovaleryl-CoA dehydrogenase (IVD, P26440); I-TAC/CXCL11 (014625); Keratin 19 (P08727); Kim-1 (Hepatitis A virus cellular receptor 1, 043656); L-arginine: glycine amidinotransferase (P50440); Leptin (P41159); Lipocalin2 (NGAL, P80188); MCP-1 (P13500); MIG (Gamma-interferon-induced monokine Q07325); MIP-la (P10147); MIP-3a (P78556); MIP-1beta (P13236); MIP-1d (Q16663); NAG (N-acetyl-beta-D-glucosaminidase, P54802); Organic ion transporter (OCT2, O15244); Osteoprotegerin (014788); P8 protein (O60356); Plasminogen activator inhibitor 1 (PAI-1, P05121); ProANP(1-98) (P01160); Proenkephalin (P01210); Protein phosphatase 1-beta (PPI-beta, P62140); Rab GDI-beta (P50395); Renal kallikrein (P06870); RT1.B-1 (alpha) chain of the integral membrane protein (Q5Y7A8); soluble tumor necrosis factor receptor superfamily member 1A (sTNFR-I, P19438); soluble tumor necrosis factor receptor superfamily member 1B (sTNFR-II, P20333); Tissue inhibitor of metalloproteinases 3 (TIMP-3, P35625); uPAR (Q03405) may be combined with the kidney injury marker assay result(s) of the present invention.

Biomarkers associated with apoptosis, necrosis, endothelial injury, cell-cell and cell-matrix adhesion, cytoprotection, oxidative processes, cell-cycle regulation, inflammation, tubular injury, immune function, and fibrosis may be suitable candidates alone or in combination for predicting and/or diagnosing renal status, including renal injury, acute renal injury, and/or persistent acute renal injury, based on plausible mechanisms of renal tissue damage and repair (Kashani K, Al-Khafaji A, Ardiles T, Artigas A, Bagshaw S M, Bell M, et al. Crit Care. 2013; 17(1):R25). C-C motif chemokine 14 (CCL14) may function as a kidney injury marker correlated to renal status, including a marker of current and/or future persistent acute kidney injury. CCL14 is a member of the chemokine family of small molecules that were initially recognized for roles in leukocyte chemotaxis and are implicated in tissue injury and repair processes. CCL14 binds with high affinity to the chemokine receptors CCR1 and CCRS and lower affinity to CCR3. (Detheux M, Standker L, Vakili J, Munch J, Forssmann U, Adermann K, et al. J Exp Med. 2000; 192(10):1501-8.) CCL14 is an important chemokine for monocyte/macrophage recruitment and is associated with pro-inflammatory chemotaxis in a variety of diseases including rheumatoid arthritis, multiple sclerosis, and lupus (Rump L, Mattey D L, Kehoe O, Middleton J. Cytokine. 2017; 97:133-40; Vyshkina T, Sylvester A, Sadiq S, Bonilla E, Perl A, Kalman B. J Neuroimmunol. 2008; 200(1-2):145-52).

Other clinical indicia which may be combined with the kidney injury marker assay result(s) of the present invention includes demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a urine total protein measurement, a glomerular filtration rate, an estimated glomerular filtration rate, a urine production rate, a serum or plasma creatinine concentration, a renal papillary antigen 1 (RPA1) measurement; a renal papillary antigen 2 (RPA2) measurement; a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatinine ratio, and/or a renal failure index calculated as urine sodium/(urine creatinine/plasma creatinine). Other measures of renal function which may be combined with the kidney injury marker assay result(s) are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

Combining assay results/clinical indicia in this manner can comprise the use of multivariate logistical regression, loglinear modeling, neural network analysis, n-of-m analysis, decision tree analysis, etc. This list is not meant to be limiting.

Where measurements or values of individual markers (e.g., assay results of individual marker concentrations in samples) and/or other clinical indicia are combined into single composite values, the single composite value may be treated as if it were an individual biomarker, such as for purposes of statistics. In some aspects (a “product model”), arithmetic operators such as “X” (multiplication) and “/” (division) may be used in their ordinary sense to combine individual parameters, such as marker levels, into a single composite value. In another aspect, logistic regression may be used to combine individual parameters into a single composite value. Logistic regression is a method widely used for models which have a binary outcome, such as a “persistent” or “non-persistent” dichotomy. By way of example, a method for logistic regression will be briefly described below, while full treatments can be found in the literature. The model may be represented by the following function:

$${\prod }_{i} ( {y}_{i} = 1 \vert {x}_{i} ) = \frac{{e}^{α + β {x}_{i}}}{1 + {e}^{α + β {x}_{i}}}$$

In this model, x and β are vectors, x representing the different observables or analyte values, yi=1 indicates a diseased state, and Π is the model probability of this state for the ith case given xi. The panel value for each sample is Π i. The log odds or logit is:

$$logit = ln [ \frac{{\prod }_{i} ( {y}_{i} = 1 \vert {x}_{i} )}{1 - {\prod }_{i} ( {y}_{i} = 1 \vert {x}_{i} )} ] = α + β {x}_{i}$$

The probability of observing the true outcome may be defined as pi:

$${p}_{i} = \{ \begin{matrix}{\prod }_{i} ( {y}_{i} = 1 \vert {x}_{i} ) & if deseased \\ 1 - {\prod }_{i} ( {y}_{i} = 1 \vert {x}_{i} ) & if non \text{-} diseased\end{matrix} \}$$

The likelihood function is the product of the probabilities of observing the true outcomes, so the log likelihood (LL) is:

LL=ln(L(α,β))=Σ ln(pᵢ)

To find the parameters, α and β, that best fit the model, the negative log likelihood (−LL) is minimized. This minimization can be performed using the Levenberg-Marquardt method (Numerical Recipes: The Art of Scientific Computing, Third Edition, Cambridge University Press, 2007). The initial point for each parameter is 0.

A commonly used statistic to compare the fit of two nested models is the likelihood ratio test. This statistic, or the deviance, is the difference in twice the negative log likelihood (−2LL) for the two model, and is asymptotically a χ2 distribution with DF equal to the change in the number of degrees of freedom (number of independent parameters) between the models. The p-value is calculated from this statistic. The null hypothesis is that the logistic model is not different than a constant model (β set to 0), is tested for each model using the likelihood ratio test. The ‘model p-value’ is defined as the probability that the null hypothesis is true. For the constant model, a closed form solution for a and −2LL can be found. It is a function of the number of diseased (#D) and the number of non-diseased (#ND) samples in the data set.

$$α = ln ( \frac{\# D}{\# ND} ) \\ - 2 LL = - 2 * [ \# ND ln ( 1 - \prod ) + \# D ln ( \prod ) ]$$

In some embodiments, CCL14 may be combined into a single composite value with one or more of cystatin C, creatinine, and urine output using a product model. By way of example, measurements of the markers may be combined as: CCL14×cystatin C, CCL14×cystatin C×creatinine, CCL14×cystatin C/urine output, CCL14×creatinine, CCL14×creatinine/urine output, CCL14/urine output, and CCL14×cystatin C×creatinine/urine output.

In some embodiments, CCL14 may be combined into a single composite value with one or more of cystatin C, creatinine, and urine output using logistic regression. In a logistic regression model, the logarithm of the odds (log-odds) of a subject having a high risk and/or a positive diagnosis, for example, (e.g., the risk of persistent AKI) is modeled by a linear combination of the predictors. Measurements of the markers may be combined, for example, as described elsewhere herein.

In some embodiments, CCL14 may be combined into a single composite value with one or more of cystatin C, creatinine, and urine output using loglinear modeling. By way of example, a method for loglinear modeling may be performed as described by Khoury et al. “Familial aggregation in chronic obstructive pulmonary disease: use of the loglinear model to analyze intermediate environmental and genetic risk factors. Genet Epidemiol. 2(2): 155-66 (1985), which is hereby incorporated by reference in its entirety.

In some embodiments, CCL14 may be combined into a single composite value with one or more of cystatin C, creatinine, and urine output using a linear discriminant. A linear discriminant models the relationship between the higher risk and lower risk subjects or subjects with positive and negative diagnoses, for example, (e.g., subjects at high and low risk of persistent AKI) by finding a linear combination of the predictors that maximizes the separation between the two groups.

In some embodiments, CCL14 may be used together with (i.e. in combination with) one or more of cystatin C, creatinine, and urine output by using measurements or assay results together in a decision tree analysis. By way of example, a method for decision tree analysis may be performed as described in Song, Y Y, and Lu Y (2015) “Decision tree methods: applications for classification and prediction.” Shanghai Arch Psychiatry. 2015 Apr. 25; 27(2): 130-135, which is hereby incorporated by reference in its entirety. A decision tree comprises of a sequence of two or more nodes (e.g., 2, 3, 4, 5, 6, 7, 8, 9, 10 nodes) where an input variable is tested and two branches extending from each node which denote the two possible outcomes of the test at the node. To classify an input, it is “put down” a tree from the top and ultimately yielding a probability classification into one of the at least two classes formed at the cumulative terminals of the tree branches. The input may comprise a measurement or assay result of CCL14 in addition to one or more measurements or assay results of additional markers or indicia (e.g., one or more of cystatin C, creatinine, and urine output). The output may comprise any one or more of the renal statuses disclosed elsewhere herein (e.g., a risk or likelihood of persistent AKI, a diagnosis of persistent AKI, a risk or likelihood of AKI, a diagnosis of AKI, an outcome related to AKI, etc.). A probability that a subject meeting the requirements of a specific branch terminus has a particular renal status may be estimated, for example, based on the proportion of a population meeting the same branch terminus requirements (e.g., from a population study) who actually have that particular renal status.

A decision tree analysis may be used to generate two or more probabilities of a renal status (e.g., persistent AKI) corresponding to the two or more branch terminals. In some embodiments, the probabilities at each terminus may be ranked in descending/ascending order and correlated to a relative risk or likelihood of the renal status. For example, a decision tree having three branch terminals may be used to classify a subject as having a relatively high, intermediate, or low risk of having a particular renal status. In some embodiments, one or more of the branch terminals may be grouped into the same category. For instance, all probabilities above or below a threshold probability may be grouped into one category. By way of example, a decision tree having three or more branch terminals may be used to assign subjects a relatively high or low risk of persistent acute kidney injury or a decision tree having four or more branch terminals may be used to assign subjects a relatively high, intermediate, or low risk of persistent acute kidney injury. In some implementations, a probability of at least 60%, 65%, 70%, 75%, 80%, 85%, 90%, or 95% may be considered sufficient for classifying a subject as being at the highest risk or likelihood. In some implementations, a probability of no more than 40%, 35%, 30%, 25%, 20%, 15%, 10%, or 5% may be considered sufficient for classifying a subject as being at the lowest risk or likelihood. In some implementations an intermediate risk or likelihood may be assigned to any subject not meeting the criteria of either the highest or lowest risk or likelihood. In some implementations, multiple branches may be collapsed into a single branch and the probability recalculated based on the adjusted proportion of subjects having the particular renal status. In some implementations, the probability of the highest risk group may be at least 1.10, 1.25, 1.5, 1.75, 2.0, 3.0, 4.0, or 5.0 times greater than the lowest risk group. In some implementations, the probability of the highest risk group may be at least 1.10, 1.25, 1.5, 1.75, 2.0, 3.0, 4.0, or 5.0 times greater than the intermediate risk group. In some implementations, each risk group may be at least 1.10, 1.25, 1.5, 1.75, 2.0, 3.0, 4.0, or 5.0 times greater than the next highest risk group.

In various embodiments of the decision tree analysis, the nodes may comprise thresholds for a marker in which one branch specifies the measurement (e.g., the marker concentration) is greater than the threshold and the other branch specifies the measurement is at or below the threshold. In some embodiments, the decision tree may comprise at least one CCL14 node and an additional node selected from a cystatin C node, a creatinine node, and a urine output node. In some embodiments, the decision tree may comprise at least one CCL14 node and two additional nodes selected from a cystatin C node, a creatinine node, and a urine output node. In some embodiments, the decision tree may comprise at least one CCL14 node, a cystatin C node, a creatinine node, and a urine output node. In some embodiments, the decision tree may comprise more than one node for the same marker. For instance, a first node in the series (i.e. toward the top of the tree) may specify a first threshold and a second node in the series (i.e. toward the bottom of the tree) may specify a second threshold which is above or below the first threshold depending on which branch of the first node the second node is positioned.

In various embodiments, one or more of the nodes may comprise a threshold test corresponding to a RIFLE or KDIGO criterion. For instance, in one embodiment, a node may test whether serum creatinine is above a threshold value that qualifies the subject as being at one of RIFLE stages R, I, or F or at one of KDIGO stages 1, 2, or 3, as defined elsewhere herein. In another embodiment, a node may test whether urine output is below a threshold value that qualifies the subject as being at one of RIFLE stages R, I, or F or at one of KDIGO stages 1, 2, or 3, as defined elsewhere herein. In general, a subject who meets both a serum creatinine criterion and a urine output criterion for one of RIFLE stages R, I, or F or one of KDIGO stages 1, 2, or 3 may be at greater risk or likelihood for an adverse renal status (e.g., persistent AKI at that RIFLE stage or progression to the next stage of AKI) than a subject who meets only the serum creatinine criterion or the urine output criterion for the same stage. In general, a subject who satisfies multiple thresholds (i.e. exceeds a threshold of a positive going marker or falls at or below a threshold of a negative going marker) for CCL14, cystatin C, creatinine, and urine output will be at a greater likelihood or risk of exhibiting the renal status defined by meeting those thresholds than if the subject only satisfied one of the thresholds. The risk or likelihood may increase as the number of thresholds satisfied increases. The specific thresholds for any node may be any of the thresholds disclosed elsewhere herein. In some implementations, the threshold may be adjusted to allow for a larger proportion of a population to meet the branch criterion than if the marker were to be used alone and yet the probability of the selected population actually exhibiting the renal status tested for may be improved by the combined use of the marker with additional markers.

In some embodiments, the input into any of the nodes may be a single measurement or assay result for CCL14, cystatin C, creatinine, and urine output. In some embodiments, the input into any of the nodes may be a composite value. In some embodiments, the input into the nodes may comprise both single measurements/assay results and composite values. The composite value may be generated from a combination of one or more measurements or assay results for CCL14, cystatin C, creatinine, and urine output as described elsewhere herein. For example, the input may comprise CCL14×cystatin C, CCL14×cystatin C×creatinine, CCL14×cystatin C/urine output, CCL14×creatinine, CCL14×creatinine/urine output, CCL14/urine output, and CCL14×cystatin C×creatinine/urine output. The composite value may be generated using logistic regression, loglinear modeling, a linear discriminant, or ratio of markers as described elsewhere herein.

In some embodiments, CCL14 may be used together with (i.e. in combination with) one or more of cystatin C, creatinine, and urine output by using measurements or assay results together in a random forests analysis. A random forests analysis comprises multiple unique decision trees each of which may measure the probability of the same outcome. The ultimate result of the random forests analysis may be determined by the number (the proportion) of trees that generate the same outcome for the same input. In some embodiments, a majority “vote” of the trees may be used to assign a renal status to the subject (e.g., the subject will be assigned a relatively high likelihood of persistent AKI if a majority of the trees predict a high likelihood of persistent AKI). In some embodiments, the risk or likelihood may be stratified according to the number of trees resulting in a particular outcome. For example, a high risk or likelihood may be assigned if at least 70% of the trees predict a high likelihood, an intermediate risk or likelihood may be assigned if between 30-70% of the trees predict a high likelihood, and a low risk or likelihood may be assigned if no more than 30% of the trees predict a high likelihood, wherein each tree predicts a high or low risk or likelihood of the renal status in question. A random forest algorithm may be generated using one or more of any of the decision trees disclosed herein. In some embodiments, the random forest algorithm comprises at least 3, 5, 10, 50, 100, 200, 300, 400, 500, or 1000 unique decision trees.

In some embodiments, CCL14 may be used together with (i.e. in combination with) one or more of cystatin C, creatinine, and urine output by using measurements or assay results together in an n-of-m (number positive) analysis. By way of example, a method of using n-of-m analysis may be performed as described by Anderberg et al. in international patent application publication number WO2012040592A1, published on Mar. 29, 2012 and which is herein incorporated by reference in its entirety. In a number positive analysis, a number of criteria are tested. A particular renal status may be assigned to the subject if at least “n” number of criteria out of a total of “m” number of criteria are satisfied. For example, a high risk or likelihood of persistent AKI may be assigned if at least n out of m criteria are satisfied. In some embodiments, one or more of the criteria may be the same as any individual node test described with respect to a decision tree analysis elsewhere herein. In some embodiments, m maybe be 2, 3, 4, 5, or more criteria. In some embodiments, n may be 1, 2, 3, 4, or more criteria (the number of positive criteria). In some embodiments, n may be equal to m-1, m-2, m-3, etc. in some embodiments, a number positive analysis comprises a CCL14 criterion and one or more additional criteria selected from a cystatin C criterion, a creatinine criterion, and a urine output criterion. In some embodiments, a number positive analysis comprises a CCL14 criterion and two or more additional criteria selected from a cystatin C criterion, a creatinine criterion, and a urine output criterion. In some embodiments, a number positive analysis comprises a CCL14 criterion, a cystatin C criterion, a creatinine criterion, and a urine output criterion. In some embodiments, a number positive analysis may comprise multiple criteria for a given marker (e.g., CCL14, cystatin C, creatinine, and urine output).

In some embodiments, CCL14 may be used together with (i.e. in combination with) one or more of cystatin C, creatinine, and urine output by using measurements or assay results together in a neural network. By way of example, a method for artificial neural network applications may be performed as described in Abiodu, O I et al. “State-of-the art in artificial neural network applications: A survey.” Heliyon. 2018 November; 4(11): e00938, which is hereby incorporated by reference in its entirety.

In some embodiments, CCL14 may be used together with (i.e. in combination with) one or more of cystatin C, creatinine, and urine output by using measurements or assay results together in a Bayesian model. By way of example, a method for Bayesian methods of analysis may be performed as described by Zhao, et al. “Evaluation of treatment efficacy using a Bayesian mixture piecewise linear model of longitudinal biomarker”. Stat Med. 2015 May 10; 34(10): 1733-1746 or by Jayson et al. “Plasma Tie2 is a tumor vascular response biomarker for VEGF inhibitors in metastatic colorectal cancer.” Nat Commun. 9: 4672 (2018), both of which are herein incorporated by reference in their entirety.

In some embodiments, CCL14 may be combined into a single composite value with one or more of cystatin C, creatinine, and urine output using a ratio of markers. By way of example, a method for using a ratio of markers may be performed by assessing a ratio of a CCL-14 level in a biological fluid to a measured urine output. In some examples, the ratio may include a positive going marker as the numerator and a negative going marker as the denominator. In this example, the composite number may be considered a positive going marker. In contrast, the ratio may include a negative going marker as the numerator and a positive going marker as the denominator. In this example, the composite number may be considered a negative going marker.

In some embodiments, CCL14 may be used together with (i.e. in combination with) one or more of cystatin C, creatinine, and urine output by using measurements or assay results together in a rule set. By way of example, a method for using rule sets for analyzing the markers may be performed as described by Woetzel et al. “Identification of rheumatoid arthritis and osteoarthritis patients by transcriptome-based rule set generation.” Arthritis Res Ther. 16(2): R84 (2014), which is hereby incorporated by reference in its entirety.

Diagnosis of Acute Renal Failure

As noted above, the terms “acute renal (or kidney) injury” and “acute renal (or kidney) failure” as used herein are defined in part in terms of changes in serum creatinine from a baseline value. Most definitions of ARF have common elements, including the use of serum creatinine and, often, urine output. Patients may present with renal dysfunction without an available baseline measure of renal function for use in this comparison. In such an event, one may estimate a baseline serum creatinine value by assuming the patient initially had a normal glomerular filtration rate (GFR). Glomerular filtration rate (GFR) is the volume of fluid filtered from the renal (kidney) glomerular capillaries into the Bowman's capsule per unit time. Glomerular filtration rate (GFR) can be calculated by measuring any chemical that has a steady level in the blood, and is freely filtered but neither reabsorbed nor secreted by the kidneys. GFR is typically expressed in units of ml/min:

$$G F R = \frac{Urine Concentration \times Urine Flow}{Plasma Concentration}$$

By normalizing the GFR to the body surface area, a GFR of approximately 75-100 ml/min per 1.73 m² can be assumed. The measured GFR therefore indicates the quantity of the substance in the urine that originated from a calculable volume of blood. There are several different techniques used to calculate or estimate the glomerular filtration rate (GFR or eGFR). In clinical practice, however, creatinine clearance is used to measure GFR. Creatinine is produced naturally by the body (creatinine is a metabolite of creatinine, which is found in muscle). It is freely filtered by the glomerulus, but also actively secreted by the renal tubules in very small amounts such that creatinine clearance overestimates actual GFR by 10-20%. This margin of error is acceptable considering the ease with which creatinine clearance is measured.

Creatinine clearance (CCr) can be calculated if values for creatinine's urine concentration (U_{Cr}), urine flow rate (V), and creatinine's plasma concentration (P_{Cr}) are known. Since the product of urine concentration and urine flow rate yields creatinine's excretion rate, creatinine clearance is also said to be its excretion rate (U_{Cr}×V) divided by its plasma concentration. This is commonly represented mathematically as:

$${C}_{Cr} = \frac{{U}_{Cr} \times V}{{P}_{Cr}}$$

Commonly a 24 hour urine collection is undertaken, from empty-bladder one morning to the contents of the bladder the following morning, with a comparative blood test then taken:

$${C}_{Cr} = \frac{{U}_{Cr} \times 24 \text{-} hour volume}{{P}_{Cr} \times 24 \times 60 mins}$$

To allow comparison of results between people of different sizes, the CCr is often corrected for the body surface area (BSA) and expressed compared to the average sized man as ml/min/1.73 m2. While most adults have a BSA that approaches 1.7 (1.6-1.9), extremely obese or slim patients should have their CCr corrected for their actual BSA:

$${C}_{Cr - corrected} = \frac{{C}_{Cr} \times 1.73}{B S A}$$

The accuracy of a creatinine clearance measurement (even when collection is complete) is limited because as glomerular filtration rate (GFR) falls creatinine secretion is increased, and thus the rise in serum creatinine is less. Thus, creatinine excretion is much greater than the filtered load, resulting in a potentially large overestimation of the GFR (as much as a twofold difference). However, for clinical purposes it is important to determine whether renal function is stable or getting worse or better. This is often determined by monitoring serum creatinine alone. Like creatinine clearance, the serum creatinine will not be an accurate reflection of GFR in the non-steady-state condition of ARF. Nonetheless, the degree to which serum creatinine changes from baseline will reflect the change in GFR. Serum creatinine is readily and easily measured and it is specific for renal function.

For purposes of determining urine output on a mL/kg/hr basis, hourly urine collection and measurement is adequate. In the case where, for example, only a cumulative 24-h output was available and no patient weights are provided, minor modifications of the RIFLE urine output criteria have been described. For example, Bagshaw et al., Nephrol. Dial. Transplant. 23: 1203-1210, 2008, assumes an average patient weight of 70 kg, and patients are assigned a RIFLE classification based on the following: <35 mL/h (Risk), <21 mL/h (Injury) or <4 mL/h (Failure).

Selecting a Treatment Regimen

Once a diagnosis is obtained, the clinician can readily select a treatment regimen that is compatible with the diagnosis, such as initiating renal replacement therapy, modifying administration of compounds known to be damaging to the kidney by adjusting the amount or selection of the compound (e.g., withdrawing or reducing delivery of compounds that are known to be damaging to the kidney), kidney transplantation, delaying or avoiding procedures that are known to be damaging to the kidney, modifying diuretic administration, initiating goal directed therapy, etc. The skilled artisan is aware of appropriate treatments for numerous diseases discussed in relation to the methods of diagnosis described herein. See, e.g., Merck Manual of Diagnosis and Therapy, 17th Ed. Merck Research Laboratories, Whitehouse Station, N J, 1999. In addition, since the methods and compositions described herein provide prognostic information, the markers of the present invention may be used to monitor a course of treatment. For example, improved or worsened prognostic state may indicate that a particular treatment is or is not efficacious. Diuretics are used in the setting of acute kidney injury (AKI) to optimize fluid management and aid in the management of electrolyte disorders. However, diuretic therapy can also have adverse effects including volume depletion, hypotension, decreased cardiac output, and worsening renal function. Decisions associated with administration, dosing, or withdrawal of diuretics depend not only on renal status but other aspects of patient status, such as fluid balance. Such factors which determine the appropriate use of diuretics in patients with or at risk for developing AKI are understood by those skilled in the art, as described, for example in Cerda, J. “Loop Diuretics in Acute Kidney Injury” Encyclopedia of Intensive Care Medicine, 2012 Ed, p 1337-1341, herein incorporated by reference in its entirety.

In some implementations, subjects assigned a relatively lower risk of renal injury, such as persistent acute kidney injury, may be treated as is known in the art according to more conservative or less aggressive treatment regimen (with respect to renal injury) which may entail less risks, costs, pain, discomfort, and/or side effects. The treatment may comprise treatments known in the art for treating an existing renal injury (e.g., acute kidney injury) when the condition is predicted to improve or not substantially worsen and/or the treatment may comprise treatments for comorbidities or other conditions in the patient. Such treatments may include treatments that may generally increase the risk for renal injury (e.g., delivery of nephrotoxic agents or ischemic procedures). Such treatments may be more aggressive/less conservative in treating the comorbidities or other conditions besides renal injury. The treatment regimen may comprise additional testing of low risk subjects after a sufficient period of time to assess whether the risk of renal injury (e.g., persistent acute kidney injury) has increased and to determine whether the low-risk subject's treatment strategy should proceed or continue. In some examples, the treatment may include modifying administration of compounds known to be damaging to the kidney by adjusting the amount or selection of the compound (e.g., withdrawing or reducing delivery of compounds that are known to be damaging to the kidney including, but not limited to, any nephrotoxic agent disclosed elsewhere herein, such as contrast agents, antibiotics, etc.), delaying or avoiding procedures that are known to be damaging to the kidney (e.g., including, but not limited to, any procedure disclosed elsewhere herein which predisposes a subject to a risk of acute kidney injury, such as vascular or cardiac surgeries or any other surgery which induces ischemia), or modifying diuretic administration. Additional examples of compounds which may be withdrawn in this example include, but are not limited to, NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

In some aspects, the treatment regimen may comprise retesting high-risk patients to confirm the results of the initial test or high risk subjects may be retested after a sufficient period of time to determine if renal status has improved, worsened, or remained relatively steady. Subsequent testing of subjects may comprise the same tests and/or different tests as initially performed to initially assess the renal status of the subject, including those described herein. Subjects may be retested as needed (e.g., approximately every 12 hours, 24 hours, 48 hours, 72 hours, 96 hours, 120 hours, 144 hours, 168 hours, etc.).

In some implementations, subjects assigned a relatively higher risk of renal injury, such as persistent acute kidney injury, may be treated as is known in the art according to less conservative or more aggressive treatment regimen (with respect to renal injury) which may entail relatively more risks, costs, pain, discomfort, and/or side effects. The treatment may comprise treatments for comorbidities or other conditions in the patient. Such treatments may be less aggressive/more conservative in treating the comorbidities or other conditions besides renal injury. The treatment may comprise treatments known in the art for treating an existing renal injury (e.g., acute kidney injury). In some embodiments, high risk subjects may also be treated by one or more of the following: initiating renal replacement therapy, modifying administration of compounds known to be damaging to the kidney by adjusting the amount or selection of the compound (e.g., withdrawing or reducing delivery of compounds that are known to be damaging to the kidney, delaying or avoiding procedures that are known to be damaging to the kidney), and modifying diuretic administration, as described elsewhere herein. High risk subjects may be treated with more aggressive/less conservative versions of these treatments. For example, the following types of renal replacement therapy may be more suitable for a high risk subject than a low risk subject: continuous renal replacement therapy (e.g., Continuous Venovenous Hemodialysis (CVVHD), Continuous Venovenous Hemofiltration (CVVH), Continuous Venovenous Hemodiafiltration (CVVHDF)), Intermittent Hemodialysis (IHD), peritoneal dialysis, and renal transplantation. In some embodiments, diuretic administration may be reduced, stopped, or paused in a high risk subject so as to avoid further aggravating renal injury.

One skilled in the art readily appreciates that the present invention is well adapted to carry out the objects and obtain the ends and advantages mentioned, as well as those inherent therein. The examples provided herein are representative of preferred aspects, are exemplary, and are not intended as limitations on the scope of the invention.

Samples included in the following Examples were collected as described in Hoste, E., A. Bihorac, A. Al-Khafaji, L. M. Ortega, M. Ostermann, M. Haase, K. Zacharowski, et al. “Identification and Validation of Biomarkers of Persistent Acute Kidney Injury: The Ruby Study.” Intensive Care Med (Feb. 6, 2020), which is hereby incorporated by reference in its entirety.

### Example 1. Immunoassay Format

Nitrocellulose membranes were pre-laminated onto backing cards and striped with test line antibody and positive control antibody. The striped nitrocellulose membranes were then cured and laminated with wicking pads and sample pads. The cards were cut into 5 mm wide test strips and placed into cartridge housings.

Purified, recombinant human C-C motif chemokine 14 protein was spiked into pooled urine and serially diluted to generate a set of standard samples covering a range of concentrations. Frozen single-use aliquots of human urine samples were thawed in a room temperature water bath for less than 20 minutes before testing.

100 μL of Test Buffer was added to a lyophilized conjugate bead containing fluorescent dye-loaded polystyrene particles coated with C-C motif chemokine 14 detect antibody. 100 μL of standard or human urine sample was added to the reconstituted conjugate solution. 100 μL of the urine sample/conjugate mixture was then loaded into the sample port of the test cartridge. At approximately 20 minutes, emitted fluorescence at 663 nm was read using a fluorescence reader upon excitation at 644 nm. A C-C motif chemokine 14 concentration was assigned to the test urine sample by comparison to a standard curve determined from the C-C motif chemokine 14 standards. Units for C-C motif chemokine 14 reported herein are ng/mL.

### Example 2: Use of C-C Motif Chemokine 14 and One or More of Serum Creatinine, Urine Output, and Cystatin C in a Product Model for Evaluating Renal Status in Patients Admitted to the ICU: Persistent at KDIGO Stage 3

Patients from the intensive care unit (ICU) who are believed to have developed KDIGO stage 2 or 3 acute kidney injury within the last 36 hours were enrolled in the following study. Patients were excluded if they had a prior kidney transplant, were receiving, or were in imminent need of renal replacement therapy, were receiving comfort measures only, or had known infection with human immunodeficiency virus or active hepatitis. Blood samples (10 mL for EDTA plasma and 3 mL for serum) and urine samples (50 mL) were collected from each patient at enrollment, and at every 12 hours up to day 3, and then every 24 hours thereafter up to day 7 while the subject was hospitalized. C-C motif chemokine 14 (CCL14) was measured in the enrollment urine samples. Cystatin C was measured in the enrollment plasma samples. Serum creatinine (sCr) was measured in enrollment serum samples by the Jaffe method on the Roche COBAS C-701 instrument and is reported in units of mg/dL. Urine flow and patient weight were recorded at the clinical sites, and weight adjusted urine output was reported in units of mL/kg/h for the time of sample collection. Records of serum creatinine levels, renal replacement therapy (RRT), and death within 90 days following enrollment were evaluated.

TABLE 1
Kidney injury markers, including units in which measurements
are reported herein unless otherwise specified, the type
of body fluid sample the marker was measured in unless otherwise
specified, and the assay used to measure the marker unless
otherwise specified. Commercial immunoassays obtained from
EMD Millipore (Billerica, MA) were measured using a Luminex
200 platform (Luminex, Austin, TX).

| Marker | Default Units | Sample Type | Assay |
|---|---|---|---|
| CCL14 | ng/mL | urine | lateral flow |
| | | | immunoassay |
| cystatin C | pg/mL | plasma | EMD Millipore |
| | | | (Billerica, MA) |
| creatinine | mg/dL | serum | Jaffe method, |
| | | | Roche COBAS C-701 |
| urine output | mL/kg/h | urine | n/a |

Kidney status was assessed by the KDIGO criteria based on serum creatinine only, based on urine output only, or based on either serum creatinine or urine output. Two cohorts were defined to represent a “persistent” and a “non-persistent” population. “Persistent” indicates those patients whose minimum KDIGO stage during a period of 24, 48, or 72 hours was stage 3, where the persistence period can start from the time of sample collection to 24, 48, 72, 96 or 168 hours after sample collection. “Non-persistent” indicates those patients who were not persistent at Stage 3 and whose minimum KDIGO stage during a period of 24, 48 or 72 hours was Stage 2 or below where the persistence period can start from the time of sample collection to 24, 48, 72, 96, or 168 hours after sample collection. If a patient reached KDIGO stage 3 and died or was placed on renal replacement therapy (RRT) during the persistence period, the patient was considered “persistent.”

The ability to distinguish the “persistent” and “non-persistent” cohorts was determined using a receiver operating characteristic (ROC) analysis. Sensitivity, specificity, and odds ratio were determined for cutoffs corresponding to the 25^{th}, 50^{th}, and 75^{th} percentiles (Quartile 2, Quartile 3, and Quartile 4) of assay results.

The individual marker assay results were combined to provide a single result as indicated herein: C-C motif chemokine 14/(Weight adjusted urine output), C-C motif chemokine 14×Serum Creatinine, C-C motif chemokine 14×Cystatin-C, C-C motif chemokine 14×Serum Creatinine/(Weight adjusted urine output), C-C motif chemokine 14×Cystatin-C/(Weight adjusted urine output). The single result was then treated as an individual biomarker using standard statistical methods. In expressing these combinations, the arithmetic operators such as “X” (multiplication) and “I” (division) are used in their ordinary mathematical sense.

TABLE 2
Baseline patient characteristics for all patients and by persistence status
as defined by persistent KDIGO stage 3 acute kidney injury lasting at
least 72 hours over a period beginning within 48 hours from enrollment.

| | All Patients | | Not Persistent | | Persistent | | P-value |
|---|---|---|---|---|---|---|---|
| Patients | 331 | | 221 | | 110 | | |
| Male | 207 | (62.5%) | 136 | (61.5%) | 71 | (64.5%) | 0.631 |
| Age (Years) | 64 | (55-73) | 64 | (54-73) | 64 | (55-71) | 0.636 |
| Body Mass Index (kg/m²) | 29 | (25-35) | 30 | (26-36) | 28 | (25-34) | 0.013 |
| Race | | | | | | | 0.371 |
| Black or African American | 34 | (10.3%) | 26 | (11.8%) | 8 | (7.3%) | |
| Other/Unknown | 17 | (5.1%) | 10 | (4.5%) | 7 | (6.4%) | |
| White or Caucasian | 280 | (84.6%) | 185 | (83.7%) | 95 | (86.4%) | |
| Chronic Comorbidities | | | | | | | |
| Chronic Kidney Disease | 58 | (17.5%) | 36 | (16.3%) | 22 | (20.0%) | 0.444 |
| Diabetes Mellitus | 109 | (32.9%) | 82 | (37.1%) | 27 | (24.5%) | 0.025 |
| Congestive Heart Failure | 74 | (22.4%) | 51 | (23.1%) | 23 | (20.9%) | 0.677 |
| Coronary Artery Disease | 117 | (35.3%) | 84 | (38.0%) | 33 | (30.0%) | 0.179 |
| Hypertension | 226 | (68.3%) | 154 | (69.7%) | 72 | (65.5%) | 0.454 |
| Chronic Obstructive Pulmonary | 55 | (16.6%) | 35 | (15.8%) | 20 | (18.2%) | 0.639 |
| Disease | | | | | | | |
| Cancer | 84 | (25.4%) | 57 | (25.8%) | 27 | (24.5%) | 0.894 |
| Reason for ICU Admission | | | | | | | |
| Respiratory | 95 | (28.7%) | 62 | (28.1%) | 33 | (30.0%) | 0.797 |
| Surgery | 105 | (31.7%) | 74 | (33.5%) | 31 | (28.2%) | 0.381 |
| Cardiovascular | 148 | (44.7%) | 96 | (43.4%) | 52 | (47.3%) | 0.558 |
| Sepsis | 74 | (22.4%) | 49 | (22.2%) | 25 | (22.7%) | >0.999 |
| Neurological | 16 | (4.8%) | 12 | (5.4%) | 4 | (3.6%) | 0.593 |
| Trauma | 7 | (2.1%) | 6 | (2.7%) | 1 | (0.9%) | 0.432 |
| Other | 107 | (32.3%) | 74 | (33.5%) | 33 | (30.0%) | 0.536 |
| Vasopressors | 210 | (63.4%) | 139 | (62.9%) | 71 | (64.5%) | 0.809 |
| Diuretics | 178 | (53.8%) | 114 | (51.6%) | 64 | (58.2%) | 0.293 |
| Baseline serum creatinine (mg/dL) | 1.0 | (0.8-1.2) | 1.0 | (0.8-1.2) | 1.0 | (0.8-1.3) | 0.083 |
| Enrollment serum creatinine (mg/dL) | 2.4 | (1.7-3.3) | 2.1 | (1.5-2.8) | 3.4 | (2.6-4.2) | <0.001 |
| Enrollment KDIGO Stage* | | | | | | | <0.001 |
| No AKI | 14 | (4.2%) | 14 | (6.3%) | 0 | (0%) | |
| Stage 1 | 39 | (11.8%) | 39 | (17.6%) | 0 | (0%) | |
| Stage 2 | 168 | (50.8%) | 129 | (58.4%) | 39 | (35.5%) | |
| Stage 3 | 110 | (33.2%) | 39 | (17.6%) | 71 | (64.5%) | |
| Enrollment non-renal APACHE III score | 54 | (43-71) | 53 | (41-69) | 58 | (45-82) | 0.017 |

Continuous and categorical baseline variables in Table 2 were compared between endpoint negative and positive patients using the Wilcoxon rank-sum test and Fisher's exact test, respectively. Of the 364 patients initially enrolled, 331 (91%) remained included in the analysis. As shown in Table 2, patients who developed persistent acute kidney injury were more likely to have a lower BMI, were less likely to have a history of diabetes mellitus, but had higher serum creatinine values and APACHE III scores at enrolment when compared to patients who did not develop persistent acute kidney injury. Patients who developed persistent stage 3 acute kidney injury were more likely to have stage 3 acute kidney injury at enrollment, which is consistent with higher enrollment levels of serum creatinine. Additionally, 128 (39%) patients experienced a major adverse kidney event at 90 (±7) days (MAKE₉₀). Major adverse kidney events were defined to consist of death, of which 108 patients (84%) were deceased; loss in estimated GFR (eGFR) calculated using the MDRD equation greater than or equal to 25%, experienced by 17 patients (13%); or dialysis, initiated in 7 patients (5%).

TABLE 3
Comparison of marker levels and the area under the ROC curve (AUC) in samples for the “persistent”
and “non-persistent” cohorts where persistence starts within 24 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| | Persistence duration (hr) | | | | |
|---|---|---|---|---|---|
| | 24 | | | 48 | |
| | sCr or | | | sCr or | |
| | UO | sCr only | UO only | UO | sCr only |
| C-C motif chemokine 14 | | | | | |
| AUC | 0.79 | 0.78 | 0.74 | 0.83 | 0.82 |
| SE | 0.027 | 0.028 | 0.045 | 0.027 | 0.028 |
| p Value | 0 | 0 | 7.0E−8 | 0 | 0 |
| nCohort Non-persistent | 210 | 215 | 285 | 224 | 227 |
| nCohort Persistent | 121 | 114 | 44 | 107 | 102 |
| Cutoff Quartile 2 | 0.725 | 0.719 | 0.732 | 0.725 | 0.719 |
| Sensitivity | 92% | 91% | 95% | 94% | 94% |
| Specificity | 35% | 33% | 28% | 34% | 33% |
| Cutoff Quartile 3 | 1.64 | 1.64 | 1.64 | 1.64 | 1.64 |
| Sensitivity | 79% | 79% | 84% | 85% | 84% |
| Specificity | 66% | 65% | 55% | 67% | 65% |
| Cutoff Quartile 4 | 5.16 | 5.09 | 5.09 | 5.16 | 5.09 |
| Sensitivity | 48% | 49% | 45% | 53% | 54% |
| Specificity | 88% | 87% | 78% | 88% | 88% |
| OR Quartile 2 | 5.91 | 5.24 | 8.20 | 8.82 | 8.05 |
| p Value | 8.2E−7 | 4.6E−6 | 0.0042 | 9.0E−7 | 2.6E−6 |
| Lower limit of 95% CI | 2.92 | 2.58 | 1.94 | 3.70 | 3.38 |
| Upper limit of 95% CI | 12.0 | 10.6 | 34.7 | 21.0 | 19.2 |
| OR Quartile 3 | 7.15 | 7.00 | 6.48 | 11.3 | 10.1 |
| p Value | 1.1E−13 | 6.5E−13 | 1.3E−5 | 2.2E−15 | 4.3E−14 |
| Lower limit of 95% CI | 4.25 | 4.12 | 2.80 | 6.20 | 5.53 |
| Upper limit of 95% CI | 12.0 | 11.9 | 15.0 | 20.6 | 18.3 |
| OR Quartile 4 | 6.81 | 6.72 | 2.94 | 8.68 | 8.32 |
| p Value | 7.5E−12 | 7.6E−12 | 0.0013 | 3.2E−14 | 7.4E−14 |
| Lower limit of 95% CI | 3.93 | 3.90 | 1.52 | 4.97 | 4.77 |
| Upper limit of 95% CI | 11.8 | 11.6 | 5.66 | 15.2 | 14.5 |
| Weight adjusted urine output | | | | | |
| AUC | 0.31 | 0.34 | 0.18 | 0.30 | 0.32 |
| SE | 0.031 | 0.032 | 0.039 | 0.032 | 0.033 |
| p Value | 3.7E−10 | 3.9E−7 | 0 | 1.5E−10 | 2.6E−8 |
| nCohort Non-persistent | 215 | 220 | 291 | 229 | 232 |
| nCohort Persistent | 122 | 115 | 46 | 108 | 103 |
| Cutoff Quartile 2 | 0.189 | 0.192 | 0.189 | 0.189 | 0.192 |
| Sensitivity | 59% | 63% | 33% | 58% | 61% |
| Specificity | 16% | 18% | 18% | 17% | 19% |
| Cutoff Quartile 3 | 0.366 | 0.368 | 0.366 | 0.366 | 0.368 |
| Sensitivity | 32% | 33% | 13% | 30% | 30% |
| Specificity | 40% | 41% | 44% | 40% | 41% |
| Cutoff Quartile 4 | 0.857 | 0.862 | 0.857 | 0.857 | 0.862 |
| Sensitivity | 16% | 17% | 4% | 16% | 17% |
| Specificity | 70% | 71% | 71% | 70% | 71% |
| OR Quartile 2 | 0.270 | 0.372 | 0.108 | 0.287 | 0.358 |
| p Value | 6.2E−7 | 1.4E−4 | 1.8E−10 | 2.1E−6 | 9.8E−5 |
| Lower limit of 95% CI | 0.162 | 0.223 | 0.0543 | 0.172 | 0.214 |
| Upper limit of 95% CI | 0.452 | 0.620 | 0.214 | 0.481 | 0.601 |
| OR Quartile 3 | 0.307 | 0.342 | 0.118 | 0.283 | 0.299 |
| p Value | 7.9E−7 | 8.4E−6 | 2.4E−6 | 4.4E−7 | 1.8E−6 |
| Lower limit of 95% CI | 0.192 | 0.213 | 0.0484 | 0.173 | 0.182 |
| Upper limit of 95% CI | 0.491 | 0.548 | 0.286 | 0.462 | 0.490 |
| OR Quartile 4 | 0.452 | 0.513 | 0.114 | 0.442 | 0.487 |
| p Value | 0.0056 | 0.020 | 0.0031 | 0.0068 | 0.017 |
| Lower limit of 95% CI | 0.258 | 0.292 | 0.0270 | 0.245 | 0.269 |
| Upper limit of 95% CI | 0.793 | 0.901 | 0.481 | 0.798 | 0.881 |
| Serum Creatinine | | | | | |
| AUC | 0.81 | 0.83 | 0.68 | 0.81 | 0.82 |
| SE | 0.025 | 0.024 | 0.042 | 0.026 | 0.026 |
| p Value | 0 | 0 | 2.8E−5 | 0 | 0 |
| nCohort Non-persistent | 218 | 223 | 296 | 232 | 235 |
| nCohort Persistent | 136 | 129 | 56 | 122 | 117 |
| Cutoff Quartile 2 | 1.64 | 1.64 | 1.65 | 1.64 | 1.64 |
| Sensitivity | 95% | 96% | 91% | 96% | 97% |
| Specificity | 38% | 37% | 28% | 36% | 36% |
| Cutoff Quartile 3 | 2.44 | 2.44 | 2.44 | 2.44 | 2.44 |
| Sensitivity | 78% | 80% | 70% | 80% | 81% |
| Specificity | 67% | 67% | 53% | 66% | 66% |
| Cutoff Quartile 4 | 3.32 | 3.31 | 3.32 | 3.32 | 3.31 |
| Sensitivity | 49% | 51% | 46% | 52% | 53% |
| Specificity | 89% | 90% | 79% | 88% | 89% |
| OR Quartile 2 | 11.1 | 14.7 | 3.97 | 13.3 | 15.7 |
| p Value | 5.3E−9 | 1.7E−8 | 0.0045 | 5.8E−8 | 1.7E−7 |
| Lower limit of 95% CI | 4.95 | 5.78 | 1.53 | 5.22 | 5.60 |
| Upper limit of 95% CI | 24.9 | 37.4 | 10.3 | 33.8 | 44.1 |
| OR Quartile 3 | 7.16 | 8.14 | 2.63 | 7.76 | 8.21 |
| p Value | 5.6E−15 | 1.1E−15 | 0.0020 | 1.4E−14 | 1.4E−14 |
| Lower limit of 95% CI | 4.37 | 4.87 | 1.42 | 4.60 | 4.80 |
| Upper limit of 95% CI | 11.7 | 13.6 | 4.85 | 13.1 | 14.0 |
| OR Quartile 4 | 8.23 | 9.57 | 3.21 | 8.11 | 9.06 |
| p Value | 4.5E−14 | 2.4E−15 | 1.2E−4 | 1.9E−14 | 2.4E−15 |
| Lower limit of 95% CI | 4.76 | 5.47 | 1.77 | 4.74 | 5.25 |
| Upper limit of 95% CI | 14.2 | 16.7 | 5.81 | 13.9 | 15.6 |
| Cystatin-C | | | | | |
| AUC | 0.75 | 0.76 | 0.73 | 0.75 | 0.76 |
| SE | 0.030 | 0.030 | 0.048 | 0.031 | 0.031 |
| p Value | 0 | 0 | 2.5E−6 | 2.2E−16 | 0 |
| nCohort Non-persistent | 193 | 196 | 267 | 207 | 208 |
| nCohort Persistent | 115 | 111 | 39 | 101 | 99 |
| Cutoff Quartile 2 | 1170000 | 1170000 | 1170000 | 1170000 | 1170000 |
| Sensitivity | 91% | 91% | 92% | 91% | 91% |
| Specificity | 34% | 34% | 28% | 32% | 32% |
| Cutoff Quartile 3 | 1660000 | 1650000 | 1660000 | 1660000 | 1650000 |
| Sensitivity | 76% | 77% | 79% | 76% | 78% |
| Specificity | 65% | 65% | 54% | 63% | 63% |
| Cutoff Quartile 4 | 2150000 | 2150000 | 2150000 | 2150000 | 2150000 |
| Sensitivity | 43% | 45% | 51% | 46% | 47% |
| Specificity | 85% | 86% | 79% | 85% | 86% |
| OR Quartile 2 | 5.46 | 5.13 | 4.60 | 4.89 | 4.75 |
| p Value | 3.2E−6 | 7.2E−6 | 0.013 | 2.9E−5 | 4.1E−5 |
| Lower limit of 95% CI | 2.67 | 2.51 | 1.37 | 2.33 | 2.26 |
| Upper limit of 95% CI | 11.1 | 10.5 | 15.4 | 10.3 | 10.0 |
| OR Quartile 3 | 5.84 | 6.02 | 4.61 | 5.42 | 5.95 |
| p Value | 2.6E−11 | 2.7E−11 | 2.3E−4 | 7.5E−10 | 2.2E−10 |
| Lower limit of 95% CI | 3.48 | 3.55 | 2.04 | 3.16 | 3.43 |
| Upper limit of 95% CI | 9.82 | 10.2 | 10.4 | 9.28 | 10.3 |
| OR Quartile 4 | 4.38 | 5.13 | 3.88 | 4.75 | 5.36 |
| p Value | 1.1E−7 | 6.4E−9 | 1.3E−4 | 2.4E−8 | 2.6E−9 |
| Lower limit of 95% CI | 2.54 | 2.95 | 1.94 | 2.75 | 3.09 |
| Upper limit of 95% CI | 7.55 | 8.91 | 7.75 | 8.20 | 9.32 |
| C-C motif chemokine 14/(Weight | | | | | |
| adjusted urine output) | | | | | |
| AUC | 0.80 | 0.77 | 0.84 | 0.83 | 0.81 |
| SE | 0.027 | 0.029 | 0.038 | 0.027 | 0.028 |
| p Value | 0 | 0 | 0 | 0 | 0 |
| nCohort Non-persistent | 208 | 213 | 284 | 222 | 225 |
| nCohort Persistent | 120 | 113 | 44 | 106 | 101 |
| Cutoff Quartile 2 | 1.26 | 1.24 | 1.26 | 1.26 | 1.24 |
| Sensitivity | 90% | 89% | 98% | 93% | 93% |
| Specificity | 34% | 33% | 29% | 34% | 33% |
| Cutoff Quartile 3 | 4.59 | 4.54 | 4.59 | 4.59 | 4.54 |
| Sensitivity | 78% | 77% | 89% | 83% | 82% |
| Specificity | 66% | 64% | 56% | 66% | 64% |
| Cutoff Quartile 4 | 24.3 | 22.9 | 24.3 | 24.3 | 22.9 |
| Sensitivity | 52% | 51% | 70% | 56% | 55% |
| Specificity | 90% | 89% | 82% | 90% | 88% |
| OR Quartile 2 | 4.57 | 4.12 | 17.2 | 7.22 | 6.71 |
| p Value | 7.0E−6 | 2.9E−5 | 0.0053 | 2.0E−6 | 4.8E−6 |
| Lower limit of 95% CI | 2.35 | 2.12 | 2.32 | 3.19 | 2.97 |
| Upper limit of 95% CI | 8.85 | 8.00 | 127 | 16.3 | 15.2 |
| OR Quartile 3 | 7.13 | 6.03 | 9.92 | 9.39 | 8.36 |
| p Value | 1.5E−13 | 1.3E−11 | 2.8E−6 | 3.0E−14 | 6.1E−13 |
| Lower limit of 95% CI | 4.23 | 3.59 | 3.80 | 5.27 | 4.69 |
| Upper limit of 95% CI | 12.0 | 10.1 | 25.9 | 16.7 | 14.9 |
| OR Quartile 4 | 10.0 | 8.30 | 10.9 | 10.9 | 9.52 |
| p Value | 9.3E−15 | 1.6E−13 | 5.9E−11 | 4.4E−16 | 6.4E−15 |
| Lower limit of 95% CI | 5.61 | 4.73 | 5.33 | 6.10 | 5.40 |
| Upper limit of 95% CI | 18.0 | 14.6 | 22.3 | 19.3 | 16.8 |
| C-C motif chemokine | | | | | |
| 14 X Serum Creatinine | | | | | |
| AUC | 0.83 | 0.83 | 0.75 | 0.86 | 0.85 |
| SE | 0.025 | 0.026 | 0.044 | 0.025 | 0.025 |
| p Value | 0 | 0 | 1.6E−8 | 0 | 0 |
| nCohort Non-persistent | 204 | 209 | 278 | 218 | 221 |
| nCohort Persistent | 120 | 113 | 44 | 106 | 101 |
| Cutoff Quartile 2 | 1.29 | 1.29 | 1.30 | 1.29 | 1.29 |
| Sensitivity | 93% | 94% | 93% | 96% | 96% |
| Specificity | 36% | 35% | 28% | 35% | 35% |
| Cutoff Quartile 3 | 3.97 | 3.91 | 3.97 | 3.97 | 3.91 |
| Sensitivity | 82% | 83% | 86% | 88% | 87% |
| Specificity | 69% | 68% | 56% | 68% | 67% |
| Cutoff Quartile 4 | 18.3 | 16.8 | 18.0 | 18.3 | 16.8 |
| Sensitivity | 51% | 52% | 55% | 57% | 57% |
| Specificity | 90% | 89% | 79% | 90% | 90% |
| OR Quartile 2 | 7.80 | 8.30 | 5.33 | 13.9 | 13.0 |
| p Value | 1.9E−7 | 3.7E−7 | 0.0063 | 6.4E−7 | 1.3E−6 |
| Lower limit of 95% CI | 3.60 | 3.67 | 1.60 | 4.94 | 4.59 |
| Upper limit of 95% CI | 16.9 | 18.8 | 17.7 | 39.3 | 36.6 |
| OR Quartile 3 | 10.6 | 10.5 | 7.98 | 15.4 | 13.7 |
| p Value | 0 | 8.9E−16 | 5.1E−6 | 0 | 2.0E−15 |
| Lower limit of 95% CI | 6.05 | 5.92 | 3.27 | 8.09 | 7.19 |
| Upper limit of 95% CI | 18.4 | 18.6 | 19.5 | 29.5 | 26.2 |
| OR Quartile 4 | 9.51 | 9.29 | 4.65 | 12.2 | 11.6 |
| p Value | 4.0E−14 | 3.3E−14 | 5.1E−6 | 0 | 2.2E−16 |
| Lower limit of 95% CI | 5.30 | 5.22 | 2.40 | 6.77 | 6.47 |
| Upper limit of 95% CI | 17.1 | 16.5 | 9.01 | 22.1 | 20.8 |
| C-C motif chemokine | | | | | |
| 14 X Cystatin-C | | | | | |
| AUC | 0.80 | 0.81 | 0.76 | 0.84 | 0.84 |
| SE | 0.027 | 0.028 | 0.047 | 0.027 | 0.027 |
| p Value | 0 | 0 | 4.0E−8 | 0 | 0 |
| nCohort Non-persistent | 193 | 196 | 267 | 207 | 208 |
| nCohort Persistent | 115 | 111 | 39 | 101 | 99 |
| Cutoff Quartile 2 | 956000 | 955000 | 958000 | 956000 | 955000 |
| Sensitivity | 92% | 92% | 95% | 94% | 94% |
| Specificity | 35% | 35% | 28% | 34% | 34% |
| Cutoff Quartile 3 | 2880000 | 2860000 | 2880000 | 2880000 | 2860000 |
| Sensitivity | 82% | 82% | 85% | 85% | 85% |
| Specificity | 69% | 68% | 55% | 67% | 66% |
| Cutoff Quartile 4 | 1.04E7 | 1.03E7 | 1.03E7 | 1.04E7 | 1.03E7 |
| Sensitivity | 49% | 50% | 54% | 55% | 57% |
| Specificity | 89% | 89% | 79% | 90% | 90% |
| OR Quartile 2 | 6.41 | 6.02 | 7.23 | 8.27 | 8.03 |
| p Value | 9.2E−7 | 2.1E−6 | 0.0074 | 2.1E−6 | 3.0E−6 |
| Lower limit of 95% CI | 3.05 | 2.87 | 1.70 | 3.45 | 3.35 |
| Upper limit of 95% CI | 13.5 | 12.6 | 30.7 | 19.8 | 19.2 |
| OR Quartile 3 | 9.92 | 9.61 | 6.74 | 11.7 | 11.0 |
| p Value | 1.3E−15 | 6.9E−15 | 3.4E−5 | 7.5E−15 | 3.2E−14 |
| Lower limit of 95% CI | 5.65 | 5.44 | 2.73 | 6.30 | 5.94 |
| Upper limit of 95% CI | 17.4 | 17.0 | 16.6 | 21.8 | 20.5 |
| OR Quartile 4 | 7.77 | 8.48 | 4.40 | 11.0 | 11.6 |
| p Value | 5.1E−12 | 8.5E−13 | 3.0E−5 | 3.6E−15 | 1.3E−15 |
| Lower limit of 95% | 4.34 | 4.72 | 2.19 | 6.06 | 6.36 |
| CI Upper limit of 95% CI | 13.9 | 15.2 | 8.81 | 20.0 | 21.2 |
| C-C motif chemokine 14 X | | | | | |
| Serum Creatinine/(Weight | | | | | |
| adjusted urine output) | | | | | |
| AUC | 0.83 | 0.82 | 0.85 | 0.86 | 0.85 |
| SE | 0.025 | 0.027 | 0.038 | 0.024 | 0.026 |
| p Value | 0 | 0 | 0 | 0 | 0 |
| nCohort Non-persistent | 202 | 207 | 277 | 216 | 219 |
| nCohort Persistent | 119 | 112 | 44 | 105 | 100 |
| Cutoff Quartile 2 | 2.17 | 2.15 | 2.17 | 2.17 | 2.15 |
| Sensitivity | 92% | 91% | 95% | 93% | 93% |
| Specificity | 35% | 34% | 28% | 34% | 33% |
| Cutoff Quartile 3 | 10.2 | 10.1 | 10.2 | 10.2 | 10.1 |
| Sensitivity | 83% | 82% | 89% | 87% | 86% |
| Specificity | 69% | 67% | 56% | 68% | 66% |
| Cutoff Quartile 4 | 69.8 | 67.6 | 69.8 | 69.8 | 67.6 |
| Sensitivity | 55% | 54% | 68% | 61% | 59% |
| Specificity | 93% | 91% | 82% | 92% | 90% |
| OR Quartile 2 | 5.78 | 5.21 | 8.23 | 7.15 | 6.64 |
| p Value | 1.3E−6 | 5.3E−6 | 0.0042 | 2.4E−6 | 5.7E−6 |
| Lower limit of 95% CI | 2.84 | 2.56 | 1.95 | 3.16 | 2.93 |
| Upper limit of 95% CI | 11.8 | 10.6 | 34.8 | 16.2 | 15.1 |
| OR Quartile 3 | 11.2 | 9.40 | 9.91 | 13.6 | 12.0 |
| p Value | 0 | 6.7E−15 | 2.9E−6 | 4.4E−16 | 1.0E−14 |
| Lower limit of 95% CI | 6.35 | 5.35 | 3.79 | 7.22 | 6.41 |
| Upper limit of 95% CI | 19.7 | 16.5 | 25.9 | 25.5 | 22.6 |
| OR Quartile 4 | 15.5 | 11.8 | 9.50 | 18.3 | 13.6 |
| p Value | 0 | 6.7E−16 | 3.6E−10 | 0 | 0 |
| Lower limit of 95% CI | 8.20 | 6.49 | 4.70 | 9.72 | 7.44 |
| Upper limit of 95% CI | 29.4 | 21.6 | 19.2 | 34.4 | 24.7 |
| C-C motif chemokine 14 X | | | | | |
| Cystatin-C/(Weight | | | | | |
| adjusted urine output) | | | | | |
| AUC | 0.80 | 0.79 | 0.83 | 0.83 | 0.83 |
| SE | 0.027 | 0.028 | 0.042 | 0.027 | 0.028 |
| p Value | 0 | 0 | 2.2E−15 | 0 | 0 |
| nCohort Non-persistent | 191 | 194 | 266 | 205 | 206 |
| nCohort Persistent | 114 | 110 | 39 | 100 | 98 |
| Cutoff Quartile 2 | 1580000 | 1580000 | 1580000 | 1580000 | 1580000 |
| Sensitivity | 90% | 90% | 95% | 93% | 93% |
| Specificity | 34% | 34% | 28% | 34% | 33% |
| Cutoff Quartile 3 | 6710000 | 6570000 | 6710000 | 6710000 | 6570000 |
| Sensitivity | 81% | 80% | 90% | 85% | 85% |
| Specificity | 68% | 67% | 56% | 67% | 67% |
| Cutoff Quartile 4 | 3.85E7 | 3.84E7 | 3.85E7 | 3.85E7 | 3.84E7 |
| Sensitivity | 51% | 50% | 67% | 55% | 54% |
| Specificity | 90% | 89% | 81% | 89% | 89% |
| OR Quartile 2 | 4.83 | 4.53 | 7.13 | 6.74 | 6.55 |
| p Value | 7.7E−6 | 1.8E−5 | 0.0078 | 5.2E−6 | 7.3E−6 |
| Lower limit of 95% CI | 2.42 | 2.27 | 1.68 | 2.97 | 2.88 |
| Upper limit of 95% CI | 9.63 | 9.05 | 30.3 | 15.3 | 14.9 |
| OR Quartile 3 | 8.91 | 8.12 | 11.0 | 11.4 | 11.0 |
| p Value | 1.2E−14 | 1.4E−13 | 9.9E−6 | 1.6E−14 | 4.0E−14 |
| Lower limit of 95% CI | 5.11 | 4.67 | 3.79 | 6.13 | 5.90 |
| Upper limit of 95% CI | 15.5 | 14.2 | 31.8 | 21.2 | 20.5 |
| OR Quartile 4 | 9.38 | 8.24 | 8.43 | 10.2 | 9.37 |
| p Value | 2.5E−13 | 1.9E−12 | 1.2E−8 | 1.7E−14 | 8.8E−14 |
| Lower limit of 95% CI | 5.15 | 4.58 | 4.05 | 5.62 | 5.20 |
| Upper limit of 95% CI | 17.1 | 14.8 | 17.5 | 18.4 | 16.9 |

| | Persistence duration (hr) | | | |
|---|---|---|---|---|
| | | 72 | | |
| | 48 | sCr or | | |
| | UO only | UO | sCr only | UO only |
| C-C motif chemokine 14 | | | | |
| AUC | 0.76 | 0.81 | 0.80 | 0.76 |
| SE | 0.045 | 0.029 | 0.030 | 0.045 |
| p Value | 1.8E−8 | 0 | 0 | 1.8E−8 |
| nCohort Non-persistent | 288 | 232 | 235 | 288 |
| nCohort Persistent | 41 | 99 | 94 | 41 |
| Cutoff Quartile 2 | 0.732 | 0.725 | 0.719 | 0.732 |
| Sensitivity | 95% | 94% | 94% | 95% |
| Specificity | 28% | 33% | 32% | 28% |
| Cutoff Quartile 3 | 1.64 | 1.64 | 1.64 | 1.64 |
| Sensitivity | 88% | 85% | 84% | 88% |
| Specificity | 55% | 65% | 63% | 55% |
| Cutoff Quartile 4 | 5.09 | 5.16 | 5.09 | 5.09 |
| Sensitivity | 49% | 52% | 52% | 49% |
| Specificity | 78% | 86% | 86% | 78% |
| OR Quartile 2 | 7.50 | 7.70 | 7.01 | 7.50 |
| p Value | 0.0062 | 4.2E−6 | 1.2E−5 | 0.0062 |
| Lower limit of 95% CI | 1.77 | 3.23 | 2.93 | 1.77 |
| Upper limit of 95% CI | 31.8 | 18.4 | 16.8 | 31.8 |
| OR Quartile 3 | 8.87 | 10.2 | 9.12 | 8.87 |
| p Value | 9.0E−6 | 9.1E−14 | 1.5E−12 | 9.0E−6 |
| Lower limit of 95% CI | 3.38 | 5.56 | 4.95 | 3.38 |
| Upper limit of 95% CI | 23.3 | 18.9 | 16.8 | 23.3 |
| OR Quartile 4 | 3.40 | 6.64 | 6.44 | 3.40 |
| p Value | 3.6E−4 | 8.1E−12 | 1.9E−11 | 3.6E−4 |
| Lower limit of 95% CI | 1.74 | 3.86 | 3.74 | 1.74 |
| Upper limit of 95% CI | 6.67 | 11.4 | 11.1 | 6.67 |
| Weight adjusted urine output | | | | |
| AUC | 0.19 | 0.29 | 0.31 | 0.19 |
| SE | 0.041 | 0.033 | 0.034 | 0.041 |
| p Value | 4.3E−14 | 1.8E−10 | 3.2E−8 | 4.3E−14 |
| nCohort Non-persistent | 294 | 237 | 240 | 294 |
| nCohort Persistent | 43 | 100 | 95 | 43 |
| Cutoff Quartile 2 | 0.189 | 0.189 | 0.192 | 0.189 |
| Sensitivity | 35% | 57% | 60% | 35% |
| Specificity | 19% | 17% | 19% | 19% |
| Cutoff Quartile 3 | 0.366 | 0.366 | 0.368 | 0.366 |
| Sensitivity | 14% | 29% | 29% | 14% |
| Specificity | 45% | 41% | 42% | 45% |
| Cutoff Quartile 4 | 0.857 | 0.857 | 0.862 | 0.857 |
| Sensitivity | 5% | 15% | 16% | 5% |
| Specificity | 72% | 70% | 71% | 72% |
| OR Quartile 2 | 0.126 | 0.277 | 0.346 | 0.126 |
| p Value | 4.3E−9 | 1.3E−6 | 7.0E−5 | 4.3E−9 |
| Lower limit of 95% CI | 0.0631 | 0.165 | 0.205 | 0.0631 |
| Upper limit of 95% CI | 0.252 | 0.466 | 0.584 | 0.252 |
| OR Quartile 3 | 0.130 | 0.283 | 0.299 | 0.130 |
| p Value | 7.7E−6 | 9.0E−7 | 3.4E−6 | 7.7E−6 |
| Lower limit of 95% CI | 0.0534 | 0.171 | 0.179 | 0.0534 |
| Upper limit of 95% CI | 0.318 | 0.468 | 0.497 | 0.318 |
| OR Quartile 4 | 0.124 | 0.421 | 0.465 | 0.124 |
| p Value | 0.0045 | 0.0059 | 0.015 | 0.0045 |
| Lower limit of 95% CI | 0.0293 | 0.227 | 0.250 | 0.0293 |
| Upper limit of 95% CI | 0.524 | 0.779 | 0.862 | 0.524 |
| Serum Creatinine | | | | |
| AUC | 0.69 | 0.80 | 0.82 | 0.68 |
| SE | 0.042 | 0.027 | 0.027 | 0.043 |
| p Value | 7.0E−6 | 0 | 0 | 1.9E−5 |
| nCohort Non-persistent | 298 | 240 | 243 | 299 |
| nCohort Persistent | 54 | 114 | 109 | 53 |
| Cutoff Quartile 2 | 1.65 | 1.64 | 1.64 | 1.65 |
| Sensitivity | 93% | 96% | 96% | 92% |
| Specificity | 28% | 35% | 35% | 28% |
| Cutoff Quartile 3 | 2.44 | 2.44 | 2.44 | 2.44 |
| Sensitivity | 72% | 81% | 82% | 72% |
| Specificity | 54% | 64% | 64% | 54% |
| Cutoff Quartile 4 | 3.32 | 3.32 | 3.31 | 3.32 |
| Sensitivity | 48% | 53% | 54% | 47% |
| Specificity | 79% | 88% | 88% | 79% |
| OR Quartile 2 | 4.91 | 11.7 | 13.9 | 4.79 |
| p Value | 0.0030 | 2.4E−7 | 6.0E−7 | 0.0035 |
| Lower limit of 95% CI | 1.72 | 4.61 | 4.94 | 1.67 |
| Upper limit of 95% CI | 14.0 | 29.9 | 39.0 | 13.7 |
| OR Quartile 3 | 3.01 | 7.49 | 7.98 | 2.92 |
| p Value | 6.9E−4 | 1.6E−13 | 1.6E−13 | 0.0010 |
| Lower limit of 95% CI | 1.59 | 4.39 | 4.60 | 1.54 |
| Upper limit of 95% CI | 5.70 | 12.8 | 13.8 | 5.53 |
| OR Quartile 4 | 3.46 | 7.78 | 8.71 | 3.28 |
| p Value | 5.2E−5 | 3.5E−14 | 4.4E−15 | 1.2E−4 |
| Lower limit of 95% CI | 1.90 | 4.58 | 5.07 | 1.79 |
| Upper limit of 95% CI | 6.32 | 13.2 | 15.0 | 6.01 |
| Cystatin-C | | | | |
| AUC | 0.72 | 0.75 | 0.75 | 0.72 |
| SE | 0.049 | 0.032 | 0.032 | 0.050 |
| p Value | 6.3E−6 | 4.5E−14 | 6.9E−15 | 1.3E−5 |
| nCohort Non-persistent | 269 | 215 | 216 | 270 |
| nCohort Persistent | 37 | 93 | 91 | 36 |
| Cutoff Quartile 2 | 1170000 | 1170000 | 1170000 | 1170000 |
| Sensitivity | 92% | 91% | 91% | 92% |
| Specificity | 28% | 32% | 31% | 27% |
| Cutoff Quartile 3 | 1660000 | 1660000 | 1650000 | 1660000 |
| Sensitivity | 78% | 76% | 78% | 78% |
| Specificity | 54% | 61% | 62% | 54% |
| Cutoff Quartile 4 | 2150000 | 2150000 | 2150000 | 2150000 |
| Sensitivity | 54% | 45% | 47% | 53% |
| Specificity | 79% | 84% | 84% | 79% |
| OR Quartile 2 | 4.30 | 4.91 | 4.77 | 4.15 |
| p Value | 0.018 | 6.3E−5 | 8.8E−5 | 0.021 |
| Lower limit of 95% CI | 1.28 | 2.25 | 2.18 | 1.24 |
| Upper limit of 95% CI | 14.4 | 10.7 | 10.4 | 14.0 |
| OR Quartile 3 | 4.24 | 5.13 | 5.69 | 4.06 |
| p Value | 5.4E−4 | 6.1E−9 | 1.8E−9 | 8.3E−4 |
| Lower limit of 95% CI | 1.87 | 2.96 | 3.23 | 1.79 |
| Upper limit of 95% CI | 9.61 | 8.91 | 10.0 | 9.23 |
| OR Quartile 4 | 4.38 | 4.24 | 4.80 | 4.09 |
| p Value | 4.6E−5 | 2.2E−7 | 2.4E−8 | 1.2E−4 |
| Lower limit of 95% CI | 2.15 | 2.45 | 2.76 | 2.00 |
| Upper limit of 95% CI | 8.90 | 7.31 | 8.32 | 8.36 |
| C-C motif chemokine 14/(Weight | | | | |
| adjusted urine output) | | | | |
| AUC | 0.84 | 0.81 | 0.80 | 0.84 |
| SE | 0.040 | 0.028 | 0.030 | 0.040 |
| p Value | 0 | 0 | 0 | 0 |
| nCohort Non-persistent | 287 | 230 | 233 | 287 |
| nCohort Persistent | 41 | 98 | 93 | 41 |
| Cutoff Quartile 2 | 1.26 | 1.26 | 1.24 | 1.26 |
| Sensitivity | 98% | 94% | 94% | 98% |
| Specificity | 28% | 33% | 33% | 28% |
| Cutoff Quartile 3 | 4.59 | 4.59 | 4.54 | 4.59 |
| Sensitivity | 88% | 83% | 82% | 88% |
| Specificity | 55% | 64% | 63% | 55% |
| Cutoff Quartile 4 | 24.3 | 24.3 | 22.9 | 24.3 |
| Sensitivity | 71% | 55% | 55% | 71% |
| Specificity | 82% | 88% | 87% | 82% |
| OR Quartile 2 | 15.7 | 7.57 | 7.02 | 15.7 |
| p Value | 0.0070 | 5.2E−6 | 1.2E−5 | 0.0070 |
| Lower limit of 95% CI | 2.13 | 3.17 | 2.94 | 2.13 |
| Upper limit of 95% CI | 116 | 18.1 | 16.8 | 116 |
| OR Quartile 3 | 8.94 | 8.44 | 7.50 | 8.94 |
| p Value | 8.4E−6 | 1.2E−12 | 2.0E−11 | 8.4E−6 |
| Lower limit of 95% CI | 3.41 | 4.69 | 4.16 | 3.41 |
| Upper limit of 95% CI | 23.5 | 15.2 | 13.5 | 23.5 |
| OR Quartile 4 | 10.7 | 8.85 | 7.91 | 10.7 |
| p Value | 2.9E−10 | 2.5E−14 | 3.2E−13 | 2.9E−10 |
| Lower limit of 95% CI | 5.11 | 5.05 | 4.54 | 5.11 |
| Upper limit of 95% CI | 22.3 | 15.5 | 13.8 | 22.3 |
| C-C motif chemokine | | | | |
| 14 X Serum Creatinine | | | | |
| AUC | 0.77 | 0.84 | 0.83 | 0.77 |
| SE | 0.045 | 0.027 | 0.028 | 0.045 |
| p Value | 3.2E−9 | 0 | 0 | 3.2E−9 |
| nCohort Non-persistent | 281 | 226 | 229 | 281 |
| nCohort Persistent | 41 | 98 | 93 | 41 |
| Cutoff Quartile 2 | 1.30 | 1.29 | 1.29 | 1.30 |
| Sensitivity | 95% | 96% | 96% | 95% |
| Specificity | 28% | 34% | 34% | 28% |
| Cutoff Quartile 3 | 3.97 | 3.97 | 3.91 | 3.97 |
| Sensitivity | 90% | 88% | 87% | 90% |
| Specificity | 56% | 66% | 65% | 56% |
| Cutoff Quartile 4 | 18.0 | 18.3 | 16.8 | 18.0 |
| Sensitivity | 56% | 54% | 55% | 56% |
| Specificity | 79% | 88% | 87% | 79% |
| OR Quartile 2 | 7.63 | 12.1 | 11.3 | 7.63 |
| p Value | 0.0058 | 2.4E−6 | 4.9E−6 | 0.0058 |
| Lower limit of 95% CI | 1.80 | 4.30 | 3.99 | 1.80 |
| Upper limit of 95% CI | 32.3 | 34.3 | 31.8 | 32.3 |
| OR Quartile 3 | 11.7 | 14.1 | 12.6 | 11.7 |
| p Value | 5.2E−6 | 5.3E−15 | 8.1E−14 | 5.2E−6 |
| Lower limit of 95% CI | 4.07 | 7.28 | 6.47 | 4.07 |
| Upper limit of 95% CI | 33.7 | 27.5 | 24.4 | 33.7 |
| OR Quartile 4 | 4.91 | 8.33 | 8.05 | 4.91 |
| p Value | 4.6E−6 | 1.3E−13 | 3.0E−13 | 4.6E−6 |
| Lower limit of 95% CI | 2.49 | 4.75 | 4.60 | 2.49 |
| Upper limit of 95% CI | 9.71 | 14.6 | 14.1 | 9.71 |
| C-C motif chemokine | | | | |
| 14 X Cystatin-C | | | | |
| AUC | 0.77 | 0.82 | 0.82 | 0.77 |
| SE | 0.048 | 0.029 | 0.029 | 0.048 |
| p Value | 1.5E−8 | 0 | 0 | 1.5E−8 |
| nCohort Non-persistent | 270 | 215 | 216 | 270 |
| nCohort Persistent | 36 | 93 | 91 | 36 |
| Cutoff Quartile 2 | 958000 | 956000 | 955000 | 958000 |
| Sensitivity | 94% | 94% | 93% | 94% |
| Specificity | 28% | 33% | 33% | 28% |
| Cutoff Quartile 3 | 2880000 | 2880000 | 2860000 | 2880000 |
| Sensitivity | 86% | 85% | 85% | 86% |
| Specificity | 55% | 65% | 64% | 55% |
| Cutoff Quartile 4 | 1.03E7 | 1.04E7 | 1.03E7 | 1.03E7 |
| Sensitivity | 58% | 54% | 55% | 58% |
| Specificity | 79% | 87% | 88% | 79% |
| OR Quartile 2 | 6.54 | 7.15 | 6.94 | 6.54 |
| p Value | 0.011 | 1.0E−5 | 1.4E−5 | 0.011 |
| Lower limit of 95% CI | 1.53 | 2.98 | 2.89 | 1.53 |
| Upper limit of 95% CI | 27.9 | 17.1 | 16.6 | 27.9 |
| OR Quartile 3 | 7.52 | 10.5 | 9.93 | 7.52 |
| p Value | 4.9E−5 | 3.3E−13 | 1.3E−12 | 4.9E−5 |
| Lower limit of 95% CI | 2.84 | 5.59 | 5.27 | 2.84 |
| Upper limit of 95% CI | 19.9 | 19.9 | 18.7 | 19.9 |
| OR Quartile 4 | 5.35 | 8.10 | 8.54 | 5.35 |
| p Value | 5.8E−6 | 8.8E−13 | 3.3E−13 | 5.8E−6 |
| Lower limit of 95% | 2.59 | 4.56 | 4.79 | 2.59 |
| CI Upper limit of 95% CI | 11.0 | 14.4 | 15.2 | 11.0 |
| C-C motif chemokine 14 X | | | | |
| Serum Creatinine/(Weight | | | | |
| adjusted urine output) | | | | |
| AUC | 0.85 | 0.85 | 0.83 | 0.85 |
| SE | 0.039 | 0.026 | 0.028 | 0.039 |
| p Value | 0 | 0 | 0 | 0 |
| nCohort Non-persistent | 280 | 224 | 227 | 280 |
| nCohort Persistent | 41 | 97 | 92 | 41 |
| Cutoff Quartile 2 | 2.17 | 2.17 | 2.15 | 2.17 |
| Sensitivity | 95% | 94% | 93% | 95% |
| Specificity | 28% | 33% | 33% | 28% |
| Cutoff Quartile 3 | 10.2 | 10.2 | 10.1 | 10.2 |
| Sensitivity | 88% | 87% | 86% | 88% |
| Specificity | 55% | 66% | 64% | 55% |
| Cutoff Quartile 4 | 69.8 | 69.8 | 67.6 | 69.8 |
| Sensitivity | 71% | 61% | 59% | 71% |
| Specificity | 81% | 90% | 89% | 81% |
| OR Quartile 2 | 7.53 | 7.48 | 6.93 | 7.53 |
| p Value | 0.0062 | 6.0E−6 | 1.4E−5 | 0.0062 |
| Lower limit of 95% CI | 1.78 | 3.13 | 2.90 | 1.78 |
| Upper limit of 95% CI | 31.9 | 17.9 | 16.6 | 31.9 |
| OR Quartile 3 | 8.93 | 12.3 | 11.0 | 8.93 |
| p Value | 8.7E−6 | 2.5E−14 | 3.9E−13 | 8.7E−6 |
| Lower limit of 95% CI | 3.40 | 6.47 | 5.74 | 3.40 |
| Upper limit of 95% CI | 23.4 | 23.5 | 20.9 | 23.4 |
| OR Quartile 4 | 10.6 | 14.3 | 11.0 | 10.6 |
| p Value | 3.5E−10 | 0 | 6.7E−16 | 3.5E−10 |
| Lower limit of 95% CI | 5.07 | 7.82 | 6.14 | 5.07 |
| Upper limit of 95% CI | 22.1 | 26.0 | 19.7 | 22.1 |
| C-C motif chemokine 14 X | | | | |
| Cystatin-C/(Weight | | | | |
| adjusted urine output) | | | | |
| AUC | 0.83 | 0.82 | 0.81 | 0.83 |
| SE | 0.043 | 0.029 | 0.030 | 0.043 |
| p Value | 5.6E−14 | 0 | 0 | 5.6E−14 |
| nCohort Non-persistent | 269 | 213 | 214 | 269 |
| nCohort Persistent | 36 | 92 | 90 | 36 |
| Cutoff Quartile 2 | 1580000 | 1580000 | 1580000 | 1580000 |
| Sensitivity | 94% | 93% | 93% | 94% |
| Specificity | 28% | 33% | 33% | 28% |
| Cutoff Quartile 3 | 6710000 | 6710000 | 6570000 | 6710000 |
| Sensitivity | 89% | 85% | 84% | 89% |
| Specificity | 55% | 65% | 64% | 55% |
| Cutoff Quartile 4 | 3.85E7 | 3.85E7 | 3.84E7 | 3.85E7 |
| Sensitivity | 67% | 54% | 53% | 67% |
| Specificity | 80% | 87% | 87% | 80% |
| OR Quartile 2 | 6.45 | 7.02 | 6.81 | 6.45 |
| p Value | 0.012 | 1.3E−5 | 1.8E−5 | 0.012 |
| Lower limit of 95% CI | 1.51 | 2.92 | 2.83 | 1.51 |
| Upper limit of 95% CI | 27.5 | 16.8 | 16.3 | 27.5 |
| OR Quartile 3 | 9.79 | 10.3 | 9.86 | 9.79 |
| p Value | 2.8E−5 | 6.6E−13 | 1.6E−12 | 2.8E−5 |
| Lower limit of 95% CI | 3.37 | 5.43 | 5.22 | 3.37 |
| Upper limit of 95% CI | 28.4 | 19.3 | 18.6 | 28.4 |
| OR Quartile 4 | 8.15 | 8.20 | 7.59 | 8.15 |
| p Value | 5.2E−8 | 7.7E−13 | 4.4E−12 | 5.2E−8 |
| Lower limit of 95% CI | 3.83 | 4.61 | 4.28 | 3.83 |
| Upper limit of 95% CI | 17.3 | 14.6 | 13.5 | 17.3 |

TABLE 4
Comparison of marker levels and the area under the ROC curve (AUC) in urine samples for the “persistent”
and “non-persistent” cohorts where persistence starts within 48 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| | Persistence duration (hr) | | | | |
|---|---|---|---|---|---|
| | 24 | | | 48 | |
| | sCr or | | | sCr or | |
| | UO | sCr only | UO only | UO | sCr only |
| C-C motif chemokine 14 | | | | | |
| AUC | 0.80 | 0.79 | 0.75 | 0.84 | 0.82 |
| SE | 0.026 | 0.027 | 0.037 | 0.025 | 0.026 |
| p Value | 0 | 0 | 1.2E−11 | 0 | 0 |
| nCohort Non-persistent | 194 | 202 | 265 | 211 | 216 |
| nCohort Persistent | 137 | 127 | 64 | 120 | 113 |
| Cutoff Quartile 2 | 0.725 | 0.719 | 0.732 | 0.725 | 0.719 |
| Sensitivity | 91% | 91% | 95% | 94% | 94% |
| Specificity | 37% | 35% | 30% | 36% | 35% |
| Cutoff Quartile 3 | 1.64 | 1.64 | 1.64 | 1.64 | 1.64 |
| Sensitivity | 78% | 78% | 86% | 84% | 83% |
| Specificity | 70% | 67% | 58% | 69% | 67% |
| Cutoff Quartile 4 | 5.16 | 5.09 | 5.09 | 5.16 | 5.09 |
| Sensitivity | 47% | 47% | 45% | 52% | 51% |
| Specificity | 90% | 89% | 80% | 90% | 88% |
| OR Quartile 2 | 6.01 | 5.08 | 8.64 | 9.09 | 8.05 |
| p Value | 1.0E−7 | 1.5E−6 | 3.8E−4 | 1.1E−7 | 5.2E−7 |
| Lower limit of 95% CI | 3.11 | 2.62 | 2.63 | 4.03 | 3.57 |
| Upper limit of 95% CI | 11.6 | 9.85 | 28.3 | 20.5 | 18.2 |
| OR Quartile 3 | 8.16 | 7.29 | 8.61 | 11.9 | 10.1 |
| p Value | 4.4E−16 | 3.0E−14 | 1.5E−8 | 0 | 1.6E−15 |
| Lower limit of 95% CI | 4.91 | 4.36 | 4.08 | 6.75 | 5.72 |
| Upper limit of 95% CI | 13.6 | 12.2 | 18.2 | 21.1 | 17.8 |
| OR Quartile 4 | 8.07 | 6.97 | 3.24 | 9.67 | 8.06 |
| p Value | 1.7E−12 | 8.1E−12 | 6.4E−5 | 1.1E−14 | 2.0E−13 |
| Lower limit of 95% CI | 4.52 | 3.99 | 1.82 | 5.44 | 4.62 |
| Upper limit of 95% CI | 14.4 | 12.2 | 5.76 | 17.2 | 14.1 |
| Weight adjusted urine output | | | | | |
| AUC | 0.34 | 0.38 | 0.27 | 0.33 | 0.35 |
| SE | 0.031 | 0.032 | 0.037 | 0.031 | 0.032 |
| p Value | 3.9E−7 | 1.7E−4 | 1.1E−9 | 8.2E−8 | 5.5E−6 |
| nCohort Non-persistent | 197 | 205 | 270 | 214 | 219 |
| nCohort Persistent | 140 | 130 | 67 | 123 | 116 |
| Cutoff Quartile 2 | 0.189 | 0.192 | 0.189 | 0.189 | 0.192 |
| Sensitivity | 63% | 67% | 48% | 63% | 66% |
| Specificity | 16% | 20% | 18% | 18% | 20% |
| Cutoff Quartile 3 | 0.366 | 0.368 | 0.366 | 0.366 | 0.368 |
| Sensitivity | 38% | 39% | 28% | 36% | 36% |
| Specificity | 41% | 43% | 44% | 42% | 42% |
| Cutoff Quartile 4 | 0.857 | 0.862 | 0.857 | 0.857 | 0.862 |
| Sensitivity | 18% | 19% | 10% | 16% | 17% |
| Specificity | 70% | 71% | 71% | 70% | 71% |
| OR Quartile 2 | 0.328 | 0.490 | 0.203 | 0.361 | 0.464 |
| p Value | 1.9E−5 | 0.0055 | 4.2E−8 | 8.1E−5 | 0.0030 |
| Lower limit of 95% CI | 0.197 | 0.297 | 0.115 | 0.218 | 0.279 |
| Upper limit of 95% CI | 0.547 | 0.811 | 0.359 | 0.600 | 0.771 |
| OR Quartile 3 | 0.425 | 0.486 | 0.317 | 0.397 | 0.419 |
| p Value | 1.6E−4 | 0.0016 | 1.1E−4 | 7.6E−5 | 2.4E−4 |
| Lower limit of 95% CI | 0.273 | 0.310 | 0.177 | 0.251 | 0.263 |
| Upper limit of 95% CI | 0.663 | 0.760 | 0.567 | 0.627 | 0.666 |
| OR Quartile 4 | 0.496 | 0.589 | 0.287 | 0.445 | 0.505 |
| p Value | 0.0094 | 0.051 | 0.0031 | 0.0047 | 0.017 |
| Lower limit of 95% CI | 0.293 | 0.347 | 0.126 | 0.254 | 0.287 |
| Upper limit of 95% CI | 0.842 | 1.00 | 0.656 | 0.780 | 0.886 |
| Serum Creatinine | | | | | |
| AUC | 0.81 | 0.82 | 0.70 | 0.82 | 0.82 |
| SE | 0.024 | 0.024 | 0.036 | 0.024 | 0.025 |
| p Value | 0 | 0 | 1.6E−8 | 0 | 0 |
| nCohort Non-persistent | 199 | 207 | 273 | 216 | 221 |
| nCohort Persistent | 155 | 145 | 79 | 138 | 131 |
| Cutoff Quartile 2 | 1.64 | 1.64 | 1.65 | 1.64 | 1.64 |
| Sensitivity | 94% | 94% | 92% | 96% | 96% |
| Specificity | 40% | 39% | 30% | 38% | 38% |
| Cutoff Quartile 3 | 2.44 | 2.44 | 2.44 | 2.44 | 2.44 |
| Sensitivity | 76% | 78% | 70% | 79% | 80% |
| Specificity | 70% | 70% | 55% | 68% | 68% |
| Cutoff Quartile 4 | 3.32 | 3.31 | 3.32 | 3.32 | 3.31 |
| Sensitivity | 46% | 48% | 46% | 49% | 50% |
| Specificity | 91% | 91% | 81% | 89% | 90% |
| OR Quartile 2 | 9.55 | 10.8 | 5.22 | 13.7 | 15.2 |
| p Value | 2.8E−10 | 1.2E−9 | 2.0E−4 | 2.7E−9 | 1.2E−8 |
| Lower limit of 95% CI | 4.74 | 5.02 | 2.18 | 5.79 | 5.95 |
| Upper limit of 95% CI | 19.2 | 23.2 | 12.5 | 32.5 | 38.6 |
| OR Quartile 3 | 7.39 | 8.07 | 2.84 | 8.01 | 8.53 |
| p Value | 2.2E−16 | 0 | 1.4E−4 | 2.2E−16 | 2.2E−16 |
| Lower limit of 95% CI | 4.58 | 4.94 | 1.66 | 4.86 | 5.10 |
| Upper limit of 95% CI | 11.9 | 13.2 | 4.85 | 13.2 | 14.3 |
| OR Quartile 4 | 8.72 | 9.80 | 3.48 | 7.92 | 8.48 |
| p Value | 2.1E−13 | 1.7E−14 | 5.0E−6 | 1.1E−13 | 2.9E−14 |
| Lower limit of 95% CI | 4.89 | 5.47 | 2.04 | 4.59 | 4.89 |
| Upper limit of 95% CI | 15.6 | 17.6 | 5.93 | 13.7 | 14.7 |
| Cystatin-C | | | | | |
| AUC | 0.75 | 0.75 | 0.74 | 0.76 | 0.76 |
| SE | 0.029 | 0.029 | 0.039 | 0.030 | 0.030 |
| p Value | 0 | 0 | 4.7E−10 | 0 | 0 |
| nCohort Non-persistent | 178 | 184 | 247 | 194 | 197 |
| nCohort Persistent | 130 | 123 | 59 | 114 | 110 |
| Cutoff Quartile 2 | 1170000 | 1170000 | 1170000 | 1170000 | 1170000 |
| Sensitivity | 92% | 91% | 93% | 91% | 91% |
| Specificity | 37% | 35% | 30% | 34% | 34% |
| Cutoff Quartile 3 | 1660000 | 1650000 | 1660000 | 1660000 | 1650000 |
| Sensitivity | 74% | 75% | 81% | 75% | 76% |
| Specificity | 67% | 66% | 57% | 64% | 64% |
| Cutoff Quartile 4 | 2150000 | 2150000 | 2150000 | 2150000 | 2150000 |
| Sensitivity | 40% | 41% | 51% | 43% | 45% |
| Specificity | 86% | 86% | 81% | 86% | 86% |
| OR Quartile 2 | 6.22 | 5.56 | 5.77 | 5.36 | 5.04 |
| p Value | 2.0E−7 | 1.1E−6 | 0.0011 | 4.0E−6 | 9.1E−6 |
| Lower limit of 95% CI | 3.12 | 2.79 | 2.02 | 2.63 | 2.47 |
| Upper limit of 95% CI | 12.4 | 11.1 | 16.5 | 10.9 | 10.3 |
| OR Quartile 3 | 5.84 | 5.84 | 5.90 | 5.31 | 5.86 |
| p Value | 5.1E−12 | 1.1E−11 | 7.2E−7 | 1.9E−10 | 5.1E−11 |
| Lower limit of 95% CI | 3.54 | 3.51 | 2.92 | 3.18 | 3.46 |
| Upper limit of 95% CI | 9.64 | 9.72 | 11.9 | 8.88 | 9.94 |
| OR Quartile 4 | 4.08 | 4.30 | 4.40 | 4.47 | 4.85 |
| p Value | 5.3E−7 | 1.8E−7 | 1.4E−6 | 7.6E−8 | 1.7E−8 |
| Lower limit of 95% CI | 2.36 | 2.49 | 2.41 | 2.59 | 2.80 |
| Upper limit of 95% CI | 7.07 | 7.45 | 8.03 | 7.71 | 8.39 |
| C-C motif chemokine 14/(Weight | | | | | |
| adjusted urine output) | | | | | |
| AUC | 0.79 | 0.76 | 0.80 | 0.82 | 0.80 |
| SE | 0.026 | 0.028 | 0.035 | 0.026 | 0.028 |
| p Value | 0 | 0 | 0 | 0 | 0 |
| nCohort Non-persistent | 192 | 200 | 264 | 209 | 214 |
| nCohort Persistent | 136 | 126 | 64 | 119 | 112 |
| Cutoff Quartile 2 | 1.26 | 1.24 | 1.26 | 1.26 | 1.24 |
| Sensitivity | 89% | 88% | 97% | 93% | 93% |
| Specificity | 35% | 34% | 30% | 35% | 35% |
| Cutoff Quartile 3 | 4.59 | 4.54 | 4.59 | 4.59 | 4.54 |
| Sensitivity | 76% | 75% | 86% | 81% | 79% |
| Specificity | 69% | 66% | 59% | 67% | 65% |
| Cutoff Quartile 4 | 24.3 | 22.9 | 24.3 | 24.3 | 22.9 |
| Sensitivity | 49% | 48% | 58% | 52% | 52% |
| Specificity | 92% | 89% | 83% | 90% | 89% |
| OR Quartile 2 | 4.32 | 3.73 | 13.5 | 7.61 | 6.87 |
| p Value | 2.9E−6 | 2.7E−5 | 3.7E−4 | 2.5E−7 | 1.0E−6 |
| Lower limit of 95% CI | 2.34 | 2.02 | 3.22 | 3.52 | 3.17 |
| Upper limit of 95% CI | 7.98 | 6.89 | 56.5 | 16.5 | 14.9 |
| OR Quartile 3 | 7.15 | 5.58 | 8.69 | 8.65 | 7.32 |
| p Value | 1.3E−14 | 1.1E−11 | 1.3E−8 | 4.4E−15 | 4.1E−13 |
| Lower limit of 95% CI | 4.34 | 3.40 | 4.12 | 5.05 | 4.27 |
| Upper limit of 95% CI | 11.8 | 9.16 | 18.3 | 14.8 | 12.5 |
| OR Quartile 4 | 10.4 | 7.36 | 6.67 | 10.3 | 8.50 |
| p Value | 7.1E−14 | 4.2E−12 | 3.1E−10 | 5.6E−15 | 9.8E−14 |
| Lower limit of 95% CI | 5.62 | 4.18 | 3.69 | 5.73 | 4.84 |
| Upper limit of 95% CI | 19.1 | 12.9 | 12.0 | 18.4 | 14.9 |
| C-C motif chemokine | | | | | |
| 14 X Serum Creatinine | | | | | |
| AUC | 0.84 | 0.83 | 0.77 | 0.87 | 0.86 |
| SE | 0.024 | 0.025 | 0.037 | 0.023 | 0.024 |
| p Value | 0 | 0 | 1.8E−13 | 0 | 0 |
| nCohort Non-persistent | 188 | 196 | 258 | 205 | 210 |
| nCohort Persistent | 136 | 126 | 64 | 119 | 112 |
| Cutoff Quartile 2 | 1.29 | 1.29 | 1.30 | 1.29 | 1.29 |
| Sensitivity | 93% | 93% | 94% | 96% | 96% |
| Specificity | 38% | 37% | 30% | 37% | 36% |
| Cutoff Quartile 3 | 3.97 | 3.91 | 3.97 | 3.97 | 3.91 |
| Sensitivity | 82% | 82% | 89% | 87% | 87% |
| Specificity | 73% | 70% | 60% | 72% | 70% |
| Cutoff Quartile 4 | 18.3 | 16.8 | 18.0 | 18.3 | 16.8 |
| Sensitivity | 49% | 49% | 52% | 55% | 54% |
| Specificity | 92% | 90% | 81% | 92% | 90% |
| OR Quartile 2 | 7.65 | 7.55 | 6.38 | 13.4 | 12.1 |
| p Value | 1.8E−8 | 7.8E−8 | 5.2E−4 | 6.0E−8 | 1.9E−7 |
| Lower limit of 95% CI | 3.77 | 3.61 | 2.24 | 5.25 | 4.74 |
| Upper limit of 95% CI | 15.5 | 15.8 | 18.2 | 34.4 | 31.1 |
| OR Quartile 3 | 11.9 | 10.7 | 12.1 | 17.6 | 14.8 |
| p Value | 0 | 0 | 3.1E−9 | 0 | 0 |
| Lower limit of 95% CI | 6.95 | 6.17 | 5.29 | 9.45 | 7.95 |
| Upper limit of 95% CI | 20.5 | 18.4 | 27.5 | 32.7 | 27.4 |
| OR Quartile 4 | 10.9 | 9.02 | 4.66 | 14.2 | 11.4 |
| p Value | 7.6E−14 | 2.3E−13 | 2.2E−7 | 0 | 8.9E−16 |
| Lower limit of 95% CI | 5.82 | 5.01 | 2.60 | 7.61 | 6.29 |
| Upper limit of 95% CI | 20.3 | 16.2 | 8.33 | 26.6 | 20.5 |
| C-C motif chemokine | | | | | |
| 14 X Cystatin-C | | | | | |
| AUC | 0.82 | 0.82 | 0.77 | 0.85 | 0.84 |
| SE | 0.025 | 0.026 | 0.038 | 0.025 | 0.025 |
| p Value | 0 | 0 | 1.6E−12 | 0 | 0 |
| nCohort Non-persistent | 178 | 184 | 247 | 194 | 197 |
| nCohort Persistent | 130 | 123 | 59 | 114 | 110 |
| Cutoff Quartile 2 | 956000 | 955000 | 958000 | 956000 | 955000 |
| Sensitivity | 92% | 92% | 95% | 94% | 94% |
| Specificity | 38% | 36% | 30% | 36% | 36% |
| Cutoff Quartile 3 | 2880000 | 2860000 | 2880000 | 2880000 | 2860000 |
| Sensitivity | 82% | 81% | 88% | 85% | 85% |
| Specificity | 73% | 71% | 59% | 71% | 69% |
| Cutoff Quartile 4 | 1.04E7 | 1.03E7 | 1.03E7 | 1.04E7 | 1.03E7 |
| Sensitivity | 47% | 48% | 53% | 54% | 54% |
| Specificity | 91% | 90% | 81% | 92% | 91% |
| OR Quartile 2 | 7.24 | 6.47 | 7.98 | 8.63 | 8.11 |
| p Value | 5.2E−8 | 2.8E−7 | 6.4E−4 | 2.5E−7 | 5.5E−7 |
| Lower limit of 95% CI | 3.55 | 3.17 | 2.42 | 3.80 | 3.57 |
| Upper limit of 95% CI | 14.8 | 13.2 | 26.3 | 19.6 | 18.4 |
| OR Quartile 3 | 12.0 | 10.5 | 10.7 | 13.7 | 12.2 |
| p Value | 0 | 0 | 2.0E−8 | 0 | 2.2E−16 |
| Lower limit of 95% CI | 6.88 | 6.02 | 4.69 | 7.52 | 6.70 |
| Upper limit of 95% CI | 20.8 | 18.2 | 24.6 | 25.0 | 22.2 |
| OR Quartile 4 | 8.95 | 8.50 | 4.84 | 12.8 | 11.5 |
| p Value | 3.7E−12 | 3.1E−12 | 3.0E−7 | 2.2E−15 | 5.6E−15 |
| Lower limit of 95% CI | 4.82 | 4.66 | 2.65 | 6.82 | 6.23 |
| Upper limit of 95% CI | 16.6 | 15.5 | 8.84 | 24.0 | 21.2 |
| C-C motif chemokine 14 X | | | | | |
| Serum Creatinine/(Weight | | | | | |
| adjusted urine output) | | | | | |
| AUC | 0.83 | 0.80 | 0.82 | 0.86 | 0.84 |
| SE | 0.024 | 0.027 | 0.034 | 0.024 | 0.026 |
| p Value | 0 | 0 | 0 | 0 | 0 |
| nCohort Non-persistent | 186 | 194 | 257 | 203 | 208 |
| nCohort Persistent | 135 | 125 | 64 | 118 | 111 |
| Cutoff Quartile 2 | 2.17 | 2.15 | 2.17 | 2.17 | 2.15 |
| Sensitivity | 91% | 90% | 95% | 93% | 93% |
| Specificity | 37% | 35% | 30% | 35% | 35% |
| Cutoff Quartile 3 | 10.2 | 10.1 | 10.2 | 10.2 | 10.1 |
| Sensitivity | 81% | 79% | 89% | 85% | 84% |
| Specificity | 72% | 69% | 60% | 70% | 68% |
| Cutoff Quartile 4 | 69.8 | 67.6 | 69.8 | 69.8 | 67.6 |
| Sensitivity | 51% | 50% | 56% | 56% | 54% |
| Specificity | 94% | 91% | 82% | 93% | 90% |
| OR Quartile 2 | 5.91 | 5.08 | 8.70 | 7.56 | 6.82 |
| p Value | 1.6E−7 | 1.6E−6 | 3.6E−4 | 2.9E−7 | 1.2E−6 |
| Lower limit of 95% CI | 3.04 | 2.62 | 2.65 | 3.49 | 3.14 |
| Upper limit of 95% CI | 11.5 | 9.87 | 28.6 | 16.4 | 14.8 |
| OR Quartile 3 | 10.8 | 8.30 | 12.0 | 12.9 | 10.9 |
| p Value | 0 | 3.8E−15 | 3.4E−9 | 0 | 8.9E−16 |
| Lower limit of 95% CI | 6.33 | 4.90 | 5.26 | 7.21 | 6.07 |
| Upper limit of 95% CI | 18.4 | 14.1 | 27.3 | 23.2 | 19.5 |
| OR Quartile 4 | 15.2 | 9.62 | 6.06 | 15.9 | 11.1 |
| p Value | 3.1E−15 | 1.2E−13 | 2.1E−9 | 0 | 2.0E−15 |
| Lower limit of 95% CI | 7.72 | 5.29 | 3.36 | 8.39 | 6.11 |
| Upper limit of 95% CI | 29.8 | 17.5 | 10.9 | 30.1 | 20.0 |
| C-C motif chemokine 14 X | | | | | |
| Cystatin-C/(Weight | | | | | |
| adjusted urine output) | | | | | |
| AUC | 0.81 | 0.79 | 0.80 | 0.83 | 0.82 |
| SE | 0.026 | 0.028 | 0.036 | 0.026 | 0.027 |
| p Value | 0 | 0 | 2.2E−16 | 0 | 0 |
| nCohort Non-persistent | 176 | 182 | 246 | 192 | 195 |
| nCohort Persistent | 129 | 122 | 59 | 113 | 109 |
| Cutoff Quartile 2 | 1580000 | 1580000 | 1580000 | 1580000 | 1580000 |
| Sensitivity | 90% | 89% | 95% | 93% | 93% |
| Specificity | 36% | 35% | 30% | 35% | 35% |
| Cutoff Quartile 3 | 6710000 | 6570000 | 6710000 | 6710000 | 6570000 |
| Sensitivity | 78% | 77% | 88% | 82% | 82% |
| Specificity | 70% | 68% | 59% | 69% | 68% |
| Cutoff Quartile 4 | 3.85E7 | 3.84E7 | 3.85E7 | 3.85E7 | 3.84E7 |
| Sensitivity | 47% | 46% | 53% | 50% | 50% |
| Specificity | 91% | 89% | 81% | 90% | 89% |
| OR Quartile 2 | 4.97 | 4.44 | 7.88 | 7.20 | 6.76 |
| p Value | 1.4E−6 | 7.3E−6 | 7.0E−4 | 6.5E−7 | 1.5E−6 |
| Lower limit of 95% CI | 2.59 | 2.31 | 2.39 | 3.31 | 3.11 |
| Upper limit of 95% CI | 9.54 | 8.51 | 26.0 | 15.7 | 14.7 |
| OR Quartile 3 | 8.60 | 7.18 | 10.7 | 10.2 | 9.32 |
| p Value | 1.6E−15 | 1.8E−13 | 2.2E−8 | 1.6E−15 | 1.7E−14 |
| Lower limit of 95% CI | 5.07 | 4.25 | 4.66 | 5.78 | 5.27 |
| Upper limit of 95% CI | 14.6 | 12.1 | 24.4 | 18.1 | 16.5 |
| OR Quartile 4 | 8.97 | 6.87 | 4.81 | 8.75 | 7.72 |
| p Value | 3.8E−12 | 1.1E−10 | 3.3E−7 | 6.8E−13 | 5.5E−12 |
| Lower limit of 95% CI | 4.83 | 3.83 | 2.63 | 4.84 | 4.32 |
| Upper limit of 95% CI | 16.7 | 12.3 | 8.80 | 15.8 | 13.8 |

| | Persistence duration (hr) | | | |
|---|---|---|---|---|
| | | 72 | | |
| | 48 | sCr or | | |
| | UO only | UO | sCr only | UO only |
| C-C motif chemokine 14 | | | | |
| AUC | 0.76 | 0.83 | 0.81 | 0.76 |
| SE | 0.038 | 0.026 | 0.028 | 0.038 |
| p Value | 3.4E−12 | 0 | 0 | 3.4E−12 |
| nCohort Non-persistent | 269 | 221 | 226 | 269 |
| nCohort Persistent | 60 | 110 | 103 | 60 |
| Cutoff Quartile 2 | 0.732 | 0.725 | 0.719 | 0.732 |
| Sensitivity | 95% | 95% | 94% | 95% |
| Specificity | 29% | 35% | 34% | 29% |
| Cutoff Quartile 3 | 1.64 | 1.64 | 1.64 | 1.64 |
| Sensitivity | 88% | 85% | 84% | 88% |
| Specificity | 58% | 67% | 65% | 58% |
| Cutoff Quartile 4 | 5.09 | 5.16 | 5.09 | 5.09 |
| Sensitivity | 48% | 51% | 50% | 48% |
| Specificity | 80% | 88% | 86% | 80% |
| OR Quartile 2 | 7.90 | 9.27 | 8.19 | 7.90 |
| p Value | 6.7E−4 | 5.0E−7 | 2.1E−6 | 6.7E−4 |
| Lower limit of 95% CI | 2.40 | 3.89 | 3.43 | 2.40 |
| Upper limit of 95% CI | 26.0 | 22.1 | 19.5 | 26.0 |
| OR Quartile 3 | 10.6 | 12.2 | 10.3 | 10.6 |
| p Value | 2.0E−8 | 4.4E−16 | 2.4E−14 | 2.0E−8 |
| Lower limit of 95% CI | 4.65 | 6.67 | 5.66 | 4.65 |
| Upper limit of 95% CI | 24.2 | 22.2 | 18.8 | 24.2 |
| OR Quartile 4 | 3.72 | 7.45 | 6.41 | 3.72 |
| p Value | 1.2E−5 | 7.8E−13 | 1.7E−11 | 1.2E−5 |
| Lower limit of 95% CI | 2.07 | 4.30 | 3.73 | 2.07 |
| Upper limit of 95% CI | 6.70 | 12.9 | 11.0 | 6.70 |
| Weight adjusted urine output | | | | |
| AUC | 0.29 | 0.33 | 0.35 | 0.29 |
| SE | 0.039 | 0.032 | 0.033 | 0.039 |
| p Value | 9.9E−8 | 7.8E−8 | 5.5E−6 | 9.9E−8 |
| nCohort Non-persistent | 274 | 225 | 230 | 274 |
| nCohort Persistent | 63 | 112 | 105 | 63 |
| Cutoff Quartile 2 | 0.189 | 0.189 | 0.192 | 0.189 |
| Sensitivity | 51% | 61% | 64% | 51% |
| Specificity | 19% | 18% | 20% | 19% |
| Cutoff Quartile 3 | 0.366 | 0.366 | 0.368 | 0.366 |
| Sensitivity | 30% | 35% | 35% | 30% |
| Specificity | 45% | 42% | 43% | 45% |
| Cutoff Quartile 4 | 0.857 | 0.857 | 0.862 | 0.857 |
| Sensitivity | 11% | 16% | 17% | 11% |
| Specificity | 72% | 70% | 71% | 72% |
| OR Quartile 2 | 0.248 | 0.334 | 0.429 | 0.248 |
| p Value | 2.2E−6 | 2.6E−5 | 0.0013 | 2.2E−6 |
| Lower limit of 95% CI | 0.139 | 0.201 | 0.256 | 0.139 |
| Upper limit of 95% CI | 0.441 | 0.557 | 0.717 | 0.441 |
| OR Quartile 3 | 0.357 | 0.390 | 0.411 | 0.357 |
| p Value | 6.0E−4 | 8.8E−5 | 2.7E−4 | 6.0E−4 |
| Lower limit of 95% CI | 0.198 | 0.244 | 0.255 | 0.198 |
| Upper limit of 95% CI | 0.643 | 0.625 | 0.663 | 0.643 |
| OR Quartile 4 | 0.314 | 0.452 | 0.514 | 0.314 |
| p Value | 0.0061 | 0.0072 | 0.025 | 0.0061 |
| Lower limit of 95% CI | 0.137 | 0.253 | 0.287 | 0.137 |
| Upper limit of 95% CI | 0.719 | 0.806 | 0.920 | 0.719 |
| Serum Creatinine | | | | |
| AUC | 0.71 | 0.81 | 0.81 | 0.71 |
| SE | 0.036 | 0.026 | 0.026 | 0.036 |
| p Value | 3.4E−9 | 0 | 0 | 1.2E−8 |
| nCohort Non-persistent | 276 | 227 | 232 | 277 |
| nCohort Persistent | 76 | 127 | 120 | 75 |
| Cutoff Quartile 2 | 1.65 | 1.64 | 1.64 | 1.65 |
| Sensitivity | 93% | 95% | 96% | 93% |
| Specificity | 30% | 37% | 36% | 30% |
| Cutoff Quartile 3 | 2.44 | 2.44 | 2.44 | 2.44 |
| Sensitivity | 71% | 80% | 81% | 71% |
| Specificity | 55% | 66% | 66% | 55% |
| Cutoff Quartile 4 | 3.32 | 3.32 | 3.31 | 3.32 |
| Sensitivity | 47% | 50% | 52% | 47% |
| Specificity | 81% | 89% | 89% | 81% |
| OR Quartile 2 | 6.11 | 11.6 | 12.8 | 5.99 |
| p Value | 1.7E−4 | 2.5E−8 | 8.9E−8 | 2.0E−4 |
| Lower limit of 95% CI | 2.38 | 4.90 | 5.03 | 2.33 |
| Upper limit of 95% CI | 15.7 | 27.6 | 32.6 | 15.4 |
| OR Quartile 3 | 3.05 | 7.57 | 8.17 | 2.97 |
| p Value | 6.9E−5 | 8.4E−15 | 7.5E−15 | 1.1E−4 |
| Lower limit of 95% CI | 1.76 | 4.54 | 4.81 | 1.71 |
| Upper limit of 95% CI | 5.29 | 12.6 | 13.9 | 5.16 |
| OR Quartile 4 | 3.79 | 7.85 | 8.47 | 3.61 |
| p Value | 1.4E−6 | 5.1E−14 | 1.2E−14 | 3.4E−6 |
| Lower limit of 95% CI | 2.20 | 4.59 | 4.92 | 2.10 |
| Upper limit of 95% CI | 6.50 | 13.4 | 14.6 | 6.22 |
| Cystatin-C | | | | |
| AUC | 0.74 | 0.75 | 0.75 | 0.74 |
| SE | 0.040 | 0.031 | 0.031 | 0.041 |
| p Value | 3.3E−9 | 3.1E−15 | 1.6E−15 | 8.0E−9 |
| nCohort Non-persistent | 250 | 204 | 207 | 251 |
| nCohort Persistent | 56 | 104 | 100 | 55 |
| Cutoff Quartile 2 | 1170000 | 1170000 | 1170000 | 1170000 |
| Sensitivity | 93% | 91% | 91% | 93% |
| Specificity | 29% | 33% | 32% | 29% |
| Cutoff Quartile 3 | 1660000 | 1660000 | 1650000 | 1660000 |
| Sensitivity | 80% | 75% | 77% | 80% |
| Specificity | 57% | 63% | 63% | 57% |
| Cutoff Quartile 4 | 2150000 | 2150000 | 2150000 | 2150000 |
| Sensitivity | 52% | 43% | 45% | 51% |
| Specificity | 81% | 84% | 85% | 80% |
| OR Quartile 2 | 5.36 | 5.16 | 4.84 | 5.23 |
| p Value | 0.0018 | 1.5E−5 | 3.3E−5 | 0.0021 |
| Lower limit of 95% CI | 1.87 | 2.45 | 2.30 | 1.82 |
| Upper limit of 95% CI | 15.4 | 10.9 | 10.2 | 15.0 |
| OR Quartile 3 | 5.38 | 5.05 | 5.65 | 5.21 |
| p Value | 2.9E−6 | 1.7E−9 | 4.5E−10 | 4.6E−6 |
| Lower limit of 95% CI | 2.66 | 2.98 | 3.28 | 2.57 |
| Upper limit of 95% CI | 10.9 | 8.56 | 9.74 | 10.6 |
| OR Quartile 4 | 4.52 | 4.10 | 4.47 | 4.28 |
| p Value | 1.3E−6 | 3.2E−7 | 7.2E−8 | 3.5E−6 |
| Lower limit of 95% CI | 2.45 | 2.39 | 2.59 | 2.31 |
| Upper limit of 95% CI | 8.33 | 7.04 | 7.72 | 7.90 |
| C-C motif chemokine 14/(Weight | | | | |
| adjusted urine output) | | | | |
| AUC | 0.79 | 0.81 | 0.79 | 0.79 |
| SE | 0.036 | 0.027 | 0.029 | 0.036 |
| p Value | 6.7E−16 | 0 | 0 | 6.7E−16 |
| nCohort Non-persistent | 268 | 219 | 224 | 268 |
| nCohort Persistent | 60 | 109 | 102 | 60 |
| Cutoff Quartile 2 | 1.26 | 1.26 | 1.24 | 1.26 |
| Sensitivity | 97% | 94% | 94% | 97% |
| Specificity | 30% | 35% | 34% | 30% |
| Cutoff Quartile 3 | 4.59 | 4.59 | 4.54 | 4.59 |
| Sensitivity | 85% | 82% | 80% | 85% |
| Specificity | 58% | 66% | 64% | 58% |
| Cutoff Quartile 4 | 24.3 | 24.3 | 22.9 | 24.3 |
| Sensitivity | 57% | 52% | 52% | 57% |
| Specificity | 82% | 89% | 87% | 82% |
| OR Quartile 2 | 12.3 | 9.12 | 8.22 | 12.3 |
| p Value | 5.9E−4 | 6.1E−7 | 2.1E−6 | 5.9E−4 |
| Lower limit of 95% CI | 2.94 | 3.83 | 3.44 | 2.94 |
| Upper limit of 95% CI | 51.8 | 21.8 | 19.6 | 51.8 |
| OR Quartile 3 | 7.77 | 8.54 | 7.24 | 7.77 |
| p Value | 8.0E−8 | 5.8E−14 | 4.1E−12 | 8.0E−8 |
| Lower limit of 95% CI | 3.68 | 4.88 | 4.14 | 3.68 |
| Upper limit of 95% CI | 16.4 | 15.0 | 12.7 | 16.4 |
| OR Quartile 4 | 5.99 | 8.51 | 7.27 | 5.99 |
| p Value | 4.5E−9 | 7.5E−14 | 1.6E−12 | 4.5E−9 |
| Lower limit of 95% CI | 3.29 | 4.85 | 4.19 | 3.29 |
| Upper limit of 95% CI | 10.9 | 14.9 | 12.6 | 10.9 |
| C-C motif chemokine | | | | |
| 14 X Serum Creatinine | | | | |
| AUC | 0.78 | 0.86 | 0.85 | 0.78 |
| SE | 0.037 | 0.024 | 0.026 | 0.037 |
| p Value | 3.5E−14 | 0 | 0 | 3.5E−14 |
| nCohort Non-persistent | 262 | 215 | 220 | 262 |
| nCohort Persistent | 60 | 109 | 102 | 60 |
| Cutoff Quartile 2 | 1.30 | 1.29 | 1.29 | 1.30 |
| Sensitivity | 95% | 96% | 96% | 95% |
| Specificity | 30% | 36% | 35% | 30% |
| Cutoff Quartile 3 | 3.97 | 3.97 | 3.91 | 3.97 |
| Sensitivity | 92% | 89% | 88% | 92% |
| Specificity | 60% | 70% | 68% | 60% |
| Cutoff Quartile 4 | 18.0 | 18.3 | 16.8 | 18.0 |
| Sensitivity | 53% | 53% | 53% | 53% |
| Specificity | 81% | 89% | 88% | 81% |
| OR Quartile 2 | 8.05 | 14.6 | 13.2 | 8.05 |
| p Value | 6.0E−4 | 3.9E−7 | 1.1E−6 | 6.0E−4 |
| Lower limit of 95% CI | 2.45 | 5.19 | 4.67 | 2.45 |
| Upper limit of 95% CI | 26.5 | 41.3 | 37.2 | 26.5 |
| OR Quartile 3 | 16.2 | 18.7 | 15.7 | 16.2 |
| p Value | 8.6E−9 | 0 | 4.4E−16 | 8.6E−9 |
| Lower limit of 95% CI | 6.27 | 9.58 | 8.09 | 6.27 |
| Upper limit of 95% CI | 41.8 | 36.3 | 30.6 | 41.8 |
| OR Quartile 4 | 4.97 | 9.49 | 8.04 | 4.97 |
| p Value | 1.3E−7 | 1.4E−14 | 2.9E−13 | 1.3E−7 |
| Lower limit of 95% CI | 2.74 | 5.35 | 4.59 | 2.74 |
| Upper limit of 95% CI | 9.00 | 16.8 | 14.1 | 9.00 |
| C-C motif chemokine | | | | |
| 14 X Cystatin-C | | | | |
| AUC | 0.78 | 0.84 | 0.83 | 0.78 |
| SE | 0.039 | 0.026 | 0.027 | 0.039 |
| p Value | 8.7E−13 | 0 | 0 | 8.7E−13 |
| nCohort Non-persistent | 251 | 204 | 207 | 251 |
| nCohort Persistent | 55 | 104 | 100 | 55 |
| Cutoff Quartile 2 | 958000 | 956000 | 955000 | 958000 |
| Sensitivity | 95% | 94% | 94% | 95% |
| Specificity | 29% | 35% | 34% | 29% |
| Cutoff Quartile 3 | 2880000 | 2880000 | 2860000 | 2880000 |
| Sensitivity | 89% | 87% | 86% | 89% |
| Specificity | 59% | 69% | 67% | 59% |
| Cutoff Quartile 4 | 1.03E7 | 1.04E7 | 1.03E7 | 1.03E7 |
| Sensitivity | 56% | 53% | 53% | 56% |
| Specificity | 82% | 89% | 88% | 82% |
| OR Quartile 2 | 7.25 | 8.72 | 8.18 | 7.25 |
| p Value | 0.0012 | 1.2E−6 | 2.4E−6 | 0.0012 |
| Lower limit of 95% CI | 2.19 | 3.64 | 3.41 | 2.19 |
| Upper limit of 95% CI | 23.9 | 20.9 | 19.6 | 23.9 |
| OR Quartile 3 | 11.5 | 14.1 | 12.6 | 11.5 |
| p Value | 5.9E−8 | 4.4E−16 | 5.8E−15 | 5.9E−8 |
| Lower limit of 95% CI | 4.77 | 7.44 | 6.65 | 4.77 |
| Upper limit of 95% CI | 27.9 | 26.6 | 23.7 | 27.9 |
| OR Quartile 4 | 5.76 | 9.29 | 8.60 | 5.76 |
| p Value | 3.4E−8 | 9.5E−14 | 3.3E−13 | 3.4E−8 |
| Lower limit of 95% CI | 3.09 | 5.17 | 4.82 | 3.09 |
| Upper limit of 95% CI | 10.7 | 16.7 | 15.3 | 10.7 |
| C-C motif chemokine 14 X | | | | |
| Serum Creatinine/(Weight | | | | |
| adjusted urine output) | | | | |
| AUC | 0.81 | 0.85 | 0.83 | 0.81 |
| SE | 0.035 | 0.025 | 0.027 | 0.035 |
| p Value | 0 | 0 | 0 | 0 |
| nCohort Non-persistent | 261 | 213 | 218 | 261 |
| nCohort Persistent | 60 | 108 | 101 | 60 |
| Cutoff Quartile 2 | 2.17 | 2.17 | 2.15 | 2.17 |
| Sensitivity | 95% | 94% | 94% | 95% |
| Specificity | 30% | 35% | 34% | 30% |
| Cutoff Quartile 3 | 10.2 | 10.2 | 10.1 | 10.2 |
| Sensitivity | 88% | 86% | 85% | 88% |
| Specificity | 59% | 68% | 66% | 59% |
| Cutoff Quartile 4 | 69.8 | 69.8 | 67.6 | 69.8 |
| Sensitivity | 57% | 56% | 54% | 57% |
| Specificity | 82% | 91% | 89% | 82% |
| OR Quartile 2 | 7.95 | 9.05 | 8.14 | 7.95 |
| p Value | 6.5E−4 | 7.0E−7 | 2.4E−6 | 6.5E−4 |
| Lower limit of 95% CI | 2.42 | 3.79 | 3.40 | 2.42 |
| Upper limit of 95% CI | 26.2 | 21.6 | 19.4 | 26.2 |
| OR Quartile 3 | 10.7 | 13.2 | 11.2 | 10.7 |
| p Value | 1.8E−8 | 2.2E−16 | 1.6E−14 | 1.8E−8 |
| Lower limit of 95% CI | 4.70 | 7.13 | 6.03 | 4.70 |
| Upper limit of 95% CI | 24.5 | 24.5 | 20.7 | 24.5 |
| OR Quartile 4 | 5.95 | 12.5 | 9.23 | 5.95 |
| p Value | 5.7E−9 | 0 | 2.6E−14 | 5.7E−9 |
| Lower limit of 95% CI | 3.27 | 6.89 | 5.21 | 3.27 |
| Upper limit of 95% CI | 10.9 | 22.8 | 16.4 | 10.9 |
| C-C motif chemokine 14 X | | | | |
| Cystatin-C/(Weight | | | | |
| adjusted urine output) | | | | |
| AUC | 0.79 | 0.82 | 0.81 | 0.79 |
| SE | 0.038 | 0.028 | 0.029 | 0.038 |
| p Value | 3.3E−14 | 0 | 0 | 3.3E−14 |
| nCohort Non-persistent | 250 | 202 | 205 | 250 |
| nCohort Persistent | 55 | 103 | 99 | 55 |
| Cutoff Quartile 2 | 1580000 | 1580000 | 1580000 | 1580000 |
| Sensitivity | 95% | 94% | 94% | 95% |
| Specificity | 29% | 35% | 34% | 29% |
| Cutoff Quartile 3 | 6710000 | 6710000 | 6570000 | 6710000 |
| Sensitivity | 87% | 83% | 83% | 87% |
| Specificity | 58% | 67% | 66% | 58% |
| Cutoff Quartile 4 | 3.85E7 | 3.85E7 | 3.84E7 | 3.85E7 |
| Sensitivity | 51% | 50% | 49% | 51% |
| Specificity | 80% | 88% | 87% | 80% |
| OR Quartile 2 | 7.15 | 8.57 | 8.04 | 7.15 |
| p Value | 0.0013 | 1.4E−6 | 3.0E−6 | 0.0013 |
| Lower limit of 95% CI | 2.16 | 3.58 | 3.35 | 2.16 |
| Upper limit of 95% CI | 23.6 | 20.5 | 19.3 | 23.6 |
| OR Quartile 3 | 9.47 | 10.2 | 9.30 | 9.47 |
| p Value | 1.2E−7 | 2.5E−14 | 2.4E−13 | 1.2E−7 |
| Lower limit of 95% CI | 4.12 | 5.61 | 5.12 | 4.12 |
| Upper limit of 95% CI | 21.8 | 18.5 | 16.9 | 21.8 |
| OR Quartile 4 | 4.25 | 7.22 | 6.46 | 4.25 |
| p Value | 3.8E−6 | 1.0E−11 | 9.6E−11 | 3.8E−6 |
| Lower limit of 95% CI | 2.30 | 4.08 | 3.67 | 2.30 |
| Upper limit of 95% CI | 7.86 | 12.8 | 11.4 | 7.86 |

TABLE 5
Comparison of marker levels and the area under the ROC curve (AUC) in urine samples for the “persistent”
and “non-persistent” cohorts where persistence starts within 72 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| | Persistence duration (hr) | | | | |
|---|---|---|---|---|---|
| | 24 | | | 48 | |
| | sCr or | | | sCr or | |
| | UO | sCr only | UO only | UO | sCr only |
| C-C motif chemokine 14 | | | | | |
| AUC | 0.79 | 0.79 | 0.72 | 0.82 | 0.82 |
| SE | 0.026 | 0.026 | 0.036 | 0.025 | 0.025 |
| p Value | 0 | 0 | 7.8E−10 | 0 | 0 |
| nCohort Non-persistent | 183 | 192 | 255 | 198 | 204 |
| nCohort Persistent | 148 | 137 | 74 | 133 | 125 |
| Cutoff Quartile 2 | 0.725 | 0.719 | 0.732 | 0.725 | 0.719 |
| Sensitivity | 90% | 90% | 92% | 92% | 93% |
| Specificity | 37% | 35% | 30% | 37% | 36% |
| Cutoff Quartile 3 | 1.64 | 1.64 | 1.64 | 1.64 | 1.64 |
| Sensitivity | 76% | 77% | 80% | 81% | 82% |
| Specificity | 71% | 69% | 58% | 71% | 69% |
| Cutoff Quartile 4 | 5.16 | 5.09 | 5.09 | 5.16 | 5.09 |
| Sensitivity | 45% | 46% | 43% | 49% | 50% |
| Specificity | 91% | 90% | 80% | 91% | 90% |
| OR Quartile 2 | 5.24 | 4.82 | 4.81 | 7.18 | 7.18 |
| p Value | 1.1E−7 | 8.9E−7 | 4.4E−4 | 4.4E−8 | 1.5E−7 |
| Lower limit of 95% CI | 2.84 | 2.57 | 2.00 | 3.54 | 3.44 |
| Upper limit of 95% CI | 9.67 | 9.02 | 11.6 | 14.6 | 15.0 |
| OR Quartile 3 | 7.92 | 7.71 | 5.53 | 10.4 | 9.93 |
| p Value | 2.2E−16 | 2.0E−15 | 6.2E−8 | 0 | 0 |
| Lower limit of 95% CI | 4.82 | 4.66 | 2.98 | 6.13 | 5.78 |
| Upper limit of 95% CI | 13.0 | 12.8 | 10.3 | 17.7 | 17.1 |
| OR Quartile 4 | 7.86 | 7.32 | 3.05 | 9.56 | 8.58 |
| p Value | 1.1E−11 | 9.1E−12 | 7.8E−5 | 7.7E−14 | 1.7E−13 |
| Lower limit of 95% CI | 4.33 | 4.13 | 1.75 | 5.29 | 4.84 |
| Upper limit of 95% CI | 14.3 | 13.0 | 5.30 | 17.3 | 15.2 |
| Weight adjusted urine output | | | | | |
| AUC | 0.36 | 0.40 | 0.29 | 0.35 | 0.38 |
| SE | 0.030 | 0.032 | 0.036 | 0.031 | 0.032 |
| p Value | 4.0E−6 | 0.0020 | 9.1E−9 | 1.2E−6 | 1.4E−4 |
| nCohort Non-persistent | 186 | 195 | 260 | 201 | 207 |
| nCohort Persistent | 151 | 140 | 77 | 136 | 128 |
| Cutoff Quartile 2 | 0.189 | 0.192 | 0.189 | 0.189 | 0.192 |
| Sensitivity | 64% | 69% | 49% | 64% | 67% |
| Specificity | 16% | 20% | 17% | 17% | 20% |
| Cutoff Quartile 3 | 0.366 | 0.368 | 0.366 | 0.366 | 0.368 |
| Sensitivity | 40% | 42% | 31% | 38% | 40% |
| Specificity | 41% | 44% | 44% | 42% | 43% |
| Cutoff Quartile 4 | 0.857 | 0.862 | 0.857 | 0.857 | 0.862 |
| Sensitivity | 20% | 21% | 13% | 18% | 19% |
| Specificity | 70% | 72% | 71% | 70% | 71% |
| OR Quartile 2 | 0.345 | 0.545 | 0.204 | 0.374 | 0.506 |
| p Value | 4.9E−5 | 0.018 | 1.5E−8 | 1.4E−4 | 0.0079 |
| Lower limit of 95% CI | 0.207 | 0.331 | 0.118 | 0.226 | 0.306 |
| Upper limit of 95% CI | 0.577 | 0.900 | 0.354 | 0.621 | 0.836 |
| OR Quartile 3 | 0.466 | 0.575 | 0.359 | 0.444 | 0.509 |
| p Value | 6.2E−4 | 0.013 | 2.1E−4 | 3.6E−4 | 0.0032 |
| Lower limit of 95% CI | 0.301 | 0.371 | 0.209 | 0.285 | 0.326 |
| Upper limit of 95% CI | 0.721 | 0.891 | 0.617 | 0.694 | 0.797 |
| OR Quartile 4 | 0.591 | 0.665 | 0.368 | 0.529 | 0.565 |
| p Value | 0.042 | 0.12 | 0.0063 | 0.018 | 0.037 |
| Lower limit of 95% CI | 0.355 | 0.398 | 0.180 | 0.312 | 0.331 |
| Upper limit of 95% CI | 0.982 | | 0.754 | 0.898 | 0.966 |
| Serum Creatinine | | | | | |
| AUC | 0.80 | 0.82 | 0.70 | 0.80 | 0.81 |
| SE | 0.024 | 0.024 | 0.034 | 0.025 | 0.024 |
| p Value | 0 | 0 | 1.7E−9 | 0 | 0 |
| nCohort Non-persistent | 188 | 197 | 262 | 203 | 209 |
| nCohort Persistent | 166 | 155 | 90 | 151 | 143 |
| Cutoff Quartile 2 | 1.64 | 1.64 | 1.65 | 1.64 | 1.64 |
| Sensitivity | 92% | 93% | 91% | 93% | 94% |
| Specificity | 40% | 39% | 31% | 39% | 38% |
| Cutoff Quartile 3 | 2.44 | 2.44 | 2.44 | 2.44 | 2.44 |
| Sensitivity | 73% | 75% | 69% | 75% | 77% |
| Specificity | 70% | 70% | 56% | 68% | 68% |
| Cutoff Quartile 4 | 3.32 | 3.31 | 3.32 | 3.32 | 3.31 |
| Sensitivity | 45% | 47% | 47% | 46% | 48% |
| Specificity | 92% | 92% | 82% | 90% | 90% |
| OR Quartile 2 | 7.21 | 8.40 | 4.51 | 8.98 | 10.5 |
| p Value | 4.4E−10 | 7.1E−10 | 1.3E−4 | 8.2E−10 | 1.9E−9 |
| Lower limit of 95% CI | 3.87 | 4.27 | 2.08 | 4.46 | 4.87 |
| Upper limit of 95% CI | 13.4 | 16.5 | 9.75 | 18.1 | 22.5 |
| OR Quartile 3 | 6.54 | 7.20 | 2.83 | 6.69 | 7.22 |
| p Value | 2.7E−15 | 4.4E−16 | 6.1E−5 | 4.2E−15 | 1.6E−15 |
| Lower limit of 95% CI | 4.10 | 4.47 | 1.70 | 4.16 | 4.44 |
| Upper limit of 95% CI | 10.4 | 11.6 | 4.71 | 10.8 | 11.7 |
| OR Quartile 4 | 9.51 | 10.8 | 4.00 | 7.91 | 8.57 |
| p Value | 4.5E−13 | 3.0E−14 | 1.8E−7 | 5.3E−13 | 1.0E−13 |
| Lower limit of 95% CI | 5.17 | 5.85 | 2.38 | 4.51 | 4.87 |
| Upper limit of 95% CI | 17.5 | 20.0 | 6.74 | 13.9 | 15.1 |
| Cystatin-C | | | | | |
| AUC | 0.76 | 0.75 | 0.74 | 0.75 | 0.76 |
| SE | 0.028 | 0.028 | 0.037 | 0.029 | 0.029 |
| p Value | 0 | 0 | 4.7E−11 | 0 | 0 |
| nCohort Non-persistent | 167 | 174 | 237 | 181 | 185 |
| nCohort Persistent | 141 | 133 | 69 | 127 | 122 |
| Cutoff Quartile 2 | 1170000 | 1170000 | 1170000 | 1170000 | 1170000 |
| Sensitivity | 91% | 90% | 93% | 91% | 90% |
| Specificity | 38% | 36% | 30% | 35% | 35% |
| Cutoff Quartile 3 | 1660000 | 1650000 | 1660000 | 1660000 | 1650000 |
| Sensitivity | 72% | 73% | 78% | 72% | 73% |
| Specificity | 69% | 67% | 58% | 65% | 65% |
| Cutoff Quartile 4 | 2150000 | 2150000 | 2150000 | 2150000 | 2150000 |
| Sensitivity | 40% | 41% | 49% | 42% | 43% |
| Specificity | 87% | 87% | 82% | 87% | 87% |
| OR Quartile 2 | 5.96 | 5.24 | 5.59 | 5.24 | 4.85 |
| p Value | 7.5E−8 | 6.0E−7 | 3.9E−4 | 1.2E−6 | 3.7E−6 |
| Lower limit of 95% CI | 3.11 | 2.73 | 2.16 | 2.69 | 2.48 |
| Upper limit of 95% CI | 11.4 | 10.0 | 14.5 | 10.2 | 9.46 |
| OR Quartile 3 | 5.78 | 5.53 | 5.02 | 4.73 | 4.98 |
| p Value | 3.1E−12 | 1.5E−11 | 4.7E−7 | 6.0E−10 | 3.3E−10 |
| Lower limit of 95% CI | 3.53 | 3.37 | 2.68 | 2.89 | 3.02 |
| Upper limit of 95% CI | 9.47 | 9.09 | 9.40 | 7.75 | 8.21 |
| OR Quartile 4 | 4.58 | 4.87 | 4.38 | 4.69 | 5.15 |
| p Value | 1.5E−7 | 3.9E−8 | 5.0E−7 | 5.1E−8 | 8.8E−9 |
| Lower limit of 95% CI | 2.59 | 2.77 | 2.46 | 2.69 | 2.95 |
| Upper limit of 95% CI | 8.09 | 8.57 | 7.80 | 8.17 | 9.01 |
| C-C motif chemokine 14/(Weight | | | | | |
| adjusted urine output) | | | | | |
| AUC | 0.78 | 0.75 | 0.77 | 0.80 | 0.78 |
| SE | 0.026 | 0.028 | 0.035 | 0.026 | 0.027 |
| p Value | 0 | 0 | 1.6E−14 | 0 | 0 |
| nCohort Non-persistent | 181 | 190 | 254 | 196 | 202 |
| nCohort Persistent | 147 | 136 | 74 | 132 | 124 |
| Cutoff Quartile 2 | 1.26 | 1.24 | 1.26 | 1.26 | 1.24 |
| Sensitivity | 88% | 88% | 92% | 91% | 91% |
| Specificity | 35% | 34% | 30% | 36% | 35% |
| Cutoff Quartile 3 | 4.59 | 4.54 | 4.59 | 4.59 | 4.54 |
| Sensitivity | 74% | 73% | 80% | 77% | 77% |
| Specificity | 70% | 66% | 59% | 68% | 66% |
| Cutoff Quartile 4 | 24.3 | 22.9 | 24.3 | 24.3 | 22.9 |
| Sensitivity | 46% | 45% | 54% | 48% | 48% |
| Specificity | 92% | 89% | 83% | 91% | 89% |
| OR Quartile 2 | 3.92 | 3.64 | 4.84 | 5.56 | 5.57 |
| p Value | 3.9E−6 | 1.8E−5 | 4.2E−4 | 3.7E−7 | 8.4E−7 |
| Lower limit of 95% CI | 2.20 | 2.02 | 2.01 | 2.87 | 2.81 |
| Upper limit of 95% CI | 7.00 | 6.57 | 11.6 | 10.8 | 11.0 |
| OR Quartile 3 | 6.57 | 5.27 | 5.58 | 7.35 | 6.46 |
| p Value | 3.3E−14 | 1.5E−11 | 5.3E−8 | 1.2E−14 | 6.2E−13 |
| Lower limit of 95% CI | 4.04 | 3.25 | 3.00 | 4.43 | 3.88 |
| Upper limit of 95% CI | 10.7 | 8.54 | 10.4 | 12.2 | 10.7 |
| OR Quartile 4 | 9.27 | 6.55 | 5.94 | 9.31 | 7.67 |
| p Value | 2.0E−12 | 7.5E−11 | 6.2E−10 | 1.6E−13 | 1.7E−12 |
| Lower limit of 95% CI | 4.99 | 3.72 | 3.38 | 5.14 | 4.36 |
| Upper limit of 95% CI | 17.2 | 11.5 | 10.4 | 16.8 | 13.5 |
| C-C motif chemokine | | | | | |
| 14 X Serum Creatinine | | | | | |
| AUC | 0.83 | 0.83 | 0.74 | 0.85 | 0.85 |
| SE | 0.024 | 0.024 | 0.036 | 0.023 | 0.023 |
| p Value | 0 | 0 | 7.5E−12 | 0 | 0 |
| nCohort Non-persistent | 177 | 186 | 248 | 192 | 198 |
| nCohort Persistent | 147 | 136 | 74 | 132 | 124 |
| Cutoff Quartile 2 | 1.29 | 1.29 | 1.30 | 1.29 | 1.29 |
| Sensitivity | 91% | 92% | 91% | 94% | 94% |
| Specificity | 38% | 38% | 30% | 38% | 37% |
| Cutoff Quartile 3 | 3.97 | 3.91 | 3.97 | 3.97 | 3.91 |
| Sensitivity | 80% | 81% | 84% | 84% | 85% |
| Specificity | 75% | 73% | 60% | 73% | 72% |
| Cutoff Quartile 4 | 18.3 | 16.8 | 18.0 | 18.3 | 16.8 |
| Sensitivity | 46% | 48% | 49% | 52% | 52% |
| Specificity | 93% | 91% | 82% | 93% | 92% |
| OR Quartile 2 | 6.43 | 6.86 | 4.07 | 9.51 | 9.97 |
| p Value | 1.5E−8 | 3.5E−8 | 8.5E−4 | 1.1E−8 | 3.2E−8 |
| Lower limit of 95% CI | 3.37 | 3.46 | 1.78 | 4.39 | 4.41 |
| Upper limit of 95% CI | 12.3 | 13.6 | 9.29 | 20.6 | 22.5 |
| OR Quartile 3 | 11.4 | 11.2 | 7.78 | 14.6 | 14.0 |
| p Value | 0 | 0 | 1.8E−9 | 0 | 0 |
| Lower limit of 95% CI | 6.77 | 6.56 | 3.99 | 8.30 | 7.86 |
| Upper limit of 95% CI | 19.3 | 19.1 | 15.2 | 25.7 | 25.0 |
| OR Quartile 4 | 10.9 | 9.73 | 4.27 | 14.6 | 12.5 |
| p Value | 7.1E−13 | 3.5E−13 | 3.5E−7 | 1.3E−15 | 1.3E−15 |
| Lower limit of 95% CI | 5.66 | 5.27 | 2.44 | 7.57 | 6.74 |
| Upper limit of 95% CI | 20.8 | 18.0 | 7.47 | 28.3 | 23.3 |
| C-C motif chemokine | | | | | |
| 14 X Cystatin-C | | | | | |
| AUC | 0.82 | 0.82 | 0.74 | 0.84 | 0.84 |
| SE | 0.025 | 0.025 | 0.037 | 0.025 | 0.025 |
| p Value | 0 | 0 | 5.4E−11 | 0 | 0 |
| nCohort Non-persistent | 167 | 174 | 237 | 181 | 185 |
| nCohort Persistent | 141 | 133 | 69 | 127 | 122 |
| Cutoff Quartile 2 | 956000 | 955000 | 958000 | 956000 | 955000 |
| Sensitivity | 91% | 91% | 91% | 92% | 93% |
| Specificity | 38% | 37% | 30% | 37% | 37% |
| Cutoff Quartile 3 | 2880000 | 2860000 | 2880000 | 2880000 | 2860000 |
| Sensitivity | 79% | 80% | 83% | 82% | 83% |
| Specificity | 75% | 73% | 59% | 72% | 71% |
| Cutoff Quartile 4 | 1.04E7 | 1.03E7 | 1.03E7 | 1.04E7 | 1.03E7 |
| Sensitivity | 45% | 47% | 51% | 50% | 52% |
| Specificity | 92% | 92% | 82% | 93% | 92% |
| OR Quartile 2 | 6.12 | 6.01 | 4.49 | 6.88 | 7.30 |
| p Value | 4.8E−8 | 1.4E−7 | 8.5E−4 | 1.1E−7 | 1.5E−7 |
| Lower limit of 95% CI | 3.19 | 3.08 | 1.86 | 3.37 | 3.48 |
| Upper limit of 95% CI | 11.7 | 11.7 | 10.9 | 14.0 | 15.3 |
| OR Quartile 3 | 11.5 | 11.1 | 6.98 | 11.8 | 12.0 |
| p Value | 0 | 0 | 1.6E−8 | 0 | 0 |
| Lower limit of 95% CI | 6.71 | 6.46 | 3.55 | 6.79 | 6.79 |
| Upper limit of 95% CI | 19.7 | 19.2 | 13.7 | 20.7 | 21.1 |
| OR Quartile 4 | 9.85 | 10.3 | 4.78 | 13.1 | 13.0 |
| p Value | 8.3E−12 | 1.3E−12 | 1.1E−7 | 2.7E−14 | 1.0E−14 |
| Lower limit of 95% CI | 5.11 | 5.40 | 2.68 | 6.77 | 6.81 |
| Upper limit of 95% CI | 19.0 | 19.6 | 8.52 | 25.5 | 25.0 |
| C-C motif chemokine 14 X | | | | | |
| Serum Creatinine/(Weight | | | | | |
| adjusted urine output) | | | | | |
| AUC | 0.81 | 0.79 | 0.79 | 0.84 | 0.82 |
| SE | 0.025 | 0.026 | 0.034 | 0.024 | 0.025 |
| p Value | 0 | 0 | 0 | 0 | 0 |
| nCohort Non-persistent | 175 | 184 | 247 | 190 | 196 |
| nCohort Persistent | 146 | 135 | 74 | 131 | 123 |
| Cutoff Quartile 2 | 2.17 | 2.15 | 2.17 | 2.17 | 2.15 |
| Sensitivity | 90% | 90% | 92% | 92% | 92% |
| Specificity | 37% | 36% | 30% | 36% | 36% |
| Cutoff Quartile 3 | 10.2 | 10.1 | 10.2 | 10.2 | 10.1 |
| Sensitivity | 78% | 77% | 84% | 81% | 80% |
| Specificity | 73% | 70% | 60% | 71% | 69% |
| Cutoff Quartile 4 | 69.8 | 67.6 | 69.8 | 69.8 | 67.6 |
| Sensitivity | 48% | 47% | 53% | 52% | 50% |
| Specificity | 94% | 91% | 83% | 93% | 91% |
| OR Quartile 2 | 5.16 | 4.83 | 4.85 | 6.22 | 6.28 |
| p Value | 1.8E−7 | 9.5E−7 | 4.3E−4 | 1.7E−7 | 3.9E−7 |
| Lower limit of 95% CI | 2.79 | 2.57 | 2.01 | 3.14 | 3.09 |
| Upper limit of 95% CI | 9.55 | 9.08 | 11.7 | 12.3 | 12.8 |
| OR Quartile 3 | 9.70 | 7.67 | 7.72 | 10.4 | 9.13 |
| p Value | 0 | 4.7E−15 | 2.0E−9 | 0 | 8.9E−16 |
| Lower limit of 95% CI | 5.80 | 4.61 | 3.96 | 6.08 | 5.33 |
| Upper limit of 95% CI | 16.2 | 12.8 | 15.1 | 17.8 | 15.6 |
| OR Quartile 4 | 13.7 | 8.60 | 5.44 | 14.7 | 10.1 |
| p Value | 1.1E−13 | 2.6E−12 | 4.1E−9 | 1.3E−15 | 4.7E−14 |
| Lower limit of 95% CI | 6.88 | 4.70 | 3.09 | 7.60 | 5.52 |
| Upper limit of 95% CI | 27.4 | 15.7 | 9.56 | 28.4 | 18.3 |
| C-C motif chemokine 14 X | | | | | |
| Cystatin-C/(Weight | | | | | |
| adjusted urine output) | | | | | |
| AUC | 0.80 | 0.78 | 0.77 | 0.81 | 0.80 |
| SE | 0.026 | 0.027 | 0.036 | 0.026 | 0.027 |
| p Value | 0 | 0 | 9.8E−14 | 0 | 0 |
| nCohort Non-persistent | 165 | 172 | 236 | 179 | 183 |
| nCohort Persistent | 140 | 132 | 69 | 126 | 121 |
| Cutoff Quartile 2 | 1580000 | 1580000 | 1580000 | 1580000 | 1580000 |
| Sensitivity | 89% | 89% | 91% | 91% | 92% |
| Specificity | 36% | 35% | 30% | 36% | 36% |
| Cutoff Quartile 3 | 6710000 | 6570000 | 6710000 | 6710000 | 6570000 |
| Sensitivity | 76% | 76% | 83% | 79% | 79% |
| Specificity | 72% | 70% | 59% | 70% | 69% |
| Cutoff Quartile 4 | 3.85E7 | 3.84E7 | 3.85E7 | 3.85E7 | 3.84E7 |
| Sensitivity | 45% | 44% | 49% | 48% | 47% |
| Specificity | 92% | 90% | 82% | 91% | 90% |
| OR Quartile 2 | 4.43 | 4.29 | 4.43 | 5.96 | 6.26 |
| p Value | 1.7E−6 | 4.5E−6 | 9.5E−4 | 3.9E−7 | 4.8E−7 |
| Lower limit of 95% CI | 2.41 | 2.30 | 1.83 | 2.99 | 3.07 |
| Upper limit of 95% CI | 8.15 | 7.98 | 10.7 | 11.9 | 12.8 |
| OR Quartile 3 | 8.39 | 7.21 | 6.93 | 9.14 | 8.71 |
| p Value | 8.9E−16 | 5.0E−14 | 1.9E−8 | 6.7E−16 | 4.4E−15 |
| Lower limit of 95% CI | 5.00 | 4.31 | 3.53 | 5.34 | 5.07 |
| Upper limit of 95% CI | 14.1 | 12.1 | 13.6 | 15.7 | 15.0 |
| OR Quartile 4 | 8.82 | 6.71 | 4.36 | 8.66 | 7.69 |
| p Value | 2.7E−11 | 4.2E−10 | 5.5E−7 | 3.9E−12 | 1.7E−11 |
| Lower limit of 95% CI | 4.65 | 3.69 | 2.45 | 4.71 | 4.24 |
| Upper limit of 95% CI | 16.8 | 12.2 | 7.76 | 15.9 | 13.9 |

| | Persistence duration (hr) | | | |
|---|---|---|---|---|
| | | 72 | | |
| | 48 | sCr or | | |
| | UO only | UO | sCr only | UO only |
| C-C motif chemokine 14 | | | | |
| AUC | 0.74 | 0.80 | 0.80 | 0.74 |
| SE | 0.037 | 0.027 | 0.027 | 0.037 |
| p Value | 5.4E−11 | 0 | 0 | 5.4E−11 |
| nCohort Non-persistent | 260 | 210 | 216 | 260 |
| nCohort Persistent | 69 | 121 | 113 | 69 |
| Cutoff Quartile 2 | 0.732 | 0.725 | 0.719 | 0.732 |
| Sensitivity | 93% | 93% | 93% | 93% |
| Specificity | 30% | 35% | 34% | 30% |
| Cutoff Quartile 3 | 1.64 | 1.64 | 1.64 | 1.64 |
| Sensitivity | 83% | 82% | 82% | 83% |
| Specificity | 58% | 68% | 67% | 58% |
| Cutoff Quartile 4 | 5.09 | 5.16 | 5.09 | 5.09 |
| Sensitivity | 46% | 48% | 49% | 46% |
| Specificity | 80% | 88% | 87% | 80% |
| OR Quartile 2 | 5.39 | 6.77 | 6.84 | 5.39 |
| p Value | 5.0E−4 | 3.5E−7 | 1.0E−6 | 5.0E−4 |
| Lower limit of 95% CI | 2.09 | 3.24 | 3.16 | 2.09 |
| Upper limit of 95% CI | 13.9 | 14.1 | 14.8 | 13.9 |
| OR Quartile 3 | 6.69 | 9.60 | 9.30 | 6.69 |
| p Value | 2.7E−8 | 4.4E−16 | 5.8E−15 | 2.7E−8 |
| Lower limit of 95% CI | 3.42 | 5.57 | 5.31 | 3.42 |
| Upper limit of 95% CI | 13.1 | 16.6 | 16.3 | 13.1 |
| OR Quartile 4 | 3.54 | 6.81 | 6.37 | 3.54 |
| p Value | 1.1E−5 | 7.5E−12 | 2.2E−11 | 1.1E−5 |
| Lower limit of 95% CI | 2.02 | 3.93 | 3.70 | 2.02 |
| Upper limit of 95% CI | 6.23 | 11.8 | 10.9 | 6.23 |
| Weight adjusted urine output | | | | |
| AUC | 0.31 | 0.35 | 0.38 | 0.31 |
| SE | 0.038 | 0.032 | 0.033 | 0.038 |
| p Value | 7.7E−7 | 1.3E−6 | 1.5E−4 | 7.7E−7 |
| nCohort Non-persistent | 265 | 214 | 220 | 265 |
| nCohort Persistent | 72 | 123 | 115 | 72 |
| Cutoff Quartile 2 | 0.189 | 0.189 | 0.192 | 0.189 |
| Sensitivity | 51% | 63% | 66% | 51% |
| Specificity | 18% | 18% | 20% | 18% |
| Cutoff Quartile 3 | 0.366 | 0.366 | 0.368 | 0.366 |
| Sensitivity | 33% | 37% | 39% | 33% |
| Specificity | 45% | 43% | 44% | 45% |
| Cutoff Quartile 4 | 0.857 | 0.857 | 0.862 | 0.857 |
| Sensitivity | 14% | 18% | 18% | 14% |
| Specificity | 72% | 71% | 71% | 72% |
| OR Quartile 2 | 0.240 | 0.361 | 0.487 | 0.240 |
| p Value | 4.9E−7 | 8.1E−5 | 0.0055 | 4.9E−7 |
| Lower limit of 95% CI | 0.137 | 0.218 | 0.293 | 0.137 |
| Upper limit of 95% CI | 0.418 | 0.600 | 0.810 | 0.418 |
| OR Quartile 3 | 0.414 | 0.442 | 0.507 | 0.414 |
| p Value | 0.0016 | 4.3E−4 | 0.0038 | 0.0016 |
| Lower limit of 95% CI | 0.240 | 0.280 | 0.320 | 0.240 |
| Upper limit of 95% CI | 0.715 | 0.697 | 0.803 | 0.715 |
| OR Quartile 4 | 0.409 | 0.522 | 0.557 | 0.409 |
| p Value | 0.015 | 0.020 | 0.039 | 0.015 |
| Lower limit of 95% CI | 0.199 | 0.302 | 0.319 | 0.199 |
| Upper limit of 95% CI | 0.839 | 0.902 | 0.971 | 0.839 |
| Serum Creatinine | | | | |
| AUC | 0.72 | 0.79 | 0.81 | 0.72 |
| SE | 0.034 | 0.026 | 0.025 | 0.034 |
| p Value | 6.0E−11 | 0 | 0 | 2.5E−10 |
| nCohort Non-persistent | 266 | 216 | 222 | 267 |
| nCohort Persistent | 86 | 138 | 130 | 85 |
| Cutoff Quartile 2 | 1.65 | 1.64 | 1.64 | 1.65 |
| Sensitivity | 93% | 93% | 95% | 93% |
| Specificity | 31% | 37% | 36% | 31% |
| Cutoff Quartile 3 | 2.44 | 2.44 | 2.44 | 2.44 |
| Sensitivity | 71% | 77% | 78% | 71% |
| Specificity | 56% | 67% | 67% | 56% |
| Cutoff Quartile 4 | 3.32 | 3.32 | 3.31 | 3.32 |
| Sensitivity | 49% | 49% | 50% | 48% |
| Specificity | 82% | 89% | 90% | 82% |
| OR Quartile 2 | 5.94 | 8.43 | 10.1 | 5.84 |
| p Value | 5.9E−5 | 1.0E−8 | 2.1E−8 | 7.0E−5 |
| Lower limit of 95% CI | 2.49 | 4.06 | 4.49 | 2.45 |
| Upper limit of 95% CI | 14.2 | 17.5 | 22.7 | 13.9 |
| OR Quartile 3 | 3.16 | 6.63 | 7.29 | 3.08 |
| p Value | 1.8E−5 | 2.5E−14 | 9.8E−15 | 2.8E−5 |
| Lower limit of 95% CI | 1.87 | 4.07 | 4.41 | 1.82 |
| Upper limit of 95% CI | 5.33 | 10.8 | 12.0 | 5.20 |
| OR Quartile 4 | 4.45 | 7.92 | 8.65 | 4.25 |
| p Value | 2.9E−8 | 1.1E−13 | 1.8E−14 | 7.7E−8 |
| Lower limit of 95% CI | 2.63 | 4.59 | 4.98 | 2.51 |
| Upper limit of 95% CI | 7.54 | 13.7 | 15.0 | 7.21 |
| Cystatin-C | | | | |
| AUC | 0.74 | 0.75 | 0.75 | 0.74 |
| SE | 0.038 | 0.030 | 0.030 | 0.038 |
| p Value | 1.3E−10 | 2.2E−16 | 2.2E−16 | 3.4E−10 |
| nCohort Non-persistent | 241 | 193 | 197 | 242 |
| nCohort Persistent | 65 | 115 | 110 | 64 |
| Cutoff Quartile 2 | 1170000 | 1170000 | 1170000 | 1170000 |
| Sensitivity | 92% | 90% | 90% | 92% |
| Specificity | 30% | 34% | 33% | 30% |
| Cutoff Quartile 3 | 1660000 | 1660000 | 1650000 | 1660000 |
| Sensitivity | 78% | 73% | 75% | 78% |
| Specificity | 58% | 64% | 63% | 57% |
| Cutoff Quartile 4 | 2150000 | 2150000 | 2150000 | 2150000 |
| Sensitivity | 51% | 43% | 45% | 50% |
| Specificity | 82% | 85% | 86% | 81% |
| OR Quartile 2 | 5.11 | 4.80 | 4.43 | 5.00 |
| p Value | 7.9E−4 | 8.2E−6 | 2.4E−5 | 9.4E−4 |
| Lower limit of 95% CI | 1.97 | 2.41 | 2.22 | 1.93 |
| Upper limit of 95% CI | 13.3 | 9.57 | 8.84 | 13.0 |
| OR Quartile 3 | 4.96 | 4.76 | 5.08 | 4.82 |
| p Value | 1.1E−6 | 1.5E−9 | 7.5E−10 | 1.8E−6 |
| Lower limit of 95% CI | 2.61 | 2.87 | 3.03 | 2.53 |
| Upper limit of 95% CI | 9.45 | 7.90 | 8.53 | 9.19 |
| OR Quartile 4 | 4.62 | 4.38 | 4.85 | 4.38 |
| p Value | 3.1E−7 | 1.1E−7 | 1.7E−8 | 8.3E−7 |
| Lower limit of 95% CI | 2.57 | 2.54 | 2.80 | 2.43 |
| Upper limit of 95% CI | 8.30 | 7.55 | 8.39 | 7.88 |
| C-C motif chemokine 14/(Weight | | | | |
| adjusted urine output) | | | | |
| AUC | 0.76 | 0.79 | 0.77 | 0.76 |
| SE | 0.036 | 0.027 | 0.029 | 0.036 |
| p Value | 1.3E−13 | 0 | 0 | 1.3E−13 |
| nCohort Non-persistent | 259 | 208 | 214 | 259 |
| nCohort Persistent | 69 | 120 | 112 | 69 |
| Cutoff Quartile 2 | 1.26 | 1.26 | 1.24 | 1.26 |
| Sensitivity | 93% | 92% | 92% | 93% |
| Specificity | 30% | 35% | 34% | 30% |
| Cutoff Quartile 3 | 4.59 | 4.59 | 4.54 | 4.59 |
| Sensitivity | 80% | 78% | 78% | 80% |
| Specificity | 58% | 66% | 64% | 58% |
| Cutoff Quartile 4 | 24.3 | 24.3 | 22.9 | 24.3 |
| Sensitivity | 54% | 48% | 48% | 54% |
| Specificity | 83% | 88% | 87% | 83% |
| OR Quartile 2 | 5.42 | 5.82 | 5.93 | 5.42 |
| p Value | 4.8E−4 | 1.1E−6 | 2.3E−6 | 4.8E−4 |
| Lower limit of 95% CI | 2.10 | 2.87 | 2.83 | 2.10 |
| Upper limit of 95% CI | 14.0 | 11.8 | 12.4 | 14.0 |
| OR Quartile 3 | 5.41 | 7.13 | 6.32 | 5.41 |
| p Value | 2.0E−7 | 1.5E−13 | 6.2E−12 | 2.0E−7 |
| Lower limit of 95% CI | 2.86 | 4.23 | 3.74 | 2.86 |
| Upper limit of 95% CI | 10.2 | 12.0 | 10.7 | 10.2 |
| OR Quartile 4 | 5.50 | 7.17 | 6.18 | 5.50 |
| p Value | 5.2E−9 | 3.8E−12 | 4.9E−11 | 5.2E−9 |
| Lower limit of 95% CI | 3.10 | 4.11 | 3.59 | 3.10 |
| Upper limit of 95% CI | 9.74 | 12.5 | 10.6 | 9.74 |
| C-C motif chemokine | | | | |
| 14 X Serum Creatinine | | | | |
| AUC | 0.76 | 0.83 | 0.84 | 0.76 |
| SE | 0.036 | 0.025 | 0.025 | 0.036 |
| p Value | 2.1E−13 | 0 | 0 | 2.1E−13 |
| nCohort Non-persistent | 253 | 204 | 210 | 253 |
| nCohort Persistent | 69 | 120 | 112 | 69 |
| Cutoff Quartile 2 | 1.30 | 1.29 | 1.29 | 1.30 |
| Sensitivity | 93% | 94% | 95% | 93% |
| Specificity | 30% | 36% | 36% | 30% |
| Cutoff Quartile 3 | 3.97 | 3.97 | 3.91 | 3.97 |
| Sensitivity | 87% | 85% | 86% | 87% |
| Specificity | 60% | 71% | 69% | 60% |
| Cutoff Quartile 4 | 18.0 | 18.3 | 16.8 | 18.0 |
| Sensitivity | 51% | 50% | 51% | 51% |
| Specificity | 82% | 90% | 89% | 82% |
| OR Quartile 2 | 5.50 | 9.19 | 9.81 | 5.50 |
| p Value | 4.3E−4 | 9.6E−8 | 2.6E−7 | 4.3E−4 |
| Lower limit of 95% CI | 2.13 | 4.07 | 4.11 | 2.13 |
| Upper limit of 95% CI | 14.2 | 20.8 | 23.4 | 14.2 |
| OR Quartile 3 | 10.0 | 13.6 | 13.4 | 10.0 |
| p Value | 1.3E−9 | 0 | 0 | 1.3E−9 |
| Lower limit of 95% CI | 4.77 | 7.58 | 7.31 | 4.77 |
| Upper limit of 95% CI | 21.1 | 24.4 | 24.5 | 21.1 |
| OR Quartile 4 | 4.63 | 8.71 | 8.03 | 4.63 |
| p Value | 1.3E−7 | 1.8E−13 | 4.4E−13 | 1.3E−7 |
| Lower limit of 95% CI | 2.62 | 4.90 | 4.57 | 2.62 |
| Upper limit of 95% CI | 8.19 | 15.5 | 14.1 | 8.19 |
| C-C motif chemokine | | | | |
| 14 X Cystatin-C | | | | |
| AUC | 0.76 | 0.82 | 0.82 | 0.76 |
| SE | 0.037 | 0.027 | 0.027 | 0.037 |
| p Value | 5.4E−12 | 0 | 0 | 5.4E−12 |
| nCohort Non-persistent | 242 | 193 | 197 | 242 |
| nCohort Persistent | 64 | 115 | 110 | 64 |
| Cutoff Quartile 2 | 958000 | 956000 | 955000 | 958000 |
| Sensitivity | 92% | 92% | 93% | 92% |
| Specificity | 30% | 35% | 35% | 30% |
| Cutoff Quartile 3 | 2880000 | 2880000 | 2860000 | 2880000 |
| Sensitivity | 84% | 83% | 84% | 84% |
| Specificity | 59% | 69% | 69% | 59% |
| Cutoff Quartile 4 | 1.03E7 | 1.04E7 | 1.03E7 | 1.03E7 |
| Sensitivity | 55% | 50% | 52% | 55% |
| Specificity | 83% | 90% | 90% | 83% |
| OR Quartile 2 | 5.00 | 6.41 | 6.87 | 5.00 |
| p Value | 9.4E−4 | 9.2E−7 | 1.2E−6 | 9.4E−4 |
| Lower limit of 95% CI | 1.93 | 3.05 | 3.16 | 1.93 |
| Upper limit of 95% CI | 13.0 | 13.5 | 14.9 | 13.0 |
| OR Quartile 3 | 7.80 | 10.8 | 11.1 | 7.80 |
| p Value | 2.4E−8 | 2.2E−16 | 8.9E−16 | 2.4E−8 |
| Lower limit of 95% CI | 3.79 | 6.09 | 6.18 | 3.79 |
| Upper limit of 95% CI | 16.1 | 19.1 | 20.0 | 16.1 |
| OR Quartile 4 | 5.75 | 9.32 | 9.52 | 5.75 |
| p Value | 7.9E−9 | 2.6E−13 | 1.1E−13 | 7.9E−9 |
| Lower limit of 95% CI | 3.17 | 5.12 | 5.25 | 3.17 |
| Upper limit of 95% CI | 10.4 | 16.9 | 17.3 | 10.4 |
| C-C motif chemokine 14 X | | | | |
| Serum Creatinine/(Weight | | | | |
| adjusted urine output) | | | | |
| AUC | 0.79 | 0.82 | 0.81 | 0.79 |
| SE | 0.034 | 0.026 | 0.027 | 0.034 |
| p Value | 0 | 0 | 0 | 0 |
| nCohort Non-persistent | 252 | 202 | 208 | 252 |
| nCohort Persistent | 69 | 119 | 111 | 69 |
| Cutoff Quartile 2 | 2.17 | 2.17 | 2.15 | 2.17 |
| Sensitivity | 93% | 92% | 93% | 93% |
| Specificity | 30% | 35% | 35% | 30% |
| Cutoff Quartile 3 | 10.2 | 10.2 | 10.1 | 10.2 |
| Sensitivity | 84% | 82% | 82% | 84% |
| Specificity | 59% | 69% | 67% | 59% |
| Cutoff Quartile 4 | 69.8 | 69.8 | 67.6 | 69.8 |
| Sensitivity | 54% | 52% | 50% | 54% |
| Specificity | 83% | 91% | 88% | 83% |
| OR Quartile 2 | 5.42 | 6.62 | 6.82 | 5.42 |
| p Value | 4.8E−4 | 5.2E−7 | 1.2E−6 | 4.8E−4 |
| Lower limit of 95% CI | 2.10 | 3.17 | 3.14 | 2.10 |
| Upper limit of 95% CI | 14.0 | 13.9 | 14.8 | 14.0 |
| OR Quartile 3 | 7.63 | 10.3 | 9.17 | 7.63 |
| p Value | 8.6E−9 | 2.2E−16 | 1.3E−14 | 8.6E−9 |
| Lower limit of 95% CI | 3.82 | 5.90 | 5.22 | 3.82 |
| Upper limit of 95% CI | 15.2 | 18.0 | 16.1 | 15.2 |
| OR Quartile 4 | 5.47 | 10.5 | 7.81 | 5.47 |
| p Value | 6.7E−9 | 8.9E−15 | 1.0E−12 | 6.7E−9 |
| Lower limit of 95% CI | 3.08 | 5.79 | 4.44 | 3.08 |
| Upper limit of 95% CI | 9.71 | 19.0 | 13.7 | 9.71 |
| C-C motif chemokine 14 X | | | | |
| Cystatin-C/(Weight | | | | |
| adjusted urine output) | | | | |
| AUC | 0.76 | 0.80 | 0.79 | 0.76 |
| SE | 0.037 | 0.028 | 0.029 | 0.037 |
| p Value | 1.0E−12 | 0 | 0 | 1.0E−12 |
| nCohort Non-persistent | 241 | 191 | 195 | 241 |
| nCohort Persistent | 64 | 114 | 109 | 64 |
| Cutoff Quartile 2 | 1580000 | 1580000 | 1580000 | 1580000 |
| Sensitivity | 92% | 92% | 93% | 92% |
| Specificity | 29% | 35% | 35% | 29% |
| Cutoff Quartile 3 | 6710000 | 6710000 | 6570000 | 6710000 |
| Sensitivity | 83% | 81% | 81% | 83% |
| Specificity | 59% | 68% | 67% | 59% |
| Cutoff Quartile 4 | 3.85E7 | 3.85E7 | 3.84E7 | 3.85E7 |
| Sensitivity | 48% | 47% | 47% | 48% |
| Specificity | 81% | 88% | 87% | 81% |
| OR Quartile 2 | 4.93 | 6.30 | 6.76 | 4.93 |
| p Value | 0.0010 | 1.2E−6 | 1.5E−6 | 0.0010 |
| Lower limit of 95% CI | 1.90 | 3.00 | 3.11 | 1.90 |
| Upper limit of 95% CI | 12.8 | 13.2 | 14.7 | 12.8 |
| OR Quartile 3 | 6.79 | 8.91 | 8.58 | 6.79 |
| p Value | 7.5E−8 | 1.2E−14 | 6.7E−14 | 7.5E−8 |
| Lower limit of 95% CI | 3.38 | 5.11 | 4.89 | 3.38 |
| Upper limit of 95% CI | 13.7 | 15.5 | 15.0 | 13.7 |
| OR Quartile 4 | 3.98 | 6.57 | 5.98 | 3.98 |
| p Value | 3.8E−6 | 9.6E−11 | 5.1E−10 | 3.8E−6 |
| Lower limit of 95% CI | 2.22 | 3.72 | 3.40 | 2.22 |
| Upper limit of 95% CI | 7.16 | 11.6 | 10.5 | 7.16 |

TABLE 6
Comparison of marker levels and the area under the ROC curve (AUC) in urine samples for the “persistent”
and “non-persistent” cohorts where persistence starts within 96 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| | Persistence duration (hr) | | | | |
|---|---|---|---|---|---|
| | 24 | | | 48 | |
| | sCr or | | | sCr or | |
| | UO | sCr only | UO only | UO | sCr only |
| C-C motif chemokine 14 | | | | | |
| AUC | 0.79 | 0.78 | 0.73 | 0.82 | 0.81 |
| SE | 0.025 | 0.026 | 0.035 | 0.025 | 0.026 |
| p Value | 0 | 0 | 2.2E−11 | 0 | 0 |
| nCohort Non-persistent | 181 | 189 | 248 | 197 | 201 |
| nCohort Persistent | 150 | 140 | 81 | 134 | 128 |
| Cutoff Quartile 2 | 0.725 | 0.719 | 0.732 | 0.725 | 0.719 |
| Sensitivity | 90% | 89% | 93% | 93% | 92% |
| Specificity | 38% | 35% | 31% | 37% | 36% |
| Cutoff Quartile 3 | 1.64 | 1.64 | 1.64 | 1.64 | 1.64 |
| Sensitivity | 76% | 76% | 79% | 81% | 80% |
| Specificity | 71% | 69% | 59% | 71% | 69% |
| Cutoff Quartile 4 | 5.16 | 5.09 | 5.09 | 5.16 | 5.09 |
| Sensitivity | 45% | 46% | 43% | 49% | 49% |
| Specificity | 91% | 90% | 81% | 91% | 90% |
| OR Quartile 2 | 5.42 | 4.58 | 5.52 | 7.30 | 6.59 |
| p Value | 6.4E−8 | 1.2E−6 | 1.3E−4 | 3.4E−8 | 1.7E−7 |
| Lower limit of 95% CI | 2.94 | 2.48 | 2.30 | 3.60 | 3.25 |
| Upper limit of 95% CI | 9.99 | 8.45 | 13.2 | 14.8 | 13.4 |
| OR Quartile 3 | 7.86 | 7.32 | 5.48 | 9.95 | 9.24 |
| p Value | 2.2E−16 | 4.7E−15 | 1.8E−8 | 0 | 2.2E−16 |
| Lower limit of 95% CI | 4.79 | 4.45 | 3.03 | 5.88 | 5.44 |
| Upper limit of 95% CI | 12.9 | 12.0 | 9.90 | 16.9 | 15.7 |
| OR Quartile 4 | 8.32 | 7.53 | 3.17 | 9.37 | 8.77 |
| p Value | 7.1E−12 | 8.2E−12 | 2.9E−5 | 1.2E−13 | 1.7E−13 |
| Lower limit of 95% CI | 4.54 | 4.22 | 1.85 | 5.19 | 4.92 |
| Upper limit of 95% CI | 15.3 | 13.4 | 5.44 | 16.9 | 15.6 |
| Weight adjusted urine output | | | | | |
| AUC | 0.36 | 0.40 | 0.28 | 0.35 | 0.38 |
| SE | 0.030 | 0.031 | 0.034 | 0.031 | 0.032 |
| p Value | 4.4E−6 | 0.0014 | 3.1E−10 | 2.6E−6 | 9.0E−5 |
| nCohort Non-persistent | 184 | 192 | 253 | 200 | 204 |
| nCohort Persistent | 153 | 143 | 84 | 137 | 131 |
| Cutoff Quartile 2 | 0.189 | 0.192 | 0.189 | 0.189 | 0.192 |
| Sensitivity | 64% | 69% | 49% | 64% | 67% |
| Specificity | 16% | 20% | 16% | 18% | 20% |
| Cutoff Quartile 3 | 0.366 | 0.368 | 0.366 | 0.366 | 0.368 |
| Sensitivity | 40% | 42% | 30% | 39% | 40% |
| Specificity | 41% | 44% | 43% | 42% | 43% |
| Cutoff Quartile 4 | 0.857 | 0.862 | 0.857 | 0.857 | 0.862 |
| Sensitivity | 20% | 21% | 13% | 19% | 19% |
| Specificity | 71% | 72% | 71% | 70% | 71% |
| OR Quartile 2 | 0.333 | 0.537 | 0.184 | 0.381 | 0.499 |
| p Value | 3.0E−5 | 0.015 | 1.0E−9 | 1.8E−4 | 0.0067 |
| Lower limit of 95% CI | 0.199 | 0.326 | 0.107 | 0.230 | 0.302 |
| Upper limit of 95% CI | 0.559 | 0.887 | 0.317 | 0.631 | 0.825 |
| OR Quartile 3 | 0.467 | 0.562 | 0.321 | 0.457 | 0.499 |
| p Value | 6.3E−4 | 0.0099 | 2.6E−5 | 5.4E−4 | 0.0023 |
| Lower limit of 95% CI | 0.301 | 0.363 | 0.189 | 0.293 | 0.320 |
| Upper limit of 95% CI | 0.722 | 0.871 | 0.545 | 0.712 | 0.780 |
| OR Quartile 4 | 0.612 | 0.678 | 0.364 | 0.560 | 0.580 |
| p Value | 0.057 | 0.14 | 0.0041 | 0.030 | 0.044 |
| Lower limit of 95% CI | 0.369 | 0.407 | 0.183 | 0.331 | 0.341 |
| Upper limit of 95% CI | 1.01 | 1.13 | 0.726 | 0.945 | 0.985 |
| Serum Creatinine | | | | | |
| AUC | 0.79 | 0.81 | 0.71 | 0.80 | 0.80 |
| SE | 0.024 | 0.024 | 0.033 | 0.025 | 0.025 |
| p Value | 0 | 0 | 1.5E−10 | 0 | 0 |
| nCohort Non-persistent | 185 | 193 | 254 | 201 | 205 |
| nCohort Persistent | 169 | 159 | 98 | 153 | 147 |
| Cutoff Quartile 2 | 1.64 | 1.64 | 1.65 | 1.64 | 1.64 |
| Sensitivity | 91% | 92% | 91% | 93% | 94% |
| Specificity | 40% | 39% | 31% | 39% | 39% |
| Cutoff Quartile 3 | 2.44 | 2.44 | 2.44 | 2.44 | 2.44 |
| Sensitivity | 72% | 74% | 68% | 75% | 76% |
| Specificity | 70% | 70% | 57% | 68% | 68% |
| Cutoff Quartile 4 | 3.32 | 3.31 | 3.32 | 3.32 | 3.31 |
| Sensitivity | 44% | 46% | 47% | 46% | 46% |
| Specificity | 92% | 92% | 83% | 90% | 90% |
| OR Quartile 2 | 6.84 | 7.96 | 4.46 | 9.26 | 9.61 |
| p Value | 5.0E−10 | 5.6E−10 | 6.7E−5 | 4.8E−10 | 1.3E−9 |
| Lower limit of 95% CI | 3.73 | 4.13 | 2.14 | 4.59 | 4.63 |
| Upper limit of 95% CI | 12.5 | 15.3 | 9.31 | 18.7 | 20.0 |
| OR Quartile 3 | 5.98 | 6.70 | 2.83 | 6.26 | 6.64 |
| p Value | 2.5E−14 | 2.2E−15 | 3.5E−5 | 1.9E−14 | 7.5E−15 |
| Lower limit of 95% CI | 3.77 | 4.19 | 1.73 | 3.91 | 4.12 |
| Upper limit of 95% CI | 9.47 | 10.7 | 4.63 | 10.0 | 10.7 |
| OR Quartile 4 | 9.04 | 10.1 | 4.34 | 7.63 | 7.96 |
| p Value | 1.4E−12 | 1.4E−13 | 2.3E−8 | 1.2E−12 | 5.5E−13 |
| Lower limit of 95% CI | 4.92 | 5.46 | 2.59 | 4.36 | 4.53 |
| Upper limit of 95% CI | 16.6 | 18.6 | 7.26 | 13.4 | 14.0 |
| Cystatin-C | | | | | |
| AUC | 0.76 | 0.75 | 0.75 | 0.75 | 0.75 |
| SE | 0.028 | 0.028 | 0.035 | 0.029 | 0.029 |
| p Value | 0 | 0 | 7.6E−13 | 0 | 0 |
| nCohort Non-persistent | 165 | 172 | 230 | 180 | 183 |
| nCohort Persistent | 143 | 135 | 76 | 128 | 124 |
| Cutoff Quartile 2 | 1170000 | 1170000 | 1170000 | 1170000 | 1170000 |
| Sensitivity | 91% | 90% | 93% | 91% | 90% |
| Specificity | 38% | 37% | 31% | 36% | 35% |
| Cutoff Quartile 3 | 1660000 | 1650000 | 1660000 | 1660000 | 1650000 |
| Sensitivity | 73% | 73% | 79% | 72% | 73% |
| Specificity | 70% | 67% | 60% | 66% | 65% |
| Cutoff Quartile 4 | 2150000 | 2150000 | 2150000 | 2150000 | 2150000 |
| Sensitivity | 39% | 41% | 49% | 41% | 43% |
| Specificity | 87% | 87% | 83% | 87% | 87% |
| OR Quartile 2 | 6.18 | 5.42 | 6.47 | 5.33 | 5.02 |
| p Value | 4.2E−8 | 3.5E−7 | 1.1E−4 | 9.1E−7 | 2.2E−6 |
| Lower limit of 95% CI | 3.22 | 2.83 | 2.51 | 2.73 | 2.57 |
| Upper limit of 95% CI | 11.8 | 10.4 | 16.7 | 10.4 | 9.79 |
| OR Quartile 3 | 6.13 | 5.49 | 5.52 | 4.86 | 4.92 |
| p Value | 7.4E−13 | 1.5E−11 | 4.2E−8 | 3.2E−10 | 3.5E−10 |
| Lower limit of 95% CI | 3.74 | 3.35 | 3.00 | 2.97 | 2.99 |
| Upper limit of 95% CI | 10.1 | 9.00 | 10.2 | 7.96 | 8.10 |
| OR Quartile 4 | 4.41 | 4.69 | 4.51 | 4.59 | 4.95 |
| p Value | 3.0E−7 | 7.9E−8 | 1.7E−7 | 7.4E−8 | 1.9E−8 |
| Lower limit of 95% CI | 2.50 | 2.67 | 2.56 | 2.64 | 2.83 |
| Upper limit of 95% CI | 7.79 | 8.24 | 7.92 | 8.00 | 8.64 |
| C-C motif chemokine 14/(Weight | | | | | |
| adjusted urine output) | | | | | |
| AUC | 0.78 | 0.75 | 0.77 | 0.79 | 0.78 |
| SE | 0.026 | 0.028 | 0.033 | 0.026 | 0.027 |
| p Value | 0 | 0 | 0 | 0 | 0 |
| nCohort Non-persistent | 179 | 187 | 247 | 195 | 199 |
| nCohort Persistent | 149 | 139 | 81 | 133 | 127 |
| Cutoff Quartile 2 | 1.26 | 1.24 | 1.26 | 1.26 | 1.24 |
| Sensitivity | 87% | 86% | 91% | 90% | 90% |
| Specificity | 35% | 34% | 30% | 35% | 35% |
| Cutoff Quartile 3 | 4.59 | 4.54 | 4.59 | 4.59 | 4.54 |
| Sensitivity | 74% | 72% | 80% | 77% | 76% |
| Specificity | 70% | 66% | 60% | 68% | 66% |
| Cutoff Quartile 4 | 24.3 | 22.9 | 24.3 | 24.3 | 22.9 |
| Sensitivity | 46% | 45% | 54% | 48% | 48% |
| Specificity | 92% | 89% | 85% | 91% | 89% |
| OR Quartile 2 | 3.72 | 3.21 | 4.61 | 5.05 | 4.65 |
| p Value | 6.6E−6 | 6.3E−5 | 2.6E−4 | 7.9E−7 | 2.8E−6 |
| Lower limit of 95% CI | 2.10 | 1.81 | 2.03 | 2.66 | 2.45 |
| Upper limit of 95% CI | 6.58 | 5.68 | 10.5 | 9.62 | 8.86 |
| OR Quartile 3 | 6.53 | 5.05 | 6.07 | 7.06 | 6.10 |
| p Value | 3.4E−14 | 3.3E−11 | 4.6E−9 | 2.5E−14 | 1.4E−12 |
| Lower limit of 95% CI | 4.02 | 3.13 | 3.32 | 4.27 | 3.70 |
| Upper limit of 95% CI | 10.6 | 8.14 | 11.1 | 11.7 | 10.1 |
| OR Quartile 4 | 9.89 | 6.72 | 6.54 | 9.12 | 7.83 |
| p Value | 1.4E−12 | 6.5E−11 | 4.0E−11 | 2.6E−13 | 1.6E−12 |
| Lower limit of 95% CI | 5.25 | 3.80 | 3.75 | 5.04 | 4.43 |
| Upper limit of 95% CI | 18.6 | 11.9 | 11.4 | 16.5 | 13.9 |
| C-C motif chemokine | | | | | |
| 14 X Cystatin-C | | | | | |
| AUC | 0.82 | 0.81 | 0.75 | 0.83 | 0.83 |
| SE | 0.025 | 0.026 | 0.035 | 0.025 | 0.025 |
| p Value | 0 | 0 | 5.2E−13 | 0 | 0 |
| nCohort Non-persistent | 165 | 172 | 230 | 180 | 183 |
| nCohort Persistent | 143 | 135 | 76 | 128 | 124 |
| Cutoff Quartile 2 | 956000 | 955000 | 958000 | 956000 | 955000 |
| Sensitivity | 91% | 90% | 92% | 92% | 92% |
| Specificity | 39% | 37% | 31% | 37% | 37% |
| Cutoff Quartile 3 | 2880000 | 2860000 | 2880000 | 2880000 | 2860000 |
| Sensitivity | 79% | 79% | 83% | 81% | 81% |
| Specificity | 75% | 73% | 61% | 72% | 71% |
| Cutoff Quartile 4 | 1.04E7 | 1.03E7 | 1.03E7 | 1.04E7 | 1.03E7 |
| Sensitivity | 45% | 47% | 50% | 50% | 51% |
| Specificity | 93% | 92% | 83% | 93% | 92% |
| OR Quartile 2 | 6.34 | 5.56 | 5.21 | 7.00 | 6.58 |
| p Value | 2.6E−8 | 2.3E−7 | 2.3E−4 | 8.8E−8 | 2.2E−7 |
| Lower limit of 95% CI | 3.31 | 2.90 | 2.16 | 3.43 | 3.23 |
| Upper limit of 95% CI | 12.1 | 10.7 | 12.6 | 14.3 | 13.4 |
| OR Quartile 3 | 11.4 | 10.2 | 7.54 | 11.3 | 10.8 |
| p Value | 0 | 0 | 1.3E−9 | 0 | 0 |
| Lower limit of 95% CI | 6.67 | 5.96 | 3.92 | 6.50 | 6.19 |
| Upper limit of 95% CI | 19.5 | 17.3 | 14.5 | 19.5 | 18.7 |
| OR Quartile 4 | 10.6 | 9.88 | 4.90 | 12.8 | 12.5 |
| p Value | 6.1E−12 | 2.9E−12 | 3.8E−8 | 4.2E−14 | 2.5E−14 |
| Lower limit of 95% CI | 5.42 | 5.19 | 2.78 | 6.62 | 6.52 |
| Upper limit of 95% CI | 20.8 | 18.8 | 8.63 | 24.9 | 23.9 |
| C-C motif chemokine | | | | | |
| 14 X Serum Creatinine | | | | | |
| AUC | 0.83 | 0.82 | 0.75 | 0.85 | 0.85 |
| SE | 0.024 | 0.024 | 0.034 | 0.023 | 0.024 |
| p Value | 0 | 0 | 8.9E−14 | 0 | 0 |
| nCohort Non-persistent | 175 | 183 | 241 | 191 | 195 |
| nCohort Persistent | 149 | 139 | 81 | 133 | 127 |
| Cutoff Quartile 2 | 1.29 | 1.29 | 1.30 | 1.29 | 1.29 |
| Sensitivity | 91% | 91% | 91% | 94% | 94% |
| Specificity | 39% | 38% | 31% | 38% | 37% |
| Cutoff Quartile 3 | 3.97 | 3.91 | 3.97 | 3.97 | 3.91 |
| Sensitivity | 79% | 80% | 84% | 83% | 83% |
| Specificity | 75% | 73% | 61% | 73% | 72% |
| Cutoff Quartile 4 | 18.3 | 16.8 | 18.0 | 18.3 | 16.8 |
| Sensitivity | 46% | 47% | 47% | 51% | 52% |
| Specificity | 93% | 92% | 82% | 93% | 92% |
| OR Quartile 2 | 6.65 | 6.41 | 4.68 | 9.67 | 8.90 |
| p Value | 8.6E−9 | 4.0E−8 | 2.3E−4 | 8.5E−9 | 2.9E−8 |
| Lower limit of 95% CI | 3.49 | 3.30 | 2.06 | 4.47 | 4.11 |
| Upper limit of 95% CI | 12.7 | 12.4 | 10.7 | 20.9 | 19.3 |
| OR Quartile 3 | 11.3 | 10.5 | 8.32 | 13.9 | 12.8 |
| p Value | 0 | 0 | 1.4E−10 | 0 | 0 |
| Lower limit of 95% CI | 6.72 | 6.23 | 4.36 | 7.92 | 7.32 |
| Upper limit of 95% CI | 19.1 | 17.9 | 15.9 | 24.2 | 22.6 |
| OR Quartile 4 | 10.5 | 10.1 | 4.07 | 14.3 | 13.0 |
| p Value | 1.5E−12 | 3.7E−13 | 4.9E−7 | 2.2E−15 | 1.8E−15 |
| Lower limit of 95% CI | 5.46 | 5.42 | 2.35 | 7.42 | 6.91 |
| Upper limit of 95% CI | 20.0 | 18.9 | 7.03 | 27.7 | 24.4 |
| C-C motif chemokine 14 X | | | | | |
| Serum Creatinine/(Weight | | | | | |
| adjusted urine output) | | | | | |
| AUC | 0.81 | 0.79 | 0.79 | 0.83 | 0.82 |
| SE | 0.025 | 0.026 | 0.032 | 0.024 | 0.026 |
| p Value | 0 | 0 | 0 | 0 | 0 |
| nCohort Non-persistent | 173 | 181 | 240 | 189 | 193 |
| nCohort Persistent | 148 | 138 | 81 | 132 | 126 |
| Cutoff Quartile 2 | 2.17 | 2.15 | 2.17 | 2.17 | 2.15 |
| Sensitivity | 89% | 88% | 91% | 91% | 90% |
| Specificity | 37% | 35% | 30% | 36% | 35% |
| Cutoff Quartile 3 | 10.2 | 10.1 | 10.2 | 10.2 | 10.1 |
| Sensitivity | 78% | 76% | 84% | 80% | 79% |
| Specificity | 73% | 70% | 61% | 71% | 69% |
| Cutoff Quartile 4 | 69.8 | 67.6 | 69.8 | 69.8 | 67.6 |
| Sensitivity | 48% | 46% | 53% | 52% | 50% |
| Specificity | 94% | 91% | 84% | 93% | 91% |
| OR Quartile 2 | 4.84 | 4.17 | 4.62 | 5.62 | 5.17 |
| p Value | 3.0E−7 | 3.5E−6 | 2.6E−4 | 3.4E−7 | 1.3E−6 |
| Lower limit of 95% CI | 2.65 | 2.28 | 2.03 | 2.89 | 2.66 |
| Upper limit of 95% CI | 8.86 | 7.63 | 10.5 | 10.9 | 10.0 |
| OR Quartile 3 | 9.62 | 7.29 | 8.27 | 9.93 | 8.53 |
| p Value | 0 | 1.0E−14 | 1.6E−10 | 0 | 1.8E−15 |
| Lower limit of 95% CI | 5.76 | 4.41 | 4.33 | 5.84 | 5.03 |
| Upper limit of 95% CI | 16.1 | 12.1 | 15.8 | 16.9 | 14.5 |
| OR Quartile 4 | 15.0 | 8.92 | 6.02 | 14.4 | 10.4 |
| p Value | 1.1E−13 | 2.5E−12 | 2.8E−10 | 2.2E−15 | 4.9E−14 |
| Lower limit of 95% CI | 7.35 | 4.83 | 3.45 | 7.44 | 5.64 |
| Upper limit of 95% CI | 30.7 | 16.5 | 10.5 | 27.8 | 19.0 |
| C-C motif chemokine 14 X | | | | | |
| Cystatin-C/(Weight | | | | | |
| adjusted urine output) | | | | | |
| AUC | 0.80 | 0.77 | 0.78 | 0.81 | 0.80 |
| SE | 0.026 | 0.028 | 0.034 | 0.026 | 0.027 |
| p Value | 0 | 0 | 2.2E−16 | 0 | 0 |
| nCohort Non-persistent | 163 | 170 | 229 | 178 | 181 |
| nCohort Persistent | 142 | 134 | 76 | 127 | 123 |
| Cutoff Quartile 2 | 1580000 | 1580000 | 1580000 | 1580000 | 1580000 |
| Sensitivity | 88% | 87% | 91% | 91% | 90% |
| Specificity | 36% | 35% | 30% | 36% | 35% |
| Cutoff Quartile 3 | 6710000 | 6570000 | 6710000 | 6710000 | 6570000 |
| Sensitivity | 76% | 75% | 83% | 79% | 78% |
| Specificity | 72% | 69% | 61% | 70% | 69% |
| Cutoff Quartile 4 | 3.85E7 | 3.84E7 | 3.85E7 | 3.85E7 | 3.84E7 |
| Sensitivity | 45% | 43% | 50% | 47% | 46% |
| Specificity | 92% | 89% | 83% | 90% | 90% |
| OR Quartile 2 | 4.17 | 3.66 | 4.25 | 5.38 | 5.06 |
| p Value | 3.0E−6 | 2.2E−5 | 6.1E−4 | 8.2E−7 | 2.0E−6 |
| Lower limit of 95% CI | 2.29 | 2.01 | 1.86 | 2.76 | 2.59 |
| Upper limit of 95% CI | 7.59 | 6.66 | 9.72 | 10.5 | 9.88 |
| OR Quartile 3 | 8.33 | 6.67 | 7.48 | 8.74 | 7.94 |
| p Value | 8.9E−16 | 2.4E−13 | 1.6E−9 | 1.6E−15 | 2.0E−14 |
| Lower limit of 95% CI | 4.97 | 4.02 | 3.89 | 5.13 | 4.67 |
| Upper limit of 95% CI | 14.0 | 11.1 | 14.4 | 14.9 | 13.5 |
| OR Quartile 4 | 9.47 | 6.44 | 4.87 | 8.48 | 7.36 |
| p Value | 1.9E−11 | 9.1E−10 | 4.3E−8 | 6.1E−12 | 4.1E−11 |
| Lower limit of 95% CI | 4.91 | 3.55 | 2.76 | 4.61 | 4.07 |
| Upper limit of 95% CI | 18.2 | 11.7 | 8.58 | 15.6 | 13.3 |

| | Persistence duration (hr) | | | |
|---|---|---|---|---|
| | | 72 | | |
| | 48 | sCr or | | |
| | UO only | UO | sCr only | UO only |
| C-C motif chemokine 14 | | | | |
| AUC | 0.74 | 0.81 | 0.80 | 0.74 |
| SE | 0.035 | 0.026 | 0.027 | 0.035 |
| p Value | 4.0E−12 | 0 | 0 | 4.0E−12 |
| nCohort Non-persistent | 254 | 208 | 212 | 254 |
| nCohort Persistent | 75 | 123 | 117 | 75 |
| Cutoff Quartile 2 | 0.732 | 0.725 | 0.719 | 0.732 |
| Sensitivity | 93% | 93% | 92% | 93% |
| Specificity | 30% | 36% | 34% | 30% |
| Cutoff Quartile 3 | 1.64 | 1.64 | 1.64 | 1.64 |
| Sensitivity | 81% | 81% | 81% | 81% |
| Specificity | 59% | 68% | 67% | 59% |
| Cutoff Quartile 4 | 5.09 | 5.16 | 5.09 | 5.09 |
| Sensitivity | 45% | 48% | 49% | 45% |
| Specificity | 81% | 88% | 88% | 81% |
| OR Quartile 2 | 6.09 | 7.00 | 6.30 | 6.09 |
| p Value | 1.8E−4 | 2.2E−7 | 9.7E−7 | 1.8E−4 |
| Lower limit of 95% CI | 2.36 | 3.35 | 3.02 | 2.36 |
| Upper limit of 95% CI | 15.7 | 14.6 | 13.2 | 15.7 |
| OR Quartile 3 | 6.28 | 9.35 | 8.76 | 6.28 |
| p Value | 1.2E−8 | 4.4E−16 | 6.0E−15 | 1.2E−8 |
| Lower limit of 95% CI | 3.34 | 5.46 | 5.08 | 3.34 |
| Upper limit of 95% CI | 11.8 | 16.0 | 15.1 | 11.8 |
| OR Quartile 4 | 3.47 | 7.07 | 6.80 | 3.47 |
| p Value | 9.7E−6 | 4.3E−12 | 6.9E−12 | 9.7E−6 |
| Lower limit of 95% CI | 2.00 | 4.06 | 3.93 | 2.00 |
| Upper limit of 95% CI | 6.02 | 12.3 | 11.8 | 6.02 |
| Weight adjusted urine output | | | | |
| AUC | 0.31 | 0.36 | 0.38 | 0.31 |
| SE | 0.036 | 0.032 | 0.033 | 0.036 |
| p Value | 9.2E−8 | 6.1E−6 | 1.9E−4 | 9.2E−8 |
| nCohort Non-persistent | 259 | 212 | 216 | 259 |
| nCohort Persistent | 78 | 125 | 119 | 78 |
| Cutoff Quartile 2 | 0.189 | 0.189 | 0.192 | 0.189 |
| Sensitivity | 51% | 63% | 66% | 51% |
| Specificity | 18% | 18% | 20% | 18% |
| Cutoff Quartile 3 | 0.366 | 0.366 | 0.368 | 0.366 |
| Sensitivity | 32% | 38% | 39% | 32% |
| Specificity | 44% | 43% | 44% | 44% |
| Cutoff Quartile 4 | 0.857 | 0.857 | 0.862 | 0.857 |
| Sensitivity | 14% | 19% | 19% | 14% |
| Specificity | 71% | 71% | 72% | 71% |
| OR Quartile 2 | 0.227 | 0.375 | 0.491 | 0.227 |
| p Value | 1.1E−7 | 1.4E−4 | 0.0059 | 1.1E−7 |
| Lower limit of 95% CI | 0.132 | 0.226 | 0.296 | 0.132 |
| Upper limit of 95% CI | 0.393 | 0.622 | 0.814 | 0.393 |
| OR Quartile 3 | 0.377 | 0.469 | 0.513 | 0.377 |
| p Value | 3.5E−4 | 0.0010 | 0.0040 | 3.5E−4 |
| Lower limit of 95% CI | 0.221 | 0.298 | 0.325 | 0.221 |
| Upper limit of 95% CI | 0.643 | 0.736 | 0.808 | 0.643 |
| OR Quartile 4 | 0.410 | 0.588 | 0.609 | 0.410 |
| p Value | 0.012 | 0.052 | 0.073 | 0.012 |
| Lower limit of 95% CI | 0.205 | 0.344 | 0.354 | 0.205 |
| Upper limit of 95% CI | 0.820 | 1.00 | 1.05 | 0.820 |
| Serum Creatinine | | | | |
| AUC | 0.73 | 0.79 | 0.80 | 0.73 |
| SE | 0.032 | 0.026 | 0.026 | 0.033 |
| p Value | 9.6E−13 | 0 | 0 | 4.7E−12 |
| nCohort Non-persistent | 259 | 213 | 217 | 260 |
| nCohort Persistent | 93 | 141 | 135 | 92 |
| Cutoff Quartile 2 | 1.65 | 1.64 | 1.64 | 1.65 |
| Sensitivity | 94% | 94% | 94% | 93% |
| Specificity | 32% | 38% | 37% | 32% |
| Cutoff Quartile 3 | 2.44 | 2.44 | 2.44 | 2.44 |
| Sensitivity | 71% | 75% | 76% | 71% |
| Specificity | 57% | 66% | 66% | 57% |
| Cutoff Quartile 4 | 3.32 | 3.32 | 3.31 | 3.32 |
| Sensitivity | 49% | 48% | 48% | 49% |
| Specificity | 83% | 89% | 89% | 83% |
| OR Quartile 2 | 6.72 | 8.82 | 9.27 | 6.60 |
| p Value | 1.7E−5 | 5.0E−9 | 1.2E−8 | 2.0E−5 |
| Lower limit of 95% CI | 2.82 | 4.25 | 4.31 | 2.77 |
| Upper limit of 95% CI | 16.0 | 18.3 | 19.9 | 15.7 |
| OR Quartile 3 | 3.26 | 5.93 | 6.35 | 3.18 |
| p Value | 5.8E−6 | 2.3E−13 | 9.5E−14 | 9.2E−6 |
| Lower limit of 95% CI | 1.96 | 3.68 | 3.90 | 1.91 |
| Upper limit of 95% CI | 5.43 | 9.55 | 10.3 | 5.31 |
| OR Quartile 4 | 4.92 | 7.48 | 7.83 | 4.70 |
| p Value | 2.2E−9 | 4.4E−13 | 1.9E−13 | 6.1E−9 |
| Lower limit of 95% CI | 2.92 | 4.34 | 4.53 | 2.79 |
| Upper limit of 95% CI | 8.29 | 12.9 | 13.6 | 7.92 |
| Cystatin-C | | | | |
| AUC | 0.75 | 0.75 | 0.75 | 0.75 |
| SE | 0.036 | 0.030 | 0.030 | 0.036 |
| p Value | 5.5E−12 | 0 | 0 | 1.6E−11 |
| nCohort Non-persistent | 235 | 191 | 194 | 236 |
| nCohort Persistent | 71 | 117 | 113 | 70 |
| Cutoff Quartile 2 | 1170000 | 1170000 | 1170000 | 1170000 |
| Sensitivity | 93% | 91% | 90% | 93% |
| Specificity | 31% | 34% | 34% | 31% |
| Cutoff Quartile 3 | 1660000 | 1660000 | 1650000 | 1660000 |
| Sensitivity | 79% | 74% | 74% | 79% |
| Specificity | 59% | 64% | 64% | 58% |
| Cutoff Quartile 4 | 2150000 | 2150000 | 2150000 | 2150000 |
| Sensitivity | 51% | 43% | 44% | 50% |
| Specificity | 83% | 86% | 86% | 82% |
| OR Quartile 2 | 5.83 | 4.97 | 4.67 | 5.71 |
| p Value | 2.8E−4 | 5.1E−6 | 1.2E−5 | 3.3E−4 |
| Lower limit of 95% CI | 2.25 | 2.50 | 2.34 | 2.21 |
| Upper limit of 95% CI | 15.1 | 9.90 | 9.31 | 14.8 |
| OR Quartile 3 | 5.31 | 5.02 | 5.13 | 5.16 |
| p Value | 1.7E−7 | 4.2E−10 | 4.4E−10 | 2.9E−7 |
| Lower limit of 95% CI | 2.84 | 3.02 | 3.07 | 2.76 |
| Upper limit of 95% CI | 9.93 | 8.33 | 8.58 | 9.66 |
| OR Quartile 4 | 4.87 | 4.53 | 4.91 | 4.62 |
| p Value | 6.7E−8 | 6.3E−8 | 1.5E−8 | 1.8E−7 |
| Lower limit of 95% CI | 2.74 | 2.62 | 2.83 | 2.60 |
| Upper limit of 95% CI | 8.64 | 7.84 | 8.51 | 8.21 |
| C-C motif chemokine 14/(Weight | | | | |
| adjusted urine output) | | | | |
| AUC | 0.77 | 0.79 | 0.77 | 0.77 |
| SE | 0.034 | 0.027 | 0.029 | 0.034 |
| p Value | 4.4E−15 | 0 | 0 | 4.4E−15 |
| nCohort Non-persistent | 253 | 206 | 210 | 253 |
| nCohort Persistent | 75 | 122 | 116 | 75 |
| Cutoff Quartile 2 | 1.26 | 1.26 | 1.24 | 1.26 |
| Sensitivity | 92% | 91% | 91% | 92% |
| Specificity | 30% | 34% | 34% | 30% |
| Cutoff Quartile 3 | 4.59 | 4.59 | 4.54 | 4.59 |
| Sensitivity | 80% | 78% | 77% | 80% |
| Specificity | 59% | 67% | 65% | 59% |
| Cutoff Quartile 4 | 24.3 | 24.3 | 22.9 | 24.3 |
| Sensitivity | 53% | 48% | 47% | 53% |
| Specificity | 83% | 88% | 87% | 83% |
| OR Quartile 2 | 4.94 | 5.31 | 4.88 | 4.94 |
| p Value | 3.6E−4 | 1.7E−6 | 5.6E−6 | 3.6E−4 |
| Lower limit of 95% CI | 2.06 | 2.68 | 2.46 | 2.06 |
| Upper limit of 95% CI | 11.9 | 10.5 | 9.66 | 11.9 |
| OR Quartile 3 | 5.73 | 6.99 | 6.06 | 5.73 |
| p Value | 3.2E−8 | 1.6E−13 | 7.3E−12 | 3.2E−8 |
| Lower limit of 95% CI | 3.09 | 4.17 | 3.62 | 3.09 |
| Upper limit of 95% CI | 10.6 | 11.7 | 10.1 | 10.6 |
| OR Quartile 4 | 5.74 | 6.87 | 6.11 | 5.74 |
| p Value | 1.1E−9 | 9.5E−12 | 7.0E−11 | 1.1E−9 |
| Lower limit of 95% CI | 3.27 | 3.95 | 3.55 | 3.27 |
| Upper limit of 95% CI | 10.1 | 12.0 | 10.5 | 10.1 |
| C-C motif chemokine | | | | |
| 14 X Cystatin-C | | | | |
| AUC | 0.76 | 0.82 | 0.82 | 0.76 |
| SE | 0.036 | 0.026 | 0.027 | 0.036 |
| p Value | 1.7E−13 | 0 | 0 | 1.7E−13 |
| nCohort Non-persistent | 236 | 191 | 194 | 236 |
| nCohort Persistent | 70 | 117 | 113 | 70 |
| Cutoff Quartile 2 | 958000 | 956000 | 955000 | 958000 |
| Sensitivity | 93% | 92% | 92% | 93% |
| Specificity | 31% | 36% | 35% | 31% |
| Cutoff Quartile 3 | 2880000 | 2880000 | 2860000 | 2880000 |
| Sensitivity | 84% | 82% | 82% | 84% |
| Specificity | 60% | 70% | 69% | 60% |
| Cutoff Quartile 4 | 1.03E7 | 1.04E7 | 1.03E7 | 1.03E7 |
| Sensitivity | 53% | 50% | 51% | 53% |
| Specificity | 83% | 91% | 90% | 83% |
| OR Quartile 2 | 5.71 | 6.63 | 6.24 | 5.71 |
| p Value | 3.3E−4 | 5.7E−7 | 1.3E−6 | 3.3E−4 |
| Lower limit of 95% CI | 2.21 | 3.16 | 2.97 | 2.21 |
| Upper limit of 95% CI | 14.8 | 13.9 | 13.1 | 14.8 |
| OR Quartile 3 | 8.10 | 10.5 | 10.1 | 8.10 |
| p Value | 3.5E−9 | 2.2E−16 | 1.8E−15 | 3.5E−9 |
| Lower limit of 95% CI | 4.05 | 5.96 | 5.73 | 4.05 |
| Upper limit of 95% CI | 16.2 | 18.4 | 17.9 | 16.2 |
| OR Quartile 4 | 5.49 | 9.78 | 9.71 | 5.49 |
| p Value | 8.3E−9 | 1.6E−13 | 1.1E−13 | 8.3E−9 |
| Lower limit of 95% CI | 3.08 | 5.33 | 5.33 | 3.08 |
| Upper limit of 95% CI | 9.81 | 17.9 | 17.7 | 9.81 |
| C-C motif chemokine | | | | |
| 14 X Serum Creatinine | | | | |
| AUC | 0.77 | 0.84 | 0.83 | 0.77 |
| SE | 0.034 | 0.025 | 0.025 | 0.034 |
| p Value | 3.3E−15 | 0 | 0 | 3.3E−15 |
| nCohort Non-persistent | 247 | 202 | 206 | 247 |
| nCohort Persistent | 75 | 122 | 116 | 75 |
| Cutoff Quartile 2 | 1.30 | 1.29 | 1.29 | 1.30 |
| Sensitivity | 93% | 94% | 94% | 93% |
| Specificity | 31% | 37% | 36% | 31% |
| Cutoff Quartile 3 | 3.97 | 3.97 | 3.91 | 3.97 |
| Sensitivity | 87% | 84% | 84% | 87% |
| Specificity | 61% | 71% | 69% | 61% |
| Cutoff Quartile 4 | 18.0 | 18.3 | 16.8 | 18.0 |
| Sensitivity | 49% | 50% | 51% | 49% |
| Specificity | 82% | 90% | 89% | 82% |
| OR Quartile 2 | 6.22 | 9.50 | 8.73 | 6.22 |
| p Value | 1.5E−4 | 6.2E−8 | 1.9E−7 | 1.5E−4 |
| Lower limit of 95% CI | 2.41 | 4.20 | 3.86 | 2.41 |
| Upper limit of 95% CI | 16.0 | 21.5 | 19.7 | 16.0 |
| OR Quartile 3 | 10.2 | 13.1 | 12.4 | 10.2 |
| p Value | 1.7E−10 | 0 | 0 | 1.7E−10 |
| Lower limit of 95% CI | 5.01 | 7.39 | 6.90 | 5.01 |
| Upper limit of 95% CI | 20.9 | 23.4 | 22.1 | 20.9 |
| OR Quartile 4 | 4.49 | 9.10 | 8.66 | 4.49 |
| p Value | 1.3E−7 | 1.1E−13 | 1.5E−13 | 1.3E−7 |
| Lower limit of 95% CI | 2.57 | 5.08 | 4.88 | 2.57 |
| Upper limit of 95% CI | 7.85 | 16.3 | 15.3 | 7.85 |
| C-C motif chemokine 14 X | | | | |
| Serum Creatinine/(Weight | | | | |
| adjusted urine output) | | | | |
| AUC | 0.79 | 0.82 | 0.81 | 0.79 |
| SE | 0.033 | 0.026 | 0.027 | 0.033 |
| p Value | 0 | 0 | 0 | 0 |
| nCohort Non-persistent | 246 | 200 | 204 | 246 |
| nCohort Persistent | 75 | 121 | 115 | 75 |
| Cutoff Quartile 2 | 2.17 | 2.17 | 2.15 | 2.17 |
| Sensitivity | 92% | 92% | 91% | 92% |
| Specificity | 30% | 35% | 34% | 30% |
| Cutoff Quartile 3 | 10.2 | 10.2 | 10.1 | 10.2 |
| Sensitivity | 84% | 82% | 81% | 84% |
| Specificity | 60% | 69% | 67% | 60% |
| Cutoff Quartile 4 | 69.8 | 69.8 | 67.6 | 69.8 |
| Sensitivity | 53% | 51% | 50% | 53% |
| Specificity | 83% | 90% | 89% | 83% |
| OR Quartile 2 | 4.95 | 5.98 | 5.49 | 4.95 |
| p Value | 3.6E−4 | 7.8E−7 | 2.6E−6 | 3.6E−4 |
| Lower limit of 95% CI | 2.06 | 2.94 | 2.70 | 2.06 |
| Upper limit of 95% CI | 11.9 | 12.1 | 11.2 | 11.9 |
| OR Quartile 3 | 7.93 | 10.0 | 8.64 | 7.93 |
| p Value | 1.2E−9 | 2.2E−16 | 1.4E−14 | 1.2E−9 |
| Lower limit of 95% CI | 4.07 | 5.78 | 4.99 | 4.07 |
| Upper limit of 95% CI | 15.5 | 17.4 | 15.0 | 15.5 |
| OR Quartile 4 | 5.71 | 10.0 | 7.73 | 5.71 |
| p Value | 1.4E−9 | 2.4E−14 | 1.6E−12 | 1.4E−9 |
| Lower limit of 95% CI | 3.25 | 5.54 | 4.39 | 3.25 |
| Upper limit of 95% CI | 10.0 | 18.1 | 13.6 | 10.0 |
| C-C motif chemokine 14 X | | | | |
| Cystatin-C/(Weight | | | | |
| adjusted urine output) | | | | |
| AUC | 0.77 | 0.79 | 0.78 | 0.77 |
| SE | 0.035 | 0.028 | 0.029 | 0.035 |
| p Value | 1.9E−14 | 0 | 0 | 1.9E−14 |
| nCohort Non-persistent | 235 | 189 | 192 | 235 |
| nCohort Persistent | 70 | 116 | 112 | 70 |
| Cutoff Quartile 2 | 1580000 | 1580000 | 1580000 | 1580000 |
| Sensitivity | 91% | 91% | 91% | 91% |
| Specificity | 30% | 35% | 34% | 30% |
| Cutoff Quartile 3 | 6710000 | 6710000 | 6570000 | 6710000 |
| Sensitivity | 83% | 80% | 79% | 83% |
| Specificity | 60% | 68% | 67% | 60% |
| Cutoff Quartile 4 | 3.85E7 | 3.85E7 | 3.84E7 | 3.85E7 |
| Sensitivity | 49% | 47% | 46% | 49% |
| Specificity | 82% | 88% | 87% | 82% |
| OR Quartile 2 | 4.53 | 5.69 | 5.34 | 4.53 |
| p Value | 8.0E−4 | 1.8E−6 | 4.3E−6 | 8.0E−4 |
| Lower limit of 95% CI | 1.87 | 2.79 | 2.61 | 1.87 |
| Upper limit of 95% CI | 10.9 | 11.6 | 10.9 | 10.9 |
| OR Quartile 3 | 7.12 | 8.69 | 7.92 | 7.12 |
| p Value | 1.1E−8 | 1.2E−14 | 1.4E−13 | 1.1E−8 |
| Lower limit of 95% CI | 3.63 | 5.02 | 4.58 | 3.63 |
| Upper limit of 95% CI | 14.0 | 15.1 | 13.7 | 14.0 |
| OR Quartile 4 | 4.22 | 6.29 | 5.58 | 4.22 |
| p Value | 8.8E−7 | 2.3E−10 | 1.9E−9 | 8.8E−7 |
| Lower limit of 95% CI | 2.38 | 3.56 | 3.19 | 2.38 |
| Upper limit of 95% CI | 7.48 | 11.1 | 9.79 | 7.48 |

TABLE 7
Comparison of marker levels and the area under the ROC curve (AUC) in urine samples for the “persistent”
and “non-persistent” cohorts where persistence starts within 168 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| | Persistence duration (hr) | | | | |
|---|---|---|---|---|---|
| | 24 | | | 48 | |
| | sCr or | | | sCr or | |
| | UO | sCr only | UO only | UO | sCr only |
| C-C motif chemokine 14 | | | | | |
| AUC | 0.79 | 0.78 | 0.75 | 0.82 | 0.81 |
| SE | 0.025 | 0.026 | 0.033 | 0.025 | 0.026 |
| p Value | 0 | 0 | 9.5E−14 | 0 | 0 |
| nCohort Non-persistent | 177 | 185 | 243 | 191 | 197 |
| nCohort Persistent | 154 | 144 | 86 | 140 | 132 |
| Cutoff Quartile 2 | 0.725 | 0.719 | 0.732 | 0.725 | 0.719 |
| Sensitivity | 90% | 89% | 93% | 92% | 92% |
| Specificity | 38% | 36% | 31% | 38% | 36% |
| Cutoff Quartile 3 | 1.64 | 1.64 | 1.64 | 1.64 | 1.64 |
| Sensitivity | 75% | 75% | 80% | 79% | 79% |
| Specificity | 71% | 69% | 60% | 71% | 69% |
| Cutoff Quartile 4 | 5.16 | 5.09 | 5.09 | 5.16 | 5.09 |
| Sensitivity | 44% | 45% | 44% | 48% | 48% |
| Specificity | 92% | 90% | 81% | 92% | 90% |
| OR Quartile 2 | 5.25 | 4.44 | 6.07 | 7.10 | 6.20 |
| p Value | 6.0E−8 | 1.2E−6 | 5.1E−5 | 1.8E−8 | 1.6E−7 |
| Lower limit of 95% CI | 2.88 | 2.43 | 2.53 | 3.59 | 3.13 |
| Upper limit of 95% CI | 9.57 | 8.09 | 14.5 | 14.0 | 12.3 |
| OR Quartile 3 | 7.29 | 6.74 | 6.22 | 8.84 | 8.28 |
| p Value | 1.3E−15 | 2.2E−14 | 1.3E−9 | 0 | 8.9E−16 |
| Lower limit of 95% CI | 4.47 | 4.13 | 3.45 | 5.31 | 4.95 |
| Upper limit of 95% CI | 11.9 | 11.0 | 11.2 | 14.7 | 13.9 |
| OR Quartile 4 | 8.54 | 7.63 | 3.48 | 10.0 | 8.82 |
| p Value | 9.7E−12 | 1.1E−11 | 4.8E−6 | 1.2E−13 | 2.6E−13 |
| Lower limit of 95% CI | 4.61 | 4.25 | 2.04 | 5.45 | 4.92 |
| Upper limit of 95% CI | 15.8 | 13.7 | 5.95 | 18.5 | 15.8 |
| Weight adjusted urine output | | | | | |
| AUC | 0.36 | 0.40 | 0.29 | 0.35 | 0.38 |
| SE | 0.030 | 0.031 | 0.034 | 0.031 | 0.032 |
| p Value | 7.1E−6 | 0.0020 | 5.2E−10 | 9.3E−7 | 1.4E−4 |
| nCohort Non-persistent | 180 | 188 | 248 | 194 | 200 |
| nCohort Persistent | 157 | 147 | 89 | 143 | 135 |
| Cutoff Quartile 2 | 0.189 | 0.192 | 0.189 | 0.189 | 0.192 |
| Sensitivity | 64% | 69% | 51% | 64% | 67% |
| Specificity | 16% | 20% | 16% | 16% | 20% |
| Cutoff Quartile 3 | 0.366 | 0.368 | 0.366 | 0.366 | 0.368 |
| Sensitivity | 41% | 43% | 30% | 39% | 41% |
| Specificity | 42% | 44% | 43% | 42% | 44% |
| Cutoff Quartile 4 | 0.857 | 0.862 | 0.857 | 0.857 | 0.862 |
| Sensitivity | 21% | 22% | 13% | 20% | 20% |
| Specificity | 71% | 72% | 71% | 71% | 72% |
| OR Quartile 2 | 0.332 | 0.538 | 0.197 | 0.346 | 0.501 |
| p Value | 3.1E−5 | 0.015 | 2.7E−9 | 4.4E−5 | 0.0070 |
| Lower limit of 95% CI | 0.198 | 0.326 | 0.115 | 0.208 | 0.303 |
| Upper limit of 95% CI | 0.558 | 0.888 | 0.336 | 0.576 | 0.828 |
| OR Quartile 3 | 0.492 | 0.593 | 0.325 | 0.461 | 0.529 |
| p Value | 0.0014 | 0.019 | 2.1E−5 | 5.8E−4 | 0.0049 |
| Lower limit of 95% CI | 0.318 | 0.384 | 0.194 | 0.297 | 0.340 |
| Upper limit of 95% CI | 0.759 | 0.916 | 0.545 | 0.717 | 0.824 |
| OR Quartile 4 | 0.655 | 0.728 | 0.374 | 0.585 | 0.627 |
| p Value | 0.098 | 0.22 | 0.0038 | 0.042 | 0.080 |
| Lower limit of 95% CI | 0.397 | 0.439 | 0.192 | 0.349 | 0.372 |
| Upper limit of 95% CI | 1.08 | 1.21 | 0.728 | 0.980 | 1.06 |
| Serum Creatinine | | | | | |
| AUC | 0.79 | 0.80 | 0.71 | 0.79 | 0.80 |
| SE | 0.024 | 0.024 | 0.032 | 0.025 | 0.025 |
| p Value | 0 | 0 | 2.0E−11 | 0 | 0 |
| nCohort Non-persistent | 181 | 189 | 249 | 195 | 201 |
| nCohort Persistent | 173 | 163 | 103 | 159 | 151 |
| Cutoff Quartile 2 | 1.64 | 1.64 | 1.65 | 1.64 | 1.64 |
| Sensitivity | 91% | 93% | 91% | 93% | 94% |
| Specificity | 41% | 40% | 32% | 40% | 39% |
| Cutoff Quartile 3 | 2.44 | 2.44 | 2.44 | 2.44 | 2.44 |
| Sensitivity | 72% | 74% | 70% | 73% | 75% |
| Specificity | 70% | 70% | 58% | 68% | 69% |
| Cutoff Quartile 4 | 3.32 | 3.31 | 3.32 | 3.32 | 3.31 |
| Sensitivity | 43% | 45% | 46% | 44% | 45% |
| Specificity | 92% | 92% | 83% | 90% | 90% |
| OR Quartile 2 | 7.28 | 8.46 | 4.85 | 8.97 | 10.2 |
| p Value | 1.4E−10 | 1.7E−10 | 2.5E−5 | 2.0E−10 | 4.6E−10 |
| Lower limit of 95% CI | 3.97 | 4.39 | 2.33 | 4.56 | 4.92 |
| Upper limit of 95% CI | 13.4 | 16.3 | 10.1 | 17.6 | 21.2 |
| OR Quartile 3 | 5.95 | 6.63 | 3.19 | 5.79 | 6.51 |
| p Value | 2.6E−14 | 2.2E−15 | 3.7E−6 | 9.3E−14 | 8.4E−15 |
| Lower limit of 95% CI | 3.76 | 4.15 | 1.95 | 3.65 | 4.06 |
| Upper limit of 95% CI | 9.42 | 10.6 | 5.20 | 9.18 | 10.5 |
| OR Quartile 4 | 8.47 | 9.41 | 4.14 | 6.88 | 7.41 |
| p Value | 5.7E−12 | 6.5E−13 | 4.9E−8 | 1.3E−11 | 2.9E−12 |
| Lower limit of 95% CI | 4.61 | 5.11 | 2.48 | 3.94 | 4.23 |
| Upper limit of 95% CI | 15.6 | 17.3 | 6.89 | 12.0 | 13.0 |
| Cystatin-C | | | | | |
| AUC | 0.76 | 0.75 | 0.74 | 0.75 | 0.75 |
| SE | 0.028 | 0.028 | 0.034 | 0.028 | 0.029 |
| p Value | 0 | 0 | 2.1E−12 | 0 | 0 |
| nCohort Non-persistent | 161 | 168 | 225 | 174 | 179 |
| nCohort Persistent | 147 | 139 | 81 | 134 | 128 |
| Cutoff Quartile 2 | 1170000 | 1170000 | 1170000 | 1170000 | 1170000 |
| Sensitivity | 91% | 91% | 94% | 91% | 91% |
| Specificity | 39% | 38% | 32% | 37% | 36% |
| Cutoff Quartile 3 | 1660000 | 1650000 | 1660000 | 1660000 | 1650000 |
| Sensitivity | 71% | 71% | 77% | 71% | 71% |
| Specificity | 70% | 67% | 60% | 66% | 65% |
| Cutoff Quartile 4 | 2150000 | 2150000 | 2150000 | 2150000 | 2150000 |
| Sensitivity | 39% | 40% | 46% | 40% | 42% |
| Specificity | 88% | 88% | 82% | 87% | 87% |
| OR Quartile 2 | 6.63 | 5.82 | 7.15 | 5.92 | 5.38 |
| p Value | 1.3E−8 | 1.1E−7 | 4.7E−5 | 1.9E−7 | 8.0E−7 |
| Lower limit of 95% CI | 3.45 | 3.03 | 2.77 | 3.03 | 2.76 |
| Upper limit of 95% CI | 12.7 | 11.1 | 18.4 | 11.5 | 10.5 |
| OR Quartile 3 | 5.71 | 5.08 | 4.81 | 4.75 | 4.53 |
| p Value | 3.3E−12 | 6.8E−11 | 1.1E−7 | 3.7E−10 | 1.5E−9 |
| Lower limit of 95% CI | 3.50 | 3.12 | 2.69 | 2.92 | 2.77 |
| Upper limit of 95% CI | 9.33 | 8.29 | 8.57 | 7.73 | 7.39 |
| OR Quartile 4 | 4.46 | 4.72 | 3.89 | 4.43 | 4.95 |
| p Value | 3.2E−7 | 9.0E−8 | 1.6E−6 | 1.7E−7 | 2.3E−8 |
| Lower limit of 95% CI | 2.52 | 2.67 | 2.23 | 2.54 | 2.82 |
| Upper limit of 95% CI | 7.93 | 8.34 | 6.77 | 7.74 | 8.67 |
| C-C motif chemokine 14/(Weight | | | | | |
| adjusted urine output) | | | | | |
| AUC | 0.77 | 0.74 | 0.78 | 0.80 | 0.78 |
| SE | 0.026 | 0.028 | 0.032 | 0.026 | 0.027 |
| p Value | 0 | 0 | 0 | 0 | 0 |
| nCohort Non-persistent | 175 | 183 | 242 | 189 | 195 |
| nCohort Persistent | 153 | 143 | 86 | 139 | 131 |
| Cutoff Quartile 2 | 1.26 | 1.24 | 1.26 | 1.26 | 1.24 |
| Sensitivity | 86% | 85% | 92% | 89% | 89% |
| Specificity | 35% | 33% | 31% | 35% | 34% |
| Cutoff Quartile 3 | 4.59 | 4.54 | 4.59 | 4.59 | 4.54 |
| Sensitivity | 73% | 71% | 81% | 76% | 75% |
| Specificity | 70% | 67% | 61% | 69% | 67% |
| Cutoff Quartile 4 | 24.3 | 22.9 | 24.3 | 24.3 | 22.9 |
| Sensitivity | 46% | 45% | 53% | 49% | 48% |
| Specificity | 93% | 90% | 85% | 93% | 90% |
| OR Quartile 2 | 3.36 | 2.90 | 5.07 | 4.54 | 4.05 |
| p Value | 1.9E−5 | 1.7E−4 | 1.0E−4 | 1.3E−6 | 8.0E−6 |
| Lower limit of 95% CI | 1.93 | 1.67 | 2.23 | 2.46 | 2.19 |
| Upper limit of 95% CI | 5.86 | 5.06 | 11.5 | 8.38 | 7.48 |
| OR Quartile 3 | 6.46 | 4.98 | 6.89 | 7.25 | 5.94 |
| p Value | 3.6E−14 | 3.6E−11 | 3.2E−10 | 6.4E−15 | 1.6E−12 |
| Lower limit of 95% CI | 3.99 | 3.09 | 3.77 | 4.41 | 3.62 |
| Upper limit of 95% CI | 10.5 | 8.00 | 12.6 | 11.9 | 9.74 |
| OR Quartile 4 | 11.5 | 7.43 | 6.58 | 12.0 | 8.58 |
| p Value | 7.7E−13 | 2.3E−11 | 2.3E−11 | 2.4E−14 | 5.6E−13 |
| Lower limit of 95% CI | 5.88 | 4.13 | 3.79 | 6.33 | 4.78 |
| Upper limit of 95% CI | 22.3 | 13.4 | 11.4 | 22.7 | 15.4 |
| C-C motif chemokine | | | | | |
| 14 X Serum Creatinine | | | | | |
| AUC | 0.82 | 0.82 | 0.77 | 0.85 | 0.84 |
| SE | 0.024 | 0.024 | 0.032 | 0.023 | 0.024 |
| p Value | 0 | 0 | 2.2E−16 | 0 | 0 |
| nCohort Non-persistent | 171 | 179 | 236 | 185 | 191 |
| nCohort Persistent | 153 | 143 | 86 | 139 | 131 |
| Cutoff Quartile 2 | 1.29 | 1.29 | 1.30 | 1.29 | 1.29 |
| Sensitivity | 91% | 91% | 92% | 94% | 93% |
| Specificity | 39% | 38% | 31% | 39% | 38% |
| Cutoff Quartile 3 | 3.97 | 3.91 | 3.97 | 3.97 | 3.91 |
| Sensitivity | 78% | 78% | 85% | 81% | 82% |
| Specificity | 75% | 73% | 63% | 74% | 72% |
| Cutoff Quartile 4 | 18.3 | 16.8 | 18.0 | 18.3 | 16.8 |
| Sensitivity | 45% | 47% | 48% | 50% | 51% |
| Specificity | 93% | 92% | 83% | 94% | 93% |
| OR Quartile 2 | 6.40 | 6.13 | 5.16 | 9.20 | 8.20 |
| p Value | 7.6E−9 | 3.6E−8 | 8.9E−5 | 3.6E−9 | 2.2E−8 |
| Lower limit of 95% CI | 3.41 | 3.21 | 2.27 | 4.40 | 3.92 |
| Upper limit of 95% CI | 12.0 | 11.7 | 11.7 | 19.2 | 17.1 |
| OR Quartile 3 | 10.4 | 9.59 | 9.44 | 12.1 | 11.3 |
| p Value | 0 | 0 | 9.8E−12 | 0 | 0 |
| Lower limit of 95% CI | 6.23 | 5.72 | 4.95 | 7.05 | 6.57 |
| Upper limit of 95% CI | 17.4 | 16.1 | 18.0 | 20.6 | 19.5 |
| OR Quartile 4 | 10.9 | 10.4 | 4.46 | 14.2 | 13.2 |
| p Value | 2.4E−12 | 5.8E−13 | 6.6E−8 | 1.1E−14 | 3.6E−15 |
| Lower limit of 95% CI | 5.58 | 5.50 | 2.59 | 7.25 | 6.96 |
| Upper limit of 95% CI | 21.2 | 19.6 | 7.68 | 27.9 | 25.2 |
| C-C motif chemokine | | | | | |
| 14 X Cystatin-C | | | | | |
| AUC | 0.81 | 0.80 | 0.77 | 0.83 | 0.82 |
| SE | 0.025 | 0.026 | 0.033 | 0.024 | 0.025 |
| p Value | 0 | 0 | 1.3E−15 | 0 | 0 |
| nCohort Non-persistent | 161 | 168 | 225 | 174 | 179 |
| nCohort Persistent | 147 | 139 | 81 | 134 | 128 |
| Cutoff Quartile 2 | 956000 | 955000 | 958000 | 956000 | 955000 |
| Sensitivity | 90% | 90% | 93% | 92% | 91% |
| Specificity | 39% | 38% | 32% | 38% | 37% |
| Cutoff Quartile 3 | 2880000 | 2860000 | 2880000 | 2880000 | 2860000 |
| Sensitivity | 78% | 78% | 84% | 80% | 80% |
| Specificity | 75% | 73% | 62% | 73% | 71% |
| Cutoff Quartile 4 | 1.04E7 | 1.03E7 | 1.03E7 | 1.04E7 | 1.03E7 |
| Sensitivity | 45% | 46% | 51% | 49% | 50% |
| Specificity | 93% | 92% | 84% | 94% | 93% |
| OR Quartile 2 | 6.11 | 5.36 | 5.76 | 6.83 | 6.21 |
| p Value | 2.4E−8 | 2.2E−7 | 9.2E−5 | 4.5E−8 | 2.0E−7 |
| Lower limit of 95% CI | 3.24 | 2.84 | 2.40 | 3.43 | 3.12 |
| Upper limit of 95% CI | 11.5 | 10.1 | 13.9 | 13.6 | 12.4 |
| OR Quartile 3 | 10.4 | 9.24 | 8.62 | 10.7 | 9.58 |
| p Value | 0 | 0 | 9.3E−11 | 0 | 2.2E−16 |
| Lower limit of 95% CI | 6.17 | 5.47 | 4.49 | 6.25 | 5.59 |
| Upper limit of 95% CI | 17.7 | 15.6 | 16.5 | 18.4 | 16.4 |
| OR Quartile 4 | 11.1 | 10.2 | 5.38 | 14.4 | 12.8 |
| p Value | 9.8E−12 | 4.5E−12 | 4.6E−9 | 7.2E−14 | 4.8E−14 |
| Lower limit of 95% CI | 5.56 | 5.27 | 3.07 | 7.15 | 6.58 |
| Upper limit of 95% CI | 22.2 | 19.6 | 9.45 | 28.9 | 24.8 |
| C-C motif chemokine 14 X | | | | | |
| Serum Creatinine/(Weight | | | | | |
| adjusted urine output) | | | | | |
| AUC | 0.81 | 0.79 | 0.80 | 0.83 | 0.81 |
| SE | 0.024 | 0.026 | 0.031 | 0.024 | 0.026 |
| p Value | 0 | 0 | 0 | 0 | 0 |
| nCohort Non-persistent | 169 | 177 | 235 | 183 | 189 |
| nCohort Persistent | 152 | 142 | 86 | 138 | 130 |
| Cutoff Quartile 2 | 2.17 | 2.15 | 2.17 | 2.17 | 2.15 |
| Sensitivity | 89% | 88% | 92% | 91% | 90% |
| Specificity | 37% | 36% | 31% | 37% | 35% |
| Cutoff Quartile 3 | 10.2 | 10.1 | 10.2 | 10.2 | 10.1 |
| Sensitivity | 77% | 75% | 85% | 80% | 78% |
| Specificity | 74% | 70% | 63% | 72% | 69% |
| Cutoff Quartile 4 | 69.8 | 67.6 | 69.8 | 69.8 | 67.6 |
| Sensitivity | 48% | 46% | 52% | 51% | 50% |
| Specificity | 95% | 92% | 85% | 95% | 92% |
| OR Quartile 2 | 4.72 | 4.06 | 5.09 | 5.55 | 4.94 |
| p Value | 2.9E−7 | 3.6E−6 | 1.0E−4 | 1.9E−7 | 1.2E−6 |
| Lower limit of 95% CI | 2.61 | 2.25 | 2.24 | 2.91 | 2.59 |
| Upper limit of 95% CI | 8.54 | 7.35 | 11.6 | 10.6 | 9.43 |
| OR Quartile 3 | 9.50 | 7.15 | 9.38 | 10.2 | 8.23 |
| p Value | 0 | 1.1E−14 | 1.1E−11 | 0 | 2.0E−15 |
| Lower limit of 95% CI | 5.70 | 4.34 | 4.91 | 6.01 | 4.89 |
| Upper limit of 95% CI | 15.8 | 11.8 | 17.9 | 17.2 | 13.8 |
| OR Quartile 4 | 18.6 | 10.1 | 6.07 | 18.3 | 11.6 |
| p Value | 1.8E−13 | 1.2E−12 | 1.6E−10 | 2.2E−15 | 2.3E−14 |
| Lower limit of 95% CI | 8.54 | 5.34 | 3.49 | 8.93 | 6.18 |
| Upper limit of 95% CI | 40.5 | 19.1 | 10.5 | 37.6 | 21.8 |
| C-C motif chemokine 14 X | | | | | |
| Cystatin-C/(Weight adjusted | | | | | |
| urine output) | | | | | |
| AUC | 0.80 | 0.77 | 0.79 | 0.81 | 0.79 |
| SE | 0.026 | 0.028 | 0.032 | 0.026 | 0.027 |
| p Value | 0 | 0 | 0 | 0 | 0 |
| nCohort Non- | 159 | 166 | 224 | 172 | 177 |
| persistent | | | | | |
| nCohort Persistent | 146 | 138 | 81 | 133 | 127 |
| Cutoff Quartile 2 | 1580000 | 1580000 | 1580000 | 1580000 | 1580000 |
| Sensitivity | 87% | 86% | 91% | 89% | 89% |
| Specificity | 36% | 34% | 31% | 36% | 35% |
| Cutoff Quartile 3 | 6710000 | 6570000 | 6710000 | 6710000 | 6570000 |
| Sensitivity | 75% | 74% | 84% | 78% | 77% |
| Specificity | 73% | 70% | 62% | 72% | 69% |
| Cutoff Quartile 4 | 3.85E7 | 3.84E7 | 3.85E7 | 3.85E7 | 3.84E7 |
| Sensitivity | 45% | 43% | 49% | 48% | 46% |
| Specificity | 93% | 90% | 83% | 92% | 90% |
| OR Quartile 2 | 3.74 | 3.28 | 4.71 | 4.79 | 4.35 |
| p Value | 8.7E−6 | 6.2E−5 | 2.3E−4 | 1.3E−6 | 5.7E−6 |
| Lower limit of 95% CI | 2.09 | 1.83 | 2.06 | 2.54 | 2.31 |
| Upper limit of 95% CI | 6.68 | 5.85 | 10.7 | 9.04 | 8.21 |
| OR Quartile 3 | 8.24 | 6.57 | 8.55 | 9.00 | 7.70 |
| p Value | 8.9E−16 | 2.5E−13 | 1.1E−10 | 4.4E−16 | 2.2E−14 |
| Lower limit of 95% CI | 4.93 | 3.97 | 4.46 | 5.31 | 4.56 |
| Upper limit of 95% CI | 13.8 | 10.9 | 16.4 | 15.3 | 13.0 |
| OR Quartile 4 | 11.1 | 7.21 | 4.93 | 11.3 | 8.17 |
| p Value | 1.1E−11 | 3.2E−10 | 2.4E−8 | 5.4E−13 | 1.5E−11 |
| Lower limit of 95% CI | 5.55 | 3.90 | 2.82 | 5.86 | 4.44 |
| Upper limit of 95% CI | 22.2 | 13.3 | 8.64 | 21.9 | 15.0 |

| | Persistence duration (hr) | | | |
|---|---|---|---|---|
| | | 72 | | |
| | 48 | sCr or | | |
| | UO only | UO | sCr only | UO only |
| C-C motif chemokine 14 | | | | |
| AUC | 0.75 | 0.80 | 0.79 | 0.75 |
| SE | 0.033 | 0.026 | 0.027 | 0.033 |
| p Value | 6.8E−14 | 0 | 0 | 6.8E−14 |
| nCohort Non-persistent | 246 | 202 | 208 | 246 |
| nCohort Persistent | 83 | 129 | 121 | 83 |
| Cutoff Quartile 2 | 0.732 | 0.725 | 0.719 | 0.732 |
| Sensitivity | 93% | 92% | 92% | 93% |
| Specificity | 31% | 36% | 35% | 31% |
| Cutoff Quartile 3 | 1.64 | 1.64 | 1.64 | 1.64 |
| Sensitivity | 81% | 79% | 79% | 81% |
| Specificity | 60% | 68% | 67% | 60% |
| Cutoff Quartile 4 | 5.09 | 5.16 | 5.09 | 5.09 |
| Sensitivity | 46% | 47% | 48% | 46% |
| Specificity | 82% | 89% | 88% | 82% |
| OR Quartile 2 | 5.74 | 6.73 | 5.88 | 5.74 |
| p Value | 8.9E−5 | 1.2E−7 | 9.2E−7 | 8.9E−5 |
| Lower limit of 95% CI | 2.39 | 3.32 | 2.90 | 2.39 |
| Upper limit of 95% CI | 13.7 | 13.6 | 11.9 | 13.7 |
| OR Quartile 3 | 6.32 | 8.15 | 7.74 | 6.32 |
| p Value | 1.9E−9 | 2.0E−15 | 2.6E−14 | 1.9E−9 |
| Lower limit of 95% CI | 3.46 | 4.85 | 4.57 | 3.46 |
| Upper limit of 95% CI | 11.5 | 13.7 | 13.1 | 11.5 |
| OR Quartile 4 | 3.77 | 7.34 | 6.74 | 3.77 |
| p Value | 1.4E−6 | 3.5E−12 | 1.0E−11 | 1.4E−6 |
| Lower limit of 95% CI | 2.20 | 4.19 | 3.89 | 2.20 |
| Upper limit of 95% CI | 6.47 | 12.9 | 11.7 | 6.47 |
| Weight adjusted urine output | | | | |
| AUC | 0.30 | 0.35 | 0.38 | 0.30 |
| SE | 0.035 | 0.031 | 0.032 | 0.035 |
| p Value | 1.3E−8 | 2.4E−6 | 2.9E−4 | 1.3E−8 |
| nCohort Non-persistent | 251 | 206 | 212 | 251 |
| nCohort Persistent | 86 | 131 | 123 | 86 |
| Cutoff Quartile 2 | 0.189 | 0.189 | 0.192 | 0.189 |
| Sensitivity | 52% | 63% | 67% | 52% |
| Specificity | 17% | 17% | 20% | 17% |
| Cutoff Quartile 3 | 0.366 | 0.366 | 0.368 | 0.366 |
| Sensitivity | 31% | 39% | 41% | 31% |
| Specificity | 43% | 43% | 44% | 43% |
| Cutoff Quartile 4 | 0.857 | 0.857 | 0.862 | 0.857 |
| Sensitivity | 14% | 20% | 20% | 14% |
| Specificity | 71% | 71% | 72% | 71% |
| OR Quartile 2 | 0.227 | 0.343 | 0.494 | 0.227 |
| p Value | 5.7E−8 | 3.5E−5 | 0.0062 | 5.7E−8 |
| Lower limit of 95% CI | 0.133 | 0.206 | 0.298 | 0.133 |
| Upper limit of 95% CI | 0.388 | 0.569 | 0.818 | 0.388 |
| OR Quartile 3 | 0.351 | 0.475 | 0.546 | 0.351 |
| p Value | 7.9E−5 | 0.0011 | 0.0084 | 7.9E−5 |
| Lower limit of 95% CI | 0.209 | 0.304 | 0.348 | 0.209 |
| Upper limit of 95% CI | 0.590 | 0.743 | 0.856 | 0.590 |
| OR Quartile 4 | 0.395 | 0.617 | 0.662 | 0.395 |
| p Value | 0.0065 | 0.071 | 0.13 | 0.0065 |
| Lower limit of 95% CI | 0.203 | 0.365 | 0.389 | 0.203 |
| Upper limit of 95% CI | 0.771 | 1.04 | 1.13 | 0.771 |
| Serum Creatinine | | | | |
| AUC | 0.72 | 0.78 | 0.79 | 0.71 |
| SE | 0.032 | 0.026 | 0.026 | 0.032 |
| p Value | 6.0E−12 | 0 | 0 | 2.6E−11 |
| nCohort Non-persistent | 251 | 207 | 213 | 252 |
| nCohort Persistent | 101 | 147 | 139 | 100 |
| Cutoff Quartile 2 | 1.65 | 1.64 | 1.64 | 1.65 |
| Sensitivity | 92% | 93% | 94% | 92% |
| Specificity | 32% | 38% | 38% | 32% |
| Cutoff Quartile 3 | 2.44 | 2.44 | 2.44 | 2.44 |
| Sensitivity | 70% | 73% | 76% | 70% |
| Specificity | 58% | 66% | 67% | 58% |
| Cutoff Quartile 4 | 3.32 | 3.32 | 3.31 | 3.32 |
| Sensitivity | 47% | 46% | 47% | 46% |
| Specificity | 83% | 89% | 89% | 83% |
| OR Quartile 2 | 5.44 | 8.46 | 9.85 | 5.35 |
| p Value | 1.6E−5 | 2.3E−9 | 4.8E−9 | 2.0E−5 |
| Lower limit of 95% CI | 2.52 | 4.20 | 4.58 | 2.48 |
| Upper limit of 95% CI | 11.7 | 17.0 | 21.2 | 11.5 |
| OR Quartile 3 | 3.24 | 5.42 | 6.18 | 3.16 |
| p Value | 3.3E−6 | 1.2E−12 | 1.1E−13 | 5.2E−6 |
| Lower limit of 95% CI | 1.97 | 3.40 | 3.82 | 1.93 |
| Upper limit of 95% CI | 5.31 | 8.64 | 9.99 | 5.19 |
| OR Quartile 4 | 4.33 | 6.70 | 7.26 | 4.14 |
| p Value | 2.1E−8 | 5.8E−12 | 1.1E−12 | 5.4E−8 |
| Lower limit of 95% CI | 2.59 | 3.90 | 4.20 | 2.48 |
| Upper limit of 95% CI | 7.23 | 11.5 | 12.5 | 6.91 |
| Cystatin-C | | | | |
| AUC | 0.74 | 0.75 | 0.74 | 0.73 |
| SE | 0.035 | 0.029 | 0.030 | 0.035 |
| p Value | 1.1E−11 | 0 | 4.4E−16 | 3.0E−11 |
| nCohort Non-persistent | 227 | 185 | 190 | 228 |
| nCohort Persistent | 79 | 123 | 117 | 78 |
| Cutoff Quartile 2 | 1170000 | 1170000 | 1170000 | 1170000 |
| Sensitivity | 94% | 91% | 91% | 94% |
| Specificity | 32% | 35% | 34% | 32% |
| Cutoff Quartile 3 | 1660000 | 1660000 | 1650000 | 1660000 |
| Sensitivity | 76% | 72% | 73% | 76% |
| Specificity | 59% | 65% | 64% | 59% |
| Cutoff Quartile 4 | 2150000 | 2150000 | 2150000 | 2150000 |
| Sensitivity | 46% | 41% | 44% | 45% |
| Specificity | 82% | 86% | 86% | 82% |
| OR Quartile 2 | 6.87 | 5.52 | 5.01 | 6.74 |
| p Value | 6.7E−5 | 1.2E−6 | 4.6E−6 | 8.0E−5 |
| Lower limit of 95% CI | 2.66 | 2.77 | 2.51 | 2.61 |
| Upper limit of 95% CI | 17.7 | 11.0 | 9.98 | 17.4 |
| OR Quartile 3 | 4.55 | 4.83 | 4.66 | 4.43 |
| p Value | 3.0E−7 | 5.3E−10 | 2.0E−9 | 5.1E−7 |
| Lower limit of 95% CI | 2.55 | 2.94 | 2.82 | 2.48 |
| Upper limit of 95% CI | 8.13 | 7.95 | 7.70 | 7.91 |
| OR Quartile 4 | 3.80 | 4.33 | 4.87 | 3.60 |
| p Value | 2.7E−6 | 1.6E−7 | 1.9E−8 | 6.6E−6 |
| Lower limit of 95% CI | 2.18 | 2.50 | 2.81 | 2.06 |
| Upper limit of 95% CI | 6.63 | 7.49 | 8.47 | 6.30 |
| C-C motif chemokine 14/(Weight | | | | |
| adjusted urine output) | | | | |
| AUC | 0.78 | 0.79 | 0.77 | 0.78 |
| SE | 0.032 | 0.027 | 0.029 | 0.032 |
| p Value | 0 | 0 | 0 | 0 |
| nCohort Non-persistent | 245 | 200 | 206 | 245 |
| nCohort Persistent | 83 | 128 | 120 | 83 |
| Cutoff Quartile 2 | 1.26 | 1.26 | 1.24 | 1.26 |
| Sensitivity | 92% | 90% | 89% | 92% |
| Specificity | 31% | 34% | 33% | 31% |
| Cutoff Quartile 3 | 4.59 | 4.59 | 4.54 | 4.59 |
| Sensitivity | 81% | 77% | 76% | 81% |
| Specificity | 60% | 68% | 65% | 60% |
| Cutoff Quartile 4 | 24.3 | 24.3 | 22.9 | 24.3 |
| Sensitivity | 53% | 48% | 48% | 53% |
| Specificity | 84% | 90% | 88% | 84% |
| OR Quartile 2 | 4.79 | 4.66 | 4.15 | 4.79 |
| p Value | 1.8E−4 | 2.8E−6 | 1.5E−5 | 1.8E−4 |
| Lower limit of 95% CI | 2.11 | 2.45 | 2.18 | 2.11 |
| Upper limit of 95% CI | 10.9 | 8.87 | 7.90 | 10.9 |
| OR Quartile 3 | 6.39 | 7.09 | 5.84 | 6.39 |
| p Value | 1.6E−9 | 4.5E−14 | 8.7E−12 | 1.6E−9 |
| Lower limit of 95% CI | 3.50 | 4.26 | 3.52 | 3.50 |
| Upper limit of 95% CI | 11.7 | 11.8 | 9.69 | 11.7 |
| OR Quartile 4 | 6.15 | 8.45 | 6.55 | 6.15 |
| p Value | 1.2E−10 | 4.4E−13 | 2.2E−11 | 1.2E−10 |
| Lower limit of 95% CI | 3.54 | 4.75 | 3.78 | 3.54 |
| Upper limit of 95% CI | 10.7 | 15.1 | 11.4 | 10.7 |
| C-C motif chemokine | | | | |
| 14 X Serum Creatinine | | | | |
| AUC | 0.77 | 0.83 | 0.83 | 0.77 |
| SE | 0.033 | 0.025 | 0.025 | 0.033 |
| p Value | 2.2E−16 | 0 | 0 | 2.2E−16 |
| nCohort Non-persistent | 239 | 196 | 202 | 239 |
| nCohort Persistent | 83 | 128 | 120 | 83 |
| Cutoff Quartile 2 | 1.30 | 1.29 | 1.29 | 1.30 |
| Sensitivity | 93% | 94% | 93% | 93% |
| Specificity | 31% | 37% | 36% | 31% |
| Cutoff Quartile 3 | 3.97 | 3.97 | 3.91 | 3.97 |
| Sensitivity | 86% | 82% | 82% | 86% |
| Specificity | 62% | 71% | 69% | 62% |
| Cutoff Quartile 4 | 18.0 | 18.3 | 16.8 | 18.0 |
| Sensitivity | 48% | 48% | 50% | 48% |
| Specificity | 83% | 90% | 90% | 83% |
| OR Quartile 2 | 5.87 | 8.90 | 7.92 | 5.87 |
| p Value | 7.3E−5 | 2.9E−8 | 1.5E−7 | 7.3E−5 |
| Lower limit of 95% CI | 2.45 | 4.11 | 3.66 | 2.45 |
| Upper limit of 95% CI | 14.1 | 19.3 | 17.2 | 14.1 |
| OR Quartile 3 | 9.80 | 11.1 | 10.6 | 9.80 |
| p Value | 1.8E−11 | 0 | 0 | 1.8E−11 |
| Lower limit of 95% CI | 5.04 | 6.45 | 6.09 | 5.04 |
| Upper limit of 95% CI | 19.1 | 19.2 | 18.6 | 19.1 |
| OR Quartile 4 | 4.49 | 8.75 | 8.62 | 4.49 |
| p Value | 7.1E−8 | 4.2E−13 | 2.4E−13 | 7.1E−8 |
| Lower limit of 95% CI | 2.60 | 4.87 | 4.84 | 2.60 |
| Upper limit of 95% CI | 7.76 | 15.7 | 15.3 | 7.76 |
| C-C motif chemokine | | | | |
| 14 X Cystatin-C | | | | |
| AUC | 0.77 | 0.81 | 0.81 | 0.77 |
| SE | 0.034 | 0.026 | 0.027 | 0.034 |
| p Value | 1.8E−15 | 0 | 0 | 1.8E−15 |
| nCohort Non-persistent | 228 | 185 | 190 | 228 |
| nCohort Persistent | 78 | 123 | 117 | 78 |
| Cutoff Quartile 2 | 958000 | 956000 | 955000 | 958000 |
| Sensitivity | 92% | 92% | 91% | 92% |
| Specificity | 31% | 36% | 35% | 31% |
| Cutoff Quartile 3 | 2880000 | 2880000 | 2860000 | 2880000 |
| Sensitivity | 85% | 80% | 80% | 85% |
| Specificity | 62% | 70% | 68% | 62% |
| Cutoff Quartile 4 | 1.03E7 | 1.04E7 | 1.03E7 | 1.03E7 |
| Sensitivity | 53% | 50% | 50% | 53% |
| Specificity | 84% | 91% | 91% | 84% |
| OR Quartile 2 | 5.43 | 6.42 | 5.83 | 5.43 |
| p Value | 1.6E−4 | 3.2E−7 | 1.3E−6 | 1.6E−4 |
| Lower limit of 95% CI | 2.25 | 3.15 | 2.86 | 2.25 |
| Upper limit of 95% CI | 13.1 | 13.1 | 11.9 | 13.1 |
| OR Quartile 3 | 8.91 | 9.75 | 8.86 | 8.91 |
| p Value | 1.6E−10 | 2.2E−16 | 6.9E−15 | 1.6E−10 |
| Lower limit of 95% CI | 4.56 | 5.65 | 5.11 | 4.56 |
| Upper limit of 95% CI | 17.4 | 16.8 | 15.3 | 17.4 |
| OR Quartile 4 | 5.91 | 10.4 | 9.72 | 5.91 |
| p Value | 9.6E−10 | 1.7E−13 | 1.9E−13 | 9.6E−10 |
| Lower limit of 95% CI | 3.34 | 5.58 | 5.30 | 3.34 |
| Upper limit of 95% CI | 10.4 | 19.4 | 17.8 | 10.4 |
| C-C motif chemokine 14 X | | | | |
| Serum Creatinine/(Weight | | | | |
| adjusted urine output) | | | | |
| AUC | 0.80 | 0.82 | 0.80 | 0.80 |
| SE | 0.031 | 0.025 | 0.027 | 0.031 |
| p Value | 0 | 0 | 0 | 0 |
| nCohort Non-persistent | 238 | 194 | 200 | 238 |
| nCohort Persistent | 83 | 127 | 119 | 83 |
| Cutoff Quartile 2 | 2.17 | 2.17 | 2.15 | 2.17 |
| Sensitivity | 92% | 91% | 91% | 92% |
| Specificity | 31% | 36% | 34% | 31% |
| Cutoff Quartile 3 | 10.2 | 10.2 | 10.1 | 10.2 |
| Sensitivity | 84% | 81% | 80% | 84% |
| Specificity | 62% | 70% | 68% | 62% |
| Cutoff Quartile 4 | 69.8 | 69.8 | 67.6 | 69.8 |
| Sensitivity | 52% | 51% | 50% | 52% |
| Specificity | 84% | 92% | 90% | 84% |
| OR Quartile 2 | 4.80 | 5.82 | 5.17 | 4.80 |
| p Value | 1.8E−4 | 4.6E−7 | 2.6E−6 | 1.8E−4 |
| Lower limit of 95% CI | 2.11 | 2.94 | 2.61 | 2.11 |
| Upper limit of 95% CI | 10.9 | 11.5 | 10.3 | 10.9 |
| OR Quartile 3 | 8.70 | 10.1 | 8.22 | 8.70 |
| p Value | 5.7E−11 | 0 | 1.4E−14 | 5.7E−11 |
| Lower limit of 95% CI | 4.55 | 5.86 | 4.81 | 4.55 |
| Upper limit of 95% CI | 16.6 | 17.3 | 14.1 | 16.6 |
| OR Quartile 4 | 5.66 | 11.7 | 8.38 | 5.66 |
| p Value | 8.1E−10 | 7.1E−15 | 5.4E−13 | 8.1E−10 |
| Lower limit of 95% CI | 3.25 | 6.28 | 4.70 | 3.25 |
| Upper limit of 95% CI | 9.83 | 21.7 | 14.9 | 9.83 |
| C-C motif chemokine 14 X | | | | |
| Cystatin-C/(Weight adjusted | | | | |
| urine output) | | | | |
| AUC | 0.78 | 0.80 | 0.78 | 0.78 |
| SE | 0.033 | 0.027 | 0.029 | 0.033 |
| p Value | 0 | 0 | 0 | 0 |
| nCohort Non- | 227 | 183 | 188 | 227 |
| persistent | | | | |
| nCohort Persistent | 78 | 122 | 116 | 78 |
| Cutoff Quartile 2 | 1580000 | 1580000 | 1580000 | 1580000 |
| Sensitivity | 91% | 90% | 90% | 91% |
| Specificity | 30% | 35% | 34% | 30% |
| Cutoff Quartile 3 | 6710000 | 6710000 | 6570000 | 6710000 |
| Sensitivity | 83% | 80% | 78% | 83% |
| Specificity | 61% | 69% | 68% | 61% |
| Cutoff Quartile 4 | 3.85E7 | 3.85E7 | 3.84E7 | 3.85E7 |
| Sensitivity | 49% | 48% | 46% | 49% |
| Specificity | 83% | 90% | 88% | 83% |
| OR Quartile 2 | 4.43 | 4.93 | 4.47 | 4.43 |
| p Value | 4.2E−4 | 2.9E−6 | 1.2E−5 | 4.2E−4 |
| Lower limit of 95% CI | 1.94 | 2.53 | 2.29 | 1.94 |
| Upper limit of 95% CI | 10.1 | 9.62 | 8.74 | 10.1 |
| OR Quartile 3 | 7.90 | 8.80 | 7.58 | 7.90 |
| p Value | 5.4E−10 | 3.1E−15 | 1.5E−13 | 5.4E−10 |
| Lower limit of 95% CI | 4.11 | 5.13 | 4.43 | 4.11 |
| Upper limit of 95% CI | 15.2 | 15.1 | 13.0 | 15.2 |
| OR Quartile 4 | 4.58 | 7.82 | 6.04 | 4.58 |
| p Value | 1.1E−7 | 1.1E−11 | 5.9E−10 | 1.1E−7 |
| Lower limit of 95% CI | 2.61 | 4.32 | 3.42 | 2.61 |
| Upper limit of 95% CI | 8.03 | 14.2 | 10.7 | 8.03 |

### Example 3. Use of C-C Motif Chemokine 14 and One or More of Serum Creatinine, Urine Output, and Cystatin C in a Logistic Regression Model for Evaluating Renal Status in Patients Admitted to the ICU: Persistent at KDIGO Stage 3

With the same study and patient cohort as in Example 2 above, a logistic regression model with combinations of C-C motif chemokine 14, serum creatinine, and Cystatin C concentrations, and weight adjusted urine output as predictors was used to distinguish between the “persistent” and “non-persistent” cohorts. The logarithm of the odds (log-odds) of a patient belonging to the “persistent” cohort was modeled by a linear combination of the predictors. The overall ability of the combination of predictors to distinguish between the “persistent” and “non-persistent” cohorts was determined using a ROC analysis, and the sensitivity and specificity were determined at cutoffs corresponding to the 25^{th}, 50^{th}, and 75^{th} percentiles of the log-odds values.

Because a logistic regression model makes no a priori assumptions about the weighting of the predictors, a proportion of the marker concentrations and weight adjusted urine output data, called the training dataset, was used to generate the model parameters, which were then used to predict the cohort classification of the remaining data, called the validation dataset. To ensure robustness of the ROC AUC, sensitivity and specificity estimates, a k-fold cross-validation technique was used, whereby the entire dataset was divided into k approximately equal subsets. Then the model parameters were estimated using k−1 subsets as the training dataset, and the cohort classifications were predicted with the remaining validation dataset. This process of training and classifying was repeated using each of the k subsets as the validation dataset, and the classification results were averaged to give an estimate of the ROC AUC, sensitivity and specificity.

The individual marker assay results were used in combinations as predictors of the regression as indicated herein: C-C motif chemokine 14 with weight adjusted urine output, C-C motif chemokine 14 with serum creatinine, C-C motif chemokine 14 with Cystatin-C, C-C motif chemokine 14 with serum creatinine and weight adjusted urine output, and C-C motif chemokine 14 with Cystatin-C and weight adjusted urine output. Assay results and weight adjusted urine output were log-transformed prior to the fitting procedure.

TABLE 8
Comparison of the ROC AUC in samples for the “persistent” and “non-persistent”
cohorts where persistence starts within 24 hours after sample collection and renal status
was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.80 | 0.78 | 0.84 | 0.83 | 0.81 | 0.81 | 0.82 | 0.81 | 0.81 |
| SE | 0.026 | 0.028 | 0.033 | 0.025 | 0.026 | 0.036 | 0.026 | 0.027 | 0.036 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.16 | 0.16 | 0.03 | 0.10 | 0.10 | 0.03 | 0.10 | 0.11 | 0.03 |
| Sensitivity | 90% | 90% | 98% | 92% | 92% | 93% | 94% | 93% | 93% |
| Specificity | 34% | 33% | 29% | 34% | 33% | 28% | 34% | 33% | 28% |
| Cutoff Quartile 3 | 0.31 | 0.29 | 0.07 | 0.24 | 0.23 | 0.07 | 0.22 | 0.21 | 0.07 |
| Sensitivity | 79% | 79% | 86% | 85% | 84% | 85% | 84% | 83% | 85% |
| Specificity | 68% | 66% | 56% | 67% | 66% | 55% | 65% | 63% | 55% |
| Cutoff Quartile 4 | 0.56 | 0.51 | 0.14 | 0.53 | 0.49 | 0.14 | 0.45 | 0.42 | 0.14 |
| Sensitivity | 52% | 53% | 70% | 58% | 55% | 68% | 56% | 53% | 68% |
| Specificity | 92% | 90% | 82% | 91% | 89% | 81% | 89% | 86% | 81% |
| | C-C motif chemokine 14 with serum creatinine | | | | | | | | |
| AUC | 0.85 | 0.86 | 0.74 | 0.87 | 0.88 | 0.76 | 0.86 | 0.86 | 0.76 |
| SE | 0.022 | 0.021 | 0.034 | 0.022 | 0.021 | 0.034 | 0.023 | 0.023 | 0.034 |
| p Value | 0 | 0 | 0 | 0 | 0 | 0 | 0 | 0 | 0 |
| nCohort Non- | 203 | 209 | 278 | 217 | 221 | 281 | 225 | 229 | 281 |
| persistent | | | | | | | | | |
| nCohort Persistent | 121 | 113 | 44 | 107 | 101 | 41 | 99 | 93 | 41 |
| Cutoff Quartile 2 | 0.10 | 0.08 | 0.06 | 0.07 | 0.06 | 0.05 | 0.07 | 0.06 | 0.05 |
| Sensitivity | 95% | 97% | 93% | 95% | 96% | 95% | 96% | 96% | 95% |
| Specificity | 37% | 37% | 28% | 35% | 35% | 28% | 34% | 34% | 28% |
| Cutoff Quartile 3 | 0.29 | 0.25 | 0.10 | 0.20 | 0.19 | 0.09 | 0.19 | 0.18 | 0.09 |
| Sensitivity | 83% | 83% | 82% | 88% | 87% | 85% | 87% | 86% | 85% |
| Specificity | 70% | 68% | 55% | 69% | 67% | 55% | 66% | 65% | 55% |
| Cutoff Quartile 4 | 0.63 | 0.61 | 0.19 | 0.58 | 0.54 | 0.18 | 0.52 | 0.48 | 0.18 |
| Sensitivity | 53% | 55% | 52% | 60% | 61% | 51% | 61% | 62% | 51% |
| Specificity | 92% | 91% | 79% | 92% | 91% | 79% | 91% | 90% | 79% |
| | C-C motif chemokine 14 with Cystatin-C | | | | | | | | |
| AUC | 0.82 | 0.82 | 0.77 | 0.85 | 0.85 | 0.79 | 0.82 | 0.83 | 0.79 |
| SE | 0.025 | 0.025 | 0.035 | 0.023 | 0.025 | 0.037 | 0.026 | 0.026 | 0.037 |
| p Value | 0 | 0 | 0 | 0 | 0 | 0 | 0 | 0 | 0 |
| nCohort Non- | 192 | 196 | 267 | 206 | 208 | 270 | 214 | 216 | 270 |
| persistent | | | | | | | | | |
| nCohort Persistent | 116 | 111 | 39 | 102 | 99 | 36 | 94 | 91 | 36 |
| Cutoff Quartile 2 | 0.12 | 0.12 | 0.04 | 0.08 | 0.07 | 0.03 | 0.08 | 0.08 | 0.03 |
| Sensitivity | 92% | 90% | 95% | 94% | 94% | 94% | 95% | 93% | 94% |
| Specificity | 35% | 34% | 28% | 34% | 34% | 28% | 34% | 33% | 28% |
| Cutoff Quartile 3 | 0.33 | 0.32 | 0.09 | 0.26 | 0.24 | 0.08 | 0.24 | 0.22 | 0.08 |
| Sensitivity | 83% | 83% | 92% | 85% | 86% | 89% | 86% | 86% | 89% |
| Specificity | 70% | 69% | 56% | 67% | 67% | 55% | 66% | 65% | 55% |
| Cutoff Quartile 4 | 0.58 | 0.56 | 0.17 | 0.52 | 0.51 | 0.16 | 0.49 | 0.45 | 0.16 |
| Sensitivity | 47% | 51% | 54% | 56% | 57% | 56% | 53% | 56% | 56% |
| Specificity | 89% | 90% | 79% | 90% | 90% | 79% | 87% | 88% | 79% |
| | C-C motif chemokine 14 with serum creatinine and weight adjusted urine output | | | | | | | | |
| AUC | 0.87 | 0.87 | 0.83 | 0.89 | 0.88 | 0.82 | 0.87 | 0.87 | 0.82 |
| SE | 0.020 | 0.021 | 0.034 | 0.020 | 0.022 | 0.035 | 0.022 | 0.023 | 0.035 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.08 | 0.07 | 0.03 | 0.05 | 0.05 | 0.03 | 0.05 | 0.05 | 0.03 |
| Sensitivity | 96% | 96% | 98% | 96% | 96% | 95% | 96% | 96% | 95% |
| Specificity | 38% | 36% | 29% | 36% | 35% | 28% | 35% | 33% | 28% |
| Cutoff Quartile 3 | 0.29 | 0.27 | 0.06 | 0.21 | 0.20 | 0.06 | 0.20 | 0.20 | 0.06 |
| Sensitivity | 87% | 88% | 86% | 89% | 89% | 85% | 89% | 88% | 85% |
| Specificity | 72% | 71% | 56% | 69% | 68% | 55% | 67% | 66% | 55% |
| Cutoff Quartile 4 | 0.64 | 0.60 | 0.15 | 0.58 | 0.55 | 0.14 | 0.50 | 0.46 | 0.14 |
| Sensitivity | 54% | 54% | 68% | 63% | 62% | 66% | 64% | 64% | 66% |
| Specificity | 93% | 91% | 82% | 94% | 92% | 81% | 92% | 91% | 81% |
| | C-C motif chemokine 14 X Cystatin-C/(Weight adjusted urine output) | | | | | | | | |
| AUC | 0.82 | 0.82 | 0.84 | 0.85 | 0.83 | 0.83 | 0.82 | 0.82 | 0.83 |
| SE | 0.026 | 0.027 | 0.036 | 0.024 | 0.025 | 0.037 | 0.027 | 0.026 | 0.037 |
| p Value | 0 | 0 | 0 | 0 | 0 | 0 | 0 | 0 | 0 |
| nCohort Non- | 187 | 191 | 262 | 201 | 203 | 265 | 209 | 211 | 265 |
| persistent | | | | | | | | | |
| nCohort Persistent | 114 | 109 | 39 | 100 | 97 | 36 | 92 | 89 | 36 |
| Cutoff Quartile 2 | 0.12 | 0.11 | 0.03 | 0.07 | 0.08 | 0.02 | 0.08 | 0.07 | 0.02 |
| Sensitivity | 90% | 91% | 95% | 92% | 93% | 94% | 92% | 93% | 94% |
| Specificity | 35% | 34% | 28% | 34% | 33% | 28% | 33% | 33% | 28% |
| Cutoff Quartile 3 | 0.31 | 0.32 | 0.06 | 0.26 | 0.24 | 0.06 | 0.24 | 0.23 | 0.06 |
| Sensitivity | 82% | 82% | 90% | 84% | 82% | 86% | 85% | 85% | 86% |
| Specificity | 70% | 68% | 56% | 67% | 66% | 55% | 66% | 65% | 55% |
| Cutoff Quartile 4 | 0.59 | 0.57 | 0.16 | 0.53 | 0.52 | 0.15 | 0.46 | 0.44 | 0.15 |
| Sensitivity | 49% | 51% | 72% | 56% | 57% | 69% | 54% | 54% | 69% |
| Specificity | 90% | 90% | 82% | 91% | 90% | 81% | 88% | 87% | 81% |

TABLE 9
Comparison of marker levels and the area under the ROC curve (AUC) in urine samples for the “persistent”
and “non-persistent” cohorts where persistence starts within 48 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.81 | 0.78 | 0.80 | 0.83 | 0.82 | 0.78 | 0.83 | 0.81 | 0.78 |
| SE | 0.026 | 0.027 | 0.031 | 0.024 | 0.025 | 0.031 | 0.025 | 0.025 | 0.031 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.20 | 0.20 | 0.07 | 0.12 | 0.13 | 0.07 | 0.11 | 0.13 | 0.07 |
| Sensitivity | 90% | 90% | 95% | 93% | 93% | 93% | 94% | 91% | 93% |
| Specificity | 37% | 35% | 30% | 36% | 35% | 30% | 35% | 33% | 30% |
| Cutoff Quartile 3 | 0.37 | 0.34 | 0.13 | 0.28 | 0.27 | 0.12 | 0.25 | 0.24 | 0.12 |
| Sensitivity | 79% | 78% | 80% | 83% | 83% | 83% | 83% | 83% | 83% |
| Specificity | 71% | 68% | 58% | 69% | 68% | 58% | 67% | 66% | 58% |
| Cutoff Quartile 4 | 0.63 | 0.57 | 0.26 | 0.60 | 0.55 | 0.24 | 0.52 | 0.48 | 0.24 |
| Sensitivity | 49% | 50% | 58% | 55% | 54% | 55% | 55% | 52% | 55% |
| Specificity | 92% | 91% | 83% | 93% | 90% | 82% | 90% | 88% | 82% |
| | C-C motif chemokine 14 with serum creatinine | | | | | | | | |
| AUC | 0.85 | 0.86 | 0.77 | 0.88 | 0.87 | 0.78 | 0.87 | 0.86 | 0.78 |
| SE | 0.021 | 0.021 | 0.030 | 0.020 | 0.021 | 0.030 | 0.021 | 0.022 | 0.030 |
| p Value | 0 | 0 | 0 | 0 | 0 | 0 | 0 | 0 | 0 |
| nCohort Non- | 188 | 196 | 258 | 205 | 210 | 262 | 215 | 220 | 262 |
| persistent | | | | | | | | | |
| nCohort Persistent | 136 | 127 | 64 | 119 | 113 | 60 | 109 | 103 | 60 |
| Cutoff Quartile 2 | 0.14 | 0.12 | 0.08 | 0.08 | 0.07 | 0.07 | 0.08 | 0.07 | 0.07 |
| Sensitivity | 95% | 95% | 94% | 96% | 96% | 95% | 96% | 96% | 95% |
| Specificity | 39% | 38% | 30% | 37% | 36% | 30% | 36% | 35% | 30% |
| Cutoff Quartile 3 | 0.35 | 0.31 | 0.14 | 0.24 | 0.24 | 0.13 | 0.22 | 0.20 | 0.13 |
| Sensitivity | 82% | 82% | 89% | 87% | 88% | 88% | 88% | 88% | 88% |
| Specificity | 73% | 71% | 60% | 72% | 71% | 59% | 69% | 68% | 59% |
| Cutoff Quartile 4 | 0.69 | 0.67 | 0.29 | 0.65 | 0.63 | 0.27 | 0.57 | 0.55 | 0.27 |
| Sensitivity | 50% | 53% | 52% | 59% | 58% | 53% | 57% | 59% | 53% |
| Specificity | 93% | 93% | 81% | 95% | 92% | 81% | 91% | 91% | 81% |
| | C-C motif chemokine 14 with Cystatin-C | | | | | | | | |
| AUC | 0.83 | 0.83 | 0.79 | 0.85 | 0.85 | 0.79 | 0.85 | 0.85 | 0.79 |
| SE | 0.024 | 0.025 | 0.029 | 0.023 | 0.023 | 0.030 | 0.023 | 0.023 | 0.030 |
| p Value | 0 | 0 | 0 | 0 | 0 | 0 | 0 | 0 | 0 |
| nCohort Non- | 178 | 184 | 247 | 194 | 197 | 251 | 204 | 207 | 251 |
| persistent | | | | | | | | | |
| nCohort Persistent | 130 | 124 | 59 | 114 | 111 | 55 | 104 | 101 | 55 |
| Cutoff Quartile 2 | 0.16 | 0.15 | 0.06 | 0.09 | 0.10 | 0.05 | 0.10 | 0.10 | 0.05 |
| Sensitivity | 92% | 92% | 95% | 91% | 93% | 95% | 94% | 94% | 95% |
| Specificity | 37% | 36% | 30% | 35% | 35% | 29% | 35% | 34% | 29% |
| Cutoff Quartile 3 | 0.40 | 0.36 | 0.15 | 0.30 | 0.30 | 0.13 | 0.27 | 0.26 | 0.13 |
| Sensitivity | 81% | 81% | 92% | 85% | 86% | 87% | 87% | 86% | 87% |
| Specificity | 72% | 71% | 60% | 71% | 70% | 58% | 69% | 68% | 58% |
| Cutoff Quartile 4 | 0.65 | 0.63 | 0.27 | 0.62 | 0.57 | 0.25 | 0.53 | 0.53 | 0.25 |
| Sensitivity | 48% | 50% | 56% | 55% | 55% | 58% | 55% | 52% | 58% |
| Specificity | 92% | 92% | 82% | 93% | 92% | 82% | 90% | 88% | 82% |
| | C-C motif chemokine 14 with serum creatinine and weight adjusted urine output | | | | | | | | |
| AUC | 0.86 | 0.85 | 0.82 | 0.88 | 0.87 | 0.80 | 0.87 | 0.86 | 0.80 |
| SE | 0.021 | 0.022 | 0.028 | 0.020 | 0.021 | 0.030 | 0.021 | 0.023 | 0.030 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.13 | 0.11 | 0.06 | 0.07 | 0.07 | 0.06 | 0.07 | 0.07 | 0.06 |
| Sensitivity | 96% | 95% | 95% | 96% | 96% | 95% | 96% | 96% | 95% |
| Specificity | 40% | 38% | 30% | 37% | 36% | 30% | 36% | 35% | 30% |
| Cutoff Quartile 3 | 0.37 | 0.34 | 0.12 | 0.26 | 0.24 | 0.12 | 0.23 | 0.23 | 0.12 |
| Sensitivity | 82% | 84% | 88% | 88% | 88% | 90% | 89% | 86% | 90% |
| Specificity | 74% | 72% | 60% | 72% | 70% | 59% | 70% | 67% | 59% |
| Cutoff Quartile 4 | 0.70 | 0.63 | 0.25 | 0.65 | 0.60 | 0.24 | 0.58 | 0.54 | 0.24 |
| Sensitivity | 50% | 52% | 58% | 57% | 58% | 53% | 60% | 60% | 53% |
| Specificity | 93% | 92% | 83% | 94% | 93% | 82% | 93% | 91% | 82% |
| | C-C motif chemokine 14 X Cystatin-C/(Weight adjusted urine output) | | | | | | | | |
| AUC | 0.83 | 0.82 | 0.79 | 0.85 | 0.85 | 0.80 | 0.84 | 0.83 | 0.80 |
| SE | 0.024 | 0.025 | 0.033 | 0.023 | 0.025 | 0.030 | 0.024 | 0.025 | 0.030 |
| p Value | 0 | 0 | 0 | 0 | 0 | 0 | 0 | 0 | 0 |
| nCohort Non- | 173 | 179 | 242 | 189 | 192 | 246 | 199 | 202 | 246 |
| persistent | | | | | | | | | |
| nCohort Persistent | 128 | 122 | 59 | 112 | 109 | 55 | 102 | 99 | 55 |
| Cutoff Quartile 2 | 0.15 | 0.13 | 0.04 | 0.09 | 0.09 | 0.04 | 0.10 | 0.09 | 0.04 |
| Sensitivity | 91% | 91% | 93% | 93% | 94% | 95% | 94% | 93% | 95% |
| Specificity | 38% | 36% | 30% | 36% | 36% | 30% | 35% | 34% | 30% |
| Cutoff Quartile 3 | 0.40 | 0.39 | 0.12 | 0.31 | 0.32 | 0.12 | 0.27 | 0.26 | 0.12 |
| Sensitivity | 81% | 80% | 85% | 83% | 83% | 89% | 87% | 85% | 89% |
| Specificity | 73% | 70% | 59% | 70% | 69% | 59% | 69% | 67% | 59% |
| Cutoff Quartile 4 | 0.65 | 0.64 | 0.28 | 0.61 | 0.58 | 0.26 | 0.53 | 0.51 | 0.26 |
| Sensitivity | 47% | 48% | 51% | 54% | 56% | 53% | 56% | 55% | 53% |
| Specificity | 91% | 91% | 81% | 93% | 93% | 81% | 91% | 90% | 81% |

TABLE 10
Comparison of marker levels and the area under the ROC curve (AUC) in urine samples for the “persistent”
and “non-persistent” cohorts where persistence starts within 72 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.79 | 0.78 | 0.75 | 0.82 | 0.82 | 0.76 | 0.81 | 0.80 | 0.76 |
| SE | 0.026 | 0.027 | 0.033 | 0.025 | 0.025 | 0.032 | 0.026 | 0.026 | 0.032 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.24 | 0.23 | 0.10 | 0.17 | 0.17 | 0.09 | 0.16 | 0.16 | 0.09 |
| Sensitivity | 89% | 90% | 93% | 92% | 92% | 93% | 92% | 93% | 93% |
| Specificity | 37% | 36% | 31% | 37% | 36% | 30% | 36% | 35% | 30% |
| Cutoff Quartile 3 | 0.41 | 0.38 | 0.17 | 0.35 | 0.32 | 0.16 | 0.30 | 0.28 | 0.16 |
| Sensitivity | 76% | 76% | 76% | 80% | 80% | 83% | 81% | 81% | 83% |
| Specificity | 72% | 69% | 58% | 71% | 69% | 59% | 68% | 67% | 59% |
| Cutoff Quartile 4 | 0.67 | 0.62 | 0.30 | 0.65 | 0.61 | 0.29 | 0.58 | 0.52 | 0.29 |
| Sensitivity | 46% | 46% | 51% | 52% | 50% | 54% | 50% | 50% | 54% |
| Specificity | 93% | 90% | 83% | 94% | 91% | 83% | 90% | 88% | 83% |
| | C-C motif chemokine 14 with serum creatinine | | | | | | | | |
| AUC | 0.84 | 0.85 | 0.75 | 0.86 | 0.87 | 0.77 | 0.84 | 0.85 | 0.77 |
| SE | 0.022 | 0.021 | 0.032 | 0.022 | 0.021 | 0.030 | 0.023 | 0.022 | 0.030 |
| p Value | 0 | 0 | 0 | 0 | 0 | 0 | 0 | 0 | 0 |
| nCohort Non- | 177 | 186 | 248 | 192 | 198 | 253 | 204 | 210 | 253 |
| persistent | | | | | | | | | |
| nCohort Persistent | 147 | 137 | 74 | 132 | 125 | 69 | 120 | 113 | 69 |
| Cutoff Quartile 2 | 0.19 | 0.14 | 0.11 | 0.12 | 0.10 | 0.09 | 0.12 | 0.09 | 0.09 |
| Sensitivity | 93% | 95% | 91% | 93% | 94% | 94% | 94% | 95% | 94% |
| Specificity | 40% | 40% | 30% | 38% | 37% | 30% | 36% | 36% | 30% |
| Cutoff Quartile 3 | 0.41 | 0.35 | 0.18 | 0.32 | 0.29 | 0.16 | 0.30 | 0.25 | 0.16 |
| Sensitivity | 80% | 81% | 84% | 83% | 86% | 84% | 84% | 87% | 84% |
| Specificity | 75% | 73% | 60% | 72% | 73% | 59% | 70% | 70% | 59% |
| Cutoff Quartile 4 | 0.72 | 0.69 | 0.32 | 0.69 | 0.67 | 0.30 | 0.61 | 0.59 | 0.30 |
| Sensitivity | 48% | 51% | 49% | 53% | 54% | 51% | 53% | 57% | 51% |
| Specificity | 94% | 94% | 82% | 94% | 93% | 82% | 92% | 92% | 82% |
| | C-C motif chemokine 14 with Cystatin-C | | | | | | | | |
| AUC | 0.82 | 0.83 | 0.76 | 0.84 | 0.84 | 0.77 | 0.82 | 0.83 | 0.77 |
| SE | 0.024 | 0.024 | 0.031 | 0.023 | 0.024 | 0.031 | 0.025 | 0.024 | 0.031 |
| p Value | 0 | 0 | 0 | 0 | 0 | 0 | 0 | 0 | 0 |
| nCohort Non- | 167 | 174 | 237 | 181 | 185 | 242 | 193 | 197 | 242 |
| persistent | | | | | | | | | |
| nCohort Persistent | 141 | 134 | 69 | 127 | 123 | 64 | 115 | 111 | 64 |
| Cutoff Quartile 2 | 0.19 | 0.17 | 0.08 | 0.13 | 0.12 | 0.07 | 0.12 | 0.11 | 0.07 |
| Sensitivity | 91% | 90% | 94% | 91% | 92% | 92% | 92% | 93% | 92% |
| Specificity | 38% | 37% | 31% | 36% | 36% | 30% | 35% | 35% | 30% |
| Cutoff Quartile 3 | 0.44 | 0.41 | 0.19 | 0.38 | 0.35 | 0.17 | 0.32 | 0.30 | 0.17 |
| Sensitivity | 79% | 81% | 83% | 82% | 81% | 83% | 83% | 84% | 83% |
| Specificity | 74% | 74% | 59% | 72% | 71% | 59% | 69% | 69% | 59% |
| Cutoff Quartile 4 | 0.70 | 0.67 | 0.31 | 0.66 | 0.64 | 0.31 | 0.58 | 0.56 | 0.31 |
| Sensitivity | 45% | 48% | 51% | 51% | 54% | 50% | 51% | 54% | 50% |
| Specificity | 92% | 93% | 82% | 93% | 94% | 81% | 91% | 91% | 81% |
| | C-C motif chemokine 14 with serum creatinine and weight adjusted urine output | | | | | | | | |
| AUC | 0.84 | 0.85 | 0.78 | 0.86 | 0.86 | 0.79 | 0.85 | 0.84 | 0.79 |
| SE | 0.022 | 0.022 | 0.031 | 0.022 | 0.021 | 0.030 | 0.023 | 0.024 | 0.030 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.18 | 0.14 | 0.08 | 0.12 | 0.10 | 0.08 | 0.11 | 0.09 | 0.08 |
| Sensitivity | 92% | 95% | 91% | 93% | 94% | 93% | 93% | 94% | 93% |
| Specificity | 40% | 40% | 30% | 38% | 37% | 30% | 36% | 35% | 30% |
| Cutoff Quartile 3 | 0.42 | 0.38 | 0.16 | 0.33 | 0.30 | 0.16 | 0.29 | 0.27 | 0.16 |
| Sensitivity | 79% | 81% | 81% | 82% | 85% | 86% | 84% | 85% | 86% |
| Specificity | 75% | 73% | 60% | 73% | 72% | 60% | 70% | 69% | 60% |
| Cutoff Quartile 4 | 0.73 | 0.69 | 0.28 | 0.70 | 0.65 | 0.28 | 0.62 | 0.57 | 0.28 |
| Sensitivity | 47% | 50% | 55% | 53% | 55% | 54% | 54% | 54% | 54% |
| Specificity | 94% | 93% | 84% | 94% | 94% | 83% | 92% | 90% | 83% |
| | C-C motif chemokine 14 with Cystatin-C and weight adjusted urine output | | | | | | | | |
| AUC | 0.83 | 0.83 | 0.79 | 0.84 | 0.85 | 0.78 | 0.82 | 0.83 | 0.78 |
| SE | 0.024 | 0.024 | 0.032 | 0.024 | 0.023 | 0.032 | 0.025 | 0.025 | 0.032 |
| p Value | 0 | 0 | 0 | 0 | 0 | 0 | 0 | 0 | 0 |
| nCohort Non- | 162 | 169 | 232 | 176 | 180 | 237 | 188 | 192 | 237 |
| persistent | | | | | | | | | |
| nCohort Persistent | 139 | 132 | 69 | 125 | 121 | 64 | 113 | 109 | 64 |
| Cutoff Quartile 2 | 0.19 | 0.16 | 0.07 | 0.12 | 0.12 | 0.06 | 0.12 | 0.10 | 0.06 |
| Sensitivity | 91% | 91% | 91% | 91% | 92% | 94% | 92% | 93% | 94% |
| Specificity | 39% | 38% | 30% | 37% | 37% | 30% | 36% | 35% | 30% |
| Cutoff Quartile 3 | 0.46 | 0.42 | 0.17 | 0.38 | 0.36 | 0.15 | 0.33 | 0.32 | 0.15 |
| Sensitivity | 79% | 78% | 81% | 82% | 83% | 83% | 82% | 83% | 83% |
| Specificity | 75% | 72% | 59% | 73% | 73% | 59% | 70% | 69% | 59% |
| Cutoff Quartile 4 | 0.70 | 0.67 | 0.32 | 0.65 | 0.64 | 0.32 | 0.57 | 0.56 | 0.32 |
| Sensitivity | 45% | 48% | 55% | 51% | 54% | 53% | 50% | 53% | 53% |
| Specificity | 93% | 93% | 84% | 94% | 94% | 83% | 90% | 91% | 83% |

### Example 4. Use of C-C Motif Chemokine 14 and One or More of Serum Creatinine, Urine Output, and Cystatin C in a Linear Discriminant for Evaluating Renal Status in Patients Admitted to the ICU: Persistent at KDIGO Stage 3

With the same study and patient cohort as in Example 2 above, linear discriminant analysis with combinations of C-C motif chemokine 14, serum creatinine, and Cystatin C concentrations, and weight adjusted urine output as predictors was used to distinguish between the “persistent” and “non-persistent” cohorts. Although similar to the logistic regression model (described in Example 3 above) in that both techniques used a linear combination of predictors to determine class membership, they differ in the fitting function that was used to model the outcome. The linear discriminant modeled the relationship between the “persistent” and “non-persistent” cohorts by finding a linear combination of the predictors that maximized the separation between the two groups, resulting in optimal classification. The overall ability of the combination of predictors to distinguish between the “persistent” and “non-persistent” cohorts was determined by using a ROC analysis, and the sensitivity and specificity were determined for cutoffs corresponding to the 25^{th}, 50^{th}, and 75^{th} percentiles of the discriminant scores.

A linear discriminant makes no a priori assumptions about the weighting of the predictors, and so a k-fold cross validation technique (described in Example 7 above) was used to ensure robustness of the ROC AUC, sensitivity and specificity estimates.

The individual marker assay results were used in combinations as predictors of the regression as indicated herein: C-C motif chemokine 14 with weight adjusted urine output, C-C motif chemokine 14 with serum creatinine, C-C motif chemokine 14 with Cystatin-C, C-C motif chemokine 14 with serum creatinine and weight adjusted urine output, and C-C motif chemokine 14 with Cystatin-C and weight adjusted urine output. Assay results and weight adjusted urine output were log-transformed prior to the fitting procedure.

TABLE 11
Comparison of the ROC AUC in samples for the “persistent” and “non-persistent”
cohorts where persistence starts within 24 hours after sample collection and renal status
was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.80 | 0.78 | 0.84 | 0.83 | 0.81 | 0.81 | 0.82 | 0.81 | 0.81 |
| SE | 0.026 | 0.028 | 0.033 | 0.025 | 0.026 | 0.036 | 0.026 | 0.027 | 0.036 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.16 | 0.16 | 0.03 | 0.10 | 0.10 | 0.03 | 0.10 | 0.11 | 0.03 |
| Sensitivity | 90% | 90% | 98% | 92% | 92% | 93% | 94% | 93% | 93% |
| Specificity | 34% | 33% | 29% | 34% | 33% | 28% | 34% | 33% | 28% |
| Cutoff Quartile 3 | 0.31 | 0.29 | 0.07 | 0.24 | 0.23 | 0.07 | 0.22 | 0.21 | 0.07 |
| Sensitivity | 79% | 79% | 86% | 85% | 84% | 85% | 84% | 83% | 85% |
| Specificity | 68% | 66% | 56% | 67% | 66% | 55% | 65% | 63% | 55% |
| Cutoff Quartile 4 | 0.56 | 0.51 | 0.14 | 0.53 | 0.49 | 0.14 | 0.45 | 0.42 | 0.14 |
| Sensitivity | 52% | 53% | 70% | 58% | 55% | 68% | 56% | 53% | 68% |
| Specificity | 92% | 90% | 82% | 91% | 89% | 81% | 89% | 86% | 81% |
| | C-C motif chemokine 14 with serum creatinine | | | | | | | | |
| AUC | 0.85 | 0.85 | 0.75 | 0.87 | 0.88 | 0.76 | 0.85 | 0.86 | 0.76 |
| SE | 0.022 | 0.022 | 0.033 | 0.022 | 0.022 | 0.034 | 0.023 | 0.023 | 0.034 |
| p Value | 0 | 0 | 0 | 0 | 0 | 0 | 0 | 0 | 0 |
| nCohort Non- | 203 | 209 | 278 | 217 | 221 | 281 | 225 | 229 | 281 |
| persistent | | | | | | | | | |
| nCohort Persistent | 121 | 113 | 44 | 107 | 101 | 41 | 99 | 93 | 41 |
| Cutoff Quartile 2 | 0.11 | 0.10 | 0.05 | 0.08 | 0.07 | 0.05 | 0.08 | 0.07 | 0.05 |
| Sensitivity | 96% | 96% | 95% | 96% | 96% | 95% | 96% | 96% | 95% |
| Specificity | 37% | 37% | 28% | 35% | 35% | 28% | 34% | 34% | 28% |
| Cutoff Quartile 3 | 0.29 | 0.26 | 0.10 | 0.21 | 0.20 | 0.09 | 0.20 | 0.19 | 0.09 |
| Sensitivity | 83% | 83% | 84% | 85% | 88% | 85% | 87% | 88% | 85% |
| Specificity | 69% | 68% | 55% | 67% | 67% | 55% | 66% | 66% | 55% |
| Cutoff Quartile 4 | 0.63 | 0.59 | 0.19 | 0.58 | 0.55 | 0.18 | 0.52 | 0.48 | 0.18 |
| Sensitivity | 55% | 56% | 57% | 61% | 63% | 56% | 60% | 60% | 56% |
| Specificity | 93% | 91% | 80% | 93% | 92% | 79% | 90% | 89% | 79% |
| | C-C motif chemokine 14 with Cystatin-C | | | | | | | | |
| AUC | 0.82 | 0.82 | 0.77 | 0.85 | 0.84 | 0.79 | 0.82 | 0.82 | 0.79 |
| SE | 0.025 | 0.025 | 0.034 | 0.024 | 0.024 | 0.036 | 0.026 | 0.027 | 0.036 |
| p Value | 0 | 0 | 0 | 0 | 0 | 0 | 0 | 0 | 0 |
| nCohort Non- | 192 | 196 | 267 | 206 | 208 | 270 | 214 | 216 | 270 |
| persistent | | | | | | | | | |
| nCohort Persistent | 116 | 111 | 39 | 102 | 99 | 36 | 94 | 91 | 36 |
| Cutoff Quartile 2 | 0.14 | 0.13 | 0.05 | 0.09 | 0.09 | 0.04 | 0.09 | 0.09 | 0.04 |
| Sensitivity | 92% | 91% | 95% | 94% | 95% | 94% | 94% | 93% | 94% |
| Specificity | 35% | 34% | 28% | 34% | 35% | 28% | 33% | 33% | 28% |
| Cutoff Quartile 3 | 0.34 | 0.31 | 0.10 | 0.26 | 0.25 | 0.08 | 0.24 | 0.24 | 0.08 |
| Sensitivity | 83% | 83% | 90% | 85% | 86% | 92% | 86% | 86% | 92% |
| Specificity | 70% | 69% | 56% | 67% | 67% | 56% | 66% | 65% | 56% |
| Cutoff Quartile 4 | 0.58 | 0.56 | 0.17 | 0.54 | 0.53 | 0.16 | 0.47 | 0.45 | 0.16 |
| Sensitivity | 48% | 51% | 51% | 56% | 57% | 56% | 54% | 55% | 56% |
| Specificity | 89% | 90% | 79% | 90% | 90% | 79% | 88% | 88% | 79% |
| | C-C motif chemokine 14 with serum creatinine and weight adjusted urine output | | | | | | | | |
| AUC | 0.87 | 0.87 | 0.85 | 0.89 | 0.88 | 0.82 | 0.87 | 0.87 | 0.82 |
| SE | 0.020 | 0.021 | 0.033 | 0.020 | 0.022 | 0.037 | 0.022 | 0.023 | 0.037 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.09 | 0.08 | 0.01 | 0.06 | 0.05 | 0.01 | 0.05 | 0.05 | 0.01 |
| Sensitivity | 96% | 96% | 98% | 96% | 96% | 95% | 96% | 96% | 95% |
| Specificity | 38% | 36% | 29% | 36% | 35% | 28% | 35% | 33% | 28% |
| Cutoff Quartile 3 | 0.28 | 0.27 | 0.03 | 0.21 | 0.19 | 0.03 | 0.19 | 0.19 | 0.03 |
| Sensitivity | 88% | 87% | 86% | 89% | 89% | 85% | 89% | 88% | 85% |
| Specificity | 73% | 70% | 56% | 69% | 68% | 55% | 67% | 66% | 55% |
| Cutoff Quartile 4 | 0.61 | 0.59 | 0.10 | 0.57 | 0.54 | 0.10 | 0.48 | 0.45 | 0.10 |
| Sensitivity | 55% | 54% | 73% | 63% | 63% | 68% | 63% | 65% | 68% |
| Specificity | 93% | 91% | 83% | 94% | 92% | 81% | 92% | 91% | 81% |
| | C-C motif chemokine 14 X Cystatin-C/(Weight adjusted urine output) | | | | | | | | |
| AUC | 0.82 | 0.82 | 0.83 | 0.85 | 0.83 | 0.83 | 0.82 | 0.83 | 0.83 |
| SE | 0.026 | 0.027 | 0.037 | 0.024 | 0.026 | 0.037 | 0.026 | 0.026 | 0.037 |
| p Value | 0 | 0 | 0 | 0 | 0 | 0 | 0 | 0 | 0 |
| nCohort Non- | 187 | 191 | 262 | 201 | 203 | 265 | 209 | 211 | 265 |
| persistent | | | | | | | | | |
| nCohort Persistent | 114 | 109 | 39 | 100 | 97 | 36 | 92 | 89 | 36 |
| Cutoff Quartile 2 | 0.13 | 0.11 | 0.02 | 0.08 | 0.08 | 0.02 | 0.08 | 0.08 | 0.02 |
| Sensitivity | 91% | 91% | 95% | 93% | 92% | 92% | 92% | 93% | 92% |
| Specificity | 35% | 34% | 28% | 34% | 33% | 28% | 33% | 33% | 28% |
| Cutoff Quartile 3 | 0.31 | 0.32 | 0.03 | 0.26 | 0.24 | 0.04 | 0.22 | 0.22 | 0.04 |
| Sensitivity | 82% | 81% | 87% | 84% | 82% | 86% | 85% | 85% | 86% |
| Specificity | 70% | 68% | 56% | 67% | 66% | 55% | 66% | 65% | 55% |
| Cutoff Quartile 4 | 0.58 | 0.56 | 0.11 | 0.54 | 0.51 | 0.11 | 0.46 | 0.44 | 0.11 |
| Sensitivity | 50% | 51% | 72% | 56% | 56% | 69% | 55% | 56% | 69% |
| Specificity | 90% | 90% | 82% | 91% | 90% | 81% | 89% | 88% | 81% |

TABLE 12
Comparison of marker levels and the area under the ROC curve (AUC) in urine samples for the “persistent”
and “non-persistent” cohorts where persistence starts within 48 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.80 | 0.78 | 0.79 | 0.83 | 0.82 | 0.78 | 0.83 | 0.82 | 0.78 |
| SE | 0.026 | 0.027 | 0.032 | 0.024 | 0.025 | 0.031 | 0.025 | 0.025 | 0.031 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.19 | 0.19 | 0.06 | 0.11 | 0.12 | 0.05 | 0.10 | 0.12 | 0.05 |
| Sensitivity | 90% | 90% | 95% | 93% | 92% | 93% | 94% | 91% | 93% |
| Specificity | 37% | 35% | 30% | 36% | 34% | 30% | 35% | 33% | 30% |
| Cutoff Quartile 3 | 0.36 | 0.33 | 0.10 | 0.26 | 0.26 | 0.10 | 0.24 | 0.23 | 0.10 |
| Sensitivity | 78% | 78% | 78% | 83% | 82% | 80% | 82% | 83% | 80% |
| Specificity | 70% | 68% | 57% | 69% | 67% | 57% | 67% | 66% | 57% |
| Cutoff Quartile 4 | 0.62 | 0.56 | 0.23 | 0.57 | 0.55 | 0.22 | 0.51 | 0.48 | 0.22 |
| Sensitivity | 49% | 50% | 56% | 55% | 54% | 53% | 55% | 52% | 53% |
| Specificity | 92% | 91% | 83% | 93% | 91% | 82% | 90% | 88% | 82% |
| | C-C motif chemokine 14 with serum creatinine | | | | | | | | |
| AUC | 0.85 | 0.86 | 0.77 | 0.88 | 0.87 | 0.78 | 0.86 | 0.86 | 0.78 |
| SE | 0.022 | 0.021 | 0.030 | 0.020 | 0.021 | 0.030 | 0.021 | 0.022 | 0.030 |
| p Value | 0 | 0 | 0 | 0 | 0 | 0 | 0 | 0 | 0 |
| nCohort Non- | 188 | 196 | 258 | 205 | 210 | 262 | 215 | 220 | 262 |
| persistent | | | | | | | | | |
| nCohort Persistent | 136 | 127 | 64 | 119 | 113 | 60 | 109 | 103 | 60 |
| Cutoff Quartile 2 | 0.14 | 0.13 | 0.08 | 0.09 | 0.08 | 0.07 | 0.09 | 0.08 | 0.07 |
| Sensitivity | 95% | 95% | 94% | 96% | 96% | 95% | 96% | 96% | 95% |
| Specificity | 39% | 38% | 30% | 37% | 36% | 30% | 36% | 35% | 30% |
| Cutoff Quartile 3 | 0.35 | 0.32 | 0.15 | 0.25 | 0.24 | 0.13 | 0.23 | 0.21 | 0.13 |
| Sensitivity | 82% | 82% | 86% | 87% | 87% | 90% | 88% | 88% | 90% |
| Specificity | 73% | 71% | 59% | 72% | 70% | 59% | 69% | 68% | 59% |
| Cutoff Quartile 4 | 0.70 | 0.66 | 0.29 | 0.65 | 0.62 | 0.27 | 0.57 | 0.55 | 0.27 |
| Sensitivity | 50% | 53% | 52% | 57% | 58% | 58% | 55% | 58% | 58% |
| Specificity | 93% | 93% | 81% | 94% | 92% | 82% | 90% | 90% | 82% |
| | C-C motif chemokine 14 with Cystatin-C | | | | | | | | |
| AUC | 0.83 | 0.83 | 0.79 | 0.85 | 0.85 | 0.79 | 0.84 | 0.84 | 0.79 |
| SE | 0.023 | 0.025 | 0.029 | 0.023 | 0.023 | 0.030 | 0.023 | 0.023 | 0.030 |
| p Value | 0 | 0 | 0 | 0 | 0 | 0 | 0 | 0 | 0 |
| nCohort Non- | 178 | 184 | 247 | 194 | 197 | 251 | 204 | 207 | 251 |
| persistent | | | | | | | | | |
| nCohort Persistent | 130 | 124 | 59 | 114 | 111 | 55 | 104 | 101 | 55 |
| Cutoff Quartile 2 | 0.17 | 0.15 | 0.07 | 0.10 | 0.10 | 0.06 | 0.10 | 0.10 | 0.06 |
| Sensitivity | 92% | 91% | 97% | 93% | 93% | 95% | 94% | 94% | 95% |
| Specificity | 38% | 36% | 30% | 36% | 35% | 29% | 35% | 34% | 29% |
| Cutoff Quartile 3 | 0.40 | 0.36 | 0.15 | 0.30 | 0.29 | 0.13 | 0.27 | 0.26 | 0.13 |
| Sensitivity | 82% | 81% | 90% | 85% | 86% | 87% | 87% | 87% | 87% |
| Specificity | 73% | 71% | 60% | 71% | 70% | 58% | 69% | 68% | 58% |
| Cutoff Quartile 4 | 0.67 | 0.64 | 0.27 | 0.62 | 0.59 | 0.26 | 0.54 | 0.53 | 0.26 |
| Sensitivity | 48% | 49% | 56% | 54% | 54% | 55% | 55% | 52% | 55% |
| Specificity | 92% | 91% | 82% | 92% | 91% | 81% | 90% | 88% | 81% |
| | C-C motif chemokine 14 with serum creatinine and weight adjusted urine output | | | | | | | | |
| AUC | 0.86 | 0.85 | 0.82 | 0.88 | 0.87 | 0.80 | 0.88 | 0.86 | 0.80 |
| SE | 0.021 | 0.022 | 0.029 | 0.021 | 0.021 | 0.031 | 0.021 | 0.023 | 0.031 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.13 | 0.12 | 0.04 | 0.07 | 0.08 | 0.04 | 0.07 | 0.07 | 0.04 |
| Sensitivity | 95% | 95% | 95% | 96% | 96% | 95% | 96% | 96% | 95% |
| Specificity | 40% | 38% | 30% | 37% | 36% | 30% | 36% | 35% | 30% |
| Cutoff Quartile 3 | 0.36 | 0.33 | 0.10 | 0.26 | 0.24 | 0.10 | 0.22 | 0.22 | 0.10 |
| Sensitivity | 81% | 83% | 84% | 87% | 88% | 85% | 87% | 86% | 85% |
| Specificity | 73% | 71% | 59% | 72% | 70% | 58% | 69% | 67% | 58% |
| Cutoff Quartile 4 | 0.70 | 0.63 | 0.22 | 0.67 | 0.61 | 0.21 | 0.58 | 0.53 | 0.21 |
| Sensitivity | 51% | 52% | 58% | 58% | 59% | 57% | 59% | 60% | 57% |
| Specificity | 94% | 93% | 83% | 94% | 93% | 82% | 92% | 91% | 82% |
| | C-C motif chemokine 14 X Cystatin-C/(Weight adjusted urine output) | | | | | | | | |
| AUC | 0.83 | 0.82 | 0.79 | 0.85 | 0.85 | 0.80 | 0.84 | 0.83 | 0.80 |
| SE | 0.024 | 0.025 | 0.033 | 0.024 | 0.024 | 0.031 | 0.024 | 0.025 | 0.031 |
| p Value | 0 | 0 | 0 | 0 | 0 | 0 | 0 | 0 | 0 |
| nCohort Non- | 173 | 179 | 242 | 189 | 192 | 246 | 199 | 202 | 246 |
| persistent | | | | | | | | | |
| nCohort Persistent | 128 | 122 | 59 | 112 | 109 | 55 | 102 | 99 | 55 |
| Cutoff Quartile 2 | 0.15 | 0.15 | 0.04 | 0.09 | 0.10 | 0.05 | 0.10 | 0.09 | 0.05 |
| Sensitivity | 92% | 92% | 93% | 92% | 93% | 95% | 94% | 93% | 95% |
| Specificity | 38% | 37% | 30% | 35% | 35% | 30% | 35% | 34% | 30% |
| Cutoff Quartile 3 | 0.40 | 0.38 | 0.11 | 0.32 | 0.30 | 0.10 | 0.27 | 0.25 | 0.10 |
| Sensitivity | 80% | 80% | 81% | 82% | 83% | 85% | 86% | 85% | 85% |
| Specificity | 73% | 70% | 58% | 69% | 69% | 58% | 69% | 67% | 58% |
| Cutoff Quartile 4 | 0.66 | 0.63 | 0.24 | 0.61 | 0.59 | 0.24 | 0.54 | 0.52 | 0.24 |
| Sensitivity | 48% | 49% | 53% | 54% | 55% | 55% | 57% | 54% | 55% |
| Specificity | 92% | 92% | 82% | 93% | 92% | 82% | 91% | 89% | 82% |

TABLE 13
Comparison of marker levels and the area under the ROC curve (AUC) in urine samples for the “persistent”
and “non-persistent” cohorts where persistence starts within 72 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.79 | 0.78 | 0.75 | 0.82 | 0.82 | 0.76 | 0.81 | 0.80 | 0.76 |
| SE | 0.027 | 0.027 | 0.033 | 0.025 | 0.025 | 0.033 | 0.026 | 0.026 | 0.033 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.24 | 0.22 | 0.09 | 0.16 | 0.16 | 0.08 | 0.15 | 0.14 | 0.08 |
| Sensitivity | 89% | 90% | 92% | 92% | 92% | 93% | 92% | 93% | 93% |
| Specificity | 37% | 36% | 30% | 37% | 36% | 30% | 36% | 35% | 30% |
| Cutoff Quartile 3 | 0.40 | 0.37 | 0.14 | 0.34 | 0.31 | 0.14 | 0.29 | 0.27 | 0.14 |
| Sensitivity | 76% | 76% | 76% | 79% | 80% | 80% | 82% | 81% | 80% |
| Specificity | 72% | 69% | 58% | 71% | 69% | 58% | 69% | 67% | 58% |
| Cutoff Quartile 4 | 0.66 | 0.62 | 0.28 | 0.64 | 0.61 | 0.26 | 0.58 | 0.52 | 0.26 |
| Sensitivity | 46% | 46% | 53% | 52% | 50% | 52% | 50% | 49% | 52% |
| Specificity | 93% | 90% | 83% | 94% | 91% | 83% | 90% | 88% | 83% |
| | C-C motif chemokine 14 with serum creatinine | | | | | | | | |
| AUC | 0.84 | 0.85 | 0.75 | 0.86 | 0.87 | 0.77 | 0.84 | 0.85 | 0.77 |
| SE | 0.022 | 0.021 | 0.032 | 0.022 | 0.021 | 0.030 | 0.023 | 0.023 | 0.030 |
| p Value | 0 | 0 | 0 | 0 | 0 | 0 | 0 | 0 | 0 |
| nCohort Non- | 177 | 186 | 248 | 192 | 198 | 253 | 204 | 210 | 253 |
| persistent | | | | | | | | | |
| nCohort Persistent | 147 | 137 | 74 | 132 | 125 | 69 | 120 | 113 | 69 |
| Cutoff Quartile 2 | 0.19 | 0.15 | 0.11 | 0.13 | 0.11 | 0.09 | 0.12 | 0.10 | 0.09 |
| Sensitivity | 93% | 94% | 91% | 94% | 94% | 94% | 94% | 94% | 94% |
| Specificity | 40% | 39% | 30% | 38% | 37% | 30% | 36% | 35% | 30% |
| Cutoff Quartile 3 | 0.41 | 0.36 | 0.18 | 0.32 | 0.29 | 0.16 | 0.29 | 0.25 | 0.16 |
| Sensitivity | 79% | 82% | 82% | 81% | 84% | 86% | 83% | 86% | 86% |
| Specificity | 74% | 74% | 60% | 71% | 72% | 60% | 70% | 70% | 60% |
| Cutoff Quartile 4 | 0.73 | 0.69 | 0.33 | 0.69 | 0.68 | 0.31 | 0.61 | 0.59 | 0.31 |
| Sensitivity | 48% | 52% | 49% | 52% | 53% | 52% | 52% | 54% | 52% |
| Specificity | 94% | 95% | 82% | 94% | 92% | 82% | 91% | 90% | 82% |
| | C-C motif chemokine 14 with Cystatin-C | | | | | | | | |
| AUC | 0.82 | 0.82 | 0.77 | 0.84 | 0.84 | 0.76 | 0.82 | 0.83 | 0.76 |
| SE | 0.024 | 0.024 | 0.031 | 0.023 | 0.024 | 0.031 | 0.025 | 0.025 | 0.031 |
| p Value | 0 | 0 | 0 | 0 | 0 | 0 | 0 | 0 | 0 |
| nCohort Non- | 167 | 174 | 237 | 181 | 185 | 242 | 193 | 197 | 242 |
| persistent | | | | | | | | | |
| nCohort Persistent | 141 | 134 | 69 | 127 | 123 | 64 | 115 | 111 | 64 |
| Cutoff Quartile 2 | 0.19 | 0.17 | 0.10 | 0.14 | 0.12 | 0.08 | 0.13 | 0.12 | 0.08 |
| Sensitivity | 91% | 91% | 93% | 92% | 93% | 91% | 93% | 93% | 91% |
| Specificity | 38% | 37% | 30% | 37% | 37% | 29% | 36% | 35% | 29% |
| Cutoff Quartile 3 | 0.44 | 0.42 | 0.20 | 0.36 | 0.34 | 0.18 | 0.32 | 0.30 | 0.18 |
| Sensitivity | 79% | 80% | 83% | 81% | 81% | 84% | 83% | 84% | 84% |
| Specificity | 74% | 73% | 59% | 72% | 71% | 59% | 69% | 69% | 59% |
| Cutoff Quartile 4 | 0.71 | 0.68 | 0.31 | 0.68 | 0.65 | 0.30 | 0.58 | 0.57 | 0.30 |
| Sensitivity | 46% | 49% | 54% | 51% | 53% | 50% | 52% | 54% | 50% |
| Specificity | 93% | 93% | 83% | 93% | 94% | 81% | 91% | 91% | 81% |
| | C-C motif chemokine 14 with serum creatinine and weight adjusted urine output | | | | | | | | |
| AUC | 0.84 | 0.85 | 0.78 | 0.86 | 0.86 | 0.78 | 0.85 | 0.85 | 0.78 |
| SE | 0.022 | 0.022 | 0.031 | 0.022 | 0.021 | 0.030 | 0.023 | 0.023 | 0.030 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.18 | 0.15 | 0.06 | 0.13 | 0.11 | 0.06 | 0.11 | 0.10 | 0.06 |
| Sensitivity | 92% | 94% | 91% | 94% | 94% | 93% | 94% | 95% | 93% |
| Specificity | 40% | 39% | 30% | 38% | 37% | 30% | 37% | 36% | 30% |
| Cutoff Quartile 3 | 0.41 | 0.37 | 0.13 | 0.32 | 0.31 | 0.13 | 0.28 | 0.27 | 0.13 |
| Sensitivity | 79% | 80% | 80% | 82% | 84% | 86% | 83% | 88% | 86% |
| Specificity | 75% | 72% | 59% | 72% | 71% | 60% | 70% | 70% | 60% |
| Cutoff Quartile 4 | 0.72 | 0.68 | 0.27 | 0.71 | 0.66 | 0.25 | 0.62 | 0.57 | 0.25 |
| Sensitivity | 48% | 49% | 54% | 53% | 55% | 54% | 54% | 54% | 54% |
| Specificity | 94% | 93% | 84% | 95% | 94% | 83% | 92% | 91% | 83% |
| | C-C motif chemokine 14 with Cystatin-C and weight adjusted urine output | | | | | | | | |
| AUC | 0.83 | 0.83 | 0.79 | 0.84 | 0.85 | 0.78 | 0.82 | 0.83 | 0.78 |
| SE | 0.024 | 0.024 | 0.032 | 0.024 | 0.023 | 0.032 | 0.025 | 0.025 | 0.032 |
| p Value | 0 | 0 | 0 | 0 | 0 | 0 | 0 | 0 | 0 |
| nCohort Non- | 162 | 169 | 232 | 176 | 180 | 237 | 188 | 192 | 237 |
| persistent | | | | | | | | | |
| nCohort Persistent | 139 | 132 | 69 | 125 | 121 | 64 | 113 | 109 | 64 |
| Cutoff Quartile 2 | 0.19 | 0.16 | 0.07 | 0.12 | 0.12 | 0.06 | 0.12 | 0.10 | 0.06 |
| Sensitivity | 91% | 91% | 91% | 91% | 92% | 94% | 92% | 93% | 94% |
| Specificity | 39% | 38% | 30% | 37% | 37% | 30% | 36% | 35% | 30% |
| Cutoff Quartile 3 | 0.46 | 0.42 | 0.17 | 0.38 | 0.36 | 0.15 | 0.33 | 0.32 | 0.15 |
| Sensitivity | 79% | 78% | 81% | 82% | 83% | 83% | 82% | 83% | 83% |
| Specificity | 75% | 72% | 59% | 73% | 73% | 59% | 70% | 69% | 59% |
| Cutoff Quartile 4 | 0.70 | 0.67 | 0.32 | 0.65 | 0.64 | 0.32 | 0.57 | 0.56 | 0.32 |
| Sensitivity | 45% | 48% | 55% | 51% | 54% | 53% | 50% | 53% | 53% |
| Specificity | 93% | 93% | 84% | 94% | 94% | 83% | 90% | 91% | 83% |

### Example 5. Use of C-C Motif Chemokine 14 and One or More of Serum Creatinine, Urine Output, and Cystatin C in a Decision Tree for Evaluating Renal Status in Patients Admitted to the ICU: Persistent at KDIGO Stage 3

With the same study and patient cohort as in Example 2 above, a decision tree algorithm was applied using C-C motif chemokine 14, serum creatinine, and Cystatin C concentrations, and weight adjusted urine output as input variables to classify samples into the “persistent” and “non-persistent” cohorts. The decision tree consisted of a sequence of nodes where an input variable was tested and branches which denote the possible outcomes of the test at the node. To classify an input, it was put down a tree from the top and ultimately yielding a probability classification into one of the two classes at a terminal branch. Using a subset of the data, the split at the nodes were adjusted to provide the optimal separation between the “persistent” and “non-persistent” cohorts. Renal status was assessed by serum creatinine or urine output KDIGO criteria.

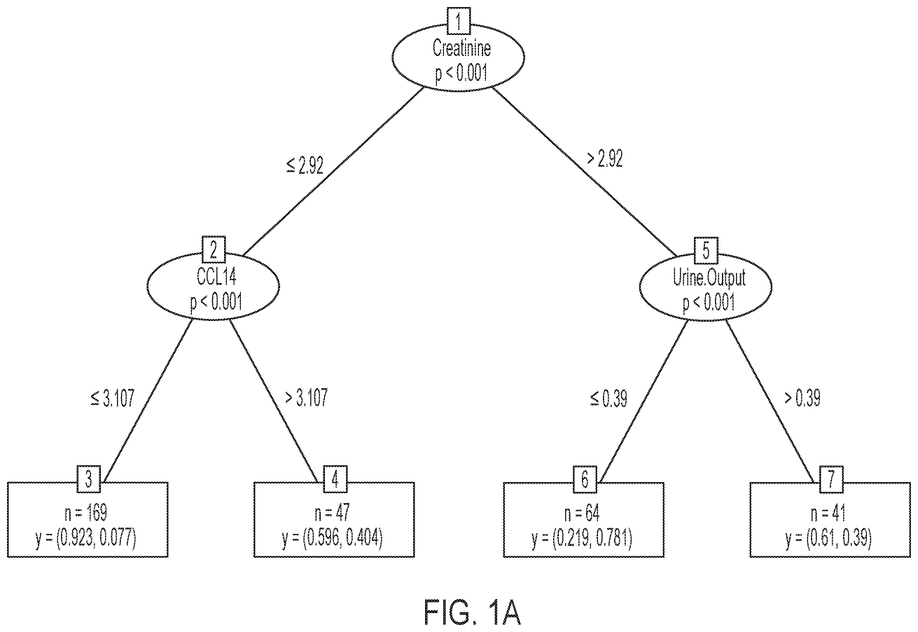

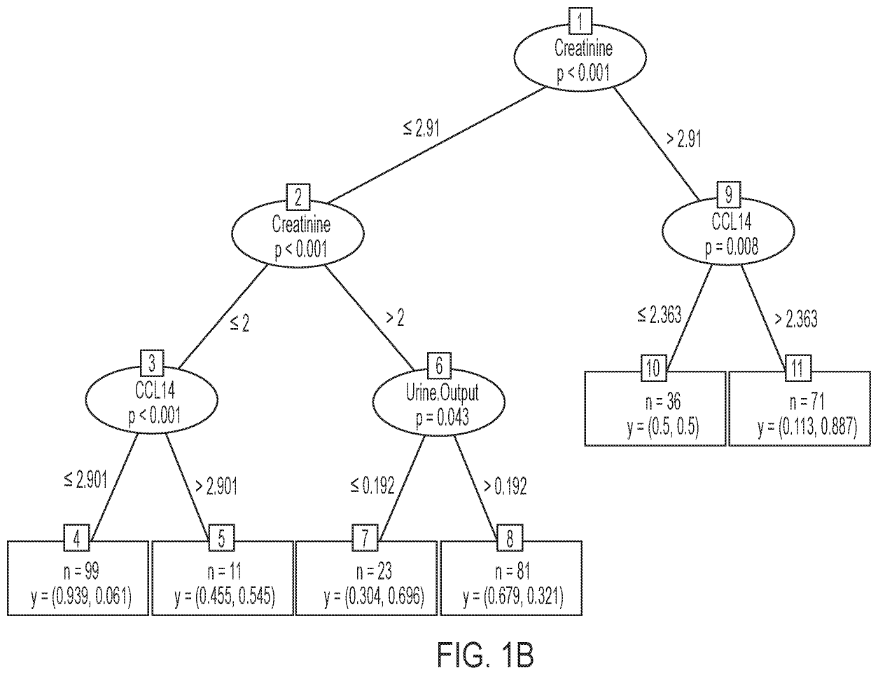

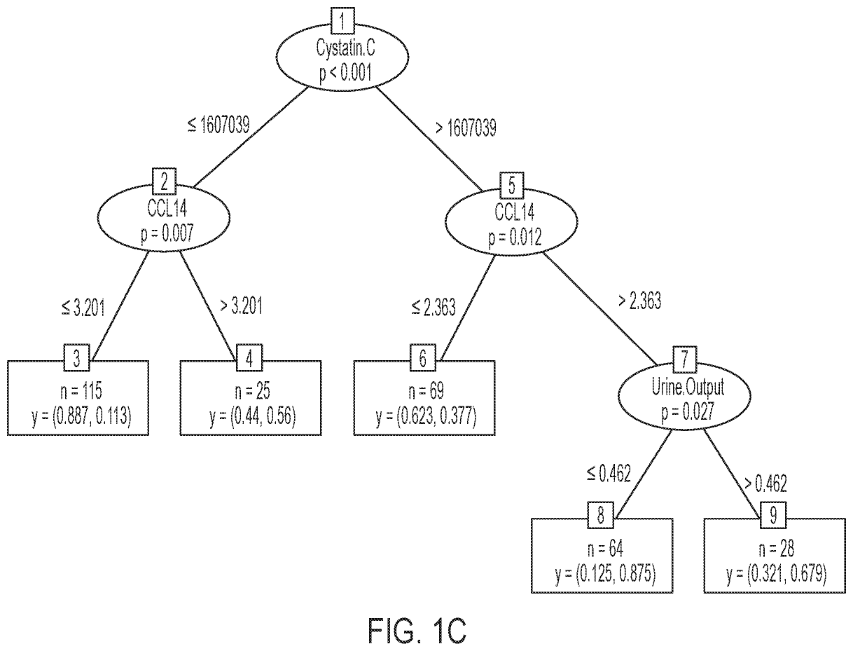

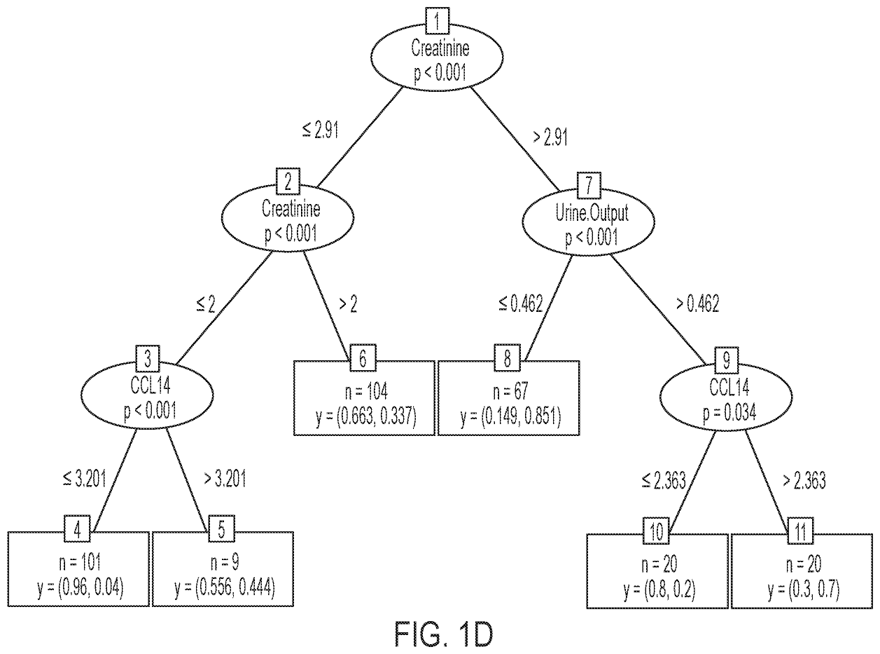

FIG. 1A illustrates an example of a decision tree for “persistent” and “non-persistent” cohorts where persistence started within 24 hours after sample collection and continued for at least 72 hours. C-C motif chemokine 14 (CCL14), serum creatinine, and weight adjusted urine output were used at the nodes of this tree. “n” denotes the number of samples at that terminal branch, and “y” denotes the proportion of “non-persistent” and “persistent” samples. Units for the biomarkers can be found in Table 1 above.

FIG. 1B provides an example of a decision tree for “persistent” and “non-persistent” cohorts where persistence started within 48 hours after sample collection and continued for at least 72 hours. C-C motif chemokine 14 (CCL14), serum creatinine, and weight adjusted urine output were used at the nodes of this tree.

FIG. 1C provides an example of a decision tree for “persistent” and “non-persistent” cohorts where persistence started within 48 hours after sample collection and continued for at least 72 hours. C-C motif chemokine 14 (CCL14), Cystatin C, and weight adjusted urine output are used at the nodes of this tree. FIG. 1D provides an example of a decision tree for “persistent” and “non-persistent” cohorts where persistence started within 72 hours after sample collection and continued for at least 72 hours. C-C motif chemokine 14 (CCL14), serum creatinine, and weight adjusted urine output were used at the nodes of this tree.

In FIGS. 1A-1D, “n” denotes the number of samples at that terminal branch, and “y” denotes the proportion of “non-persistent” and “persistent” samples (i.e. y=(proportion non-persistent, proportion persistent)). Units for the biomarkers can be found in Table 1 above.

### Example 6. Use of C-C Motif Chemokine 14 and One or More of Serum Creatinine, Urine Output, and Cystatin C in Random Forests Algorithm for Evaluating Renal Status in Patients Admitted to the ICU: Persistent at KDIGO Stage 3

With the same study and patient cohort as in Example 2 above, a random forests algorithm was applied using C-C motif chemokine 14, serum creatinine, and Cystatin C concentrations, and weight adjusted urine output as input variables to classify samples into the “persistent” and “non-persistent” cohorts. An ensemble of classification trees were grown using a random sampling of the dataset. Each individual tree was distinct from the others, and each node represents a test on one of the input variables while each branch denotes the outcome of the test. To classify an input, it was put down each tree giving a classification, or “vote”, for one of the classes. In turn, the forest rendered the ultimate classification to the class with the most “votes” from all the trees. The proportion of votes for a class approximates the probability of the sample belonging to that class and was used in an ROC analysis to determine the overall ability of the inputs to distinguish between the “persistent” and “non-persistent” cohorts. In addition, the sensitivity and specificity were determined for cutoffs corresponding to the 25^{th}, 50^{th}, and 75^{th} percentiles of the proportions of votes.

Similar to the logistic regression and linear discriminant analyses in Examples 3 and 4, respectively, a k-fold cross validation was used to ensure robustness of the ROC AUC, sensitivity and specificity estimates. In addition, a minimum of 500 trees was used for each calculation of the random forests algorithm for the combination of markers and persistence start windows in Tables 14-16 below.

The individual marker assay results were used in combinations as input variables to the random forests algorithm as indicated herein: C-C motif chemokine 14 with weight adjusted urine output, C-C motif chemokine 14 with serum creatinine, C-C motif chemokine 14 with Cystatin-C, C-C motif chemokine 14 with serum creatinine and weight adjusted urine output, and C-C motif chemokine 14 with Cystatin-C and weight adjusted urine output.

TABLE 14
Comparison of the ROC AUC in samples for the “persistent” and “non-persistent”
cohorts where persistence starts within 24 hours after sample collection and renal status
was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.733 | 0.684 | 0.722 | 0.794 | 0.785 | 0.709 | 0.778 | 0.772 | 0.709 |
| SE | 0.030 | 0.034 | 0.050 | 0.028 | 0.028 | 0.050 | 0.030 | 0.030 | 0.050 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.120 | 0.092 | 0.002 | 0.070 | 0.045 | 0.002 | 0.056 | 0.051 | 0.002 |
| Sensitivity | 85% | 82% | 89% | 91% | 91% | 85% | 89% | 91% | 85% |
| Specificity | 32% | 29% | 31% | 33% | 32% | 30% | 31% | 32% | 30% |
| Cutoff Quartile 3 | 0.318 | 0.298 | 0.028 | 0.200 | 0.212 | 0.030 | 0.218 | 0.182 | 0.030 |
| Sensitivity | 68% | 67% | 75% | 79% | 77% | 71% | 81% | 77% | 71% |
| Specificity | 62% | 59% | 55% | 65% | 63% | 54% | 64% | 61% | 54% |
| Cutoff Quartile 4 | 0.662 | 0.576 | 0.178 | 0.574 | 0.561 | 0.160 | 0.574 | 0.458 | 0.160 |
| Sensitivity | 47% | 43% | 52% | 51% | 54% | 51% | 52% | 51% | 51% |
| Specificity | 88% | 86% | 80% | 88% | 88% | 79% | 87% | 86% | 79% |
| | C-C motif chemokine 14 with serum creatinine | | | | | | | | |
| AUC | 0.788 | 0.782 | 0.685 | 0.823 | 0.807 | 0.680 | 0.791 | 0.780 | 0.680 |
| SE | 0.025 | 0.026 | 0.043 | 0.025 | 0.028 | 0.049 | 0.029 | 0.032 | 0.049 |
| p Value | 0 | 0 | 0 | 0 | 0 | 0 | 0 | 0 | 0 |
| nCohort Non- | 203 | 209 | 278 | 217 | 221 | 281 | 225 | 229 | 281 |
| persistent | | | | | | | | | |
| nCohort Persistent | 121 | 113 | 44 | 107 | 101 | 41 | 99 | 93 | 41 |
| Cutoff Quartile 2 | 0.056 | 0.058 | 0.008 | 0.042 | 0.030 | 0.002 | 0.023 | 0.022 | 0.002 |
| Sensitivity | 94% | 94% | 93% | 94% | 92% | 85% | 92% | 90% | 85% |
| Specificity | 37% | 36% | 28% | 35% | 33% | 29% | 32% | 33% | 29% |
| Cutoff Quartile 3 | 0.310 | 0.266 | 0.074 | 0.218 | 0.177 | 0.050 | 0.189 | 0.161 | 0.050 |
| Sensitivity | 76% | 78% | 73% | 81% | 80% | 76% | 80% | 80% | 76% |
| Specificity | 66% | 65% | 54% | 66% | 64% | 54% | 63% | 62% | 54% |
| Cutoff Quartile 4 | 0.620 | 0.604 | 0.217 | 0.568 | 0.536 | 0.220 | 0.565 | 0.483 | 0.220 |
| Sensitivity | 45% | 46% | 43% | 53% | 55% | 41% | 53% | 54% | 41% |
| Specificity | 87% | 86% | 78% | 89% | 89% | 77% | 87% | 86% | 77% |
| | C-C motif chemokine 14 with Cystatin-C | | | | | | | | |
| AUC | 0.746 | 0.755 | 0.535 | 0.782 | 0.768 | 0.629 | 0.779 | 0.773 | 0.629 |
| SE | 0.029 | 0.029 | 0.047 | 0.029 | 0.029 | 0.051 | 0.029 | 0.030 | 0.051 |
| p Value | 0 | 0 | 0.45 | 0 | 0 | 0.011 | 0 | 0 | 0.011 |
| nCohort Non- | 192 | 196 | 267 | 206 | 208 | 270 | 214 | 216 | 270 |
| persistent | | | | | | | | | |
| nCohort Persistent | 116 | 111 | 39 | 102 | 99 | 36 | 94 | 91 | 36 |
| Cutoff Quartile 2 | 0.087 | 0.079 | 0.004 | 0.058 | 0.048 | 0.002 | 0.042 | 0.038 | 0.002 |
| Sensitivity | 91% | 91% | 79% | 90% | 90% | 89% | 93% | 91% | 89% |
| Specificity | 35% | 34% | 26% | 33% | 33% | 27% | 33% | 32% | 27% |
| Cutoff Quartile 3 | 0.315 | 0.300 | 0.057 | 0.234 | 0.214 | 0.044 | 0.201 | 0.174 | 0.044 |
| Sensitivity | 74% | 76% | 56% | 81% | 81% | 69% | 80% | 81% | 69% |
| Specificity | 65% | 65% | 51% | 66% | 65% | 53% | 63% | 64% | 53% |
| Cutoff Quartile 4 | 0.655 | 0.664 | 0.181 | 0.660 | 0.568 | 0.192 | 0.550 | 0.562 | 0.192 |
| Sensitivity | 42% | 44% | 23% | 50% | 48% | 36% | 48% | 48% | 36% |
| Specificity | 85% | 86% | 75% | 87% | 86% | 76% | 85% | 85% | 76% |
| | C-C motif chemokine 14 with serum creatinine and weight adjusted urine output | | | | | | | | |
| AUC | 0.839 | 0.809 | 0.739 | 0.848 | 0.851 | 0.750 | 0.833 | 0.838 | 0.750 |
| SE | 0.023 | 0.026 | 0.048 | 0.024 | 0.025 | 0.044 | 0.027 | 0.027 | 0.044 |
| p Value | 0 | 0 | 0 | 0 | 0 | 0 | 0 | 0 | 0 |
| nCohort Non- | 201 | 207 | 277 | 215 | 219 | 280 | 223 | 227 | 280 |
| persistent | | | | | | | | | |
| nCohort Persistent | 120 | 112 | 44 | 106 | 100 | 41 | 98 | 92 | 41 |
| Cutoff Quartile 2 | 0.096 | 0.074 | 0.010 | 0.068 | 0.058 | 0.008 | 0.056 | 0.055 | 0.008 |
| Sensitivity | 95% | 92% | 86% | 94% | 93% | 93% | 91% | 91% | 93% |
| Specificity | 37% | 35% | 29% | 35% | 35% | 29% | 33% | 32% | 29% |
| Cutoff Quartile 3 | 0.294 | 0.298 | 0.052 | 0.220 | 0.198 | 0.052 | 0.202 | 0.196 | 0.052 |
| Sensitivity | 81% | 80% | 75% | 84% | 86% | 83% | 84% | 87% | 83% |
| Specificity | 69% | 67% | 55% | 67% | 67% | 55% | 65% | 66% | 55% |
| Cutoff Quartile 4 | 0.626 | 0.572 | 0.176 | 0.552 | 0.481 | 0.192 | 0.514 | 0.428 | 0.192 |
| Sensitivity | 52% | 54% | 57% | 57% | 60% | 54% | 60% | 62% | 54% |
| Specificity | 91% | 90% | 80% | 91% | 91% | 79% | 91% | 90% | 79% |
| | C-C motif chemokine 14 X Cystatin-C/(Weight adjusted urine output) | | | | | | | | |
| AUC | 0.77 | 0.78 | 0.76 | 0.81 | 0.79 | 0.75 | 0.79 | 0.79 | 0.75 |
| SE | 0.029 | 0.030 | 0.045 | 0.028 | 0.029 | 0.052 | 0.031 | 0.030 | 0.052 |
| p Value | 0 | 0 | 0 | 0 | 0 | 0 | 0 | 0 | 0 |
| nCohort Non- | 187 | 191 | 262 | 201 | 203 | 265 | 209 | 211 | 265 |
| persistent | | | | | | | | | |
| nCohort Persistent | 114 | 109 | 39 | 100 | 97 | 36 | 92 | 89 | 36 |
| Cutoff Quartile 2 | 0.12 | 0.12 | 0.01 | 0.08 | 0.07 | 0.01 | 0.08 | 0.07 | 0.01 |
| Sensitivity | 89% | 87% | 90% | 92% | 91% | 89% | 89% | 90% | 89% |
| Specificity | 34% | 32% | 28% | 35% | 33% | 27% | 32% | 31% | 27% |
| Cutoff Quartile 3 | 0.31 | 0.24 | 0.05 | 0.24 | 0.20 | 0.06 | 0.21 | 0.18 | 0.06 |
| Sensitivity | 77% | 76% | 82% | 78% | 80% | 83% | 80% | 82% | 83% |
| Specificity | 67% | 65% | 56% | 65% | 65% | 56% | 64% | 64% | 56% |
| Cutoff Quartile 4 | 0.59 | 0.59 | 0.17 | 0.57 | 0.54 | 0.16 | 0.51 | 0.47 | 0.16 |
| Sensitivity | 48% | 50% | 54% | 56% | 53% | 56% | 57% | 52% | 56% |
| Specificity | 90% | 90% | 80% | 91% | 88% | 79% | 89% | 86% | 79% |

TABLE 15
Comparison of marker levels and the area under the ROC curve (AUC) in urine samples for the “persistent”
and “non-persistent” cohorts where persistence starts within 48 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.72 | 0.69 | 0.73 | 0.79 | 0.76 | 0.70 | 0.76 | 0.74 | 0.70 |
| SE | 0.030 | 0.031 | 0.040 | 0.027 | 0.031 | 0.038 | 0.030 | 0.030 | 0.038 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.14 | 0.11 | 0.01 | 0.08 | 0.09 | 0.01 | 0.07 | 0.07 | 0.01 |
| Sensitivity | 84% | 83% | 89% | 91% | 87% | 92% | 89% | 87% | 92% |
| Specificity | 32% | 30% | 31% | 34% | 32% | 30% | 32% | 31% | 30% |
| Cutoff Quartile 3 | 0.35 | 0.31 | 0.08 | 0.24 | 0.24 | 0.09 | 0.22 | 0.25 | 0.09 |
| Sensitivity | 70% | 67% | 75% | 77% | 79% | 73% | 75% | 75% | 73% |
| Specificity | 66% | 61% | 57% | 66% | 65% | 56% | 63% | 62% | 56% |
| Cutoff Quartile 4 | 0.67 | 0.62 | 0.29 | 0.66 | 0.62 | 0.25 | 0.56 | 0.53 | 0.25 |
| Sensitivity | 42% | 46% | 52% | 53% | 52% | 47% | 47% | 48% | 47% |
| Specificity | 88% | 89% | 82% | 91% | 90% | 80% | 86% | 86% | 80% |
| | C-C motif chemokine 14 with serum creatinine | | | | | | | | |
| AUC | 0.78 | 0.80 | 0.68 | 0.83 | 0.83 | 0.69 | 0.79 | 0.79 | 0.69 |
| SE | 0.025 | 0.025 | 0.034 | 0.024 | 0.026 | 0.036 | 0.026 | 0.028 | 0.036 |
| p Value | 0 | 0 | 0 | 0 | 0 | 0 | 0 | 0 | 0 |
| nCohort Non- | 188 | 196 | 258 | 205 | 210 | 262 | 215 | 220 | 262 |
| persistent | | | | | | | | | |
| nCohort Persistent | 136 | 127 | 64 | 119 | 113 | 60 | 109 | 103 | 60 |
| Cutoff Quartile 2 | 0.07 | 0.06 | 0.01 | 0.04 | 0.05 | 0.01 | 0.03 | 0.04 | 0.01 |
| Sensitivity | 92% | 92% | 92% | 93% | 92% | 92% | 92% | 93% | 92% |
| Specificity | 38% | 36% | 30% | 36% | 34% | 29% | 34% | 34% | 29% |
| Cutoff Quartile 3 | 0.32 | 0.37 | 0.13 | 0.27 | 0.26 | 0.09 | 0.21 | 0.19 | 0.09 |
| Sensitivity | 72% | 74% | 70% | 79% | 81% | 80% | 78% | 78% | 80% |
| Specificity | 66% | 66% | 56% | 67% | 67% | 57% | 65% | 63% | 57% |
| Cutoff Quartile 4 | 0.74 | 0.66 | 0.31 | 0.64 | 0.59 | 0.28 | 0.59 | 0.56 | 0.28 |
| Sensitivity | 45% | 49% | 34% | 56% | 57% | 43% | 49% | 51% | 43% |
| Specificity | 89% | 91% | 77% | 93% | 92% | 79% | 87% | 87% | 79% |
| | C-C motif chemokine 14 with Cystatin-C | | | | | | | | |
| AUC | 0.769 | 0.779 | 0.697 | 0.801 | 0.779 | 0.669 | 0.784 | 0.777 | 0.669 |
| SE | 0.027 | 0.027 | 0.036 | 0.027 | 0.028 | 0.037 | 0.028 | 0.028 | 0.037 |
| p Value | 0 | 0 | 0 | 0 | 0 | 0 | 0 | 0 | 0 |
| nCohort Non- | 178 | 184 | 247 | 194 | 197 | 251 | 204 | 207 | 251 |
| persistent | | | | | | | | | |
| nCohort Persistent | 130 | 124 | 59 | 114 | 111 | 55 | 104 | 101 | 55 |
| Cutoff Quartile 2 | 0.130 | 0.112 | 0.006 | 0.072 | 0.069 | 0.004 | 0.034 | 0.048 | 0.004 |
| Sensitivity | 92% | 91% | 93% | 93% | 91% | 91% | 91% | 93% | 91% |
| Specificity | 38% | 36% | 30% | 37% | 34% | 29% | 33% | 34% | 29% |
| Cutoff Quartile 3 | 0.410 | 0.316 | 0.093 | 0.213 | 0.217 | 0.090 | 0.211 | 0.190 | 0.090 |
| Sensitivity | 75% | 77% | 76% | 78% | 76% | 76% | 80% | 80% | 76% |
| Specificity | 69% | 68% | 56% | 66% | 64% | 57% | 65% | 65% | 57% |
| Cutoff Quartile 4 | 0.753 | 0.711 | 0.304 | 0.699 | 0.676 | 0.339 | 0.670 | 0.592 | 0.339 |
| Sensitivity | 42% | 44% | 41% | 49% | 50% | 36% | 47% | 47% | 36% |
| Specificity | 88% | 88% | 79% | 89% | 89% | 77% | 86% | 86% | 77% |
| | C-C motif chemokine 14 with serum creatinine and weight adjusted urine output | | | | | | | | |
| AUC | 0.82 | 0.79 | 0.74 | 0.83 | 0.83 | 0.71 | 0.82 | 0.80 | 0.71 |
| SE | 0.023 | 0.026 | 0.036 | 0.025 | 0.025 | 0.039 | 0.026 | 0.028 | 0.039 |
| p Value | 0 | 0 | 0 | 0 | 0 | 0 | 0 | 0 | 0 |
| nCohort Non- | 186 | 194 | 257 | 203 | 208 | 261 | 213 | 218 | 261 |
| persistent | | | | | | | | | |
| nCohort Persistent | 135 | 126 | 64 | 118 | 112 | 60 | 108 | 102 | 60 |
| Cutoff Quartile 2 | 0.11 | 0.11 | 0.02 | 0.07 | 0.08 | 0.02 | 0.06 | 0.07 | 0.02 |
| Sensitivity | 95% | 93% | 94% | 92% | 93% | 92% | 94% | 90% | 92% |
| Specificity | 40% | 37% | 30% | 35% | 35% | 30% | 35% | 33% | 30% |
| Cutoff Quartile 3 | 0.36 | 0.30 | 0.11 | 0.24 | 0.26 | 0.11 | 0.22 | 0.21 | 0.11 |
| Sensitivity | 78% | 74% | 73% | 86% | 86% | 78% | 82% | 80% | 78% |
| Specificity | 70% | 65% | 56% | 71% | 69% | 57% | 67% | 64% | 57% |
| Cutoff Quartile 4 | 0.69 | 0.63 | 0.30 | 0.63 | 0.58 | 0.27 | 0.56 | 0.56 | 0.27 |
| Sensitivity | 50% | 47% | 48% | 53% | 51% | 47% | 52% | 54% | 47% |
| Specificity | 93% | 89% | 81% | 92% | 89% | 80% | 89% | 89% | 80% |
| | C-C motif chemokine 14 X Cystatin-C/(Weight adjusted urine output) | | | | | | | | |
| AUC | 0.77 | 0.79 | 0.75 | 0.81 | 0.81 | 0.78 | 0.79 | 0.79 | 0.78 |
| SE | 0.029 | 0.028 | 0.033 | 0.027 | 0.028 | 0.033 | 0.028 | 0.028 | 0.033 |
| p Value | 0 | 0 | 0 | 0 | 0 | 0 | 0 | 0 | 0 |
| nCohort Non- | 173 | 179 | 242 | 189 | 192 | 246 | 199 | 202 | 246 |
| persistent | | | | | | | | | |
| nCohort Persistent | 128 | 122 | 59 | 112 | 109 | 55 | 102 | 99 | 55 |
| Cutoff Quartile 2 | 0.15 | 0.13 | 0.02 | 0.08 | 0.10 | 0.02 | 0.08 | 0.08 | 0.02 |
| Sensitivity | 91% | 89% | 97% | 92% | 93% | 96% | 91% | 92% | 96% |
| Specificity | 37% | 36% | 31% | 35% | 35% | 30% | 34% | 35% | 30% |
| Cutoff Quartile 3 | 0.36 | 0.34 | 0.12 | 0.28 | 0.27 | 0.10 | 0.27 | 0.24 | 0.10 |
| Sensitivity | 75% | 81% | 81% | 82% | 81% | 82% | 84% | 85% | 82% |
| Specificity | 69% | 72% | 58% | 69% | 68% | 57% | 68% | 67% | 57% |
| Cutoff Quartile 4 | 0.70 | 0.65 | 0.27 | 0.64 | 0.63 | 0.27 | 0.58 | 0.61 | 0.27 |
| Sensitivity | 44% | 46% | 47% | 51% | 50% | 49% | 49% | 44% | 49% |
| Specificity | 89% | 89% | 81% | 90% | 90% | 81% | 87% | 85% | 81% |

TABLE 16
Comparison of marker levels and the area under the ROC curve (AUC) in urine samples for the “persistent”
and “non-persistent” cohorts where persistence starts within 72 hours after sample collection
and renal status was assessed by serum creatinine (sCr) only, urine output (UO) only, or serum creatinine
or urine output KDIGO criteria. A 4-fold cross-validation was used to estimate the statistics shown.

| Persistence | 24 | | | 48 | | | 72 | | |
|---|---|---|---|---|---|---|---|---|---|
| duration | sCr or | | | sCr or | | | sCr or | | |
| (hr) | UO | sCr only | UO only | UO | sCr only | UO only | UO | sCr only | UO only |
| | C-C motif chemokine 14 with weight adjusted urine output | | | | | | | | |
| AUC | 0.68 | 0.74 | 0.68 | 0.75 | 0.77 | 0.69 | 0.73 | 0.76 | 0.69 |
| SE | 0.030 | 0.029 | 0.038 | 0.029 | 0.028 | 0.037 | 0.030 | 0.029 | 0.037 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.18 | 0.15 | 0.04 | 0.12 | 0.08 | 0.03 | 0.09 | 0.08 | 0.03 |
| Sensitivity | 83% | 86% | 85% | 87% | 89% | 88% | 87% | 90% | 88% |
| Specificity | 32% | 33% | 29% | 34% | 34% | 30% | 32% | 34% | 30% |
| Cutoff Quartile 3 | 0.40 | 0.39 | 0.14 | 0.32 | 0.30 | 0.13 | 0.29 | 0.26 | 0.13 |
| Sensitivity | 67% | 71% | 68% | 75% | 77% | 71% | 73% | 77% | 71% |
| Specificity | 65% | 66% | 55% | 67% | 67% | 56% | 64% | 64% | 56% |
| Cutoff Quartile 4 | 0.74 | 0.70 | 0.36 | 0.73 | 0.63 | 0.31 | 0.65 | 0.62 | 0.31 |
| Sensitivity | 40% | 46% | 46% | 49% | 47% | 43% | 45% | 47% | 43% |
| Specificity | 87% | 91% | 81% | 92% | 89% | 80% | 87% | 87% | 80% |
| | C-C motif chemokine 14 with serum creatinine | | | | | | | | |
| AUC | 0.75 | 0.79 | 0.65 | 0.80 | 0.80 | 0.63 | 0.75 | 0.79 | 0.63 |
| SE | 0.027 | 0.025 | 0.036 | 0.026 | 0.027 | 0.038 | 0.029 | 0.028 | 0.038 |
| p Value | 0 | 0 | 0 | 0 | 0 | 0 | 0 | 0 | 0 |
| nCohort Non- | 177 | 186 | 248 | 192 | 198 | 253 | 204 | 210 | 253 |
| persistent | | | | | | | | | |
| nCohort Persistent | 147 | 137 | 74 | 132 | 125 | 69 | 120 | 113 | 69 |
| Cutoff Quartile 2 | 0.14 | 0.08 | 0.04 | 0.08 | 0.06 | 0.02 | 0.09 | 0.06 | 0.02 |
| Sensitivity | 89% | 91% | 85% | 89% | 90% | 87% | 88% | 91% | 87% |
| Specificity | 37% | 37% | 28% | 35% | 34% | 28% | 33% | 34% | 28% |
| Cutoff Quartile 3 | 0.42 | 0.34 | 0.15 | 0.33 | 0.30 | 0.14 | 0.28 | 0.24 | 0.14 |
| Sensitivity | 71% | 73% | 68% | 77% | 76% | 64% | 69% | 74% | 64% |
| Specificity | 67% | 67% | 55% | 69% | 67% | 54% | 61% | 63% | 54% |
| Cutoff Quartile 4 | 0.73 | 0.73 | 0.34 | 0.68 | 0.66 | 0.37 | 0.62 | 0.63 | 0.37 |
| Sensitivity | 43% | 47% | 36% | 51% | 50% | 36% | 48% | 53% | 36% |
| Specificity | 90% | 91% | 79% | 93% | 91% | 78% | 88% | 90% | 78% |
| | C-C motif chemokine 14 with Cystatin-C | | | | | | | | |
| AUC | 0.79 | 0.77 | 0.69 | 0.78 | 0.78 | 0.68 | 0.75 | 0.76 | 0.68 |
| SE | 0.026 | 0.027 | 0.033 | 0.027 | 0.027 | 0.037 | 0.028 | 0.028 | 0.037 |
| p Value | 0 | 0 | 0 | 0 | 0 | 0 | 0 | 0 | 0 |
| nCohort Non- | 167 | 174 | 237 | 181 | 185 | 242 | 193 | 197 | 242 |
| persistent | | | | | | | | | |
| nCohort Persistent | 141 | 134 | 69 | 127 | 123 | 64 | 115 | 111 | 64 |
| Cutoff Quartile 2 | 0.16 | 0.11 | 0.03 | 0.11 | 0.09 | 0.02 | 0.07 | 0.07 | 0.02 |
| Sensitivity | 92% | 90% | 94% | 91% | 93% | 88% | 90% | 92% | 88% |
| Specificity | 40% | 36% | 31% | 36% | 37% | 29% | 35% | 35% | 29% |
| Cutoff Quartile 3 | 0.41 | 0.40 | 0.14 | 0.32 | 0.32 | 0.15 | 0.30 | 0.20 | 0.15 |
| Sensitivity | 76% | 74% | 74% | 76% | 74% | 70% | 73% | 78% | 70% |
| Specificity | 72% | 68% | 57% | 69% | 66% | 55% | 64% | 66% | 55% |
| Cutoff Quartile 4 | 0.76 | 0.75 | 0.40 | 0.75 | 0.71 | 0.34 | 0.66 | 0.65 | 0.34 |
| Sensitivity | 40% | 43% | 39% | 45% | 45% | 42% | 40% | 43% | 42% |
| Specificity | 89% | 90% | 79% | 90% | 88% | 80% | 85% | 85% | 80% |
| | C-C motif chemokine 14 with serum creatinine and weight adjusted urine output | | | | | | | | |
| AUC | 0.76 | 0.80 | 0.68 | 0.80 | 0.82 | 0.71 | 0.76 | 0.78 | 0.71 |
| SE | 0.026 | 0.026 | 0.037 | 0.027 | 0.025 | 0.034 | 0.029 | 0.028 | 0.034 |
| p Value | 0 | 0 | 0 | 0 | 0 | 0 | 0 | 0 | 0 |
| nCohort Non- | 175 | 184 | 247 | 190 | 196 | 252 | 202 | 208 | 252 |
| persistent | | | | | | | | | |
| nCohort Persistent | 146 | 136 | 74 | 131 | 124 | 69 | 119 | 112 | 69 |
| Cutoff Quartile 2 | 0.19 | 0.15 | 0.06 | 0.12 | 0.11 | 0.05 | 0.10 | 0.09 | 0.05 |
| Sensitivity | 90% | 92% | 86% | 90% | 92% | 91% | 89% | 90% | 91% |
| Specificity | 38% | 38% | 30% | 36% | 36% | 30% | 34% | 33% | 30% |
| Cutoff Quartile 3 | 0.44 | 0.36 | 0.15 | 0.30 | 0.31 | 0.15 | 0.27 | 0.26 | 0.15 |
| Sensitivity | 70% | 77% | 73% | 79% | 81% | 68% | 76% | 78% | 68% |
| Specificity | 67% | 70% | 57% | 70% | 70% | 55% | 65% | 65% | 55% |
| Cutoff Quartile 4 | 0.73 | 0.69 | 0.32 | 0.69 | 0.68 | 0.29 | 0.62 | 0.54 | 0.29 |
| Sensitivity | 45% | 46% | 45% | 52% | 51% | 42% | 50% | 49% | 42% |
| Specificity | 92% | 91% | 81% | 94% | 91% | 80% | 90% | 88% | 80% |
| | C-C motif chemokine 14 with Cystatin-C and weight adjusted urine output | | | | | | | | |
| AUC | 0.76 | 0.77 | 0.70 | 0.77 | 0.79 | 0.74 | 0.77 | 0.79 | 0.74 |
| SE | 0.029 | 0.028 | 0.037 | 0.028 | 0.027 | 0.038 | 0.029 | 0.028 | 0.038 |
| p Value | 0 | 0 | 0 | 0 | 0 | 0 | 0 | 0 | 0 |
| nCohort Non- | 162 | 169 | 232 | 176 | 180 | 237 | 188 | 192 | 237 |
| persistent | | | | | | | | | |
| nCohort Persistent | 139 | 132 | 69 | 125 | 121 | 64 | 113 | 109 | 64 |
| Cutoff Quartile 2 | 0.19 | 0.17 | 0.06 | 0.14 | 0.13 | 0.05 | 0.13 | 0.12 | 0.05 |
| Sensitivity | 86% | 86% | 90% | 88% | 91% | 94% | 90% | 89% | 94% |
| Specificity | 35% | 34% | 30% | 35% | 36% | 31% | 35% | 33% | 31% |
| Cutoff Quartile 3 | 0.39 | 0.40 | 0.16 | 0.33 | 0.32 | 0.12 | 0.31 | 0.27 | 0.12 |
| Sensitivity | 74% | 76% | 67% | 75% | 78% | 75% | 79% | 80% | 75% |
| Specificity | 71% | 70% | 56% | 68% | 69% | 57% | 68% | 67% | 57% |
| Cutoff Quartile 4 | 0.74 | 0.69 | 0.34 | 0.71 | 0.66 | 0.30 | 0.61 | 0.61 | 0.30 |
| Sensitivity | 40% | 46% | 45% | 46% | 48% | 55% | 43% | 48% | 55% |
| Specificity | 89% | 92% | 81% | 90% | 91% | 83% | 86% | 89% | 83% |

### Example 7. Use of C-C Motif Chemokine 14 and One or More of Serum Creatinine, Urine Output, and Cystatin C in an “n-of-m” (i.e. “Number Positive”) Analysis for Evaluating Renal Status in Patients Admitted to the ICU: Persistent at KDIGO Stage 3

With the same study and patient cohort as in Example 2 above, an “n-of-m” analysis was applied using C-C motif chemokine 14, serum creatinine and Cystatin C concentrations, and weight adjusted urine output as input variables to stratify the risk of being in the “persistent” cohort, where risk was defined as the percentage of patients who belong to the “persistent” cohort in a given population. In the “n-of-m” analysis, a sample concentration or weight adjusted urine output was compared against a cutoff. For C-C motif chemokine 14, serum creatinine and Cystatin C, concentrations above the cutoff were associated with an increased risk of “persistence”, while for weight adjusted urine output, a value below the cutoff was associated with an increase in risk. In this model, the risk from individual input variables was assumed to be cumulative, and thus, the number of “positives”, i.e. concentrations above the cutoff for C-C motif chemokine 14, serum creatinine and Cystatin C and values below the cutoff for weight adjusted urine output, stratified the risk of “persistence”.

For C-C motif chemokine 14 and Cystatin C, the 25^{th}, 50^{th} and 75^{th} percentiles of the respective concentrations were used as cutoffs. For serum creatinine, the cutoff was set at twice the patient's baseline level. For weight adjusted urine output, the cutoff was at less than 0.5 mL/kg/h of urine for at least 12 hours.

TABLE 17
The risk of being “persistent” with 0, 1, 2, or 3 “positives”
where persistence started within 24 hours after sample collection and renal
status was assessed by serum creatinine or urine output KDIGO criteria. Units
for C-C motif chemokine 14 and Cystatin C can be found in Table 1 above.
C-C motif chemokine 14 with serum creatinine and weight adjusted urine output

| | | | | | | | Cochran- |
|---|---|---|---|---|---|---|---|
| | | | | | | | Armitage |
| Persistence | | | | | | | Test for |
| Duration | Quartile | CCL14 | | | | | Trend |
| (h) | Cutoff | Cutoff | 0 Positive | 1 Positive | 2 Positives | 3 Positives | p-value |
| 24 | 2 | 0.73 | 0% | 13% | 41% | 79% | <0.001 |
| | 3 | 1.64 | 0% | 18% | 53% | 83% | <0.001 |
| | 4 | 5.16 | 0% | 29% | 60% | 91% | <0.001 |
| 48 | 2 | 0.73 | 0% | 8.2% | 37.1% | 73.6% | <0.001 |
| | 3 | 1.64 | 0% | 12% | 48% | 81% | <0.001 |
| | 4 | 5.16 | 0% | 23% | 57% | 88% | <0.001 |
| 72 | 2 | 0.73 | 0% | 8.2% | 33.5% | 69.8% | <0.001 |
| | 3 | 1.64 | 0% | 12% | 44% | 77% | <0.001 |
| | 4 | 5.16 | 0% | 22% | 51% | 82% | <0.001 |

C-C motif chemokine 14 with Cystatin C and weight adjusted urine output

| | | | | | | | | Cochran- |
|---|---|---|---|---|---|---|---|---|
| | | | | | | | | Armitage |
| Persistence | | | | | | | | Test for |
| Duration | Quartile | CCL14 | Cystatin C | | | | | Trend |
| (h) | Cutoff | Cutoff | Cutoff | 0 Positive | 1 Positive | 2 Positives | 3 Positives | p-value |
| 24 | 2 | 0.73 | 1169436 | 0% | 17% | 36% | 57% | <0.001 |
| | 3 | 1.70 | 1656049 | 0% | 18% | 40% | 69% | <0.001 |
| | 4 | 5.42 | 2146685 | 0% | 28% | 37% | 88% | <0.001 |
| 48 | 2 | 0.73 | 1169436 | 0% | 10% | 33% | 51% | <0.001 |
| | 3 | 1.70 | 1656049 | 0% | 13% | 35% | 68% | <0.001 |
| | 4 | 5.42 | 2146685 | 0% | 22% | 34% | 85% | <0.001 |
| 72 | 2 | 0.73 | 1169436 | 0% | 10% | 29% | 48% | <0.001 |
| | 3 | 1.70 | 1656049 | 0% | 12% | 31% | 64% | <0.001 |
| | 4 | 5.42 | 2146685 | 0% | 21% | 30% | 79% | <0.001 |

TABLE 18
The risk of being “persistent” with 0, 1, 2, or 3 “positives”
where persistence started within 48 hours after sample collection and renal
status was assessed by serum creatinine or urine output KDIGO criteria. Units
for C-C motif chemokine 14 and Cystatin C can be found in Table 1 above.
C-C motif chemokine 14 with serum creatinine and weight adjusted urine output

| | | | | | | | Cochran- |
|---|---|---|---|---|---|---|---|
| | | | | | | | Armitage |
| Persistence | | | | | | | Test for |
| Duration | Quartile | CCL14 | | | | | Trend |
| (h) | Cutoff | Cutoff | 0 Positive | 1 Positive | 2 Positives | 3 Positives | p-value |
| 24 | 2 | 0.73 | 0% | 8.2% | 37.1% | 74% | <0.001 |
| | 3 | 1.64 | 0% | 12% | 48% | 81% | <0.001 |
| | 4 | 5.16 | 0% | 23% | 57% | 88% | <0.001 |
| 48 | 2 | 0.73 | 0% | 8.2% | 34% | 70% | <0.001 |
| | 3 | 1.64 | 0% | 12% | 44% | 77% | <0.001 |
| | 4 | 5.16 | 0% | 22% | 51% | 82% | <0.001 |
| 72 | 2 | 0.73 | 0% | 16% | 49% | 79% | <0.001 |
| | 3 | 1.64 | 0% | 22% | 62% | 83% | <0.001 |
| | 4 | 5.16 | 0% | 35% | 67% | 91% | <0.001 |

C-C motif chemokine 14 with Cystatin C and weight adjusted urine output

| | | | | | | | | Cochran- |
|---|---|---|---|---|---|---|---|---|
| | | | | | | | | Armitage |
| Persistence | | | | | | | | Test for |
| Duration | Quartile | CCL14 | Cystatin C | | | | | Trend |
| (h) | Cutoff | Cutoff | Cutoff | 0 Positive | 1 Positive | 2 Positives | 3 Positives | p-value |
| 24 | 2 | 0.73 | 1169436 | 0% | 10% | 33% | 51% | <0.001 |
| | 3 | 1.70 | 1656049 | 0% | 13% | 35% | 68% | <0.001 |
| | 4 | 5.42 | 2146685 | 0% | 22% | 34% | 85% | <0.001 |
| 48 | 2 | 0.73 | 1169436 | 0% | 10% | 29% | 48% | <0.001 |
| | 3 | 1.70 | 1656049 | 0% | 12% | 31% | 64% | <0.001 |
| | 4 | 5.42 | 2146685 | 0% | 21% | 30% | 79% | <0.001 |
| 72 | 2 | 0.73 | 1169436 | 0% | 18% | 43% | 58% | <0.001 |
| | 3 | 1.70 | 1656049 | 0% | 21% | 48% | 71% | <0.001 |
| | 4 | 5.42 | 2146685 | 0% | 32% | 43% | 88% | <0.001 |

TABLE 19
The risk of being “persistent” with 0, 1, 2, or 3 “positives”
where persistence started within 72 hours after sample collection and renal
status was assessed serum creatinine or urine output KDIGO criteria. Units
for C-C motif chemokine 14 and Cystatin C can be found in Table 15 above.
C-C motif chemokine 14 with serum creatinine and weight adjusted urine output

| | | | | | | | Cochran- |
|---|---|---|---|---|---|---|---|
| | | | | | | | Armitage |
| Persistence | | | | | | | Test for |
| Duration | Quartile | CCL14 | | | | | Trend |
| (h) | Cutoff | Cutoff | 0 Positive | 1 Positive | 2 Positives | 3 Positives | p-value |
| 24 | 2 | 0.73 | 0% | 8.2% | 33.5% | 70% | <0.001 |
| | 3 | 1.64 | 0% | 12% | 44% | 77% | <0.001 |
| | 4 | 5.16 | 0% | 22% | 51% | 82% | <0.001 |
| 48 | 2 | 0.73 | 0% | 16% | 49% | 79% | <0.001 |
| | 3 | 1.64 | 0% | 22% | 62% | 83% | <0.001 |
| | 4 | 5.16 | 0% | 35% | 67% | 91% | <0.001 |
| 72 | 2 | 0.73 | 0% | 9.4% | 43.1% | 75.5% | <0.001 |
| | 3 | 1.64 | 0% | 14% | 57% | 81% | <0.001 |
| | 4 | 5.16 | 0% | 26% | 65% | 88% | <0.001 |

C-C motif chemokine 14 with Cystatin C and weight adjusted urine output

| | | | | | | | | Cochran- |
|---|---|---|---|---|---|---|---|---|
| | | | | | | | | Armitage |
| Persistence | | | | | | | | Test for |
| Duration | Quartile | CCL14 | Cystatin C | | | | | Trend |
| (h) | Cutoff | Cutoff | Cutoff | 0 Positive | 1 Positive | 2 Positives | 3 Positives | p-value |
| 24 | 2 | 0.73 | 1169436 | 0% | 10% | 29% | 48% | <0.001 |
| | 3 | 1.70 | 1656049 | 0% | 12% | 31% | 64% | <0.001 |
| | 4 | 5.42 | 2146685 | 0% | 21% | 30% | 79% | <0.001 |
| 48 | 2 | 0.73 | 1169436 | 0% | 18% | 43% | 58% | <0.001 |
| | 3 | 1.70 | 1656049 | 0% | 21% | 48% | 71% | <0.001 |
| | 4 | 5.42 | 2146685 | 0% | 32% | 43% | 88% | <0.001 |
| 72 | 2 | 0.73 | 1169436 | 0% | 12% | 39% | 52% | <0.001 |
| | 3 | 1.70 | 1656049 | 0% | 15% | 43% | 68% | <0.001 |
| | 4 | 5.42 | 2146685 | 0% | 26% | 38% | 85% | <0.001 |

### Example 8: Use of C-C Motif Chemokine 14 and One or More of Serum Creatinine, Urine Output, and Cystatin C in a Product Model for Evaluating Renal Status in Patients Admitted to the ICU: Persistent at KDIGO Stage 2 or 3

With the same study and patient cohort as in Example 2 above, kidney status was assessed by the KDIGO criteria based on serum creatinine only, based on urine output only, or based on either serum creatinine or urine output. Two cohorts were defined to represent a “persistent” and a “non-persistent” population. “Persistent” indicates those patients whose minimum KDIGO stage during a period of 24, 48 or 72 hours was Stage 2 or 3 where the persistence period can start from the time of sample collection to 24, 48, 72, 96 or 168 hours after sample collection. “Non-persistent” indicates those patients who were not persistent at Stage 2 or 3 and whose minimum KDIGO stage during a period of 24, 48 or 72 hours was No AKI or Stage 1 where the persistence period can start from the time of sample collection to 24, 48, 72, 96 or 168 hours after sample collection. If a patient died or was placed on renal replacement therapy (RRT) during the persistence period after reaching KDIGO Stage 2 or 3, the patient was considered “persistent.”

The ability to distinguish the “persistent” and “non-persistent” cohorts was determined using a receiver operating characteristic (ROC) analysis. Sensitivity, specificity, and odds ratio were determined for cutoffs corresponding to the 25^{th}, 50^{th}, and 75^{th} percentiles (Quartile 2, Quartile 3, and Quartile 4) of assay results.

TABLE 20
Comparison of marker levels and the area under the ROC curve (AUC) in samples for the “persistent”
and “non-persistent” cohorts where persistence starts within 24 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| Persistence duration (hr) | | | | |
|---|---|---|---|---|
| 24 | | | 48 | |
| sCr or UO | sCr only | UO only | sCr or UO | sCr only |

C-C motif chemokine 14

| | | | | | |
|---|---|---|---|---|---|
| AUC | 0.80 | 0.79 | 0.72 | 0.79 | 0.79 |
| SE | 0.024 | 0.024 | 0.035 | 0.025 | 0.025 |
| p Value | 0 | 0 | 2.4E−10 | 0 | 0 |
| nCohort Non-persistent | 122 | 137 | 248 | 158 | 165 |
| nCohort Persistent | 209 | 192 | 81 | 173 | 164 |
| Cutoff Quartile 2 | 0.725 | 0.719 | 0.732 | 0.725 | 0.719 |
| Sensitivity | 88% | 89% | 94% | 89% | 89% |
| Specificity | 48% | 45% | 31% | 41% | 39% |
| Cutoff Quartile 3 | 1.64 | 1.64 | 1.64 | 1.64 | 1.64 |
| Sensitivity | 67% | 69% | 78% | 72% | 73% |
| Specificity | 79% | 76% | 59% | 74% | 72% |
| Cutoff Quartile 4 | 5.16 | 5.09 | 5.09 | 5.16 | 5.09 |
| Sensitivity | 37% | 40% | 40% | 42% | 43% |
| Specificity | 96% | 95% | 79% | 93% | 92% |
| OR Quartile 2 | 6.67 | 6.54 | 6.84 | 5.52 | 5.14 |
| p Value | 1.2E−11 | 7.2E−11 | 6.5E−5 | 5.1E−9 | 3.4E−8 |
| Lower limitof 95% CI | 3.85 | 3.72 | 2.66 | 3.11 | 2.87 |
| Upper limit of 95% CI | 11.5 | 11.5 | 17.6 | 9.78 | 9.19 |
| OR Quartile 3 | 7.49 | 6.93 | 5.01 | 7.43 | 6.84 |
| p Value | 3.4E−14 | 2.1E−14 | 5.7E−8 | 6.7E−16 | 6.2E−15 |
| Lower limit of 95% CI | 4.45 | 4.22 | 2.80 | 4.57 | 4.22 |
| Upper limit of 95% CI | 12.6 | 11.4 | 8.96 | 12.1 | 11.1 |
| OR Quartile 4 | 13.9 | 12.2 | 2.52 | 9.53 | 8.71 |
| p Value | 3.7E−8 | 1.8E−9 | 8.1E−4 | 1.0E−10 | 5.0E−11 |
| Lower limit of 95% CI | 5.45 | 5.39 | 1.47 | 4.81 | 4.57 |
| Upper limit of 95% CI | 35.6 | 27.5 | 4.34 | 18.9 | 16.6 |

Weight adjusted urine output

| | | | | | |
|---|---|---|---|---|---|
| AUC | 0.40 | 0.46 | 0.17 | 0.37 | 0.40 |
| SE | 0.031 | 0.032 | 0.029 | 0.030 | 0.031 |
| p Value | 0.0021 | 0.19 | 0 | 8.7E−6 | 0.0019 |
| nCohort Non-persistent | 124 | 140 | 253 | 161 | 169 |
| nCohort Persistent | 213 | 195 | 84 | 176 | 166 |
| Cutoff Quartile 2 | 0.189 | 0.192 | 0.189 | 0.189 | 0.192 |
| Sensitivity | 70% | 73% | 40% | 66% | 69% |
| Specificity | 17% | 21% | 13% | 15% | 19% |
| Cutoff Quartile 3 | 0.366 | 0.368 | 0.366 | 0.366 | 0.368 |
| Sensitivity | 46% | 48% | 15% | 41% | 43% |
| Specificity | 42% | 47% | 38% | 40% | 43% |
| Cutoff Quartile 4 | 0.857 | 0.862 | 0.857 | 0.857 | 0.862 |
| Sensitivity | 23% | 25% | 5% | 20% | 21% |
| Specificity | 71% | 74% | 68% | 70% | 71% |
| OR Quartile 2 | 0.485 | 0.731 | 0.106 | 0.339 | 0.527 |
| p Value | 0.011 | 0.23 | 6.9E−15 | 7.1E−5 | 0.013 |
| Lower limit of 95% CI | 0.279 | 0.438 | 0.0599 | 0.199 | 0.317 |
| Upper limit of 95% CI | 0.845 | 1.22 | 0.186 | 0.578 | 0.874 |
| OR Quartile 3 | 0.604 | 0.830 | 0.114 | 0.480 | 0.582 |
| p Value | 0.027 | 0.40 | 3.6E−11 | 9.3E−4 | 0.014 |
| Lower limit of 95% CI | 0.386 | 0.537 | 0.0598 | 0.311 | 0.378 |
| Upper limit of 95% CI | 0.944 | 1.28 | 0.217 | 0.741 | 0.897 |
| OR Quartile 4 | 0.730 | 0.943 | 0.106 | 0.588 | 0.654 |
| p Value | 0.22 | 0.82 | 2.3E−5 | 0.036 | 0.096 |
| Lower limit of 95% CI | 0.442 | 0.572 | 0.0376 | 0.358 | 0.397 |
| Upper limit of 95% CI | 1.21 | 1.55 | 0.300 | 0.966 | 1.08 |

Serum Creatinine

| | | | | | |
|---|---|---|---|---|---|
| AUC | 0.82 | 0.84 | 0.69 | 0.79 | 0.80 |
| SE | 0.021 | 0.021 | 0.033 | 0.024 | 0.023 |
| p Value | 0 | 0 | 1.9E−8 | 0 | 0 |
| nCohort Non-persistent | 122 | 138 | 257 | 160 | 168 |
| nCohort Persistent | 232 | 214 | 95 | 194 | 184 |
| Cutoff Quartile 2 | 1.64 | 1.64 | 1.65 | 1.64 | 1.64 |
| Sensitivity | 89% | 92% | 88% | 90% | 92% |
| Specificity | 52% | 51% | 30% | 44% | 44% |
| Cutoff Quartile 3 | 2.44 | 2.44 | 2.44 | 2.44 | 2.44 |
| Sensitivity | 67% | 70% | 69% | 69% | 71% |
| Specificity | 82% | 81% | 57% | 72% | 73% |
| Cutoff Quartile 4 | 3.32 | 3.31 | 3.32 | 3.32 | 3.31 |
| Sensitivity | 36% | 38% | 46% | 40% | 41% |
| Specificity | 95% | 96% | 82% | 92% | 93% |
| OR Quartile 2 | 8.46 | 11.2 | 3.27 | 7.16 | 9.56 |
| p Value | 1.0E−14 | 6.7E−16 | 6.8E−4 | 1.0E−11 | 1.4E−12 |
| Lower limit of 95% CI | 4.93 | 6.23 | 1.65 | 4.06 | 5.12 |
| Upper limit of 95% CI | 14.5 | 20.2 | 6.47 | 12.6 | 17.8 |
| OR Quartile 3 | 9.33 | 10.1 | 2.99 | 5.89 | 6.76 |
| p Value | 4.4E−16 | 0 | 1.8E−5 | 5.2E−14 | 1.1E−15 |
| Lower limit of 95% CI | 5.45 | 6.02 | 1.81 | 3.71 | 4.23 |
| Upper limit of 95% CI | 16.0 | 16.9 | 4.94 | 9.34 | 10.8 |
| OR Quartile 4 | 11.0 | 13.7 | 4.06 | 8.29 | 9.15 |
| p Value | 5.3E−8 | 2.9E−9 | 9.9E−8 | 2.4E−10 | 3.9E−11 |
| Lower limit of 95% CI | 4.63 | 5.76 | 2.43 | 4.31 | 4.75 |
| Upper limit of 95% CI | 26.0 | 32.4 | 6.81 | 16.0 | 17.6 |

Cystatin-C

| | | | | | |
|---|---|---|---|---|---|
| AUC | 0.77 | 0.76 | 0.70 | 0.78 | 0.76 |
| SE | 0.027 | 0.027 | 0.037 | 0.026 | 0.027 |
| p Value | 0 | 0 | 4.6E−8 | 0 | 0 |
| nCohort Non-persistent | 107 | 120 | 232 | 142 | 148 |
| nCohort Persistent | 201 | 187 | 74 | 166 | 159 |
| Cutoff Quartile 2 | 1170000 | 1170000 | 1170000 | 1170000 | 1170000 |
| Sensitivity | 87% | 88% | 89% | 89% | 89% |
| Specificity | 46% | 44% | 30% | 41% | 39% |
| Cutoff Quartile 3 | 1660000 | 1650000 | 1660000 | 1660000 | 1650000 |
| Sensitivity | 65% | 66% | 76% | 70% | 70% |
| Specificity | 78% | 74% | 58% | 73% | 71% |
| Cutoff Quartile 4 | 2150000 | 2150000 | 2150000 | 2150000 | 2150000 |
| Sensitivity | 33% | 35% | 45% | 39% | 40% |
| Specificity | 91% | 91% | 81% | 92% | 91% |
| OR Quartile 2 | 5.44 | 5.64 | 3.49 | 5.68 | 5.05 |
| p Value | 2.3E−9 | 2.1E−9 | 0.0018 | 9.4E−9 | 8.0E−8 |
| Lower limit of 95% CI | 3.12 | 3.20 | 1.59 | 3.14 | 2.79 |
| Upper limit of 95% CI | 9.49 | 9.93 | 7.66 | 10.3 | 9.12 |
| OR Quartile 3 | 6.33 | 5.52 | 4.3.3 | 6.35 | 5.65 |
| p Value | 1.9E−11 | 4.5E−11 | 1.2E−6 | 3.5E−13 | 4.6E−12 |
| Lower limit of 95% CI | 3.70 | 3.32 | 2.40 | 3.86 | 3.46 |
| Upper limit of 95% CI | 10.9 | 9.17 | 7.82 | 10.4 | 9.22 |
| OR Quartile 4 | 4.85 | 5.40 | 3.44 | 6.97 | 7.00 |
| p Value | 1.5E−5 | 1.6E−6 | 1.8E−5 | 1.2E−8 | 4.8E−9 |
| Lower limit of 95% CI | 2.37 | 2.71 | 1.96 | 3.57 | 3.65 |
| Upper limit of 95% CI | 9.90 | 10.8 | 6.04 | 13.6 | 13.4 |

C-C motif chemokine 14/(Weight adjusted urine output)

| | | | | | |
|---|---|---|---|---|---|
| AUC | 0.76 | 0.72 | 0.82 | 0.77 | 0.75 |
| SE | 0.026 | 0.028 | 0.030 | 0.026 | 0.027 |
| p Value | 0 | 4.4E−16 | 0 | 0 | 0 |
| nCohort Non-persistent | 120 | 135 | 247 | 156 | 163 |
| nCohort Persistent | 208 | 191 | 81 | 172 | 163 |
| Cutoff Quartile 2 | 1.26 | 1.24 | 1.26 | 1.26 | 1.24 |
| Sensitivity | 85% | 84% | 99% | 87% | 86% |
| Specificity | 42% | 38% | 33% | 38% | 36% |
| Cutoff Quartile 3 | 4.59 | 4.54 | 4.59 | 4.59 | 4.54 |
| Sensitivity | 65% | 65% | 84% | 70% | 69% |
| Specificity | 76% | 71% | 61% | 72% | 69% |
| Cutoff Quartile 4 | 24.3 | 22.9 | 24.3 | 24.3 | 22.9 |
| Sensitivity | 36% | 36% | 54% | 42% | 41% |
| Specificity | 93% | 90% | 85% | 94% | 91% |
| OR Quartile 2 | 4.22 | 3.13 | 39.0 | 3.94 | 3.45 |
| p Value | 8.1E−8 | 1.6E−5 | 3.1E−4 | 8.3E−7 | 8.2E−6 |
| Lower limit of 95% CI | 2.49 | 1.87 | 5.34 | 2.28 | 2.00 |
| Upper limit of 95% CI | 7.14 | 5.26 | 286 | 6.80 | 5.95 |
| OR Quartile 3 | 5.80 | 4.56 | 8.23 | 6.23 | 5.11 |
| p Value | 9.4E−12 | 4.4E−10 | 1.6E−10 | 7.8E−14 | 1.1E−11 |
| Lower limit of 95% CI | 3.50 | 2.83 | 4.31 | 3.86 | 3.19 |
| Upper limit of 95% CI | 9.62 | 7.33 | 15.7 | 10.1 | 8.18 |
| OR Quartile 4 | 7.73 | 5.31 | 6.54 | 10.5 | 6.89 |
| p Value | 2.0E−7 | 3.7E−7 | 4.0E−11 | 7.7E−11 | 8.3E−10 |
| Lower limit of 95% CI | 3.57 | 2.79 | 3.75 | 5.17 | 3.72 |
| Upper limit of 95% CI | 16.7 | 10.1 | 11.4 | 21.4 | 12.7 |

C-C motif chemokine 14 X Serum Creatinine

| | | | | | |
|---|---|---|---|---|---|
| AUC | 0.84 | 0.84 | 0.73 | 0.83 | 0.83 |
| SE | 0.021 | 0.022 | 0.035 | 0.023 | 0.023 |
| p Value | 0 | 0 | 3.5E−11 | 0 | 0 |
| nCohort Non-persistent | 117 | 132 | 242 | 153 | 160 |
| nCohort Persistent | 207 | 190 | 80 | 171 | 162 |
| Cutoff Quartile 2 | 1.29 | 1.29 | 1.30 | 1.29 | 1.29 |
| Sensitivity | 88% | 90% | 90% | 90% | 91% |
| Specificity | 49% | 47% | 30% | 42% | 41% |
| Cutoff Quartile 3 | 3.97 | 3.91 | 3.97 | 3.97 | 3.91 |
| Sensitivity | 70% | 72% | 81% | 75% | 76% |
| Specificity | 85% | 82% | 60% | 78% | 76% |
| Cutoff Quartile 4 | 18.3 | 16.8 | 18.0 | 18.3 | 16.8 |
| Sensitivity | 37% | 40% | 45% | 42% | 44% |
| Specificity | 97% | 96% | 81% | 94% | 94% |
| OR Quartile 2 | 7.24 | 7.97 | 3.89 | 6.51 | 6.88 |
| p Value | 3.8E−12 | 3.3E−12 | 6.5E−4 | 6.7E−10 | 9.2E−10 |
| Lower limit of 95% CI | 4.14 | 4.44 | 1.78 | 3.59 | 3.71 |
| Upper limit of 95% CI | 12.7 | 14.3 | 8.48 | 11.8 | 12.8 |
| OR Quartile 3 | 13.8 | 11.6 | 6.59 | 10.4 | 10.1 |
| p Value | 0 | 0 | 2.2E−9 | 0 | 0 |
| Lower limit of 95% CI | 7.60 | 6.75 | 3.55 | 6.23 | 6.07 |
| Upper limit of 95% CI | 24.9 | 20.0 | 12.2 | 17.4 | 16.9 |
| OR Quartile 4 | 16.7 | 16.9 | 3.58 | 11.6 | 11.7 |
| p Value | 9.9E−8 | 3.6E−9 | 4.8E−6 | 7.4E−11 | 1.2E−11 |
| Lower limit of 95% CI | 5.94 | 6.62 | 2.07 | 5.56 | 5.75 |
| Upper limit of 95% CI | 47.2 | 43.3 | 6.19 | 24.4 | 23.8 |

C-C motif chemokine 14 X Cystatin-C

| | | | | | |
|---|---|---|---|---|---|
| AUC | 0.83 | 0.83 | 0.72 | 0.82 | 0.82 |
| SE | 0.023 | 0.023 | 0.036 | 0.024 | 0.024 |
| p Value | 0 | 0 | 7.6E−10 | 0 | 0 |
| nCohort Non-persistent | 107 | 120 | 232 | 142 | 148 |
| nCohort Persistent | 201 | 187 | 74 | 166 | 159 |
| Cutoff Quartile 2 | 956000 | 955000 | 958000 | 956000 | 955000 |
| Sensitivity | 88% | 89% | 92% | 89% | 89% |
| Specificity | 50% | 47% | 31% | 42% | 41% |
| Cutoff Quartile 3 | 2880000 | 2860000 | 2880000 | 2880000 | 2860000 |
| Sensitivity | 68% | 70% | 77% | 75% | 75% |
| Specificity | 83% | 81% | 59% | 79% | 76% |
| Cutoff Quartile 4 | 1.04E7 | 1.03E7 | 1.03E7 | 1.04E7 | 1.03E7 |
| Sensitivity | 37% | 39% | 46% | 42% | 43% |
| Specificity | 97% | 97% | 81% | 95% | 95% |
| OR Quartile 2 | 7.24 | 6.92 | 5.00 | 5.84 | 5.70 |
| p Value | 1.0E−11 | 5.7E−11 | 3.4E−4 | 5.1E−9 | 1.4E−8 |
| Lower limit of 95% CI | 4.09 | 3.88 | 2.07 | 3.23 | 3.12 |
| Upper limit of 95% CI | 12.8 | 12.3 | 12.1 | 10.6 | 10.4 |
| OR Quartile 3 | 10.3 | 9.87 | 4.75 | 11.0 | 9.60 |
| p Value | 5.8E−15 | 4.4E−16 | 3.8E−7 | 0 | 0 |
| Lower limit of 95% CI | 5.76 | 5.68 | 2.60 | 6.46 | 5.70 |
| Upper limit of 95% CI | 18.6 | 17.1 | 8.67 | 18.8 | 16.2 |
| OR Quartile 4 | 20.2 | 18.6 | 3.74 | 14.1 | 13.4 |
| p Value | 6.4E−7 | 3.6E−8 | 4.7E−6 | 2.6E−10 | 6.3E−11 |
| Lower limit of 95% CI | 6.19 | 6.57 | 2.12 | 6.19 | 6.16 |
| Upper limit of 95% CI | 65.9 | 52.5 | 6.57 | 31.9 | 29.2 |

C-C motif chemokine 14 X Serum Creatinine/(Weight adjusted urine output)

| | | | | | |
|---|---|---|---|---|---|
| AUC | 0.81 | 0.78 | 0.83 | 0.81 | 0.79 |
| SE | 0.024 | 0.025 | 0.030 | 0.024 | 0.025 |
| p Value | 0 | 0 | 0 | 0 | 0 |
| nCohort Non-persistent | 115 | 130 | 241 | 151 | 158 |
| nCohort Persistent | 206 | 189 | 80 | 170 | 161 |
| Cutoff Quartile 2 | 2.17 | 2.15 | 2.17 | 2.17 | 2.15 |
| Sensitivity | 87% | 86% | 96% | 89% | 88% |
| Specificity | 46% | 42% | 32% | 40% | 39% |
| Cutoff Quartile 3 | 10.2 | 10.1 | 10.2 | 10.2 | 10.1 |
| Sensitivity | 67% | 68% | 85% | 74% | 73% |
| Specificity | 81% | 75% | 61% | 76% | 73% |
| Cutoff Quartile 4 | 69.8 | 67.6 | 69.8 | 69.8 | 67.6 |
| Sensitivity | 37% | 38% | 56% | 44% | 43% |
| Specificity | 97% | 94% | 85% | 96% | 94% |
| OR Quartile 2 | 5.67 | 4.45 | 12.1 | 5.39 | 4.70 |
| p Value | 4.8E−10 | 6.3E−8 | 3.8E−5 | 1.1E−8 | 1.4E−7 |
| Lower limit of 95% CI | 3.28 | 2.59 | 3.69 | 3.02 | 2.64 |
| Upper limit of 95% CI | 9.78 | 7.65 | 39.4 | 9.59 | 8.36 |
| OR Quartile 3 | 8.77 | 6.43 | 9.02 | 8.87 | 7.11 |
| p Value | 8.7E−15 | 3.9E−13 | 9.8E−11 | 0 | 6.2E−15 |
| Lower limit of 95% CI | 5.07 | 3.89 | 4.63 | 5.35 | 4.34 |
| Upper limit of 95% CI | 15.2 | 10.6 | 17.6 | 14.7 | 11.6 |
| OR Quartile 4 | 16.6 | 9.38 | 7.32 | 19.1 | 11.4 |
| p Value | 1.1E−7 | 1.4E−8 | 5.5E−12 | 3.2E−11 | 2.2E−11 |
| Lower limit of 95% CI | 5.87 | 4.33 | 4.16 | 7.99 | 5.58 |
| Upper limit of 95% CI | 46.7 | 20.3 | 12.9 | 45.6 | 23.2 |

C-C motif chemokine 14 X Cystatin-C/(Weight adjusted urine output)

| | | | | | |
|---|---|---|---|---|---|
| AUC | 0.79 | 0.76 | 0.81 | 0.80 | 0.78 |
| SE | 0.026 | 0.027 | 0.032 | 0.025 | 0.026 |
| p Value | 0 | 0 | 0 | 0 | 0 |
| nCohort Non-persistent | 105 | 118 | 231 | 140 | 146 |
| nCohort Persistent | 200 | 186 | 74 | 165 | 158 |
| Cutoff Quartile 2 | 1580000 | 1580000 | 1580000 | 1580000 | 1580000 |
| Sensitivity | 86% | 85% | 96% | 87% | 87% |
| Specificity | 45% | 41% | 32% | 39% | 38% |
| Cutoff Quartile 3 | 6710000 | 6570000 | 6710000 | 6710000 | 6570000 |
| Sensitivity | 66% | 67% | 82% | 72% | 72% |
| Specificity | 79% | 76% | 60% | 76% | 73% |
| Cutoff Quartile 4 | 3.85E7 | 3.84E7 | 3.85E7 | 3.85E7 | 3.84E7 |
| Sensitivity | 36% | 35% | 54% | 42% | 41% |
| Specificity | 94% | 92% | 84% | 94% | 92% |
| OR Quartile 2 | 4.78 | 3.87 | 10.9 | 4.44 | 3.94 |
| p Value | 2.5E−8 | 1.1E−6 | 7.9E−5 | 3.0E−7 | 2.2E−6 |
| Lower limit of 95% CI | 2.76 | 2.24 | 3.33 | 2.51 | 2.23 |
| Upper limit of 95% CI | 8.28 | 6.67 | 35.9 | 7.84 | 6.96 |
| OR Quartile 3 | 7.16 | 6.43 | 7.09 | 8.07 | 6.89 |
| p Value | 3.0E−12 | 2.9E−12 | 4.4E−9 | 2.0E−15 | 6.0E−14 |
| Lower limit of 95% CI | 4.12 | 3.81 | 3.69 | 4.82 | 4.16 |
| Upper limit of 95% CI | 12.5 | 10.8 | 13.6 | 13.5 | 11.4 |
| OR Quartile 4 | 9.08 | 5.94 | 6.17 | 11.9 | 7.60 |
| p Value | 7.4E−7 | 1.0E−6 | 6.3E−10 | 4.6E−10 | 3.0E−9 |
| Lower limit of 95% CI | 3.79 | 2.91 | 3.46 | 5.45 | 3.89 |
| Upper limit of 95% CI | 21.8 | 12.1 | 11.0 | 25.8 | 14.9 |

| | Persistence duration (hr) | | | |
|---|---|---|---|---|
| | 48 | 72 | | |
| | UO only | sCr or UO | sCr only | UO only |
| C-C motif chemokine 14 | | | | |
| AUC | 0.73 | 0.80 | 0.80 | 0.74 |
| SE | 0.038 | 0.024 | 0.025 | 0.040 |
| p Value | 7.0E−10 | 0 | 0 | 8.3E−10 |
| nCohort Non-persistent | 265 | 174 | 180 | 272 |
| nCohort Persistent | 64 | 157 | 149 | 57 |
| Cutoff Quartile 2 | 0.732 | 0.725 | 0.719 | 0.732 |
| Sensitivity | 94% | 92% | 91% | 95% |
| Specificity | 29% | 40% | 38% | 29% |
| Cutoff Quartile 3 | 1.64 | 1.64 | 1.64 | 1.64 |
| Sensitivity | 83% | 76% | 76% | 84% |
| Specificity | 58% | 73% | 71% | 57% |
| Cutoff Quartile 4 | 5.09 | 5.16 | 5.09 | 5.09 |
| Sensitivity | 44% | 44% | 45% | 47% |
| Specificity | 79% | 92% | 91% | 79% |
| OR Quartile 2 | 6.26 | 7.46 | 6.50 | 7.37 |
| p Value | 5.9E−4 | 9.4E−10 | 1.2E−8 | 0.0010 |
| Lower limitof 95% CI | 2.20 | 3.92 | 3.42 | 2.24 |
| Upper limit of 95% CI | 17.8 | 14.2 | 12.4 | 24.3 |
| OR Quartile 3 | 6.58 | 8.46 | 7.73 | 7.07 |
| p Value | 1.0E−7 | 0 | 4.4E−16 | 3.4E−7 |
| Lower limit of 95% CI | 3.29 | 5.16 | 4.71 | 3.33 |
| Upper limit of 95% CI | 13.2 | 13.9 | 12.7 | 15.0 |
| OR Quartile 4 | 2.97 | 8.96 | 8.38 | 3.47 |
| p Value | 2.1E−4 | 9.4E−12 | 6.5E−12 | 4.4E−5 |
| Lower limit of 95% CI | 1.67 | 4.77 | 4.57 | 1.91 |
| Upper limit of 95% CI | 5.28 | 16.8 | 15.4 | 6.31 |
| Weight adjusted urine output | | | | |
| AUC | 0.19 | 0.36 | 0.39 | 0.21 |
| SE | 0.034 | 0.030 | 0.031 | 0.036 |
| p Value | 0 | 5.3E−6 | 3.0E−4 | 4.4E−16 |
| nCohort Non-persistent | 270 | 177 | 184 | 277 |
| nCohort Persistent | 67 | 160 | 151 | 60 |
| Cutoff Quartile 2 | 0.189 | 0.189 | 0.192 | 0.189 |
| Sensitivity | 39% | 66% | 68% | 40% |
| Specificity | 16% | 16% | 19% | 17% |
| Cutoff Quartile 3 | 0.366 | 0.366 | 0.368 | 0.366 |
| Sensitivity | 18% | 40% | 41% | 18% |
| Specificity | 42% | 41% | 42% | 43% |
| Cutoff Quartile 4 | 0.857 | 0.857 | 0.862 | 0.857 |
| Sensitivity | 6% | 20% | 21% | 7% |
| Specificity | 70% | 70% | 71% | 71% |
| OR Quartile 2 | 0.120 | 0.374 | 0.504 | 0.140 |
| p Value | 1.9E−12 | 1.8E−4 | 0.0076 | 1.6E−10 |
| Lower limit of 95% CI | 0.0666 | 0.224 | 0.305 | 0.0765 |
| Upper limit of 95% CI | 0.217 | 0.626 | 0.833 | 0.255 |
| OR Quartile 3 | 0.157 | 0.457 | 0.513 | 0.169 |
| p Value | 6.0E−8 | 4.3E−4 | 0.0027 | 5.5E−7 |
| Lower limit of 95% CI | 0.0804 | 0.296 | 0.331 | 0.0843 |
| Upper limit of 95% CI | 0.307 | 0.707 | 0.793 | 0.339 |
| OR Quartile 4 | 0.148 | 0.585 | 0.639 | 0.173 |
| p Value | 3.4E−4 | 0.037 | 0.083 | 0.0010 |
| Lower limit of 95% CI | 0.0522 | 0.354 | 0.384 | 0.0607 |
| Upper limit of 95% CI | 0.421 | 0.968 | 1.06 | 0.492 |
| Serum Creatinine | | | | |
| AUC | 0.68 | 0.76 | 0.77 | 0.68 |
| SE | 0.036 | 0.025 | 0.025 | 0.038 |
| p Value | 9.1E−7 | 0 | 0 | 3.8E−6 |
| nCohort Non-persistent | 274 | 177 | 184 | 282 |
| nCohort Persistent | 78 | 177 | 168 | 70 |
| Cutoff Quartile 2 | 1.65 | 1.64 | 1.64 | 1.65 |
| Sensitivity | 88% | 90% | 92% | 89% |
| Specificity | 29% | 41% | 41% | 28% |
| Cutoff Quartile 3 | 2.44 | 2.44 | 2.44 | 2.44 |
| Sensitivity | 68% | 70% | 71% | 69% |
| Specificity | 55% | 69% | 70% | 54% |
| Cutoff Quartile 4 | 3.32 | 3.32 | 3.31 | 3.32 |
| Sensitivity | 47% | 40% | 41% | 49% |
| Specificity | 81% | 89% | 90% | 80% |
| OR Quartile 2 | 3.11 | 6.45 | 8.20 | 3.07 |
| p Value | 0.0028 | 3.6E−10 | 9.9E−11 | 0.0049 |
| Lower limit of 95% CI | 1.48 | 3.60 | 4.34 | 1.41 |
| Upper limit of 95% CI | 6.52 | 11.6 | 15.5 | 6.70 |
| OR Quartile 3 | 2.56 | 5.33 | 5.71 | 2.59 |
| p Value | 5.2E−4 | 4.9E−13 | 9.8E−14 | 8.1E−4 |
| Lower limit of 95% CI | 1.51 | 3.39 | 3.61 | 1.48 |
| Upper limit of 95% CI | 4.36 | 8.39 | 9.04 | 4.51 |
| OR Quartile 4 | 3.85 | 5.57 | 6.05 | 3.90 |
| p Value | 8.7E−7 | 2.2E−9 | 4.4E−10 | 1.5E−6 |
| Lower limit of 95% CI | 2.25 | 3.17 | 3.44 | 2.24 |
| Upper limit of 95% CI | 6.59 | 9.78 | 10.7 | 6.78 |
| Cystatin-C | | | | |
| AUC | 0.71 | 0.75 | 0.75 | 0.71 |
| SE | 0.041 | 0.028 | 0.028 | 0.043 |
| p Value | 2.9E−7 | 0 | 0 | 1.1E−6 |
| nCohort Non-persistent | 248 | 158 | 163 | 254 |
| nCohort Persistent | 58 | 150 | 144 | 52 |
| Cutoff Quartile 2 | 1170000 | 1170000 | 1170000 | 1170000 |
| Sensitivity | 90% | 89% | 88% | 88% |
| Specificity | 29% | 37% | 36% | 28% |
| Cutoff Quartile 3 | 1660000 | 1660000 | 1650000 | 1660000 |
| Sensitivity | 78% | 69% | 70% | 77% |
| Specificity | 56% | 68% | 67% | 56% |
| Cutoff Quartile 4 | 2150000 | 2150000 | 2150000 | 2150000 |
| Sensitivity | 48% | 39% | 40% | 50% |
| Specificity | 80% | 89% | 88% | 80% |
| OR Quartile 2 | 3.48 | 4.66 | 4.24 | 2.97 |
| p Value | 0.0060 | 4.7E−7 | 2.3E−6 | 0.017 |
| Lower limit of 95% CI | 1.43 | 2.56 | 2.33 | 1.22 |
| Upper limit of 95% CI | 8.46 | 8.49 | 7.71 | 7.27 |
| OR Quartile 3 | 4.49 | 4.88 | 4.87 | 4.16 |
| p Value | 1.0E−5 | 1.2E−10 | 1.5E−10 | 5.3E−5 |
| Lower limit of 95% CI | 2.30 | 3.01 | 3.00 | 2.08 |
| Upper limit of 95% CI | 8.74 | 7.91 | 7.91 | 8.30 |
| OR Quartile 4 | 3.79 | 5.04 | 5.11 | 3.98 |
| p Value | 1.5E−5 | 7.7E−8 | 4.1E−8 | 1.4E−5 |
| Lower limit of 95% CI | 2.08 | 2.80 | 2.85 | 2.13 |
| Upper limit of 95% CI | 6.92 | 9.10 | 9.16 | 7.43 |
| C-C motif chemokine 14/(Weight adjusted urine output) | | | | |
| AUC | 0.82 | 0.78 | 0.76 | 0.82 |
| SE | 0.034 | 0.026 | 0.027 | 0.035 |
| p Value | 0 | 0 | 0 | 0 |
| nCohort Non-persistent | 264 | 172 | 178 | 271 |
| nCohort Persistent | 64 | 156 | 148 | 57 |
| Cutoff Quartile 2 | 1.26 | 1.26 | 1.24 | 1.26 |
| Sensitivity | 98% | 88% | 88% | 98% |
| Specificity | 31% | 37% | 36% | 30% |
| Cutoff Quartile 3 | 4.59 | 4.59 | 4.54 | 4.59 |
| Sensitivity | 84% | 73% | 72% | 84% |
| Specificity | 58% | 71% | 69% | 57% |
| Cutoff Quartile 4 | 24.3 | 24.3 | 22.9 | 24.3 |
| Sensitivity | 61% | 44% | 43% | 63% |
| Specificity | 84% | 92% | 90% | 83% |
| OR Quartile 2 | 27.9 | 4.17 | 4.05 | 23.9 |
| p Value | 0.0011 | 9.8E−7 | 2.3E−6 | 0.0018 |
| Lower limit of 95% CI | 3.80 | 2.35 | 2.27 | 3.25 |
| Upper limit of 95% CI | 205 | 7.38 | 7.24 | 175 |
| OR Quartile 3 | 7.56 | 6.62 | 5.69 | 7.13 |
| p Value | 3.3E−8 | 1.7E−14 | 1.2E−12 | 3.0E−7 |
| Lower limit of 95% CI | 3.69 | 4.09 | 3.52 | 3.36 |
| Upper limit of 95% CI | 15.5 | 10.7 | 9.18 | 15.1 |
| OR Quartile 4 | 8.02 | 8.72 | 6.77 | 8.39 |
| p Value | 9.7E−12 | 1.8E−11 | 1.6E−10 | 2.5E−11 |
| Lower limit of 95% CI | 4.40 | 4.64 | 3.77 | 4.49 |
| Upper limit of 95% CI | 14.6 | 16.4 | 12.2 | 15.7 |
| C-C motif chemokine 14 X Serum Creatinine | | | | |
| AUC | 0.74 | 0.83 | 0.82 | 0.75 |
| SE | 0.038 | 0.023 | 0.024 | 0.040 |
| p Value | 2.3E−10 | 0 | 0 | 3.5E−10 |
| nCohort Non-persistent | 259 | 169 | 175 | 266 |
| nCohort Persistent | 63 | 155 | 147 | 56 |
| Cutoff Quartile 2 | 1.30 | 1.29 | 1.29 | 1.30 |
| Sensitivity | 90% | 92% | 92% | 91% |
| Specificity | 29% | 40% | 39% | 29% |
| Cutoff Quartile 3 | 3.97 | 3.97 | 3.91 | 3.97 |
| Sensitivity | 84% | 77% | 78% | 86% |
| Specificity | 58% | 75% | 74% | 58% |
| Cutoff Quartile 4 | 18.0 | 18.3 | 16.8 | 18.0 |
| Sensitivity | 51% | 45% | 46% | 54% |
| Specificity | 81% | 93% | 93% | 81% |
| OR Quartile 2 | 3.87 | 7.35 | 7.32 | 4.08 |
| p Value | 0.0027 | 1.4E−9 | 4.1E−9 | 0.0040 |
| Lower limit of 95% CI | 1.60 | 3.86 | 3.77 | 1.57 |
| Upper limit of 95% CI | 9.36 | 14.0 | 14.2 | 10.6 |
| OR Quartile 3 | 7.41 | 10.4 | 10.1 | 8.12 |
| p Value | 4.9E−8 | 0 | 0 | 1.8E−7 |
| Lower limit of 95% CI | 3.61 | 6.20 | 6.01 | 3.70 |
| Upper limit of 95% CI | 15.2 | 17.3 | 16.9 | 17.8 |
| OR Quartile 4 | 4.42 | 10.5 | 10.7 | 4.86 |
| p Value | 5.9E−7 | 4.9E−12 | 9.4E−13 | 3.3E−7 |
| Lower limit of 95% CI | 2.47 | 5.39 | 5.59 | 2.65 |
| Upper limit of 95% CI | 7.93 | 20.5 | 20.6 | 8.93 |
| C-C motif chemokine 14 X Cystatin-C | | | | |
| AUC | 0.74 | 0.82 | 0.82 | 0.75 |
| SE | 0.040 | 0.024 | 0.024 | 0.041 |
| p Value | 3.4E−9 | 0 | 0 | 1.1E−9 |
| nCohort Non-persistent | 248 | 158 | 163 | 254 |
| nCohort Persistent | 58 | 150 | 144 | 52 |
| Cutoff Quartile 2 | 958000 | 956000 | 955000 | 958000 |
| Sensitivity | 90% | 91% | 91% | 90% |
| Specificity | 29% | 41% | 39% | 28% |
| Cutoff Quartile 3 | 2880000 | 2880000 | 2860000 | 2880000 |
| Sensitivity | 83% | 77% | 77% | 83% |
| Specificity | 58% | 75% | 74% | 57% |
| Cutoff Quartile 4 | 1.03E7 | 1.04E7 | 1.03E7 | 1.03E7 |
| Sensitivity | 52% | 45% | 47% | 58% |
| Specificity | 81% | 94% | 94% | 81% |
| OR Quartile 2 | 3.48 | 7.18 | 6.51 | 3.72 |
| p Value | 0.0060 | 3.1E−9 | 1.7E−8 | 0.0074 |
| Lower limit of 95% CI | 1.43 | 3.74 | 3.40 | 1.42 |
| Upper limit of 95% CI | 8.46 | 13.8 | 12.5 | 9.73 |
| OR Quartile 3 | 6.54 | 10.0 | 9.39 | 6.25 |
| p Value | 4.1E−7 | 0 | 0 | 2.3E−6 |
| Lower limit of 95% CI | 3.16 | 5.94 | 5.57 | 2.92 |
| Upper limit of 95% CI | 13.5 | 16.9 | 15.8 | 13.4 |
| OR Quartile 4 | 4.58 | 13.7 | 13.3 | 6.01 |
| p Value | 8.2E−7 | 5.7E−12 | 1.7E−12 | 3.1E−8 |
| Lower limit of 95% CI | 2.50 | 6.51 | 6.49 | 3.18 |
| Upper limit of 95% CI | 8.39 | 28.9 | 27.3 | 11.3 |
| C-C motif chemokine 14 X Serum Creatinine/(Weight adjusted urine output) | | | | |
| AUC | 0.83 | 0.81 | 0.80 | 0.83 |
| SE | 0.033 | 0.024 | 0.026 | 0.035 |
| p Value | 0 | 0 | 0 | 0 |
| nCohort Non-persistent | 258 | 167 | 173 | 265 |
| nCohort Persistent | 63 | 154 | 146 | 56 |
| Cutoff Quartile 2 | 2.17 | 2.17 | 2.15 | 2.17 |
| Sensitivity | 95% | 90% | 90% | 95% |
| Specificity | 30% | 39% | 38% | 29% |
| Cutoff Quartile 3 | 10.2 | 10.2 | 10.1 | 10.2 |
| Sensitivity | 86% | 76% | 75% | 86% |
| Specificity | 59% | 74% | 71% | 57% |
| Cutoff Quartile 4 | 69.8 | 69.8 | 67.6 | 69.8 |
| Sensitivity | 62% | 47% | 46% | 66% |
| Specificity | 84% | 95% | 92% | 83% |
| OR Quartile 2 | 8.51 | 5.91 | 5.26 | 7.24 |
| p Value | 4.2E−4 | 1.7E−8 | 1.3E−7 | 0.0011 |
| Lower limit of 95% CI | 2.59 | 3.19 | 2.84 | 2.19 |
| Upper limit of 95% CI | 28.0 | 10.9 | 9.74 | 23.9 |
| OR Quartile 3 | 8.47 | 8.84 | 7.52 | 8.07 |
| p Value | 2.2E−8 | 0 | 2.4E−15 | 2.0E−7 |
| Lower limit of 95% CI | 4.01 | 5.33 | 4.56 | 3.67 |
| Upper limit of 95% CI | 17.9 | 14.6 | 12.4 | 17.7 |
| OR Quartile 4 | 8.36 | 15.4 | 10.4 | 9.78 |
| p Value | 6.8E−12 | 5.2E−13 | 1.8E−12 | 3.1E−12 |
| Lower limit of 95% CI | 4.56 | 7.34 | 5.44 | 5.15 |
| Upper limit of 95% CI | 15.3 | 32.4 | 20.0 | 18.6 |
| C-C motif chemokine 14 X Cystatin-C/(Weight adjusted urine output) | | | | |
| AUC | 0.81 | 0.80 | 0.79 | 0.81 |
| SE | 0.036 | 0.026 | 0.027 | 0.038 |
| p Value | 0 | 0 | 0 | 2.2E−16 |
| nCohort Non-persistent | 247 | 156 | 161 | 253 |
| nCohort Persistent | 58 | 149 | 143 | 52 |
| Cutoff Quartile 2 | 1580000 | 1580000 | 1580000 | 1580000 |
| Sensitivity | 95% | 89% | 88% | 94% |
| Specificity | 30% | 38% | 37% | 29% |
| Cutoff Quartile 3 | 6710000 | 6710000 | 6570000 | 6710000 |
| Sensitivity | 86% | 75% | 75% | 87% |
| Specificity | 58% | 74% | 72% | 57% |
| Cutoff Quartile 4 | 3.85E7 | 3.85E7 | 3.84E7 | 3.85E7 |
| Sensitivity | 59% | 44% | 43% | 60% |
| Specificity | 83% | 92% | 91% | 82% |
| OR Quartile 2 | 7.69 | 4.72 | 4.29 | 6.62 |
| p Value | 8.1E−4 | 3.9E−7 | 1.9E−6 | 0.0020 |
| Lower limit of 95% CI | 2.33 | 2.59 | 2.35 | 2.00 |
| Upper limit of 95% CI | 25.4 | 8.60 | 7.81 | 21.9 |
| OR Quartile 3 | 8.74 | 8.49 | 7.66 | 8.63 |
| p Value | 7.0E−8 | 4.4E−16 | 5.8E−15 | 4.1E−7 |
| Lower limit of 95% CI | 3.97 | 5.07 | 4.60 | 3.75 |
| Upper limit of 95% CI | 19.2 | 14.2 | 12.8 | 19.9 |
| OR Quartile 4 | 6.72 | 9.29 | 7.24 | 6.64 |
| p Value | 1.5E−9 | 8.1E−11 | 5.8E−10 | 6.5E−9 |
| Lower limit of 95% CI | 3.62 | 4.74 | 3.87 | 3.50 |
| Upper limit of 95% CI | 12.5 | 18.2 | 13.5 | 12.6 |

TABLE 21
Comparison of marker levels and the area under the ROC curve (AUC) in urine samples for
the “persistent” and “non-persistent” cohorts where persistence starts
within 48 hours after sample collection and renal status was assessed by serum creatinine
(sCr) only, urine output (UO) only, or by either serum creatinine or urine output KDIGO
criteria. Markers are either positive going or negative going which can be discerned from
the numerical value of the AUC. More specifically, an AUC value >0.5 indicates a positive
going marker and an AUC value <0.5 indicates a negative going marker.

| Persistence duration (hr) | | | | |
|---|---|---|---|---|
| 24 | | | 48 | |
| sCr or UO | sCr only | UO only | sCr or UO | sCr only |

C-C motif chemokine 14

| | | | | | |
|---|---|---|---|---|---|
| AUC | 0.79 | 0.80 | 0.72 | 0.79 | 0.79 |
| SE | 0.024 | 0.024 | 0.032 | 0.025 | 0.025 |
| p Value | 0 | 0 | 1.5E−11 | 0 | 0 |
| nCohort Non-persistent | 115 | 133 | 230 | 153 | 162 |
| nCohort Persistent | 216 | 196 | 99 | 178 | 167 |
| Cutoff Quartile 2 | 0.725 | 0.719 | 0.732 | 0.725 | 0.719 |
| Sensitivity | 88% | 89% | 92% | 89% | 89% |
| Specificity | 49% | 46% | 32% | 41% | 40% |
| Cutoff Quartile 3 | 1.64 | 1.64 | 1.64 | 1.64 | 1.64 |
| Sensitivity | 66% | 68% | 77% | 71% | 72% |
| Specificity | 79% | 77% | 61% | 75% | 73% |
| Cutoff Quartile 4 | 5.16 | 5.09 | 5.09 | 5.16 | 5.09 |
| Sensitivity | 36% | 39% | 38% | 40% | 42% |
| Specificity | 96% | 95% | 80% | 93% | 92% |
| OR Quartile 2 | 6.64 | 7.06 | 5.40 | 5.53 | 5.41 |
| p Value | 9.2E−12 | 1.4E−11 | 2.0E−5 | 3.1E−9 | 1.3E−8 |
| Lower limit of 95% CI | 3.86 | 4.01 | 2.49 | 3.14 | 3.02 |
| Upper limit of 95% CI | 11.5 | 12.4 | 11.7 | 9.74 | 9.67 |
| OR Quartile 3 | 7.28 | 7.11 | 5.23 | 7.28 | 7.05 |
| p Value | 2.2E−13 | 1.9E−14 | 1.5E−9 | 1.6E−15 | 2.9E−15 |
| Lower limit of 95% CI | 4.28 | 4.30 | 3.06 | 4.47 | 4.34 |
| Upper limit of 95% CI | 12.4 | 11.8 | 8.95 | 11.9 | 11.5 |
| OR Quartile 4 | 12.4 | 11.4 | 2.56 | 8.77 | 8.27 |
| p Value | 1.4E−7 | 4.5E−9 | 3.9E−4 | 4.5E−10 | 1.3E−10 |
| Lower limit of 95% CI | 4.87 | 5.05 | 1.52 | 4.43 | 4.34 |
| Upper limit of 95% CI | 31.8 | 25.7 | 4.31 | 17.4 | 15.8 |

Weight adjusted urine output

| | | | | | |
|---|---|---|---|---|---|
| AUC | 0.40 | 0.46 | 0.23 | 0.36 | 0.40 |
| SE | 0.032 | 0.032 | 0.030 | 0.030 | 0.031 |
| p Value | 0.0023 | 0.27 | 0 | 3.2E−6 | 0.0020 |
| nCohort Non-persistent | 117 | 136 | 234 | 155 | 165 |
| nCohort Persistent | 220 | 199 | 103 | 182 | 170 |
| Cutoff Quartile 2 | 0.189 | 0.192 | 0.189 | 0.189 | 0.192 |
| Sensitivity | 71% | 73% | 50% | 66% | 70% |
| Specificity | 17% | 22% | 14% | 15% | 19% |
| Cutoff Quartile 3 | 0.366 | 0.368 | 0.366 | 0.366 | 0.368 |
| Sensitivity | 46% | 49% | 25% | 41% | 44% |
| Specificity | 42% | 48% | 39% | 39% | 43% |
| Cutoff Quartile 4 | 0.857 | 0.862 | 0.857 | 0.857 | 0.862 |
| Sensitivity | 23% | 25% | 8% | 20% | 21% |
| Specificity | 70% | 74% | 67% | 68% | 70% |
| OR Quartile 2 | 0.503 | 0.780 | 0.155 | 0.346 | 0.561 |
| p Value | 0.017 | 0.34 | 1.1E−11 | 1.1E−4 | 0.025 |
| Lower limit of 95% CI | 0.286 | 0.467 | 0.0908 | 0.202 | 0.338 |
| Upper limit of 95% CI | 0.882 | 1.30 | 0.266 | 0.593 | 0.932 |
| OR Quartile 3 | 0.612 | 0.871 | 0.215 | 0.455 | 0.582 |
| p Value | 0.033 | 0.53 | 5.4E−9 | 4.1E−4 | 0.014 |
| Lower limit of 95% CI | 0.389 | 0.563 | 0.128 | 0.294 | 0.378 |
| Upper limit of 95% CI | 0.962 | 1.35 | 0.360 | 0.704 | 0.897 |
| OR Quartile 4 | 0.689 | 0.943 | 0.172 | 0.533 | 0.614 |
| p Value | 0.15 | 0.82 | 7.6E−6 | 0.013 | 0.056 |
| Lower limit of 95% CI | 0.415 | 0.571 | 0.0794 | 0.324 | 0.372 |
| Upper limit of 95% CI | 1.14 | 1.56 | 0.371 | 0.877 | 1.01 |

Serum Creatinine

| | | | | | |
|---|---|---|---|---|---|
| AUC | 0.82 | 0.84 | 0.69 | 0.78 | 0.80 |
| SE | 0.022 | 0.021 | 0.031 | 0.024 | 0.023 |
| p Value | 0 | 0 | 1.6E−9 | 0 | 0 |
| nCohort Non-persistent | 115 | 134 | 236 | 154 | 164 |
| nCohort Persistent | 239 | 218 | 116 | 200 | 188 |
| Cutoff Quartile 2 | 1.64 | 1.64 | 1.65 | 1.64 | 1.64 |
| Sensitivity | 88% | 91% | 88% | 90% | 92% |
| Specificity | 52% | 51% | 31% | 44% | 45% |
| Cutoff Quartile 3 | 2.44 | 2.44 | 2.44 | 2.44 | 2.44 |
| Sensitivity | 66% | 69% | 67% | 68% | 71% |
| Specificity | 82% | 81% | 58% | 73% | 74% |
| Cutoff Quartile 4 | 3.32 | 3.31 | 3.32 | 3.32 | 3.31 |
| Sensitivity | 36% | 38% | 44% | 40% | 41% |
| Specificity | 96% | 96% | 84% | 93% | 93% |
| OR Quartile 2 | 7.90 | 11.1 | 3.33 | 6.74 | 9.25 |
| p Value | 3.1E−14 | 4.4E−16 | 1.5E−4 | 1.3E−11 | 9.4E−13 |
| Lower limit of 95% CI | 4.63 | 6.23 | 1.79 | 3.88 | 5.02 |
| Upper limit of 95% CI | 13.5 | 19.9 | 6.20 | 11.7 | 17.0 |
| OR Quartile 3 | 8.57 | 9.83 | 2.84 | 5.67 | 6.80 |
| p Value | 9.1E−15 | 0 | 1.1E−5 | 2.0E−13 | 1.1E−15 |
| Lower limit of 95% CI | 4.98 | 5.83 | 1.78 | 3.57 | 4.26 |
| Upper limit of 95% CI | 14.8 | 16.5 | 4.53 | 9.00 | 10.9 |
| OR Quartile 4 | 12.1 | 15.9 | 4.09 | 8.49 | 9.65 |
| p Value | 1.6E−7 | 6.7E−9 | 4.6E−8 | 5.5E−10 | 5.4E−11 |
| Lower limit of 95% CI | 4.77 | 6.23 | 2.47 | 4.32 | 4.90 |
| Upper limit of 95% CI | 30.9 | 40.4 | 6.77 | 16.7 | 19.0 |

Cystatin-C

| | | | | | |
|---|---|---|---|---|---|
| AUC | 0.77 | 0.76 | 0.72 | 0.77 | 0.76 |
| SE | 0.027 | 0.027 | 0.034 | 0.026 | 0.027 |
| p Value | 0 | 0 | 9.5E−11 | 0 | 0 |
| nCohort Non-persistent | 101 | 116 | 215 | 137 | 145 |
| nCohort Persistent | 207 | 191 | 91 | 171 | 162 |
| Cutoff Quartile 2 | 1170000 | 1170000 | 1170000 | 1170000 | 1170000 |
| Sensitivity | 86% | 87% | 90% | 88% | 88% |
| Specificity | 47% | 44% | 32% | 41% | 39% |
| Cutoff Quartile 3 | 1660000 | 1650000 | 1660000 | 1660000 | 1650000 |
| Sensitivity | 64% | 65% | 76% | 69% | 69% |
| Specificity | 79% | 75% | 61% | 74% | 71% |
| Cutoff Quartile 4 | 2150000 | 2150000 | 2150000 | 2150000 | 2150000 |
| Sensitivity | 32% | 35% | 43% | 38% | 40% |
| Specificity | 90% | 91% | 82% | 91% | 91% |
| OR Quartile 2 | 5.34 | 5.21 | 4.21 | 5.22 | 4.47 |
| p Value | 3.1E−9 | 6.7E−9 | 1.6E−4 | 2.0E−8 | 3.4E−7 |
| Lower limit of 95% CI | 3.07 | 2.98 | 2.00 | 2.93 | 2.51 |
| Upper limit of 95% CI | 9.30 | 9.10 | 8.89 | 9.30 | 7.94 |
| OR Quartile 3 | 6.85 | 5.68 | 4.89 | 6.25 | 5.49 |
| p Value | 1.4E−11 | 3.9E−11 | 1.8E−8 | 6.7E−13 | 9.3E−12 |
| Lower limit of 95% CI | 3.92 | 3.39 | 2.81 | 3.79 | 3.37 |
| Upper limit of 95% CI | 12.0 | 9.51 | 8.50 | 10.3 | 8.96 |
| OR Quartile 4 | 4.35 | 5.04 | 3.49 | 6.39 | 6.63 |
| p Value | 5.5E−5 | 4.2E−6 | 6.4E−6 | 5.3E−8 | 1.2E−8 |
| Lower limit of 95% CI | 2.13 | 2.53 | 2.03 | 3.28 | 3.46 |
| Upper limit of 95% CI | 8.90 | 10.0 | 6.01 | 12.5 | 12.7 |

C-C motif chemokine 14/(Weight adjusted urine output)

| | | | | | |
|---|---|---|---|---|---|
| AUC | 0.75 | 0.72 | 0.79 | 0.77 | 0.75 |
| SE | 0.026 | 0.028 | 0.029 | 0.026 | 0.027 |
| p Value | 0 | 1.1E−15 | 0 | 0 | 0 |
| nCohort Non-persistent | 113 | 131 | 229 | 151 | 160 |
| nCohort Persistent | 215 | 195 | 99 | 177 | 166 |
| Cutoff Quartile 2 | 1.26 | 1.24 | 1.26 | 1.26 | 1.24 |
| Sensitivity | 84% | 84% | 96% | 86% | 86% |
| Specificity | 42% | 38% | 34% | 38% | 37% |
| Cutoff Quartile 3 | 4.59 | 4.54 | 4.59 | 4.59 | 4.54 |
| Sensitivity | 64% | 65% | 81% | 70% | 69% |
| Specificity | 77% | 72% | 63% | 74% | 70% |
| Cutoff Quartile 4 | 24.3 | 22.9 | 24.3 | 24.3 | 22.9 |
| Sensitivity | 34% | 35% | 47% | 41% | 40% |
| Specificity | 93% | 90% | 85% | 93% | 91% |
| OR Quartile 2 | 3.93 | 3.14 | 12.3 | 3.98 | 3.63 |
| p Value | 2.9E−7 | 1.4E−5 | 2.2E−6 | 5.7E−7 | 3.5E−6 |
| Lower limit of 95% CI | 2.33 | 1.87 | 4.35 | 2.31 | 2.11 |
| Upper limit of 95% CI | 6.63 | 5.28 | 34.6 | 6.83 | 6.26 |
| OR Quartile 3 | 6.00 | 4.64 | 7.27 | 6.49 | 5.26 |
| p Value | 1.4E−11 | 3.9E−10 | 7.6E−12 | 3.5E−14 | 5.6E−12 |
| Lower limit of 95% CI | 3.57 | 2.87 | 4.12 | 4.00 | 3.28 |
| Upper limit of 95% CI | 10.1 | 7.50 | 12.8 | 10.5 | 8.44 |
| OR Quartile 4 | 6.89 | 4.97 | 5.01 | 9.67 | 6.54 |
| p Value | 9.6E−7 | 1.0E−6 | 3.3E−9 | 3.4E−10 | 2.2E−9 |
| Lower limit of 95% CI | 3.18 | 2.61 | 2.94 | 4.76 | 3.54 |
| Upper limit of 95% CI | 14.9 | 9.46 | 8.55 | 19.6 | 12.1 |

C-C motif chemokine 14 X Serum Creatinine

| | | | | | |
|---|---|---|---|---|---|
| AUC | 0.83 | 0.84 | 0.73 | 0.82 | 0.83 |
| SE | 0.022 | 0.021 | 0.032 | 0.023 | 0.023 |
| p Value | 0 | 0 | 8.2E−13 | 0 | 0 |
| nCohort Non-persistent | 110 | 128 | 224 | 148 | 157 |
| nCohort Persistent | 214 | 194 | 98 | 176 | 165 |
| Cutoff Quartile 2 | 1.29 | 1.29 | 1.30 | 1.29 | 1.29 |
| Sensitivity | 88% | 90% | 89% | 90% | 91% |
| Specificity | 50% | 48% | 31% | 43% | 42% |
| Cutoff Quartile 3 | 3.97 | 3.91 | 3.97 | 3.97 | 3.91 |
| Sensitivity | 68% | 71% | 81% | 73% | 75% |
| Specificity | 85% | 82% | 63% | 78% | 76% |
| Cutoff Quartile 4 | 18.3 | 16.8 | 18.0 | 18.3 | 16.8 |
| Sensitivity | 36% | 39% | 42% | 41% | 43% |
| Specificity | 96% | 96% | 82% | 94% | 94% |
| OR Quartile 2 | 7.23 | 8.65 | 3.60 | 6.51 | 7.25 |
| p Value | 2.8E−12 | 5.7E−13 | 2.7E−4 | 3.9E−10 | 3.3E−10 |
| Lower limit of 95% CI | 4.15 | 4.81 | 1.81 | 3.62 | 3.91 |
| Upper limit of 95% CI | 12.6 | 15.6 | 7.15 | 11.7 | 13.5 |
| OR Quartile 3 | 12.6 | 11.2 | 7.20 | 9.56 | 9.81 |
| p Value | 2.2E−16 | 0 | 1.1E−11 | 0 | 0 |
| Lower limit of 95% CI | 6.90 | 6.51 | 4.07 | 5.74 | 5.89 |
| Upper limit of 95% CI | 23.1 | 19.5 | 12.7 | 15.9 | 16.3 |
| OR Quartile 4 | 14.9 | 15.8 | 3.31 | 10.7 | 11.1 |
| p Value | 3.3E−7 | 8.2E−9 | 8.7E−6 | 3.1E−10 | 3.2E−11 |
| Lower limit of 95% CI | 5.28 | 6.19 | 1.95 | 5.11 | 5.45 |
| Upper limit of 95% CI | 42.0 | 40.5 | 5.61 | 22.4 | 22.6 |

C-C motif chemokine 14 X Cystatin-C

| | | | | | |
|---|---|---|---|---|---|
| AUC | 0.82 | 0.82 | 0.73 | 0.81 | 0.81 |
| SE | 0.023 | 0.023 | 0.033 | 0.024 | 0.024 |
| p Value | 0 | 0 | 3.0E−12 | 0 | 0 |
| nCohort Non-persistent | 101 | 116 | 215 | 137 | 145 |
| nCohort Persistent | 207 | 191 | 91 | 171 | 162 |
| Cutoff Quartile 2 | 956000 | 955000 | 958000 | 956000 | 955000 |
| Sensitivity | 87% | 88% | 91% | 88% | 89% |
| Specificity | 50% | 47% | 32% | 42% | 41% |
| Cutoff Quartile 3 | 2880000 | 2860000 | 2880000 | 2880000 | 2860000 |
| Sensitivity | 66% | 69% | 78% | 73% | 74% |
| Specificity | 83% | 81% | 62% | 79% | 77% |
| Cutoff Quartile 4 | 1.04E7 | 1.03E7 | 1.03E7 | 1.04E7 | 1.03E7 |
| Sensitivity | 36% | 38% | 44% | 41% | 43% |
| Specificity | 97% | 97% | 83% | 95% | 94% |
| OR Quartile 2 | 7.10 | 6.93 | 4.90 | 5.38 | 5.49 |
| p Value | 1.2E−11 | 4.1E−11 | 6.5E−5 | 1.1E−8 | 1.7E−8 |
| Lower limit of 95% CI | 4.03 | 3.90 | 2.25 | 3.02 | 3.04 |
| Upper limit of 95% CI | 12.5 | 12.3 | 10.7 | 9.58 | 9.92 |
| OR Quartile 3 | 9.67 | 9.56 | 5.76 | 10.1 | 9.33 |
| p Value | 8.1E−14 | 1.8E−15 | 1.5E−9 | 0 | 0 |
| Lower limit of 95% CI | 5.33 | 5.48 | 3.26 | 5.95 | 5.54 |
| Upper limit of 95% CI | 17.5 | 16.7 | 10.2 | 17.2 | 15.7 |
| OR Quartile 4 | 18.2 | 17.3 | 3.77 | 12.9 | 12.7 |
| p Value | 1.6E−6 | 7.5E−8 | 1.8E−6 | 9.8E−10 | 1.5E−10 |
| Lower limit of 95% CI | 5.57 | 6.13 | 2.19 | 5.67 | 5.84 |
| Upper limit of 95% CI | 59.4 | 49.0 | 6.51 | 29.2 | 27.7 |

C-C motif chemokine 14 X Serum Creatinine/(Weight adjusted urine output)

| | | | | | |
|---|---|---|---|---|---|
| AUC | 0.80 | 0.78 | 0.80 | 0.81 | 0.79 |
| SE | 0.024 | 0.025 | 0.029 | 0.024 | 0.025 |
| p Value | 0 | 0 | 0 | 0 | 0 |
| nCohort Non-persistent | 108 | 126 | 223 | 146 | 155 |
| nCohort Persistent | 213 | 193 | 98 | 175 | 164 |
| Cutoff Quartile 2 | 2.17 | 2.15 | 2.17 | 2.17 | 2.15 |
| Sensitivity | 86% | 86% | 94% | 89% | 88% |
| Specificity | 46% | 42% | 33% | 41% | 39% |
| Cutoff Quartile 3 | 10.2 | 10.1 | 10.2 | 10.2 | 10.1 |
| Sensitivity | 67% | 67% | 84% | 73% | 73% |
| Specificity | 82% | 76% | 65% | 77% | 74% |
| Cutoff Quartile 4 | 69.8 | 67.6 | 69.8 | 69.8 | 67.6 |
| Sensitivity | 36% | 37% | 48% | 43% | 43% |
| Specificity | 96% | 94% | 85% | 96% | 94% |
| OR Quartile 2 | 5.26 | 4.46 | 7.62 | 5.41 | 4.95 |
| p Value | 1.8E−9 | 5.3E−8 | 5.0E−6 | 6.7E−9 | 5.4E−8 |
| Lower limit of 95% CI | 3.06 | 2.60 | 3.19 | 3.06 | 2.78 |
| Upper limit of 95% CI | 9.03 | 7.65 | 18.2 | 9.57 | 8.82 |
| OR Quartile 3 | 9.37 | 6.60 | 9.34 | 9.33 | 7.35 |
| p Value | 1.7E−14 | 3.5E−13 | 3.4E−13 | 0 | 2.9E−15 |
| Lower limit of 95% CI | 5.29 | 3.97 | 5.12 | 5.59 | 4.48 |
| Upper limit of 95% CI | 16.6 | 11.0 | 17.1 | 15.6 | 12.1 |
| OR Quartile 4 | 14.7 | 8.78 | 5.12 | 17.5 | 10.8 |
| p Value | 3.7E−7 | 3.7E−8 | 2.8E−9 | 1.1E−10 | 5.6E−11 |
| Lower limit of 95% CI | 5.22 | 4.05 | 2.99 | 7.33 | 5.30 |
| Upper limit of 95% CI | 41.5 | 19.0 | 8.78 | 41.8 | 22.0 |

C-C motif chemokine 14 X Cystatin-C/(Weight adjusted urine output)

| | | | | | |
|---|---|---|---|---|---|
| AUC | 0.78 | 0.75 | 0.79 | 0.79 | 0.77 |
| SE | 0.026 | 0.027 | 0.031 | 0.025 | 0.027 |
| p Value | 0 | 0 | 0 | 0 | 0 |
| nCohort Non-persistent | 99 | 114 | 214 | 135 | 143 |
| nCohort Persistent | 206 | 190 | 91 | 170 | 161 |
| Cutoff Quartile 2 | 1580000 | 1580000 | 1580000 | 1580000 | 1580000 |
| Sensitivity | 84% | 84% | 95% | 86% | 86% |
| Specificity | 44% | 40% | 33% | 39% | 38% |
| Cutoff Quartile 3 | 6710000 | 6570000 | 6710000 | 6710000 | 6570000 |
| Sensitivity | 65% | 66% | 81% | 71% | 71% |
| Specificity | 80% | 76% | 63% | 76% | 73% |
| Cutoff Quartile 4 | 3.85E7 | 3.84E7 | 3.85E7 | 3.85E7 | 3.84E7 |
| Sensitivity | 34% | 35% | 46% | 41% | 40% |
| Specificity | 94% | 91% | 84% | 94% | 92% |
| OR Quartile 2 | 4.35 | 3.61 | 8.54 | 4.13 | 3.83 |
| p Value | 1.4E−7 | 3.3E−6 | 8.7E−6 | 6.6E−7 | 2.9E−6 |
| Lower limit of 95% CI | 2.52 | 2.10 | 3.32 | 2.36 | 2.18 |
| Upper limit of 95% CI | 7.52 | 6.19 | 22.0 | 7.22 | 6.73 |
| OR Quartile 3 | 7.20 | 6.20 | 7.44 | 7.95 | 6.70 |
| p Value | 9.5E−12 | 1.0E−11 | 4.1E−11 | 4.0E−15 | 1.3E−13 |
| Lower limit of 95% CI | 4.08 | 3.66 | 4.10 | 4.74 | 4.05 |
| Upper limit of 95% CI | 12.7 | 10.5 | 13.5 | 13.3 | 11.1 |
| OR Quartile 4 | 8.15 | 5.54 | 4.38 | 10.8 | 7.20 |
| p Value | 2.5E−6 | 2.7E−6 | 1.3E−7 | 1.8E−9 | 7.7E−9 |
| Lower limit of 95% CI | 3.40 | 2.71 | 2.53 | 4.99 | 3.69 |
| Upper limit of 95% CI | 19.5 | 11.3 | 7.59 | 23.6 | 14.1 |

| | Persistence duration (hr) | | | |
|---|---|---|---|---|
| | 48 | 72 | | |
| | UO only | sCr or UO | sCr only | UO only |
| C-C motif chemokine 14 | | | | |
| AUC | 0.74 | 0.80 | 0.80 | 0.74 |
| SE | 0.034 | 0.025 | 0.025 | 0.035 |
| p Value | 4.2E−12 | 0 | 0 | 8.3E−12 |
| nCohort Non-persistent | 247 | 169 | 177 | 254 |
| nCohort Persistent | 82 | 162 | 152 | 75 |
| Cutoff Quartile 2 | 0.732 | 0.725 | 0.719 | 0.732 |
| Sensitivity | 93% | 91% | 91% | 93% |
| Specificity | 31% | 41% | 39% | 30% |
| Cutoff Quartile 3 | 1.64 | 1.64 | 1.64 | 1.64 |
| Sensitivity | 82% | 75% | 76% | 83% |
| Specificity | 60% | 73% | 72% | 59% |
| Cutoff Quartile 4 | 5.09 | 5.16 | 5.09 | 5.09 |
| Sensitivity | 43% | 43% | 44% | 45% |
| Specificity | 81% | 92% | 91% | 81% |
| OR Quartile 2 | 5.63 | 7.29 | 6.83 | 6.09 |
| p Value | 1.1E−4 | 5.6E−10 | 4.9E−9 | 1.8E−4 |
| Lower limit of 95% CI | 2.35 | 3.89 | 3.59 | 2.36 |
| Upper limit of 95% CI | 13.5 | 13.7 | 13.0 | 15.7 |
| OR Quartile 3 | 6.79 | 8.13 | 7.89 | 6.99 |
| p Value | 1.0E−9 | 0 | 2.2E−16 | 4.1E−9 |
| Lower limit of 95% CI | 3.67 | 4.97 | 4.82 | 3.66 |
| Upper limit of 95% CI | 12.6 | 13.3 | 12.9 | 13.4 |
| OR Quartile 4 | 3.09 | 8.21 | 7.93 | 3.47 |
| p Value | 4.2E−5 | 5.5E−11 | 2.0E−11 | 9.7E−6 |
| Lower limit of 95% CI | 1.80 | 4.38 | 4.33 | 2.00 |
| Upper limit of 95% CI | 5.29 | 15.4 | 14.5 | 6.02 |
| Weight adjusted urine output | | | | |
| AUC | 0.25 | 0.36 | 0.39 | 0.27 |
| SE | 0.033 | 0.030 | 0.031 | 0.035 |
| p Value | 9.8E−14 | 2.1E−6 | 3.2E−4 | 6.7E−11 |
| nCohort Non-persistent | 251 | 171 | 180 | 258 |
| nCohort Persistent | 86 | 166 | 155 | 79 |
| Cutoff Quartile 2 | 0.189 | 0.189 | 0.192 | 0.189 |
| Sensitivity | 49% | 66% | 69% | 51% |
| Specificity | 16% | 16% | 19% | 17% |
| Cutoff Quartile 3 | 0.366 | 0.366 | 0.368 | 0.366 |
| Sensitivity | 28% | 40% | 41% | 29% |
| Specificity | 42% | 40% | 42% | 43% |
| Cutoff Quartile 4 | 0.857 | 0.857 | 0.862 | 0.857 |
| Sensitivity | 9% | 19% | 20% | 10% |
| Specificity | 69% | 69% | 71% | 70% |
| OR Quartile 2 | 0.181 | 0.385 | 0.538 | 0.217 |
| p Value | 6.0E−10 | 2.9E−4 | 0.016 | 4.0E−8 |
| Lower limit of 95% CI | 0.105 | 0.229 | 0.326 | 0.126 |
| Upper limit of 95% CI | 0.311 | 0.645 | 0.889 | 0.374 |
| OR Quartile 3 | 0.283 | 0.436 | 0.514 | 0.315 |
| p Value | 3.5E−6 | 1.9E−4 | 0.0027 | 3.2E−5 |
| Lower limit of 95% CI | 0.166 | 0.282 | 0.332 | 0.183 |
| Upper limit of 95% CI | 0.483 | 0.674 | 0.794 | 0.543 |
| OR Quartile 4 | 0.232 | 0.532 | 0.599 | 0.265 |
| p Value | 2.2E−4 | 0.014 | 0.048 | 8.2E−4 |
| Lower limit of 95% CI | 0.107 | 0.321 | 0.361 | 0.122 |
| Upper limit of 95% CI | 0.503 | 0.880 | 0.995 | 0.577 |
| Serum Creatinine | | | | |
| AUC | 0.69 | 0.75 | 0.77 | 0.68 |
| SE | 0.033 | 0.026 | 0.025 | 0.034 |
| p Value | 1.8E−8 | 0 | 0 | 1.1E−7 |
| nCohort Non-persistent | 253 | 171 | 180 | 261 |
| nCohort Persistent | 99 | 183 | 172 | 91 |
| Cutoff Quartile 2 | 1.65 | 1.64 | 1.64 | 1.65 |
| Sensitivity | 89% | 90% | 92% | 89% |
| Specificity | 30% | 41% | 41% | 30% |
| Cutoff Quartile 3 | 2.44 | 2.44 | 2.44 | 2.44 |
| Sensitivity | 67% | 69% | 71% | 67% |
| Specificity | 56% | 70% | 70% | 56% |
| Cutoff Quartile 4 | 3.32 | 3.32 | 3.31 | 3.32 |
| Sensitivity | 45% | 39% | 41% | 46% |
| Specificity | 83% | 89% | 90% | 82% |
| OR Quartile 2 | 3.50 | 5.98 | 7.88 | 3.45 |
| p Value | 3.2E−4 | 5.2E−10 | 7.8E−11 | 6.1E−4 |
| Lower limit of 95% CI | 1.77 | 3.40 | 4.23 | 1.70 |
| Upper limit of 95% CI | 6.92 | 10.5 | 14.7 | 7.01 |
| OR Quartile 3 | 2.56 | 5.06 | 5.69 | 2.54 |
| p Value | 1.5E−4 | 2.0E−12 | 1.0E−13 | 2.6E−4 |
| Lower limit of 95% CI | 1.57 | 3.22 | 3.60 | 1.54 |
| Upper limit of 95% CI | 4.16 | 7.95 | 9.00 | 4.19 |
| OR Quartile 4 | 3.96 | 5.51 | 6.18 | 3.90 |
| p Value | 1.4E−7 | 4.7E−9 | 5.1E−10 | 2.7E−7 |
| Lower limit of 95% CI | 2.37 | 3.11 | 3.48 | 2.32 |
| Upper limit of 95% CI | 6.61 | 9.76 | 11.0 | 6.56 |
| Cystatin-C | | | | |
| AUC | 0.72 | 0.74 | 0.74 | 0.72 |
| SE | 0.036 | 0.028 | 0.029 | 0.037 |
| p Value | 7.8E−10 | 0 | 2.2E−16 | 4.5E−9 |
| nCohort Non-persistent | 230 | 153 | 160 | 236 |
| nCohort Persistent | 76 | 155 | 147 | 70 |
| Cutoff Quartile 2 | 1170000 | 1170000 | 1170000 | 1170000 |
| Sensitivity | 91% | 88% | 87% | 90% |
| Specificity | 30% | 37% | 36% | 30% |
| Cutoff Quartile 3 | 1660000 | 1660000 | 1650000 | 1660000 |
| Sensitivity | 78% | 68% | 69% | 77% |
| Specificity | 59% | 69% | 68% | 58% |
| Cutoff Quartile 4 | 2150000 | 2150000 | 2150000 | 2150000 |
| Sensitivity | 45% | 38% | 39% | 46% |
| Specificity | 81% | 88% | 88% | 81% |
| OR Quartile 2 | 4.31 | 4.25 | 3.73 | 3.80 |
| p Value | 5.3E−4 | 1.1E−6 | 8.8E−6 | 0.0016 |
| Lower limit of 95% CI | 1.89 | 2.38 | 2.09 | 1.66 |
| Upper limit of 95% CI | 9.86 | 7.60 | 6.66 | 8.70 |
| OR Quartile 3 | 5.02 | 4.73 | 4.71 | 4.67 |
| p Value | 1.4E−7 | 2.4E−10 | 3.0E−10 | 9.0E−7 |
| Lower limit of 95% CI | 2.76 | 2.93 | 2.91 | 2.53 |
| Upper limit of 95% CI | 9.15 | 7.65 | 7.62 | 8.64 |
| OR Quartile 4 | 3.52 | 4.61 | 4.84 | 3.57 |
| p Value | 1.1E−5 | 3.7E−7 | 1.1E−7 | 1.3E−5 |
| Lower limit of 95% CI | 2.01 | 2.56 | 2.70 | 2.02 |
| Upper limit of 95% CI | 6.17 | 8.31 | 8.66 | 6.33 |
| C-C motif chemokine 14/(Weight adjusted urine output) | | | | |
| AUC | 0.79 | 0.78 | 0.76 | 0.79 |
| SE | 0.032 | 0.026 | 0.027 | 0.033 |
| p Value | 0 | 0 | 0 | 0 |
| nCohort Non-persistent | 246 | 167 | 175 | 253 |
| nCohort Persistent | 82 | 161 | 151 | 75 |
| Cutoff Quartile 2 | 1.26 | 1.26 | 1.24 | 1.26 |
| Sensitivity | 96% | 88% | 88% | 96% |
| Specificity | 32% | 37% | 37% | 31% |
| Cutoff Quartile 3 | 4.59 | 4.59 | 4.54 | 4.59 |
| Sensitivity | 82% | 73% | 72% | 81% |
| Specificity | 61% | 72% | 69% | 59% |
| Cutoff Quartile 4 | 24.3 | 24.3 | 22.9 | 24.3 |
| Sensitivity | 52% | 42% | 42% | 53% |
| Specificity | 84% | 92% | 90% | 83% |
| OR Quartile 2 | 12.5 | 4.16 | 4.26 | 10.9 |
| p Value | 3.0E−5 | 7.1E−7 | 9.9E−7 | 7.8E−5 |
| Lower limit of 95% CI | 3.81 | 2.37 | 2.38 | 3.33 |
| Upper limit of 95% CI | 40.7 | 7.32 | 7.61 | 35.6 |
| OR Quartile 3 | 6.86 | 6.79 | 5.82 | 6.35 |
| p Value | 8.6E−10 | 8.4E−15 | 6.0E−13 | 1.0E−8 |
| Lower limit of 95% CI | 3.71 | 4.19 | 3.60 | 3.37 |
| Upper limit of 95% CI | 12.7 | 11.0 | 9.39 | 11.9 |
| OR Quartile 4 | 5.85 | 7.99 | 6.42 | 5.74 |
| p Value | 3.6E−10 | 1.0E−10 | 4.7E−10 | 1.1E−9 |
| Lower limit of 95% CI | 3.37 | 4.25 | 3.58 | 3.27 |
| Upper limit of 95% CI | 10.2 | 15.0 | 11.5 | 10.1 |
| C-C motif chemokine 14 X Serum Creatinine | | | | |
| AUC | 0.75 | 0.82 | 0.82 | 0.75 |
| SE | 0.034 | 0.024 | 0.024 | 0.035 |
| p Value | 4.5E−13 | 0 | 0 | 1.2E−12 |
| nCohort Non-persistent | 241 | 164 | 172 | 248 |
| nCohort Persistent | 81 | 160 | 150 | 74 |
| Cutoff Quartile 2 | 1.30 | 1.29 | 1.29 | 1.30 |
| Sensitivity | 90% | 91% | 92% | 91% |
| Specificity | 30% | 41% | 40% | 30% |
| Cutoff Quartile 3 | 3.97 | 3.97 | 3.91 | 3.97 |
| Sensitivity | 84% | 76% | 77% | 85% |
| Specificity | 61% | 75% | 74% | 60% |
| Cutoff Quartile 4 | 18.0 | 18.3 | 16.8 | 18.0 |
| Sensitivity | 47% | 43% | 45% | 49% |
| Specificity | 82% | 93% | 92% | 82% |
| OR Quartile 2 | 3.97 | 7.20 | 7.70 | 4.07 |
| p Value | 5.4E−4 | 8.4E−10 | 1.7E−9 | 8.5E−4 |
| Lower limit of 95% CI | 1.82 | 3.83 | 3.97 | 1.78 |
| Upper limit of 95% CI | 8.65 | 13.5 | 15.0 | 9.29 |
| OR Quartile 3 | 8.32 | 9.31 | 9.63 | 8.77 |
| p Value | 1.4E−10 | 0 | 0 | 6.7E−10 |
| Lower limit of 95% CI | 4.36 | 5.62 | 5.77 | 4.40 |
| Upper limit of 95% CI | 15.9 | 15.4 | 16.1 | 17.5 |
| OR Quartile 4 | 4.07 | 9.60 | 10.1 | 4.27 |
| p Value | 4.9E−7 | 2.7E−11 | 2.9E−12 | 3.5E−7 |
| Lower limit of 95% CI | 2.35 | 4.94 | 5.29 | 2.44 |
| Upper limit of 95% CI | 7.03 | 18.7 | 19.4 | 7.47 |
| C-C motif chemokine 14 X Cystatin-C | | | | |
| AUC | 0.74 | 0.81 | 0.82 | 0.75 |
| SE | 0.035 | 0.024 | 0.025 | 0.036 |
| p Value | 9.1E−12 | 0 | 0 | 4.7E−12 |
| nCohort Non-persistent | 230 | 153 | 160 | 236 |
| nCohort Persistent | 76 | 155 | 147 | 70 |
| Cutoff Quartile 2 | 958000 | 956000 | 955000 | 958000 |
| Sensitivity | 89% | 90% | 90% | 90% |
| Specificity | 30% | 41% | 39% | 30% |
| Cutoff Quartile 3 | 2880000 | 2880000 | 2860000 | 2880000 |
| Sensitivity | 83% | 75% | 76% | 83% |
| Specificity | 61% | 75% | 74% | 60% |
| Cutoff Quartile 4 | 1.03E7 | 1.04E7 | 1.03E7 | 1.03E7 |
| Sensitivity | 49% | 44% | 46% | 53% |
| Specificity | 83% | 94% | 94% | 83% |
| OR Quartile 2 | 3.64 | 6.36 | 6.17 | 3.80 |
| p Value | 0.0012 | 5.8E−9 | 2.0E−8 | 0.0016 |
| Lower limit of 95% CI | 1.66 | 3.41 | 3.27 | 1.66 |
| Upper limit of 95% CI | 7.99 | 11.9 | 11.6 | 8.70 |
| OR Quartile 3 | 7.54 | 9.00 | 8.99 | 7.17 |
| p Value | 1.3E−9 | 0 | 0 | 1.0E−8 |
| Lower limit of 95% CI | 3.92 | 5.37 | 5.36 | 3.66 |
| Upper limit of 95% CI | 14.5 | 15.1 | 15.1 | 14.1 |
| OR Quartile 4 | 4.51 | 12.5 | 12.6 | 5.49 |
| p Value | 1.7E−7 | 2.9E−11 | 4.8E−12 | 8.3E−9 |
| Lower limit of 95% CI | 2.56 | 5.94 | 6.13 | 3.08 |
| Upper limit of 95% CI | 7.92 | 26.3 | 25.8 | 9.81 |
| C-C motif chemokine 14 X Serum Creatinine/(Weight adjusted urine output) | | | | |
| AUC | 0.80 | 0.81 | 0.79 | 0.80 |
| SE | 0.031 | 0.024 | 0.026 | 0.033 |
| p Value | 0 | 0 | 0 | 0 |
| nCohort Non-persistent | 240 | 162 | 170 | 247 |
| nCohort Persistent | 81 | 159 | 149 | 74 |
| Cutoff Quartile 2 | 2.17 | 2.17 | 2.15 | 2.17 |
| Sensitivity | 94% | 90% | 90% | 93% |
| Specificity | 31% | 40% | 38% | 30% |
| Cutoff Quartile 3 | 10.2 | 10.2 | 10.1 | 10.2 |
| Sensitivity | 85% | 75% | 75% | 85% |
| Specificity | 62% | 75% | 72% | 60% |
| Cutoff Quartile 4 | 69.8 | 69.8 | 67.6 | 69.8 |
| Sensitivity | 52% | 45% | 45% | 54% |
| Specificity | 84% | 94% | 92% | 83% |
| OR Quartile 2 | 6.91 | 5.84 | 5.53 | 6.02 |
| p Value | 6.1E−5 | 1.1E−8 | 5.5E−8 | 2.0E−4 |
| Lower limit of 95% CI | 2.68 | 3.19 | 2.98 | 2.33 |
| Upper limit of 95% CI | 17.8 | 10.7 | 10.2 | 15.5 |
| OR Quartile 3 | 9.25 | 9.08 | 7.69 | 8.71 |
| p Value | 5.9E−11 | 0 | 1.1E−15 | 7.6E−10 |
| Lower limit of 95% CI | 4.75 | 5.48 | 4.67 | 4.37 |
| Upper limit of 95% CI | 18.0 | 15.1 | 12.7 | 17.3 |
| OR Quartile 4 | 5.55 | 14.1 | 9.87 | 5.91 |
| p Value | 1.4E−9 | 2.7E−12 | 5.6E−12 | 8.1E−10 |
| Lower limit of 95% CI | 3.19 | 6.70 | 5.14 | 3.35 |
| Upper limit of 95% CI | 9.66 | 29.5 | 18.9 | 10.4 |
| C-C motif chemokine 14 X Cystatin-C/(Weight adjusted urine output) | | | | |
| AUC | 0.78 | 0.79 | 0.78 | 0.78 |
| SE | 0.033 | 0.026 | 0.027 | 0.035 |
| p Value | 0 | 0 | 0 | 4.4E−16 |
| nCohort Non-persistent | 229 | 151 | 158 | 235 |
| nCohort Persistent | 76 | 154 | 146 | 70 |
| Cutoff Quartile 2 | 1580000 | 1580000 | 1580000 | 1580000 |
| Sensitivity | 93% | 88% | 88% | 93% |
| Specificity | 31% | 38% | 37% | 30% |
| Cutoff Quartile 3 | 6710000 | 6710000 | 6570000 | 6710000 |
| Sensitivity | 84% | 74% | 74% | 84% |
| Specificity | 61% | 74% | 72% | 60% |
| Cutoff Quartile 4 | 3.85E7 | 3.85E7 | 3.84E7 | 3.85E7 |
| Sensitivity | 49% | 42% | 42% | 49% |
| Specificity | 83% | 92% | 91% | 82% |
| OR Quartile 2 | 6.38 | 4.31 | 4.12 | 5.63 |
| p Value | 1.3E−4 | 8.8E−7 | 2.5E−6 | 3.7E−4 |
| Lower limit of 95% CI | 2.47 | 2.41 | 2.29 | 2.17 |
| Upper limit of 95% CI | 16.5 | 7.71 | 7.44 | 14.6 |
| OR Quartile 3 | 8.39 | 8.18 | 7.36 | 8.05 |
| p Value | 5.3E−10 | 8.9E−16 | 1.3E−14 | 4.0E−9 |
| Lower limit of 95% CI | 4.29 | 4.90 | 4.43 | 4.02 |
| Upper limit of 95% CI | 16.4 | 13.7 | 12.2 | 16.1 |
| OR Quartile 4 | 4.48 | 8.46 | 6.84 | 4.22 |
| p Value | 1.9E−7 | 4.4E−10 | 1.7E−9 | 8.8E−7 |
| Lower limit of 95% CI | 2.55 | 4.33 | 3.66 | 2.38 |
| Upper limit of 95% CI | 7.88 | 16.5 | 12.8 | 7.48 |

TABLE 22
Comparison of marker levels and the area under the ROC curve (AUC) in urine samples for the “persistent”
and “non-persistent” cohorts where persistence starts within 72 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| Persistence duration (hr) | | | | |
|---|---|---|---|---|
| 24 | | | 48 | |
| sCr or UO | sCr only | UO only | sCr or UO | sCr only |

C-C motif chemokine 14

| | | | | | |
|---|---|---|---|---|---|
| AUC | 0.79 | 0.79 | 0.70 | 0.78 | 0.78 |
| SE | 0.024 | 0.024 | 0.032 | 0.025 | 0.025 |
| p Value | 0 | 0 | 7.6E−10 | 0 | 0 |
| nCohort Non-persistent | 113 | 131 | 220 | 150 | 159 |
| nCohort Persistent | 218 | 198 | 109 | 181 | 170 |
| Cutoff Quartile 2 | 0.725 | 0.719 | 0.732 | 0.725 | 0.719 |
| Sensitivity | 87% | 88% | 90% | 88% | 88% |
| Specificity | 48% | 45% | 32% | 41% | 39% |
| Cutoff Quartile 3 | 1.64 | 1.64 | 1.64 | 1.64 | 1.64 |
| Sensitivity | 65% | 68% | 73% | 71% | 72% |
| Specificity | 79% | 76% | 61% | 75% | 73% |
| Cutoff Quartile 4 | 5.16 | 5.09 | 5.09 | 5.16 | 5.09 |
| Sensitivity | 36% | 38% | 36% | 40% | 42% |
| Specificity | 96% | 95% | 80% | 93% | 92% |
| OR Quartile 2 | 5.96 | 6.23 | 4.25 | 4.95 | 4.79 |
| p Value | 7.5E−11 | 9.8E−11 | 3.5E−5 | 1.4E−8 | 5.4E−8 |
| Lower limit of 95% CI | 3.48 | 3.58 | 2.14 | 2.85 | 2.72 |
| Upper limit of 95% CI | 10.2 | 10.9 | 8.42 | 8.60 | 8.43 |
| OR Quartile 3 | 6.93 | 6.75 | 4.38 | 7.12 | 6.86 |
| p Value | 8.1E−13 | 7.9E−14 | 9.3E−9 | 3.1E−15 | 6.2E−15 |
| Lower limit of 95% CI | 4.08 | 4.09 | 2.65 | 4.37 | 4.23 |
| Upper limit of 95% CI | 11.8 | 11.1 | 7.25 | 11.6 | 11.1 |
| OR Quartile 4 | 12.0 | 11.0 | 2.23 | 9.46 | 8.79 |
| p Value | 2.0E−7 | 7.3E−9 | 0.0022 | 4.6E−10 | 1.3E−10 |
| Lower limit of 95% CI | 4.71 | 4.89 | 1.34 | 4.67 | 4.53 |
| Upper limit of 95% CI | 30.8 | 24.9 | 3.72 | 19.2 | 17.0 |

Weight adjusted urine output

| | | | | | |
|---|---|---|---|---|---|
| AUC | 0.41 | 0.47 | 0.26 | 0.37 | 0.41 |
| SE | 0.032 | 0.032 | 0.030 | 0.030 | 0.031 |
| p Value | 0.0034 | 0.29 | 2.9E−15 | 2.0E−5 | 0.0046 |
| nCohort Non-persistent | 115 | 134 | 224 | 152 | 162 |
| nCohort Persistent | 222 | 201 | 113 | 185 | 173 |
| Cutoff Quartile 2 | 0.189 | 0.192 | 0.189 | 0.189 | 0.192 |
| Sensitivity | 71% | 74% | 53% | 67% | 71% |
| Specificity | 17% | 22% | 14% | 15% | 20% |
| Cutoff Quartile 3 | 0.366 | 0.368 | 0.366 | 0.366 | 0.368 |
| Sensitivity | 46% | 49% | 29% | 42% | 44% |
| Specificity | 42% | 48% | 39% | 40% | 43% |
| Cutoff Quartile 4 | 0.857 | 0.862 | 0.857 | 0.857 | 0.862 |
| Sensitivity | 23% | 25% | 12% | 21% | 21% |
| Specificity | 70% | 75% | 68% | 69% | 71% |
| OR Quartile 2 | 0.520 | 0.806 | 0.182 | 0.362 | 0.589 |
| p Value | 0.023 | 0.41 | 2.8E−10 | 2.3E−4 | 0.040 |
| Lower limit of 95% CI | 0.296 | 0.482 | 0.107 | 0.211 | 0.355 |
| Upper limit of 95% CI | 0.913 | 1.35 | 0.309 | 0.621 | 0.977 |
| OR Quartile 3 | 0.609 | 0.870 | 0.267 | 0.489 | 0.596 |
| p Value | 0.033 | 0.53 | 1.0E−7 | 0.0013 | 0.019 |
| Lower limit of 95% CI | 0.386 | 0.562 | 0.164 | 0.316 | 0.387 |
| Upper limit of 95% CI | 0.960 | 1.35 | 0.434 | 0.756 | 0.918 |
| OR Quartile 4 | 0.711 | 0.974 | 0.274 | 0.578 | 0.666 |
| p Value | 0.19 | 0.92 | 8.0E−5 | 0.030 | 0.11 |
| Lower limit of 95% CI | 0.427 | 0.589 | 0.144 | 0.352 | 0.405 |
| Upper limit of 95% CI | 1.18 | 1.61 | 0.522 | 0.948 | 1.09 |

Serum Creatinine

| | | | | | |
|---|---|---|---|---|---|
| AUC | 0.81 | 0.83 | 0.69 | 0.78 | 0.80 |
| SE | 0.022 | 0.021 | 0.030 | 0.024 | 0.023 |
| p Value | 0 | 0 | 5.8E−10 | 0 | 0 |
| nCohort Non-persistent | 114 | 132 | 227 | 151 | 161 |
| nCohort Persistent | 240 | 220 | 125 | 203 | 191 |
| Cutoff Quartile 2 | 1.64 | 1.64 | 1.65 | 1.64 | 1.64 |
| Sensitivity | 88% | 90% | 87% | 89% | 91% |
| Specificity | 52% | 51% | 32% | 44% | 44% |
| Cutoff Quartile 3 | 2.44 | 2.44 | 2.44 | 2.44 | 2.44 |
| Sensitivity | 65% | 69% | 67% | 67% | 70% |
| Specificity | 82% | 81% | 59% | 73% | 74% |
| Cutoff Quartile 4 | 3.32 | 3.31 | 3.32 | 3.32 | 3.31 |
| Sensitivity | 35% | 38% | 43% | 39% | 40% |
| Specificity | 96% | 96% | 85% | 93% | 93% |
| OR Quartile 2 | 7.51 | 9.77 | 3.16 | 6.08 | 8.07 |
| p Value | 9.3E−14 | 2.4E−15 | 1.5E−4 | 5.8E−11 | 3.1E−12 |
| Lower limit of 95% CI | 4.42 | 5.55 | 1.75 | 3.54 | 4.49 |
| Upper limit of 95% CI | 12.8 | 17.2 | 5.73 | 10.4 | 14.5 |
| OR Quartile 3 | 8.38 | 9.37 | 2.95 | 5.57 | 6.66 |
| p Value | 1.7E−14 | 0 | 3.5E−6 | 3.8E−13 | 2.2E−15 |
| Lower limit of 95% CI | 4.87 | 5.57 | 1.87 | 3.50 | 4.17 |
| Upper limit of 95% CI | 14.4 | 15.8 | 4.67 | 8.85 | 10.6 |
| OR Quartile 4 | 12.0 | 15.4 | 4.17 | 8.11 | 9.21 |
| p Value | 2.0E−7 | 9.8E−9 | 3.0E−8 | 1.3E−9 | 1.3E−10 |
| Lower limit of 95% CI | 4.70 | 6.05 | 2.52 | 4.13 | 4.68 |
| Upper limit of 95% CI | 30.4 | 39.2 | 6.91 | 15.9 | 18.1 |

Cystatin-C

| | | | | | |
|---|---|---|---|---|---|
| AUC | 0.76 | 0.75 | 0.72 | 0.77 | 0.75 |
| SE | 0.027 | 0.027 | 0.032 | 0.026 | 0.027 |
| p Value | 0 | 0 | 4.0E−12 | 0 | 0 |
| nCohort Non-persistent | 100 | 114 | 206 | 135 | 142 |
| nCohort Persistent | 208 | 193 | 100 | 173 | 165 |
| Cutoff Quartile 2 | 1170000 | 1170000 | 1170000 | 1170000 | 1170000 |
| Sensitivity | 86% | 87% | 89% | 88% | 87% |
| Specificity | 46% | 44% | 32% | 41% | 39% |
| Cutoff Quartile 3 | 1660000 | 1650000 | 1660000 | 1660000 | 1650000 |
| Sensitivity | 64% | 65% | 74% | 69% | 68% |
| Specificity | 79% | 75% | 62% | 74% | 71% |
| Cutoff Quartile 4 | 2150000 | 2150000 | 2150000 | 2150000 | 2150000 |
| Sensitivity | 32% | 34% | 43% | 38% | 39% |
| Specificity | 90% | 90% | 83% | 91% | 91% |
| OR Quartile 2 | 5.05 | 5.02 | 3.81 | 4.98 | 4.33 |
| p Value | 8.6E−9 | 1.2E−8 | 1.5E−4 | 3.6E−8 | 4.3E−7 |
| Lower limit of 95% CI | 2.91 | 2.88 | 1.91 | 2.81 | 2.45 |
| Upper limit of 95% CI | 8.77 | 8.74 | 7.62 | 8.81 | 7.66 |
| OR Quartile 3 | 6.67 | 5.39 | 4.58 | 6.30 | 5.35 |
| p Value | 2.7E−11 | 1.4E−10 | 1.6E−8 | 6.5E−13 | 1.8E−11 |
| Lower limit of 95% CI | 3.82 | 3.22 | 2.70 | 3.81 | 3.28 |
| Upper limit of 95% CI | 11.7 | 9.01 | 7.76 | 10.4 | 8.73 |
| OR Quartile 4 | 4.28 | 4.87 | 3.82 | 6.17 | 6.29 |
| p Value | 6.8E−5 | 6.8E−6 | 1.2E−6 | 9.3E−8 | 3.0E−8 |
| Lower limit of 95% CI | 2.09 | 2.44 | 2.22 | 3.16 | 3.28 |
| Upper limit of 95% CI | 8.74 | 9.69 | 6.55 | 12.0 | 12.1 |

C-C motif chemokine 14/(Weight adjusted urine output)

| | | | | | |
|---|---|---|---|---|---|
| AUC | 0.75 | 0.72 | 0.76 | 0.76 | 0.74 |
| SE | 0.027 | 0.028 | 0.030 | 0.026 | 0.027 |
| p Value | 0 | 1.4E−14 | 0 | 0 | 0 |
| nCohort Non-persistent | 111 | 129 | 219 | 148 | 157 |
| nCohort Persistent | 217 | 197 | 109 | 180 | 169 |
| Cutoff Quartile 2 | 1.26 | 1.24 | 1.26 | 1.26 | 1.24 |
| Sensitivity | 84% | 83% | 93% | 86% | 85% |
| Specificity | 42% | 37% | 34% | 38% | 36% |
| Cutoff Quartile 3 | 4.59 | 4.54 | 4.59 | 4.59 | 4.54 |
| Sensitivity | 64% | 64% | 75% | 69% | 68% |
| Specificity | 77% | 71% | 63% | 73% | 69% |
| Cutoff Quartile 4 | 24.3 | 22.9 | 24.3 | 24.3 | 22.9 |
| Sensitivity | 34% | 35% | 44% | 40% | 40% |
| Specificity | 93% | 90% | 84% | 93% | 90% |
| OR Quartile 2 | 3.82 | 2.84 | 6.44 | 3.61 | 3.28 |
| p Value | 4.9E−7 | 6.8E−5 | 2.3E−6 | 2.3E−6 | 1.3E−5 |
| Lower limit of 95% CI | 2.27 | 1.70 | 2.98 | 2.12 | 1.92 |
| Upper limit of 95% CI | 6.44 | 4.75 | 13.9 | 6.14 | 5.61 |
| OR Quartile 3 | 5.71 | 4.41 | 5.07 | 5.98 | 4.84 |
| p Value | 4.7E−11 | 1.3E−9 | 5.8E−10 | 3.1E−13 | 4.4E−11 |
| Lower limit of 95% CI | 3.40 | 2.73 | 3.04 | 3.70 | 3.03 |
| Upper limit of 95% CI | 9.60 | 7.13 | 8.48 | 9.67 | 7.73 |
| OR Quartile 4 | 6.66 | 4.81 | 4.28 | 9.20 | 6.22 |
| p Value | 1.5E−6 | 1.7E−6 | 6.0E−8 | 8.0E−10 | 5.7E−9 |
| Lower limit of 95% CI | 3.08 | 2.53 | 2.53 | 4.53 | 3.36 |
| Upper limit of 95% CI | 14.4 | 9.16 | 7.25 | 18.7 | 11.5 |

C-C motif chemokine 14 X Serum Creatinine

| | | | | | |
|---|---|---|---|---|---|
| AUC | 0.83 | 0.83 | 0.72 | 0.82 | 0.82 |
| SE | 0.022 | 0.022 | 0.032 | 0.023 | 0.023 |
| p Value | 0 | 0 | 6.2E−12 | 0 | 0 |
| nCohort Non-persistent | 109 | 126 | 215 | 145 | 154 |
| nCohort Persistent | 215 | 196 | 107 | 179 | 168 |
| Cutoff Quartile 2 | 1.29 | 1.29 | 1.30 | 1.29 | 1.29 |
| Sensitivity | 87% | 89% | 88% | 89% | 90% |
| Specificity | 50% | 48% | 32% | 42% | 42% |
| Cutoff Quartile 3 | 3.97 | 3.91 | 3.97 | 3.97 | 3.91 |
| Sensitivity | 68% | 70% | 78% | 73% | 74% |
| Specificity | 85% | 82% | 64% | 78% | 77% |
| Cutoff Quartile 4 | 18.3 | 16.8 | 18.0 | 18.3 | 16.8 |
| Sensitivity | 36% | 39% | 39% | 41% | 43% |
| Specificity | 96% | 96% | 82% | 94% | 94% |
| OR Quartile 2 | 6.84 | 7.58 | 3.34 | 5.77 | 6.32 |
| p Value | 8.1E−12 | 3.9E−12 | 2.6E−4 | 1.7E−9 | 1.3E−9 |
| Lower limit of 95% CI | 3.94 | 4.28 | 1.75 | 3.26 | 3.48 |
| Upper limit of 95% CI | 11.9 | 13.4 | 6.39 | 10.2 | 11.5 |
| OR Quartile 3 | 12.3 | 10.7 | 6.07 | 9.37 | 9.53 |
| p Value | 4.4E−16 | 0 | 3.2E−11 | 0 | 0 |
| Lower limit of 95% CI | 6.73 | 6.17 | 3.57 | 5.62 | 5.73 |
| Upper limit of 95% CI | 22.5 | 18.4 | 10.3 | 15.6 | 15.9 |
| OR Quartile 4 | 14.6 | 15.3 | 2.92 | 11.8 | 12.1 |
| p Value | 3.9E−7 | 1.2E−8 | 5.6E−5 | 3.9E−10 | 4.0E−11 |
| Lower limit of 95% CI | 5.19 | 5.99 | 1.73 | 5.45 | 5.77 |
| Upper limit of 95% CI | 41.3 | 39.2 | 4.91 | 25.5 | 25.3 |

C-C motif chemokine 14 X Cystatin-C

| | | | | | |
|---|---|---|---|---|---|
| AUC | 0.82 | 0.82 | 0.72 | 0.81 | 0.81 |
| SE | 0.024 | 0.024 | 0.033 | 0.024 | 0.024 |
| p Value | 0 | 0 | 1.7E−11 | 0 | 0 |
| nCohort Non-persistent | 100 | 114 | 206 | 135 | 142 |
| nCohort Persistent | 208 | 193 | 100 | 173 | 165 |
| Cutoff Quartile 2 | 956000 | 955000 | 958000 | 956000 | 955000 |
| Sensitivity | 87% | 88% | 90% | 88% | 88% |
| Specificity | 50% | 46% | 33% | 41% | 40% |
| Cutoff Quartile 3 | 2880000 | 2860000 | 2880000 | 2880000 | 2860000 |
| Sensitivity | 66% | 68% | 75% | 73% | 73% |
| Specificity | 83% | 81% | 62% | 79% | 77% |
| Cutoff Quartile 4 | 1.04E7 | 1.03E7 | 1.03E7 | 1.04E7 | 1.03E7 |
| Sensitivity | 36% | 38% | 42% | 41% | 42% |
| Specificity | 97% | 96% | 83% | 96% | 95% |
| OR Quartile 2 | 6.70 | 6.12 | 4.34 | 5.13 | 4.86 |
| p Value | 3.6E−11 | 3.1E−10 | 5.8E−5 | 1.9E−8 | 7.2E−8 |
| Lower limit of 95% CI | 3.82 | 3.48 | 2.12 | 2.90 | 2.73 |
| Upper limit of 95% CI | 11.8 | 10.8 | 8.87 | 9.08 | 8.64 |
| OR Quartile 3 | 9.42 | 9.05 | 4.92 | 10.2 | 9.08 |
| p Value | 1.5E−13 | 7.5E−15 | 4.6E−9 | 0 | 0 |
| Lower limit of 95% CI | 5.19 | 5.19 | 2.89 | 6.01 | 5.40 |
| Upper limit of 95% CI | 17.1 | 15.8 | 8.39 | 17.5 | 15.3 |
| OR Quartile 4 | 17.9 | 16.7 | 3.54 | 15.0 | 14.2 |
| p Value | 1.8E−6 | 1.1E−7 | 4.2E−6 | 1.2E−9 | 2.3E−10 |
| Lower limit of 95% CI | 5.47 | 5.92 | 2.06 | 6.25 | 6.26 |
| Upper limit of 95% CI | 58.3 | 47.3 | 6.06 | 35.8 | 32.3 |

C-C motif chemokine 14 X Serum Creatinine/(Weight adjusted urine output)

| | | | | | |
|---|---|---|---|---|---|
| AUC | 0.80 | 0.77 | 0.78 | 0.80 | 0.78 |
| SE | 0.024 | 0.026 | 0.029 | 0.024 | 0.026 |
| p Value | 0 | 0 | 0 | 0 | 0 |
| nCohort Non-persistent | 107 | 124 | 214 | 143 | 152 |
| nCohort Persistent | 214 | 195 | 107 | 178 | 167 |
| Cutoff Quartile 2 | 2.17 | 2.15 | 2.17 | 2.17 | 2.15 |
| Sensitivity | 86% | 85% | 92% | 88% | 87% |
| Specificity | 46% | 41% | 33% | 41% | 39% |
| Cutoff Quartile 3 | 10.2 | 10.1 | 10.2 | 10.2 | 10.1 |
| Sensitivity | 66% | 67% | 79% | 72% | 71% |
| Specificity | 82% | 76% | 64% | 77% | 73% |
| Cutoff Quartile 4 | 69.8 | 67.6 | 69.8 | 69.8 | 67.6 |
| Sensitivity | 36% | 37% | 45% | 42% | 42% |
| Specificity | 96% | 94% | 85% | 96% | 93% |
| OR Quartile 2 | 4.99 | 4.00 | 5.41 | 4.84 | 4.41 |
| p Value | 4.8E−9 | 3.4E−7 | 7.7E−6 | 3.0E−8 | 2.3E−7 |
| Lower limit of 95% CI | 2.91 | 2.35 | 2.58 | 2.77 | 2.51 |
| Upper limit of 95% CI | 8.54 | 6.81 | 11.3 | 8.45 | 7.74 |
| OR Quartile 3 | 9.13 | 6.27 | 6.50 | 8.53 | 6.71 |
| p Value | 3.2E−14 | 1.4E−12 | 1.0E−11 | 2.2E−16 | 2.8E−14 |
| Lower limit of 95% CI | 5.16 | 3.77 | 3.79 | 5.13 | 4.11 |
| Upper limit of 95% CI | 16.2 | 10.4 | 11.1 | 14.2 | 11.0 |
| OR Quartile 4 | 14.5 | 8.49 | 4.46 | 16.6 | 10.2 |
| p Value | 4.4E−7 | 5.9E−8 | 3.5E−8 | 2.4E−10 | 1.4E−10 |
| Lower limit of 95% CI | 5.13 | 3.92 | 2.62 | 6.97 | 5.03 |
| Upper limit of 95% CI | 40.8 | 18.4 | 7.59 | 39.7 | 20.9 |

C-C motif chemokine 14 X Cystatin-C/(Weight adjusted urine output)

| | | | | | |
|---|---|---|---|---|---|
| AUC | 0.78 | 0.75 | 0.77 | 0.79 | 0.76 |
| SE | 0.026 | 0.028 | 0.031 | 0.026 | 0.027 |
| p Value | 0 | 0 | 0 | 0 | 0 |
| nCohort Non-persistent | 98 | 112 | 205 | 133 | 140 |
| nCohort Persistent | 207 | 192 | 100 | 172 | 164 |
| Cutoff Quartile 2 | 1580000 | 1580000 | 1580000 | 1580000 | 1580000 |
| Sensitivity | 84% | 83% | 93% | 86% | 85% |
| Specificity | 44% | 39% | 34% | 39% | 37% |
| Cutoff Quartile 3 | 6710000 | 6570000 | 6710000 | 6710000 | 6570000 |
| Sensitivity | 64% | 65% | 77% | 70% | 70% |
| Specificity | 80% | 76% | 63% | 76% | 73% |
| Cutoff Quartile 4 | 3.85E7 | 3.84E7 | 3.85E7 | 3.85E7 | 3.84E7 |
| Sensitivity | 34% | 34% | 43% | 40% | 39% |
| Specificity | 94% | 91% | 83% | 94% | 91% |
| OR Quartile 2 | 4.12 | 3.24 | 6.74 | 3.96 | 3.45 |
| p Value | 3.6E−7 | 1.8E−5 | 5.2E−6 | 1.1E−6 | 1.1E−5 |
| Lower limit of 95% CI | 2.39 | 1.89 | 2.97 | 2.27 | 1.98 |
| Upper limit of 95% CI | 7.11 | 5.53 | 15.3 | 6.89 | 5.99 |
| OR Quartile 3 | 7.01 | 5.87 | 5.68 | 7.49 | 6.12 |
| p Value | 1.8E−11 | 3.8E−11 | 4.2E−10 | 1.8E−14 | 1.1E−12 |
| Lower limit of 95% CI | 3.97 | 3.47 | 3.29 | 4.47 | 3.71 |
| Upper limit of 95% CI | 12.4 | 9.93 | 9.80 | 12.5 | 10.1 |
| OR Quartile 4 | 8.00 | 5.34 | 3.79 | 10.5 | 6.83 |
| p Value | 3.1E−6 | 4.3E−6 | 1.3E−6 | 3.2E−9 | 1.9E−8 |
| Lower limit of 95% CI | 3.34 | 2.61 | 2.21 | 4.81 | 3.49 |
| Upper limit of 95% CI | 19.2 | 10.9 | 6.51 | 22.8 | 13.3 |

| | Persistence duration (hr) | | | |
|---|---|---|---|---|
| | 48 | 72 | | |
| | UO only | sCr or UO | sCr only | UO only |
| C-C motif chemokine 14 | | | | |
| AUC | 0.70 | 0.79 | 0.79 | 0.70 |
| SE | 0.034 | 0.025 | 0.025 | 0.035 |
| p Value | 6.0E−9 | 0 | 0 | 1.6E−8 |
| nCohort Non-persistent | 235 | 165 | 172 | 242 |
| nCohort Persistent | 94 | 166 | 157 | 87 |
| Cutoff Quartile 2 | 0.732 | 0.725 | 0.719 | 0.732 |
| Sensitivity | 89% | 90% | 90% | 90% |
| Specificity | 31% | 41% | 39% | 30% |
| Cutoff Quartile 3 | 1.64 | 1.64 | 1.64 | 1.64 |
| Sensitivity | 76% | 73% | 74% | 76% |
| Specificity | 60% | 73% | 72% | 59% |
| Cutoff Quartile 4 | 5.09 | 5.16 | 5.09 | 5.09 |
| Sensitivity | 38% | 42% | 43% | 40% |
| Specificity | 80% | 92% | 91% | 80% |
| OR Quartile 2 | 3.71 | 6.41 | 6.04 | 3.74 |
| p Value | 3.1E−4 | 1.4E−9 | 9.4E−9 | 4.9E−4 |
| Lower limit of 95% CI | 1.82 | 3.51 | 3.27 | 1.78 |
| Upper limit of 95% CI | 7.56 | 11.7 | 11.2 | 7.87 |
| OR Quartile 3 | 4.63 | 7.62 | 7.10 | 4.54 |
| p Value | 2.3E−8 | 2.2E−16 | 2.7E−15 | 8.6E−8 |
| Lower limit of 95% CI | 2.70 | 4.68 | 4.37 | 2.61 |
| Upper limit of 95% CI | 7.93 | 12.4 | 11.5 | 7.90 |
| OR Quartile 4 | 2.48 | 8.53 | 8.00 | 2.72 |
| p Value | 6.8E−4 | 7.3E−11 | 3.9E−11 | 2.3E−4 |
| Lower limit of 95% CI | 1.47 | 4.47 | 4.32 | 1.60 |
| Upper limit of 95% CI | 4.19 | 16.2 | 14.8 | 4.63 |
| Weight adjusted urine output | | | | |
| AUC | 0.29 | 0.37 | 0.40 | 0.31 |
| SE | 0.033 | 0.030 | 0.031 | 0.034 |
| p Value | 2.9E−10 | 2.0E−5 | 0.0016 | 5.4E−8 |
| nCohort Non-persistent | 239 | 167 | 175 | 246 |
| nCohort Persistent | 98 | 170 | 160 | 91 |
| Cutoff Quartile 2 | 0.189 | 0.189 | 0.192 | 0.189 |
| Sensitivity | 54% | 67% | 70% | 56% |
| Specificity | 16% | 17% | 20% | 18% |
| Cutoff Quartile 3 | 0.366 | 0.366 | 0.368 | 0.366 |
| Sensitivity | 33% | 41% | 42% | 34% |
| Specificity | 43% | 41% | 43% | 44% |
| Cutoff Quartile 4 | 0.857 | 0.857 | 0.862 | 0.857 |
| Sensitivity | 13% | 20% | 21% | 14% |
| Specificity | 70% | 69% | 71% | 71% |
| OR Quartile 2 | 0.230 | 0.410 | 0.583 | 0.278 |
| p Value | 4.0E−8 | 7.2E−4 | 0.035 | 1.9E−6 |
| Lower limit of 95% CI | 0.136 | 0.245 | 0.353 | 0.164 |
| Upper limit of 95% CI | 0.388 | 0.688 | 0.963 | 0.470 |
| OR Quartile 3 | 0.361 | 0.481 | 0.554 | 0.404 |
| p Value | 5.3E−5 | 9.5E−4 | 0.0076 | 4.0E−4 |
| Lower limit of 95% CI | 0.220 | 0.311 | 0.359 | 0.245 |
| Upper limit of 95% CI | 0.592 | 0.742 | 0.855 | 0.668 |
| OR Quartile 4 | 0.355 | 0.569 | 0.632 | 0.403 |
| p Value | 0.0017 | 0.027 | 0.074 | 0.0060 |
| Lower limit of 95% CI | 0.186 | 0.345 | 0.382 | 0.211 |
| Upper limit of 95% CI | 0.677 | 0.937 | 1.04 | 0.770 |
| Serum Creatinine | | | | |
| AUC | 0.68 | 0.75 | 0.77 | 0.67 |
| SE | 0.032 | 0.026 | 0.025 | 0.0.33 |
| p Value | 2.4E−8 | 0 | 0 | 1.6E−7 |
| nCohort Non-persistent | 242 | 167 | 175 | 250 |
| nCohort Persistent | 110 | 187 | 177 | 102 |
| Cutoff Quartile 2 | 1.65 | 1.64 | 1.64 | 1.65 |
| Sensitivity | 87% | 89% | 91% | 87% |
| Specificity | 31% | 41% | 41% | 30% |
| Cutoff Quartile 3 | 2.44 | 2.44 | 2.44 | 2.44 |
| Sensitivity | 66% | 68% | 71% | 67% |
| Specificity | 57% | 70% | 71% | 56% |
| Cutoff Quartile 4 | 3.32 | 3.32 | 3.31 | 3.32 |
| Sensitivity | 44% | 39% | 40% | 44% |
| Specificity | 83% | 90% | 90% | 82% |
| OR Quartile 2 | 3.02 | 5.43 | 7.03 | 2.93 |
| p Value | 5.1E−4 | 1.5E−9 | 1.4E−10 | 0.0010 |
| Lower limit of 95% CI | 1.62 | 3.14 | 3.88 | 1.54 |
| Upper limit of 95% CI | 5.64 | 9.40 | 12.8 | 5.57 |
| OR Quartile 3 | 2.62 | 5.08 | 5.84 | 2.59 |
| p Value | 6.1E−5 | 2.0E−12 | 4.9E−14 | 1.1E−4 |
| Lower limit of 95% CI | 1.64 | 3.23 | 3.69 | 1.60 |
| Upper limit of 95% CI | 4.19 | 7.98 | 9.25 | 4.19 |
| OR Quartile 4 | 3.80 | 5.65 | 6.23 | 3.70 |
| p Value | 2.2E−7 | 5.3E−9 | 8.2E−10 | 4.7E−7 |
| Lower limit of 95% CI | 2.29 | 3.16 | 3.47 | 2.22 |
| Upper limit of 95% CI | 6.29 | 10.1 | 11.2 | 6.15 |
| Cystatin-C | | | | |
| AUC | 0.72 | 0.75 | 0.73 | 0.72 |
| SE | 0.034 | 0.028 | 0.028 | 0.036 |
| p Value | 1.3E−10 | 0 | 2.2E−16 | 9.4E−10 |
| nCohort Non-persistent | 220 | 150 | 155 | 226 |
| nCohort Persistent | 86 | 158 | 152 | 80 |
| Cutoff Quartile 2 | 1170000 | 1170000 | 1170000 | 1170000 |
| Sensitivity | 90% | 87% | 87% | 89% |
| Specificity | 31% | 37% | 36% | 30% |
| Cutoff Quartile 3 | 1660000 | 1660000 | 1650000 | 1660000 |
| Sensitivity | 74% | 68% | 68% | 74% |
| Specificity | 60% | 69% | 68% | 58% |
| Cutoff Quartile 4 | 2150000 | 2150000 | 2150000 | 2150000 |
| Sensitivity | 44% | 38% | 39% | 45% |
| Specificity | 82% | 89% | 88% | 82% |
| OR Quartile 2 | 3.83 | 4.11 | 3.73 | 3.40 |
| p Value | 4.3E−4 | 1.4E−6 | 6.7E−6 | 0.0014 |
| Lower limit of 95% CI | 1.81 | 2.32 | 2.10 | 1.60 |
| Upper limit of 95% CI | 8.08 | 7.30 | 6.62 | 7.18 |
| OR Quartile 3 | 4.28 | 4.88 | 4.55 | 3.95 |
| p Value | 2.7E−7 | 1.2E−10 | 6.1E−10 | 1.8E−6 |
| Lower limit of 95% CI | 2.46 | 3.01 | 2.82 | 2.24 |
| Upper limit of 95% CI | 7.45 | 7.91 | 7.35 | 6.93 |
| OR Quartile 4 | 3.67 | 4.79 | 4.83 | 3.69 |
| p Value | 3.3E−6 | 2.9E−7 | 1.7E−7 | 4.0E−6 |
| Lower limit of 95% CI | 2.12 | 2.63 | 2.68 | 2.12 |
| Upper limit of 95% CI | 6.36 | 8.71 | 8.71 | 6.43 |
| C-C motif chemokine 14/(Weight adjusted urine output) | | | | |
| AUC | 0.74 | 0.76 | 0.75 | 0.73 |
| SE | 0.032 | 0.026 | 0.027 | 0.034 |
| p Value | 7.1E−14 | 0 | 0 | 8.5E−12 |
| nCohort Non-persistent | 234 | 163 | 170 | 241 |
| nCohort Persistent | 94 | 165 | 156 | 87 |
| Cutoff Quartile 2 | 1.26 | 1.26 | 1.24 | 1.26 |
| Sensitivity | 91% | 87% | 87% | 91% |
| Specificity | 32% | 37% | 36% | 31% |
| Cutoff Quartile 3 | 4.59 | 4.59 | 4.54 | 4.59 |
| Sensitivity | 74% | 71% | 70% | 74% |
| Specificity | 60% | 71% | 68% | 59% |
| Cutoff Quartile 4 | 24.3 | 24.3 | 22.9 | 24.3 |
| Sensitivity | 47% | 41% | 41% | 47% |
| Specificity | 84% | 91% | 89% | 83% |
| OR Quartile 2 | 4.97 | 3.79 | 3.60 | 4.38 |
| p Value | 5.0E−5 | 2.1E−6 | 6.5E−6 | 2.0E−4 |
| Lower limit of 95% CI | 2.29 | 2.18 | 2.06 | 2.01 |
| Upper limit of 95% CI | 10.8 | 6.56 | 6.28 | 9.52 |
| OR Quartile 3 | 4.34 | 6.02 | 4.98 | 3.92 |
| p Value | 6.3E−8 | 1.7E−13 | 2.2E−11 | 7.3E−7 |
| Lower limit of 95% CI | 2.55 | 3.73 | 3.11 | 2.28 |
| Upper limit of 95% CI | 7.40 | 9.70 | 7.97 | 6.74 |
| OR Quartile 4 | 4.54 | 7.46 | 5.87 | 4.35 |
| p Value | 2.8E−8 | 3.9E−10 | 2.7E−9 | 8.9E−8 |
| Lower limit of 95% CI | 2.66 | 3.98 | 3.28 | 2.54 |
| Upper limit of 95% CI | 7.74 | 14.0 | 10.5 | 7.45 |
| C-C motif chemokine 14 X Serum Creatinine | | | | |
| AUC | 0.72 | 0.81 | 0.81 | 0.72 |
| SE | 0.033 | 0.024 | 0.024 | 0.035 |
| p Value | 9.8E−11 | 0 | 0 | 3.6E−10 |
| nCohort Non-persistent | 230 | 160 | 167 | 237 |
| nCohort Persistent | 92 | 164 | 155 | 85 |
| Cutoff Quartile 2 | 1.30 | 1.29 | 1.29 | 1.30 |
| Sensitivity | 88% | 90% | 91% | 88% |
| Specificity | 30% | 41% | 40% | 30% |
| Cutoff Quartile 3 | 3.97 | 3.97 | 3.91 | 3.97 |
| Sensitivity | 78% | 75% | 76% | 79% |
| Specificity | 61% | 76% | 74% | 60% |
| Cutoff Quartile 4 | 18.0 | 18.3 | 16.8 | 18.0 |
| Sensitivity | 42% | 43% | 45% | 44% |
| Specificity | 82% | 93% | 93% | 81% |
| OR Quartile 2 | 3.22 | 6.33 | 6.75 | 3.21 |
| p Value | 8.8E−4 | 2.2E−9 | 2.9E−9 | 0.0014 |
| Lower limit of 95% CI | 1.62 | 3.46 | 3.59 | 1.57 |
| Upper limit of 95% CI | 6.42 | 11.6 | 12.7 | 6.56 |
| OR Quartile 3 | 5.70 | 9.31 | 9.20 | 5.66 |
| p Value | 1.3E−9 | 0 | 0 | 5.2E−9 |
| Lower limit of 95% CI | 3.25 | 5.62 | 5.54 | 3.16 |
| Upper limit of 95% CI | 10.0 | 15.4 | 15.3 | 10.1 |
| OR Quartile 4 | 3.29 | 10.1 | 10.4 | 3.38 |
| p Value | 1.1E−5 | 4.0E−11 | 6.5E−12 | 9.6E−6 |
| Lower limit of 95% CI | 1.94 | 5.08 | 5.32 | 1.97 |
| Upper limit of 95% CI | 5.61 | 20.0 | 20.2 | 5.80 |
| C-C motif chemokine 14 X Cystatin-C | | | | |
| AUC | 0.72 | 0.81 | 0.81 | 0.72 |
| SE | 0.034 | 0.024 | 0.025 | 0.035 |
| p Value | 2.3E−10 | 0 | 0 | 2.0E−10 |
| nCohort Non-persistent | 220 | 150 | 155 | 226 |
| nCohort Persistent | 86 | 158 | 152 | 80 |
| Cutoff Quartile 2 | 958000 | 956000 | 955000 | 958000 |
| Sensitivity | 88% | 90% | 89% | 89% |
| Specificity | 30% | 41% | 39% | 30% |
| Cutoff Quartile 3 | 2880000 | 2880000 | 2860000 | 2880000 |
| Sensitivity | 78% | 75% | 75% | 78% |
| Specificity | 61% | 76% | 74% | 60% |
| Cutoff Quartile 4 | 1.03E7 | 1.04E7 | 1.03E7 | 1.03E7 |
| Sensitivity | 45% | 44% | 45% | 49% |
| Specificity | 83% | 95% | 94% | 83% |
| OR Quartile 2 | 3.33 | 6.08 | 5.52 | 3.40 |
| p Value | 0.0010 | 7.0E−9 | 4.1E−8 | 0.0014 |
| Lower limit of 95% CI | 1.62 | 3.30 | 3.00 | 1.60 |
| Upper limit of 95% CI | 6.83 | 11.2 | 10.2 | 7.18 |
| OR Quartile 3 | 5.49 | 9.34 | 8.62 | 5.11 |
| p Value | 7.1E−9 | 0 | 2.2E−16 | 5.5E−8 |
| Lower limit of 95% CI | 3.09 | 5.56 | 5.16 | 2.84 |
| Upper limit of 95% CI | 9.78 | 15.7 | 14.4 | 9.20 |
| OR Quartile 4 | 3.97 | 13.8 | 13.1 | 4.71 |
| p Value | 8.8E−7 | 4.1E−11 | 1.3E−11 | 6.0E−8 |
| Lower limit of 95% CI | 2.29 | 6.32 | 6.23 | 2.69 |
| Upper limit of 95% CI | 6.89 | 30.0 | 27.7 | 8.24 |
| C-C motif chemokine 14 X Serum Creatinine/(Weight adjusted urine output) | | | | |
| AUC | 0.76 | 0.79 | 0.78 | 0.75 |
| SE | 0.032 | 0.025 | 0.026 | 0.033 |
| p Value | 2.2E−16 | 0 | 0 | 2.8E−14 |
| nCohort Non-persistent | 229 | 158 | 165 | 236 |
| nCohort Persistent | 92 | 163 | 154 | 85 |
| Cutoff Quartile 2 | 2.17 | 2.17 | 2.15 | 2.17 |
| Sensitivity | 90% | 89% | 89% | 89% |
| Specificity | 31% | 39% | 38% | 30% |
| Cutoff Quartile 3 | 10.2 | 10.2 | 10.1 | 10.2 |
| Sensitivity | 78% | 74% | 73% | 78% |
| Specificity | 61% | 74% | 71% | 60% |
| Cutoff Quartile 4 | 69.8 | 69.8 | 67.6 | 69.8 |
| Sensitivity | 47% | 44% | 44% | 48% |
| Specificity | 83% | 94% | 92% | 83% |
| OR Quartile 2 | 4.14 | 5.20 | 4.98 | 3.63 |
| p Value | 1.8E−4 | 3.2E−8 | 1.2E−7 | 6.9E−4 |
| Lower limit of 95% CI | 1.97 | 2.90 | 2.75 | 1.73 |
| Upper limit of 95% CI | 8.71 | 9.34 | 9.01 | 7.65 |
| OR Quartile 3 | 5.66 | 7.96 | 6.50 | 5.16 |
| p Value | 1.5E−9 | 2.2E−16 | 5.9E−14 | 2.0E−8 |
| Lower limit of 95% CI | 3.23 | 4.84 | 3.99 | 2.91 |
| Upper limit of 95% CI | 9.94 | 13.1 | 10.6 | 9.14 |
| OR Quartile 4 | 4.41 | 13.1 | 9.00 | 4.57 |
| p Value | 6.3E−8 | 9.8E−12 | 3.4E−11 | 4.6E−8 |
| Lower limit of 95% CI | 2.58 | 6.25 | 4.70 | 2.65 |
| Upper limit of 95% CI | 7.55 | 27.5 | 17.2 | 7.87 |
| C-C motif chemokine 14 X Cystatin-C/(Weight adjusted urine output) | | | | |
| AUC | 0.75 | 0.78 | 0.76 | 0.74 |
| SE | 0.033 | 0.026 | 0.027 | 0.035 |
| p Value | 7.4E−14 | 0 | 0 | 2.3E−12 |
| nCohort Non-persistent | 219 | 148 | 153 | 225 |
| nCohort Persistent | 86 | 157 | 151 | 80 |
| Cutoff Quartile 2 | 1580000 | 1580000 | 1580000 | 1580000 |
| Sensitivity | 92% | 87% | 87% | 91% |
| Specificity | 32% | 38% | 37% | 31% |
| Cutoff Quartile 3 | 6710000 | 6710000 | 6570000 | 6710000 |
| Sensitivity | 78% | 73% | 72% | 78% |
| Specificity | 61% | 74% | 71% | 60% |
| Cutoff Quartile 4 | 3.85E7 | 3.85E7 | 3.84E7 | 3.85E7 |
| Sensitivity | 44% | 41% | 40% | 44% |
| Specificity | 82% | 92% | 90% | 81% |
| OR Quartile 2 | 5.19 | 4.17 | 3.78 | 4.61 |
| p Value | 8.9E−5 | 1.1E−6 | 5.6E−6 | 2.8E−4 |
| Lower limit of 95% CI | 2.28 | 2.35 | 2.13 | 2.02 |
| Upper limit of 95% CI | 11.8 | 7.41 | 6.71 | 10.5 |
| OR Quartile 3 | 5.45 | 7.41 | 6.22 | 5.07 |
| p Value | 8.4E−9 | 9.5E−15 | 5.9E−13 | 6.4E−8 |
| Lower limit of 95% CI | 3.06 | 4.46 | 3.78 | 2.82 |
| Upper limit of 95% CI | 9.71 | 12.3 | 10.2 | 9.14 |
| OR Quartile 4 | 3.65 | 8.01 | 6.24 | 3.39 |
| p Value | 3.7E−6 | 1.2E−9 | 9.1E−9 | 1.6E−5 |
| Lower limit of 95% CI | 2.11 | 4.10 | 3.34 | 1.95 |
| Upper limit of 95% CI | 6.32 | 15.7 | 11.6 | 5.90 |

TABLE 23
Comparison of marker levels and the area under the ROC curve (AUC) in urine samples for the “persistent”
and “non-persistent” cohorts where persistence starts within 96 hours after sample collection
and renal status was assessed by serum creatinine (sCr) only, urine output (UO) only, or by either
serum creatinine or urine output KDIGO criteria. Markers are either positive going or negative going
which can be discerned from the numerical value of the AUC. More specifically, an AUC value >0.5
indicates a positive going marker and an AUC value <0.5 indicates a negative going marker.

| Persistence duration (hr) | | | | |
|---|---|---|---|---|
| 24 | | | 48 | |
| sCr or UO | sCr only | UO only | sCr or UO | sCr only |

C-C motif chemokine 14

| | | | | | |
|---|---|---|---|---|---|
| AUC | 0.78 | 0.79 | 0.68 | 0.78 | 0.78 |
| SE | 0.025 | 0.024 | 0.032 | 0.025 | 0.025 |
| p Value | 0 | 0 | 7.4E−9 | 0 | 0 |
| nCohort Non-persistent | 112 | 131 | 216 | 149 | 158 |
| nCohort Persistent | 219 | 198 | 113 | 182 | 171 |
| Cutoff Quartile 2 | 0.725 | 0.719 | 0.732 | 0.725 | 0.719 |
| Sensitivity | 86% | 88% | 88% | 88% | 88% |
| Specificity | 47% | 45% | 32% | 41% | 39% |
| Cutoff Quartile 3 | 1.64 | 1.64 | 1.64 | 1.64 | 1.64 |
| Sensitivity | 65% | 68% | 72% | 70% | 71% |
| Specificity | 79% | 76% | 61% | 74% | 73% |
| Cutoff Quartile 4 | 5.16 | 5.09 | 5.09 | 5.16 | 5.09 |
| Sensitivity | 36% | 38% | 35% | 40% | 42% |
| Specificity | 96% | 95% | 80% | 93% | 92% |
| OR Quartile 2 | 5.66 | 6.23 | 3.61 | 5.04 | 4.88 |
| p Value | 2.1E−10 | 9.8E−11 | 9.5E−5 | 9.5E−9 | 3.9E−8 |
| Lower limit of 95% CI | 3.32 | 3.58 | 1.89 | 2.90 | 2.77 |
| Upper limit of 95% CI | 9.66 | 10.9 | 6.88 | 8.76 | 8.58 |
| OR Quartile 3 | 6.76 | 6.75 | 3.98 | 6.92 | 6.66 |
| p Value | 1.5E−12 | 7.9E−14 | 3.9E−8 | 6.7E−15 | 1.3E−14 |
| Lower limit of 95% CI | 3.98 | 4.09 | 2.43 | 4.26 | 4.11 |
| Upper limit of 95% CI | 11.5 | 11.1 | 6.51 | 11.3 | 10.8 |
| OR Quartile 4 | 11.8 | 11.0 | 2.06 | 9.31 | 8.64 |
| p Value | 2.5E−7 | 7.3E−9 | 0.0055 | 6.2E−10 | 1.8E−10 |
| Lower limit of 95% CI | 4.63 | 4.89 | 1.24 | 4.59 | 4.45 |
| Upper limit of 95% CI | 30.3 | 24.9 | 3.43 | 18.9 | 16.8 |

Weight adjusted urine output

| | | | | | |
|---|---|---|---|---|---|
| AUC | 0.41 | 0.47 | 0.28 | 0.38 | 0.42 |
| SE | 0.032 | 0.032 | 0.030 | 0.030 | 0.031 |
| p Value | 0.0060 | 0.29 | 5.7E−13 | 3.9E−5 | 0.0071 |
| nCohort Non-persistent | 114 | 134 | 220 | 151 | 161 |
| nCohort Persistent | 223 | 201 | 117 | 186 | 174 |
| Cutoff Quartile 2 | 0.189 | 0.192 | 0.189 | 0.189 | 0.192 |
| Sensitivity | 71% | 74% | 55% | 67% | 71% |
| Specificity | 18% | 22% | 14% | 15% | 20% |
| Cutoff Quartile 3 | 0.366 | 0.368 | 0.366 | 0.366 | 0.368 |
| Sensitivity | 46% | 49% | 31% | 42% | 44% |
| Specificity | 42% | 48% | 40% | 40% | 43% |
| Cutoff Quartile 4 | 0.857 | 0.862 | 0.857 | 0.857 | 0.862 |
| Sensitivity | 23% | 25% | 14% | 21% | 22% |
| Specificity | 71% | 75% | 69% | 70% | 71% |
| OR Quartile 2 | 0.529 | 0.806 | 0.198 | 0.368 | 0.598 |
| p Value | 0.026 | 0.41 | 1.6E−9 | 2.8E−4 | 0.047 |
| Lower limit of 95% CI | 0.301 | 0.482 | 0.117 | 0.215 | 0.361 |
| Upper limit of 95% CI | 0.928 | 1.35 | 0.335 | 0.631 | 0.993 |
| OR Quartile 3 | 0.624 | 0.870 | 0.291 | 0.500 | 0.611 |
| p Value | 0.043 | 0.53 | 3.8E−7 | 0.0019 | 0.025 |
| Lower limit of 95% CI | 0.396 | 0.562 | 0.181 | 0.324 | 0.396 |
| Upper limit of 95% CI | 0.984 | 1.35 | 0.468 | 0.774 | 0.941 |
| OR Quartile 4 | 0.746 | 0.974 | 0.347 | 0.606 | 0.699 |
| p Value | 0.26 | 0.92 | 5.3E−4 | 0.047 | 0.16 |
| Lower limit of 95% CI | 0.448 | 0.589 | 0.190 | 0.369 | 0.425 |
| Upper limit of 95% CI | 1.24 | 1.61 | 0.631 | 0.993 | 1.15 |

Serum Creatinine

| | | | | | |
|---|---|---|---|---|---|
| AUC | 0.81 | 0.83 | 0.68 | 0.78 | 0.80 |
| SE | 0.022 | 0.021 | 0.030 | 0.024 | 0.023 |
| p Value | 0 | 0 | 1.6E−9 | 0 | 0 |
| nCohort Non-persistent | 113 | 132 | 222 | 149 | 159 |
| nCohort Persistent | 241 | 220 | 130 | 205 | 193 |
| Cutoff Quartile 2 | 1.64 | 1.64 | 1.65 | 1.64 | 1.64 |
| Sensitivity | 87% | 90% | 87% | 89% | 91% |
| Specificity | 51% | 51% | 32% | 44% | 45% |
| Cutoff Quartile 3 | 2.44 | 2.44 | 2.44 | 2.44 | 2.44 |
| Sensitivity | 65% | 69% | 66% | 67% | 69% |
| Specificity | 81% | 81% | 59% | 72% | 74% |
| Cutoff Quartile 4 | 3.32 | 3.31 | 3.32 | 3.32 | 3.31 |
| Sensitivity | 35% | 38% | 42% | 39% | 40% |
| Specificity | 96% | 96% | 84% | 93% | 93% |
| OR Quartile 2 | 7.14 | 9.77 | 3.13 | 6.29 | 8.35 |
| p Value | 2.8E−13 | 2.4E−15 | 1.3E−4 | 2.7E−11 | 1.5E−12 |
| Lower limit of 95% CI | 4.22 | 5.55 | 1.75 | 3.66 | 4.64 |
| Upper limit of 95% CI | 12.1 | 17.2 | 5.60 | 10.8 | 15.0 |
| OR Quartile 3 | 8.19 | 9.37 | 2.81 | 5.31 | 6.33 |
| p Value | 3.2E−14 | 0 | 7.0E−6 | 1.5E−12 | 9.8E−15 |
| Lower limit of 95% CI | 4.76 | 5.57 | 1.79 | 3.34 | 3.97 |
| Upper limit of 95% CI | 14.1 | 15.8 | 4.42 | 8.43 | 10.1 |
| OR Quartile 4 | 11.8 | 15.4 | 3.80 | 7.87 | 8.93 |
| p Value | 2.3E−7 | 9.8E−9 | 1.9E−7 | 2.2E−9 | 2.3E−10 |
| Lower limit of 95% CI | 4.62 | 6.05 | 2.30 | 4.00 | 4.54 |
| Upper limit of 95% CI | 30.0 | 39.2 | 6.27 | 15.5 | 17.6 |

Cystatin-C

| | | | | | |
|---|---|---|---|---|---|
| AUC | 0.76 | 0.75 | 0.71 | 0.77 | 0.75 |
| SE | 0.027 | 0.027 | 0.032 | 0.026 | 0.027 |
| p Value | 0 | 0 | 4.4E−11 | 0 | 0 |
| nCohort Non-persistent | 99 | 114 | 202 | 134 | 141 |
| nCohort Persistent | 209 | 193 | 104 | 174 | 166 |
| Cutoff Quartile 2 | 1170000 | 1170000 | 1170000 | 1170000 | 1170000 |
| Sensitivity | 86% | 87% | 88% | 88% | 87% |
| Specificity | 46% | 44% | 32% | 41% | 39% |
| Cutoff Quartile 3 | 1660000 | 1650000 | 1660000 | 1660000 | 1650000 |
| Sensitivity | 64% | 65% | 72% | 69% | 69% |
| Specificity | 79% | 75% | 61% | 75% | 72% |
| Cutoff Quartile 4 | 2150000 | 2150000 | 2150000 | 2150000 | 2150000 |
| Sensitivity | 32% | 34% | 41% | 37% | 39% |
| Specificity | 90% | 90% | 83% | 91% | 91% |
| OR Quartile 2 | 5.18 | 5.02 | 3.64 | 5.07 | 4.42 |
| p Value | 5.5E−9 | 1.2E−8 | 1.6E−4 | 2.6E−8 | 3.1E−7 |
| Lower limit of 95% CI | 2.98 | 2.88 | 1.86 | 2.86 | 2.50 |
| Upper limit of 95% CI | 9.00 | 8.74 | 7.11 | 8.98 | 7.80 |
| OR Quartile 3 | 6.5v0 | 5.39 | 4.11 | 6.54 | 5.54 |
| p Value | 5.0E−11 | 1.4E−10 | 6.9E−8 | 3.0E−13 | 8.9E−12 |
| Lower limit of 95% CI | 3.72 | 3.22 | 2.46 | 3.95 | 3.39 |
| Upper limit of 95% CI | 11.4 | 9.01 | 6.87 | 10.8 | 9.05 |
| OR Quartile 4 | 4.20 | 4.87 | 3.48 | 6.06 | 6.18 |
| p Value | 8.4E−5 | 6.8E−6 | 5.2E−6 | 1.2E−7 | 4.1E−8 |
| Lower limit of 95% CI | 2.05 | 2.44 | 2.04 | 3.11 | 3.22 |
| Upper limit of 95% CI | 8.59 | 9.69 | 5.96 | 11.8 | 11.8 |

C-C motif chemokine 14/(Weight adjusted urine output)

| | | | | | |
|---|---|---|---|---|---|
| AUC | 0.74 | 0.72 | 0.74 | 0.76 | 0.73 |
| SE | 0.027 | 0.028 | 0.030 | 0.026 | 0.027 |
| p Value | 0 | 1.4E−14 | 2.2E−15 | 0 | 0 |
| nCohort Non-persistent | 110 | 129 | 215 | 147 | 156 |
| nCohort Persistent | 218 | 197 | 113 | 181 | 170 |
| Cutoff Quartile 2 | 1.26 | 1.24 | 1.26 | 1.26 | 1.24 |
| Sensitivity | 83% | 83% | 90% | 85% | 85% |
| Specificity | 42% | 37% | 33% | 37% | 36% |
| Cutoff Quartile 3 | 4.59 | 4.54 | 4.59 | 4.59 | 4.54 |
| Sensitivity | 63% | 64% | 73% | 69% | 68% |
| Specificity | 76% | 71% | 62% | 73% | 69% |
| Cutoff Quartile 4 | 24.3 | 22.9 | 24.3 | 24.3 | 22.9 |
| Sensitivity | 34% | 35% | 42% | 40% | 39% |
| Specificity | 93% | 90% | 84% | 93% | 90% |
| OR Quartile 2 | 3.63 | 2.84 | 4.57 | 3.41 | 3.10 |
| p Value | 1.2E−6 | 6.8E−5 | 1.3E−5 | 5.3E−6 | 2.9E−5 |
| Lower limit of 95% CI | 2.16 | 1.70 | 2.31 | 2.01 | 1.82 |
| Upper limit of 95% CI | 6.12 | 4.75 | 9.06 | 5.78 | 5.27 |
| OR Quartile 3 | 5.57 | 4.41 | 4.58 | 5.82 | 4.70 |
| p Value | 8.7E−11 | 1.3E−9 | 2.6E−9 | 6.4E−13 | 8.7E−11 |
| Lower limit of 95% CI | 3.32 | 2.73 | 2.77 | 3.60 | 2.95 |
| Upper limit of 95% CI | 9.36 | 7.13 | 7.55 | 9.40 | 7.51 |
| OR Quartile 4 | 6.55 | 4.81 | 3.93 | 9.05 | 6.11 |
| p Value | 1.8E−6 | 1.7E−6 | 2.9E−7 | 1.1E−9 | 7.8E−9 |
| Lower limit of 95% CI | 3.03 | 2.53 | 2.33 | 4.46 | 3.31 |
| Upper limit of 95% CI | 14.2 | 9.16 | 6.63 | 18.4 | 11.3 |

C-C motif chemokine 14 X Serum Creatinine

| | | | | | |
|---|---|---|---|---|---|
| AUC | 0.83 | 0.83 | 0.71 | 0.81 | 0.82 |
| SE | 0.022 | 0.022 | 0.032 | 0.023 | 0.023 |
| p Value | 0 | 0 | 7.9E−11 | 0 | 0 |
| nCohort Non-persistent | 108 | 126 | 211 | 144 | 153 |
| nCohort Persistent | 216 | 196 | 111 | 180 | 169 |
| Cutoff Quartile 2 | 1.29 | 1.29 | 1.30 | 1.29 | 1.29 |
| Sensitivity | 87% | 89% | 87% | 89% | 90% |
| Specificity | 49% | 48% | 32% | 42% | 42% |
| Cutoff Quartile 3 | 3.97 | 3.91 | 3.97 | 3.97 | 3.91 |
| Sensitivity | 68% | 70% | 76% | 72% | 74% |
| Specificity | 85% | 82% | 64% | 78% | 76% |
| Cutoff Quartile 4 | 18.3 | 16.8 | 18.0 | 18.3 | 16.8 |
| Sensitivity | 36% | 39% | 38% | 41% | 43% |
| Specificity | 96% | 96% | 82% | 94% | 94% |
| OR Quartile 2 | 6.47 | 7.58 | 3.22 | 5.88 | 6.43 |
| p Value | 2.4E−11 | 3.9E−12 | 2.8E−4 | 1.1E−9 | 9.0E−10 |
| Lower limit of 95% CI | 3.74 | 4.28 | 1.72 | 3.32 | 3.55 |
| Upper limit of 95% CI | 11.2 | 13.4 | 6.06 | 10.4 | 11.7 |
| OR Quartile 3 | 12.0 | 10.7 | 5.41 | 9.10 | 9.23 |
| p Value | 6.7E−16 | 0 | 1.4E−10 | 0 | 0 |
| Lower limit of 95% CI | 6.57 | 6.17 | 3.23 | 5.46 | 5.56 |
| Upper limit of 95% CI | 21.9 | 18.4 | 9.07 | 15.2 | 15.3 |
| OR Quartile 4 | 14.4 | 15.3 | 2.68 | 11.6 | 11.9 |
| p Value | 4.6E−7 | 1.2E−8 | 1.8E−4 | 5.1E−10 | 5.3E−11 |
| Lower limit of 95% CI | 5.11 | 5.99 | 1.60 | 5.36 | 5.67 |
| Upper limit of 95% CI | 40.6 | 39.2 | 4.50 | 25.1 | 24.9 |

C-C motif chemokine 14 X Cystatin-C

| | | | | | |
|---|---|---|---|---|---|
| AUC | 0.81 | 0.82 | 0.71 | 0.81 | 0.81 |
| SE | 0.024 | 0.024 | 0.033 | 0.024 | 0.025 |
| p Value | 0 | 0 | 3.3E−10 | 0 | 0 |
| nCohort Non-persistent | 99 | 114 | 202 | 134 | 141 |
| nCohort Persistent | 209 | 193 | 104 | 174 | 166 |
| Cutoff Quartile 2 | 956000 | 955000 | 958000 | 956000 | 955000 |
| Sensitivity | 87% | 88% | 88% | 88% | 88% |
| Specificity | 49% | 46% | 32% | 42% | 40% |
| Cutoff Quartile 3 | 2880000 | 2860000 | 2880000 | 2880000 | 2860000 |
| Sensitivity | 66% | 68% | 73% | 72% | 73% |
| Specificity | 83% | 81% | 62% | 79% | 77% |
| Cutoff Quartile 4 | 1.04E7 | 1.03E7 | 1.03E7 | 1.04E7 | 1.03E7 |
| Sensitivity | 35% | 38% | 40% | 41% | 42% |
| Specificity | 97% | 96% | 83% | 96% | 95% |
| OR Quartile 2 | 6.34 | 6.12 | 3.64 | 5.23 | 4.95 |
| p Value | 1.0E−10 | 3.1E−10 | 1.6E−4 | 1.3E−8 | 5.1E−8 |
| Lower limit of 95% CI | 3.62 | 3.48 | 1.86 | 2.96 | 2.79 |
| Upper limit of 95% CI | 11.1 | 10.8 | 7.11 | 9.26 | 8.81 |
| OR Quartile 3 | 9.18 | 9.05 | 4.41 | 9.94 | 8.80 |
| p Value | 2.9E−13 | 7.5E−15 | 2.0E−8 | 0 | 2.2E−16 |
| Lower limit of 95% CI | 5.06 | 5.19 | 2.62 | 5.83 | 5.24 |
| Upper limit of 95% CI | 16.6 | 15.8 | 7.40 | 16.9 | 14.8 |
| OR Quartile 4 | 17.5 | 16.7 | 3.23 | 14.7 | 14.0 |
| p Value | 2.1E−6 | 1.1E−7 | 1.7E−5 | 1.6E−9 | 3.0E−10 |
| Lower limit of 95% CI | 5.37 | 5.92 | 1.89 | 6.14 | 6.15 |
| Upper limit of 95% CI | 57.3 | 47.3 | 5.52 | 35.2 | 31.7 |

C-C motif chemokine 14 X Serum Creatinine/(Weight adjusted urine output)

| | | | | | |
|---|---|---|---|---|---|
| AUC | 0.79 | 0.77 | 0.76 | 0.79 | 0.77 |
| SE | 0.025 | 0.026 | 0.030 | 0.025 | 0.026 |
| p Value | 0 | 0 | 0 | 0 | 0 |
| nCohort Non-persistent | 106 | 124 | 210 | 142 | 151 |
| nCohort Persistent | 215 | 195 | 111 | 179 | 168 |
| Cutoff Quartile 2 | 2.17 | 2.15 | 2.17 | 2.17 | 2.15 |
| Sensitivity | 85% | 85% | 89% | 87% | 87% |
| Specificity | 45% | 41% | 32% | 40% | 38% |
| Cutoff Quartile 3 | 10.2 | 10.1 | 10.2 | 10.2 | 10.1 |
| Sensitivity | 66% | 67% | 77% | 72% | 71% |
| Specificity | 82% | 76% | 64% | 77% | 73% |
| Cutoff Quartile 4 | 69.8 | 67.6 | 69.8 | 69.8 | 67.6 |
| Sensitivity | 36% | 37% | 43% | 42% | 42% |
| Specificity | 96% | 94% | 84% | 96% | 93% |
| OR Quartile 2 | 4.73 | 4.00 | 3.95 | 4.55 | 4.14 |
| p Value | 1.3E−8 | 3.4E−7 | 5.2E−5 | 7.3E−8 | 5.4E−7 |
| Lower limit of 95% CI | 2.77 | 2.35 | 2.03 | 2.62 | 2.38 |
| Upper limit of 95% CI | 8.09 | 6.81 | 7.68 | 7.90 | 7.21 |
| OR Quartile 3 | 8.91 | 6.27 | 5.76 | 8.29 | 6.52 |
| p Value | 6.1E−14 | 1.4E−12 | 4.7E−11 | 2.2E−16 | 6.0E−14 |
| Lower limit of 95% CI | 5.03 | 3.77 | 3.42 | 4.99 | 3.99 |
| Upper limit of 95% CI | 15.8 | 10.4 | 9.71 | 13.8 | 10.6 |
| OR Quartile 4 | 14.2 | 8.49 | 4.09 | 16.3 | 10.1 |
| p Value | 5.2E−7 | 5.9E−8 | 1.8E−7 | 3.1E−10 | 1.9E−10 |
| Lower limit of 95% CI | 5.04 | 3.92 | 2.41 | 6.85 | 4.95 |
| Upper limit of 95% CI | 40.1 | 18.4 | 6.93 | 39.0 | 20.5 |

C-C motif chemokine 14 X Cystatin-C/(Weight adjusted urine output)

| | | | | | |
|---|---|---|---|---|---|
| AUC | 0.77 | 0.75 | 0.75 | 0.78 | 0.76 |
| SE | 0.027 | 0.028 | 0.031 | 0.026 | 0.027 |
| p Value | 0 | 0 | 2.4E−15 | 0 | 0 |
| nCohort Non-persistent | 97 | 112 | 201 | 132 | 139 |
| nCohort Persistent | 208 | 192 | 104 | 173 | 165 |
| Cutoff Quartile 2 | 1580000 | 1580000 | 1580000 | 1580000 | 1580000 |
| Sensitivity | 84% | 83% | 90% | 86% | 85% |
| Specificity | 43% | 39% | 33% | 39% | 37% |
| Cutoff Quartile 3 | 6710000 | 6570000 | 6710000 | 6710000 | 6570000 |
| Sensitivity | 64% | 65% | 75% | 70% | 69% |
| Specificity | 79% | 76% | 63% | 76% | 73% |
| Cutoff Quartile 4 | 3.85E7 | 3.84E7 | 3.85E7 | 3.85E7 | 3.84E7 |
| Sensitivity | 34% | 34% | 41% | 40% | 39% |
| Specificity | 94% | 91% | 83% | 94% | 91% |
| OR Quartile 2 | 3.91 | 3.24 | 4.60 | 3.73 | 3.25 |
| p Value | 9.2E−7 | 1.8E−5 | 2.9E−5 | 2.7E−6 | 2.5E−5 |
| Lower limit of 95% CI | 2.27 | 1.89 | 2.25 | 2.15 | 1.88 |
| Upper limit of 95% CI | 6.74 | 5.53 | 9.40 | 6.46 | 5.61 |
| OR Quartile 3 | 6.83 | 5.87 | 5.04 | 7.27 | 5.94 |
| p Value | 3.3E−11 | 3.8E−11 | 1.9E−9 | 3.8E−14 | 2.4E−12 |
| Lower limit of 95% CI | 3.87 | 3.47 | 2.97 | 4.35 | 3.61 |
| Upper limit of 95% CI | 12.0 | 9.93 | 8.54 | 12.2 | 9.78 |
| OR Quartile 4 | 7.86 | 5.34 | 3.46 | 10.3 | 6.71 |
| p Value | 3.8E−6 | 4.3E−6 | 5.8E−6 | 4.1E−9 | 2.5E−8 |
| Lower limit of 95% CI | 3.28 | 2.61 | 2.02 | 4.73 | 3.43 |
| Upper limit of 95% CI | 18.8 | 10.9 | 5.92 | 22.4 | 13.1 |

| | Persistence duration (hr) | | | |
|---|---|---|---|---|
| | 48 | 72 | | |
| | UO only | sCr or UO | sCr only | UO only |
| C-C motif chemokine 14 | | | | |
| AUC | 0.69 | 0.79 | 0.79 | 0.69 |
| SE | 0.033 | 0.025 | 0.025 | 0.034 |
| p Value | 4.2E−9 | 0 | 0 | 1.5E−8 |
| nCohort Non-persistent | 229 | 163 | 171 | 235 |
| nCohort Persistent | 100 | 168 | 158 | 94 |
| Cutoff Quartile 2 | 0.732 | 0.725 | 0.719 | 0.732 |
| Sensitivity | 89% | 90% | 91% | 89% |
| Specificity | 31% | 41% | 39% | 31% |
| Cutoff Quartile 3 | 1.64 | 1.64 | 1.64 | 1.64 |
| Sensitivity | 74% | 73% | 73% | 73% |
| Specificity | 60% | 73% | 71% | 59% |
| Cutoff Quartile 4 | 5.09 | 5.16 | 5.09 | 5.09 |
| Sensitivity | 37% | 42% | 43% | 38% |
| Specificity | 80% | 92% | 91% | 80% |
| OR Quartile 2 | 3.64 | 6.63 | 6.14 | 3.71 |
| p Value | 2.3E−4 | 7.5E−10 | 6.9E−9 | 3.1E−4 |
| Lower limit of 95% CI | 1.83 | 3.63 | 3.32 | 1.82 |
| Upper limit of 95% CI | 7.22 | 12.1 | 11.4 | 7.56 |
| OR Quartile 3 | 4.32 | 7.17 | 6.88 | 4.00 |
| p Value | 3.4E−8 | 1.6E−15 | 6.0E−15 | 2.5E−7 |
| Lower limit of 95% CI | 2.57 | 4.42 | 4.24 | 2.36 |
| Upper limit of 95% CI | 7.26 | 11.6 | 11.2 | 6.76 |
| OR Quartile 4 | 2.34 | 8.24 | 7.86 | 2.48 |
| p Value | 0.0013 | 1.4E−10 | 5.5E−11 | 6.8E−4 |
| Lower limit of 95% CI | 1.39 | 4.33 | 4.24 | 1.47 |
| Upper limit of 95% CI | 3.93 | 15.7 | 14.6 | 4.19 |
| Weight adjusted urine output | | | | |
| AUC | 0.30 | 0.37 | 0.41 | 0.31 |
| SE | 0.032 | 0.030 | 0.031 | 0.033 |
| p Value | 2.7E−10 | 1.7E−5 | 0.0025 | 1.6E−8 |
| nCohort Non-persistent | 233 | 165 | 174 | 239 |
| nCohort Persistent | 104 | 172 | 161 | 98 |
| Cutoff Quartile 2 | 0.189 | 0.189 | 0.192 | 0.189 |
| Sensitivity | 55% | 67% | 70% | 56% |
| Specificity | 16% | 16% | 20% | 17% |
| Cutoff Quartile 3 | 0.366 | 0.366 | 0.368 | 0.366 |
| Sensitivity | 33% | 41% | 43% | 34% |
| Specificity | 42% | 41% | 43% | 43% |
| Cutoff Quartile 4 | 0.857 | 0.857 | 0.862 | 0.857 |
| Sensitivity | 14% | 20% | 21% | 15% |
| Specificity | 70% | 70% | 71% | 71% |
| OR Quartile 2 | 0.229 | 0.395 | 0.593 | 0.265 |
| p Value | 3.1E−8 | 4.6E−4 | 0.041 | 6.0E−7 |
| Lower limit of 95% CI | 0.136 | 0.235 | 0.359 | 0.157 |
| Upper limit of 95% CI | 0.386 | 0.664 | 0.979 | 0.446 |
| OR Quartile 3 | 0.353 | 0.481 | 0.568 | 0.385 |
| p Value | 2.6E−5 | 9.5E−4 | 0.010 | 1.4E−4 |
| Lower limit of 95% CI | 0.217 | 0.311 | 0.369 | 0.235 |
| Upper limit of 95% CI | 0.573 | 0.742 | 0.876 | 0.628 |
| OR Quartile 4 | 0.392 | 0.588 | 0.664 | 0.436 |
| p Value | 0.0029 | 0.036 | 0.11 | 0.0084 |
| Lower limit of 95% CI | 0.212 | 0.357 | 0.402 | 0.236 |
| Upper limit of 95% CI | 0.726 | 0.967 | 1.10 | 0.808 |
| Serum Creatinine | | | | |
| AUC | 0.69 | 0.75 | 0.77 | 0.68 |
| SE | 0.031 | 0.026 | 0.025 | 0.032 |
| p Value | 1.9E−9 | 0 | 0 | 1.9E−8 |
| nCohort Non-persistent | 235 | 164 | 173 | 242 |
| nCohort Persistent | 117 | 190 | 179 | 110 |
| Cutoff Quartile 2 | 1.65 | 1.64 | 1.64 | 1.65 |
| Sensitivity | 88% | 89% | 91% | 88% |
| Specificity | 31% | 41% | 42% | 31% |
| Cutoff Quartile 3 | 2.44 | 2.44 | 2.44 | 2.44 |
| Sensitivity | 67% | 67% | 70% | 66% |
| Specificity | 58% | 70% | 71% | 57% |
| Cutoff Quartile 4 | 3.32 | 3.32 | 3.31 | 3.32 |
| Sensitivity | 44% | 38% | 40% | 44% |
| Specificity | 84% | 90% | 90% | 83% |
| OR Quartile 2 | 3.38 | 5.70 | 7.26 | 3.35 |
| p Value | 1.2E−4 | 5.4E−10 | 6.9E−11 | 2.1E−4 |
| Lower limit of 95% CI | 1.81 | 3.29 | 4.00 | 1.77 |
| Upper limit of 95% CI | 6.30 | 9.88 | 13.2 | 6.35 |
| OR Quartile 3 | 2.75 | 4.71 | 5.54 | 2.62 |
| p Value | 1.9E−5 | 1.5E−11 | 2.1E−13 | 6.1E−5 |
| Lower limit of 95% CI | 1.73 | 3.00 | 3.51 | 1.64 |
| Upper limit of 95% CI | 4.37 | 7.38 | 8.74 | 4.19 |
| OR Quartile 4 | 4.01 | 5.40 | 6.03 | 3.80 |
| p Value | 6.8E−8 | 1.3E−8 | 1.6E−9 | 2.2E−7 |
| Lower limit of 95% CI | 2.42 | 3.02 | 3.37 | 2.29 |
| Upper limit of 95% CI | 6.63 | 9.65 | 10.8 | 6.29 |
| Cystatin-C | | | | |
| AUC | 0.72 | 0.75 | 0.74 | 0.72 |
| SE | 0.034 | 0.028 | 0.028 | 0.034 |
| p Value | 6.3E−11 | 0 | 0 | 2.7E−10 |
| nCohort Non-persistent | 214 | 148 | 154 | 219 |
| nCohort Persistent | 92 | 160 | 153 | 87 |
| Cutoff Quartile 2 | 1170000 | 1170000 | 1170000 | 1170000 |
| Sensitivity | 89% | 88% | 87% | 89% |
| Specificity | 31% | 38% | 36% | 31% |
| Cutoff Quartile 3 | 1660000 | 1660000 | 1650000 | 1660000 |
| Sensitivity | 74% | 69% | 69% | 74% |
| Specificity | 60% | 70% | 68% | 59% |
| Cutoff Quartile 4 | 2150000 | 2150000 | 2150000 | 2150000 |
| Sensitivity | 43% | 38% | 39% | 44% |
| Specificity | 83% | 89% | 88% | 82% |
| OR Quartile 2 | 3.74 | 4.26 | 3.80 | 3.39 |
| p Value | 3.2E−4 | 7.6E−7 | 5.0E−6 | 8.6E−4 |
| Lower limit of 95% CI | 1.82 | 2.40 | 2.14 | 1.65 |
| Upper limit of 95% CI | 7.66 | 7.57 | 6.74 | 6.96 |
| OR Quartile 3 | 4.30 | 5.20 | 4.69 | 4.06 |
| p Value | 1.2E−7 | 2.9E−11 | 3.1E−10 | 5.2E−7 |
| Lower limit of 95% CI | 2.51 | 3.20 | 2.90 | 2.35 |
| Upper limit of 95% CI | 7.38 | 8.45 | 7.58 | 7.03 |
| OR Quartile 4 | 3.68 | 4.62 | 4.74 | 3.58 |
| p Value | 2.6E−6 | 5.2E−7 | 2.3E−7 | 4.9E−6 |
| Lower limit of 95% CI | 2.14 | 2.54 | 2.63 | 2.07 |
| Upper limit of 95% CI | 6.34 | 8.41 | 8.55 | 6.19 |
| C-C motif chemokine 14/(Weight adjusted urine output) | | | | |
| AUC | 0.74 | 0.76 | 0.74 | 0.73 |
| SE | 0.032 | 0.026 | 0.027 | 0.033 |
| p Value | 6.3E−14 | 0 | 0 | 3.3E−12 |
| nCohort Non-persistent | 228 | 161 | 169 | 234 |
| nCohort Persistent | 100 | 167 | 157 | 94 |
| Cutoff Quartile 2 | 1.26 | 1.26 | 1.24 | 1.26 |
| Sensitivity | 90% | 86% | 86% | 89% |
| Specificity | 32% | 37% | 36% | 31% |
| Cutoff Quartile 3 | 4.59 | 4.59 | 4.54 | 4.59 |
| Sensitivity | 74% | 71% | 69% | 73% |
| Specificity | 61% | 71% | 68% | 59% |
| Cutoff Quartile 4 | 24.3 | 24.3 | 22.9 | 24.3 |
| Sensitivity | 46% | 41% | 41% | 47% |
| Specificity | 84% | 92% | 89% | 84% |
| OR Quartile 2 | 4.15 | 3.62 | 3.38 | 3.73 |
| p Value | 8.6E−5 | 3.6E−6 | 1.4E−5 | 2.9E−4 |
| Lower limit of 95% CI | 2.04 | 2.10 | 1.95 | 1.83 |
| Upper limit of 95% CI | 8.45 | 6.24 | 5.85 | 7.61 |
| OR Quartile 3 | 4.36 | 6.02 | 4.84 | 4.04 |
| p Value | 2.8E−8 | 1.7E−13 | 4.4E−11 | 2.1E−7 |
| Lower limit of 95% CI | 2.60 | 3.74 | 3.03 | 2.38 |
| Upper limit of 95% CI | 7.34 | 9.70 | 7.73 | 6.84 |
| OR Quartile 4 | 4.54 | 8.02 | 5.77 | 4.54 |
| p Value | 2.2E−8 | 2.6E−10 | 3.8E−9 | 2.8E−8 |
| Lower limit of 95% CI | 2.67 | 4.20 | 3.22 | 2.66 |
| Upper limit of 95% CI | 7.72 | 15.3 | 10.3 | 7.74 |
| C-C motif chemokine 14 X Serum Creatinine | | | | |
| AUC | 0.72 | 0.81 | 0.81 | 0.71 |
| SE | 0.033 | 0.024 | 0.024 | 0.034 |
| p Value | 2.6E−11 | 0 | 0 | 1.4E−10 |
| nCohort Non-persistent | 224 | 158 | 166 | 230 |
| nCohort Persistent | 98 | 166 | 156 | 92 |
| Cutoff Quartile 2 | 1.30 | 1.29 | 1.29 | 1.30 |
| Sensitivity | 89% | 90% | 91% | 89% |
| Specificity | 31% | 41% | 40% | 31% |
| Cutoff Quartile 3 | 3.97 | 3.97 | 3.91 | 3.97 |
| Sensitivity | 78% | 74% | 76% | 77% |
| Specificity | 62% | 75% | 74% | 61% |
| Cutoff Quartile 4 | 18.0 | 18.3 | 16.8 | 18.0 |
| Sensitivity | 41% | 42% | 44% | 41% |
| Specificity | 82% | 93% | 93% | 81% |
| OR Quartile 2 | 3.60 | 6.55 | 6.86 | 3.66 |
| p Value | 2.7E−4 | 1.1E−9 | 2.1E−9 | 3.6E−4 |
| Lower limit of 95% CI | 1.81 | 3.58 | 3.65 | 1.79 |
| Upper limit of 95% CI | 7.15 | 12.0 | 12.9 | 7.47 |
| OR Quartile 3 | 5.65 | 8.73 | 8.88 | 5.26 |
| p Value | 5.1E−10 | 0 | 0 | 4.3E−9 |
| Lower limit of 95% CI | 3.27 | 5.29 | 5.36 | 3.02 |
| Upper limit of 95% CI | 9.75 | 14.4 | 14.7 | 9.15 |
| OR Quartile 4 | 3.08 | 9.74 | 10.2 | 3.06 |
| p Value | 2.8E−5 | 7.7E−11 | 9.2E−12 | 3.7E−5 |
| Lower limit of 95% CI | 1.82 | 4.91 | 5.22 | 1.80 |
| Upper limit of 95% CI | 5.21 | 19.3 | 19.8 | 5.21 |
| C-C motif chemokine 14 X Cystatin-C | | | | |
| AUC | 0.72 | 0.81 | 0.81 | 0.72 |
| SE | 0.034 | 0.024 | 0.025 | 0.034 |
| p Value | 1.3E−10 | 0 | 0 | 1.4E−10 |
| nCohort Non-persistent | 214 | 148 | 154 | 219 |
| nCohort Persistent | 92 | 160 | 153 | 87 |
| Cutoff Quartile 2 | 958000 | 956000 | 955000 | 958000 |
| Sensitivity | 88% | 90% | 90% | 89% |
| Specificity | 31% | 41% | 40% | 31% |
| Cutoff Quartile 3 | 2880000 | 2880000 | 2860000 | 2880000 |
| Sensitivity | 77% | 74% | 75% | 77% |
| Specificity | 62% | 76% | 74% | 61% |
| Cutoff Quartile 4 | 1.03E7 | 1.04E7 | 1.03E7 | 1.03E7 |
| Sensitivity | 43% | 43% | 44% | 46% |
| Specificity | 83% | 95% | 94% | 83% |
| OR Quartile 2 | 3.28 | 6.31 | 5.62 | 3.39 |
| p Value | 7.8E−4 | 3.5E−9 | 3.0E−8 | 8.6E−4 |
| Lower limit of 95% CI | 1.64 | 3.42 | 3.05 | 1.65 |
| Upper limit of 95% CI | 6.57 | 11.6 | 10.3 | 6.96 |
| OR Quartile 3 | 5.44 | 9.37 | 8.33 | 5.18 |
| p Value | 2.9E−9 | 0 | 4.4E−16 | 1.4E−8 |
| Lower limit of 95% CI | 3.11 | 5.57 | 4.99 | 2.93 |
| Upper limit of 95% CI | 9.52 | 15.8 | 13.9 | 9.15 |
| OR Quartile 4 | 3.68 | 13.3 | 12.9 | 4.19 |
| p Value | 2.6E−6 | 7.4E−11 | 1.8E−11 | 3.4E−7 |
| Lower limit of 95% CI | 2.14 | 6.09 | 6.12 | 2.41 |
| Upper limit of 95% CI | 6.34 | 28.9 | 27.1 | 7.26 |
| C-C motif chemokine 14 X Serum Creatinine/(Weight adjusted urine output) | | | | |
| AUC | 0.76 | 0.79 | 0.77 | 0.75 |
| SE | 0.031 | 0.025 | 0.026 | 0.032 |
| p Value | 0 | 0 | 0 | 6.7E−15 |
| nCohort Non-persistent | 223 | 156 | 164 | 229 |
| nCohort Persistent | 98 | 165 | 155 | 92 |
| Cutoff Quartile 2 | 2.17 | 2.17 | 2.15 | 2.17 |
| Sensitivity | 89% | 88% | 88% | 88% |
| Specificity | 31% | 39% | 38% | 30% |
| Cutoff Quartile 3 | 10.2 | 10.2 | 10.1 | 10.2 |
| Sensitivity | 78% | 73% | 72% | 77% |
| Specificity | 62% | 74% | 71% | 61% |
| Cutoff Quartile 4 | 69.8 | 69.8 | 67.6 | 69.8 |
| Sensitivity | 46% | 44% | 43% | 47% |
| Specificity | 84% | 94% | 92% | 83% |
| OR Quartile 2 | 3.54 | 4.93 | 4.63 | 3.18 |
| p Value | 3.2E−4 | 5.6E−8 | 2.7E−7 | 0.0010 |
| Lower limit of 95% CI | 1.78 | 2.77 | 2.58 | 1.59 |
| Upper limit of 95% CI | 7.05 | 8.78 | 8.30 | 6.33 |
| OR Quartile 3 | 5.61 | 7.98 | 6.29 | 5.22 |
| p Value | 6.1E−10 | 2.2E−16 | 1.3E−13 | 5.1E−9 |
| Lower limit of 95% CI | 3.25 | 4.85 | 3.87 | 3.00 |
| Upper limit of 95% CI | 9.68 | 13.1 | 10.2 | 9.09 |
| OR Quartile 4 | 4.41 | 12.6 | 8.84 | 4.41 |
| p Value | 5.1E−8 | 1.8E−11 | 4.8E−11 | 6.3E−8 |
| Lower limit of 95% CI | 2.59 | 6.03 | 4.62 | 2.58 |
| Upper limit of 95% CI | 7.52 | 26.5 | 16.9 | 7.55 |
| C-C motif chemokine 14 X Cystatin-C/(Weight adjusted urine output) | | | | |
| AUC | 0.75 | 0.78 | 0.76 | 0.74 |
| SE | 0.033 | 0.026 | 0.027 | 0.034 |
| p Value | 4.9E−14 | 0 | 0 | 6.1E−13 |
| nCohort Non-persistent | 213 | 146 | 152 | 218 |
| nCohort Persistent | 92 | 159 | 152 | 87 |
| Cutoff Quartile 2 | 1580000 | 1580000 | 1580000 | 1580000 |
| Sensitivity | 90% | 87% | 86% | 90% |
| Specificity | 31% | 38% | 36% | 31% |
| Cutoff Quartile 3 | 6710000 | 6710000 | 6570000 | 6710000 |
| Sensitivity | 77% | 72% | 71% | 77% |
| Specificity | 62% | 74% | 71% | 61% |
| Cutoff Quartile 4 | 3.85E7 | 3.85E7 | 3.84E7 | 3.85E7 |
| Sensitivity | 43% | 42% | 40% | 44% |
| Specificity | 83% | 92% | 90% | 82% |
| OR Quartile 2 | 4.23 | 3.97 | 3.54 | 3.85 |
| p Value | 1.5E−4 | 2.0E−6 | 1.3E−5 | 4.1E−4 |
| Lower limit of 95% CI | 2.01 | 2.25 | 2.01 | 1.82 |
| Upper limit of 95% CI | 8.93 | 7.01 | 6.24 | 8.12 |
| OR Quartile 3 | 5.40 | 7.43 | 6.02 | 5.14 |
| p Value | 3.5E−9 | 9.3E−15 | 1.2E−12 | 1.6E−8 |
| Lower limit of 95% CI | 3.09 | 4.47 | 3.67 | 2.91 |
| Upper limit of 95% CI | 9.45 | 12.3 | 9.89 | 9.08 |
| OR Quartile 4 | 3.66 | 8.71 | 6.12 | 3.56 |
| p Value | 2.9E−6 | 8.2E−10 | 1.3E−8 | 5.4E−6 |
| Lower limit of 95% CI | 2.12 | 4.37 | 3.28 | 2.06 |
| Upper limit of 95% CI | 6.30 | 17.4 | 11.4 | 6.15 |

TABLE 24
Comparison of marker levels and the area under the ROC curve (AUC) in urine samples for
the “persistent” and “non-persistent” cohorts where persistence starts
within 168 hours after sample collection and renal status was assessed by serum creatinine
(sCr) only, urine output (UO) only, or by either serum creatinine or urine output KDIGO
criteria. Markers are either positive going or negative going which can be discerned from
the numerical value of the AUC. More specifically, an AUC value >0.5 indicates a positive
going marker and an AUC value <0.5 indicates a negative going marker.

| Persistence duration (hr) | | | | |
|---|---|---|---|---|
| 24 | | | 48 | |
| sCr or UO | sCr only | UO only | sCr or UO | sCr only |

C-C motif chemokine 14

| | | | | | |
|---|---|---|---|---|---|
| AUC | 0.79 | 0.79 | 0.70 | 0.79 | 0.79 |
| SE | 0.024 | 0.024 | 0.031 | 0.025 | 0.025 |
| p Value | 0 | 0 | 7.5E−11 | 0 | 0 |
| nCohort Non-persistent | 109 | 128 | 209 | 144 | 154 |
| nCohort Persistent | 222 | 201 | 120 | 187 | 175 |
| Cutoff Quartile 2 | 0.725 | 0.719 | 0.732 | 0.725 | 0.719 |
| Sensitivity | 86% | 89% | 89% | 88% | 89% |
| Specificity | 49% | 46% | 33% | 42% | 40% |
| Cutoff Quartile 3 | 1.64 | 1.64 | 1.64 | 1.64 | 1.64 |
| Sensitivity | 65% | 68% | 72% | 70% | 71% |
| Specificity | 80% | 77% | 63% | 75% | 73% |
| Cutoff Quartile 4 | 5.16 | 5.09 | 5.09 | 5.16 | 5.09 |
| Sensitivity | 36% | 38% | 35% | 40% | 41% |
| Specificity | 96% | 95% | 80% | 94% | 93% |
| OR Quartile 2 | 6.06 | 6.62 | 4.06 | 5.09 | 5.22 |
| p Value | 5.2E−11 | 2.8E−11 | 2.0E−5 | 5.8E−9 | 1.1E−8 |
| Lower limit of 95% CI | 3.54 | 3.79 | 2.13 | 2.94 | 2.96 |
| Upper limit of 95% CI | 10.4 | 11.5 | 7.72 | 8.81 | 9.20 |
| OR Quartile 3 | 7.30 | 7.14 | 4.43 | 6.84 | 6.70 |
| p Value | 7.1E−13 | 3.5E−14 | 2.5E−9 | 1.3E−14 | 1.3E−14 |
| Lower limit of 95% CI | 4.24 | 4.30 | 2.72 | 4.20 | 4.13 |
| Upper limit of 95% CI | 12.6 | 11.9 | 7.22 | 11.2 | 10.9 |
| OR Quartile 4 | 14.5 | 12.6 | 2.21 | 9.82 | 9.09 |
| p Value | 4.2E−7 | 1.0E−8 | 0.0022 | 1.2E−9 | 2.4E−10 |
| Lower limit of 95% CI | 5.15 | 5.30 | 1.33 | 4.71 | 4.59 |
| Upper limit of 95% CI | 40.8 | 30.1 | 3.66 | 20.5 | 18.0 |

Weight adjusted urine output

| | | | | | |
|---|---|---|---|---|---|
| AUC | 0.42 | 0.47 | 0.28 | 0.37 | 0.42 |
| SE | 0.032 | 0.032 | 0.030 | 0.030 | 0.031 |
| p Value | 0.012 | 0.40 | 1.2E−13 | 2.9E−5 | 0.0076 |
| nCohort Non-persistent | 111 | 131 | 213 | 146 | 157 |
| nCohort Persistent | 226 | 204 | 124 | 191 | 178 |
| Cutoff Quartile 2 | 0.189 | 0.192 | 0.189 | 0.189 | 0.192 |
| Sensitivity | 72% | 74% | 56% | 68% | 71% |
| Specificity | 18% | 23% | 14% | 15% | 20% |
| Cutoff Quartile 3 | 0.366 | 0.368 | 0.366 | 0.366 | 0.368 |
| Sensitivity | 47% | 50% | 31% | 43% | 45% |
| Specificity | 43% | 49% | 39% | 40% | 44% |
| Cutoff Quartile 4 | 0.857 | 0.862 | 0.857 | 0.857 | 0.862 |
| Sensitivity | 23% | 25% | 14% | 21% | 22% |
| Specificity | 71% | 75% | 68% | 69% | 71% |
| OR Quartile 2 | 0.556 | 0.846 | 0.198 | 0.369 | 0.596 |
| p Value | 0.042 | 0.52 | 1.8E−9 | 3.4E−4 | 0.046 |
| Lower limit of 95% CI | 0.317 | 0.506 | 0.117 | 0.214 | 0.358 |
| Upper limit of 95% CI | 0.978 | 1.41 | 0.335 | 0.637 | 0.991 |
| OR Quartile 3 | 0.673 | 0.937 | 0.293 | 0.510 | 0.640 |
| p Value | 0.090 | 0.77 | 2.8E−7 | 0.0026 | 0.043 |
| Lower limit of 95% CI | 0.426 | 0.604 | 0.183 | 0.329 | 0.416 |
| Upper limit of 95% CI | 1.06 | 1.45 | 0.468 | 0.790 | 0.986 |
| OR Quartile 4 | 0.756 | 0.990 | 0.339 | 0.595 | 0.698 |
| p Value | 0.29 | 0.97 | 3.0E−4 | 0.039 | 0.16 |
| Lower limit of 95% CI | 0.453 | 0.597 | 0.188 | 0.362 | 0.425 |
| Upper limit of 95% CI | 1.26 | 1.64 | 0.609 | 0.975 | 1.15 |

Serum Creatinine

| | | | | | |
|---|---|---|---|---|---|
| AUC | 0.81 | 0.83 | 0.68 | 0.77 | 0.79 |
| SE | 0.023 | 0.021 | 0.030 | 0.024 | 0.024 |
| p Value | 0 | 0 | 1.5E−9 | 0 | 0 |
| nCohort Non-persistent | 110 | 129 | 216 | 144 | 155 |
| nCohort Persistent | 244 | 223 | 136 | 210 | 197 |
| Cutoff Quartile 2 | 1.64 | 1.64 | 1.65 | 1.64 | 1.64 |
| Sensitivity | 87% | 90% | 87% | 89% | 91% |
| Specificity | 52% | 51% | 32% | 45% | 45% |
| Cutoff Quartile 3 | 2.44 | 2.44 | 2.44 | 2.44 | 2.44 |
| Sensitivity | 64% | 68% | 66% | 66% | 68% |
| Specificity | 81% | 81% | 60% | 72% | 73% |
| Cutoff Quartile 4 | 3.32 | 3.31 | 3.32 | 3.32 | 3.31 |
| Sensitivity | 35% | 37% | 40% | 38% | 39% |
| Specificity | 95% | 96% | 84% | 92% | 93% |
| OR Quartile 2 | 7.12 | 9.57 | 3.14 | 6.38 | 8.19 |
| p Value | 2.9E−13 | 2.4E−15 | 8.7E−5 | 1.4E−11 | 1.1E−12 |
| Lower limit of 95% CI | 4.21 | 5.47 | 1.77 | 3.73 | 4.59 |
| Upper limit of 95% CI | 12.1 | 16.7 | 5.57 | 10.9 | 14.6 |
| OR Quartile 3 | 7.65 | 8.72 | 2.90 | 4.98 | 5.72 |
| p Value | 2.1E−13 | 2.2E−16 | 3.1E−6 | 1.0E−11 | 1.7E−13 |
| Lower limit of 95% CI | 4.44 | 5.19 | 1.85 | 3.14 | 3.60 |
| Upper limit of 95% CI | 13.2 | 14.7 | 4.54 | 7.92 | 9.10 |
| OR Quartile 4 | 11.2 | 14.7 | 3.41 | 7.29 | 8.40 |
| p Value | 4.0E−7 | 1.7E−8 | 1.5E−6 | 8.1E−9 | 7.1E−10 |
| Lower limit of 95% CI | 4.41 | 5.78 | 2.07 | 3.71 | 4.27 |
| Upper limit of 95% CI | 28.6 | 37.4 | 5.61 | 14.3 | 16.5 |

Cystatin-C

| | | | | | |
|---|---|---|---|---|---|
| AUC | 0.75 | 0.74 | 0.71 | 0.76 | 0.74 |
| SE | 0.028 | 0.028 | 0.032 | 0.027 | 0.028 |
| p Value | 0 | 0 | 8.7E−11 | 0 | 0 |
| nCohort Non-persistent | 96 | 111 | 195 | 129 | 137 |
| nCohort Persistent | 212 | 196 | 111 | 179 | 170 |
| Cutoff Quartile 2 | 1170000 | 1170000 | 1170000 | 1170000 | 1170000 |
| Sensitivity | 85% | 86% | 89% | 87% | 86% |
| Specificity | 46% | 43% | 33% | 41% | 39% |
| Cutoff Quartile 3 | 1660000 | 1650000 | 1660000 | 1660000 | 1650000 |
| Sensitivity | 63% | 64% | 70% | 68% | 67% |
| Specificity | 78% | 74% | 62% | 74% | 71% |
| Cutoff Quartile 4 | 2150000 | 2150000 | 2150000 | 2150000 | 2150000 |
| Sensitivity | 32% | 34% | 40% | 36% | 38% |
| Specificity | 90% | 90% | 83% | 91% | 91% |
| OR Quartile 2 | 4.76 | 4.57 | 4.12 | 4.73 | 4.03 |
| p Value | 2.7E−8 | 5.7E−8 | 3.3E−5 | 5.7E−8 | 9.7E−7 |
| Lower limit of 95% CI | 2.75 | 2.64 | 2.11 | 2.70 | 2.31 |
| Upper limit of 95% CI | 8.25 | 7.91 | 8.05 | 8.29 | 7.05 |
| OR Quartile 3 | 6.01 | 4.98 | 3.78 | 6.07 | 4.94 |
| p Value | 3.0E−10 | 9.3E−10 | 1.7E−7 | 2.4E−12 | 1.4E−10 |
| Lower limit of 95% CI | 3.44 | 2.98 | 2.30 | 3.66 | 3.03 |
| Upper limit of 95% CI | 10.5 | 8.32 | 6.23 | 10.1 | 8.04 |
| OR Quartile 4 | 3.97 | 4.62 | 3.22 | 5.56 | 5.76 |
| p Value | 1.6E−4 | 1.4E−5 | 1.7E−5 | 4.8E−7 | 1.3E−7 |
| Lower limit of 95% CI | 1.94 | 2.32 | 1.89 | 2.85 | 3.01 |
| Upper limit of 95% CI | 8.13 | 9.20 | 5.50 | 10.8 | 11.0 |

C-C motif chemokine 14/(Weight adjusted urine output)

| | | | | | |
|---|---|---|---|---|---|
| AUC | 0.75 | 0.72 | 0.76 | 0.76 | 0.74 |
| SE | 0.027 | 0.028 | 0.029 | 0.026 | 0.027 |
| p Value | 0 | 1.1E−14 | 0 | 0 | 0 |
| nCohort Non-persistent | 107 | 126 | 208 | 142 | 152 |
| nCohort Persistent | 221 | 200 | 120 | 186 | 174 |
| Cutoff Quartile 2 | 1.26 | 1.24 | 1.26 | 1.26 | 1.24 |
| Sensitivity | 83% | 82% | 91% | 85% | 84% |
| Specificity | 42% | 37% | 34% | 38% | 36% |
| Cutoff Quartile 3 | 4.59 | 4.54 | 4.59 | 4.59 | 4.54 |
| Sensitivity | 63% | 64% | 74% | 68% | 68% |
| Specificity | 77% | 72% | 64% | 73% | 70% |
| Cutoff Quartile 4 | 24.3 | 22.9 | 24.3 | 24.3 | 22.9 |
| Sensitivity | 34% | 35% | 42% | 40% | 40% |
| Specificity | 93% | 90% | 85% | 94% | 91% |
| OR Quartile 2 | 3.61 | 2.80 | 5.14 | 3.46 | 3.09 |
| p Value | 1.4E−6 | 8.2E−5 | 2.7E−6 | 3.6E−6 | 2.8E−5 |
| Lower limit of 95% CI | 2.14 | 1.68 | 2.59 | 2.05 | 1.82 |
| Upper limit of 95% CI | 6.08 | 4.69 | 10.2 | 5.86 | 5.23 |
| OR Quartile 3 | 5.56 | 4.62 | 5.09 | 5.75 | 5.01 |
| p Value | 1.4E−10 | 6.2E−10 | 1.4E−10 | 1.2E−12 | 2.1E−11 |
| Lower limit of 95% CI | 3.29 | 2.85 | 3.10 | 3.55 | 3.13 |
| Upper limit of 95% CI | 9.39 | 7.51 | 8.37 | 9.31 | 8.03 |
| OR Quartile 4 | 7.34 | 5.12 | 4.22 | 11.1 | 7.03 |
| p Value | 1.7E−6 | 1.3E−6 | 8.1E−8 | 1.0E−9 | 3.0E−9 |
| Lower limit of 95% CI | 3.25 | 2.64 | 2.49 | 5.12 | 3.69 |
| Upper limit of 95% CI | 16.6 | 9.92 | 7.14 | 23.9 | 13.4 |

C-C motif chemokine 14 X Serum Creatinine

| | | | | | |
|---|---|---|---|---|---|
| AUC | 0.83 | 0.84 | 0.71 | 0.82 | 0.82 |
| SE | 0.022 | 0.022 | 0.031 | 0.023 | 0.023 |
| p Value | 0 | 0 | 3.2E−12 | 0 | 0 |
| nCohort Non-persistent | 105 | 123 | 205 | 139 | 149 |
| nCohort Persistent | 219 | 199 | 117 | 185 | 173 |
| Cutoff Quartile 2 | 1.29 | 1.29 | 1.30 | 1.29 | 1.29 |
| Sensitivity | 87% | 89% | 88% | 89% | 90% |
| Specificity | 50% | 49% | 33% | 44% | 43% |
| Cutoff Quartile 3 | 3.97 | 3.91 | 3.97 | 3.97 | 3.91 |
| Sensitivity | 67% | 70% | 75% | 71% | 73% |
| Specificity | 86% | 82% | 64% | 78% | 77% |
| Cutoff Quartile 4 | 18.3 | 16.8 | 18.0 | 18.3 | 16.8 |
| Sensitivity | 36% | 39% | 38% | 40% | 42% |
| Specificity | 97% | 97% | 82% | 95% | 95% |
| OR Quartile 2 | 6.95 | 8.07 | 3.57 | 6.45 | 6.91 |
| p Value | 5.3E−12 | 1.0E−12 | 7.5E−5 | 1.7E−10 | 2.1E−10 |
| Lower limit of 95% CI | 4.01 | 4.55 | 1.90 | 3.64 | 3.81 |
| Upper limit of 95% CI | 12.1 | 14.3 | 6.71 | 11.4 | 12.5 |
| OR Quartile 3 | 12.2 | 10.6 | 5.49 | 8.45 | 8.73 |
| p Value | 1.3E−15 | 0 | 5.0E−11 | 2.2E−16 | 0 |
| Lower limit of 95% CI | 6.62 | 6.13 | 3.30 | 5.08 | 5.27 |
| Upper limit of 95% CI | 22.7 | 18.5 | 9.12 | 14.1 | 14.5 |
| OR Quartile 4 | 18.8 | 18.8 | 2.74 | 12.6 | 12.9 |
| p Value | 1.1E−6 | 2.9E−8 | 1.3E−4 | 1.2E−9 | 9.7E−11 |
| Lower limit of 95% CI | 5.77 | 6.66 | 1.63 | 5.56 | 5.94 |
| Upper limit of 95% CI | 61.3 | 52.9 | 4.59 | 28.4 | 27.9 |

C-C motif chemokine 14 X Cystatin-C

| | | | | | |
|---|---|---|---|---|---|
| AUC | 0.82 | 0.82 | 0.72 | 0.81 | 0.81 |
| SE | 0.024 | 0.023 | 0.031 | 0.024 | 0.024 |
| p Value | 0 | 0 | 3.4E−12 | 0 | 0 |
| nCohort Non-persistent | 96 | 111 | 195 | 129 | 137 |
| nCohort Persistent | 212 | 196 | 111 | 179 | 170 |
| Cutoff Quartile 2 | 956000 | 955000 | 958000 | 956000 | 955000 |
| Sensitivity | 87% | 88% | 89% | 88% | 88% |
| Specificity | 51% | 48% | 33% | 43% | 42% |
| Cutoff Quartile 3 | 2880000 | 2860000 | 2880000 | 2880000 | 2860000 |
| Sensitivity | 65% | 68% | 73% | 71% | 72% |
| Specificity | 83% | 81% | 63% | 79% | 77% |
| Cutoff Quartile 4 | 1.04E7 | 1.03E7 | 1.03E7 | 1.04E7 | 1.03E7 |
| Sensitivity | 35% | 37% | 41% | 40% | 41% |
| Specificity | 97% | 96% | 84% | 95% | 95% |
| OR Quartile 2 | 6.85 | 6.55 | 4.12 | 5.77 | 5.34 |
| p Value | 2.3E−11 | 8.0E−11 | 3.3E−5 | 2.0E−9 | 1.3E−8 |
| Lower limit of 95% CI | 3.90 | 3.72 | 2.11 | 3.25 | 3.00 |
| Upper limit of 95% CI | 12.0 | 11.5 | 8.05 | 10.2 | 9.52 |
| OR Quartile 3 | 9.32 | 9.05 | 4.61 | 9.23 | 8.34 |
| p Value | 5.4E−13 | 1.5E−14 | 4.2E−9 | 2.2E−16 | 8.9E−16 |
| Lower limit of 95% CI | 5.08 | 5.16 | 2.77 | 5.41 | 4.97 |
| Upper limit of 95% CI | 17.1 | 15.9 | 7.68 | 15.7 | 14.0 |
| OR Quartile 4 | 16.6 | 15.9 | 3.47 | 13.5 | 13.0 |
| p Value | 3.3E−6 | 1.8E−7 | 5.3E−6 | 5.1E−9 | 8.6E−10 |
| Lower limit of 95% CI | 5.09 | 5.62 | 2.03 | 5.63 | 5.73 |
| Upper limit of 95% CI | 54.3 | 44.9 | 5.94 | 32.2 | 29.5 |

C-C motif chemokine 14 X Serum Creatinine/(Weight adjusted urine output)

| | | | | | |
|---|---|---|---|---|---|
| AUC | 0.80 | 0.77 | 0.77 | 0.80 | 0.78 |
| SE | 0.025 | 0.026 | 0.029 | 0.024 | 0.026 |
| p Value | 0 | 0 | 0 | 0 | 0 |
| nCohort Non-persistent | 103 | 121 | 204 | 137 | 147 |
| nCohort Persistent | 218 | 198 | 117 | 184 | 172 |
| Cutoff Quartile 2 | 2.17 | 2.15 | 2.17 | 2.17 | 2.15 |
| Sensitivity | 85% | 85% | 90% | 87% | 87% |
| Specificity | 46% | 41% | 33% | 41% | 39% |
| Cutoff Quartile 3 | 10.2 | 10.1 | 10.2 | 10.2 | 10.1 |
| Sensitivity | 66% | 66% | 77% | 71% | 70% |
| Specificity | 83% | 76% | 65% | 77% | 73% |
| Cutoff Quartile 4 | 69.8 | 67.6 | 69.8 | 69.8 | 67.6 |
| Sensitivity | 35% | 36% | 43% | 41% | 41% |
| Specificity | 96% | 93% | 85% | 96% | 94% |
| OR Quartile 2 | 4.71 | 3.94 | 4.38 | 4.61 | 4.10 |
| p Value | 1.5E−8 | 4.1E−7 | 1.3E−5 | 4.6E−8 | 5.0E−7 |
| Lower limit of 95% CI | 2.75 | 2.32 | 2.25 | 2.67 | 2.37 |
| Upper limit of 95% CI | 8.04 | 6.71 | 8.50 | 7.97 | 7.11 |
| OR Quartile 3 | 9.00 | 6.20 | 6.24 | 8.23 | 6.57 |
| p Value | 1.1E−13 | 2.5E−12 | 4.1E−12 | 6.7E−16 | 5.8E−14 |
| Lower limit of 95% CI | 5.04 | 3.72 | 3.72 | 4.94 | 4.02 |
| Upper limit of 95% CI | 16.1 | 10.3 | 10.5 | 13.7 | 10.7 |
| OR Quartile 4 | 13.5 | 8.07 | 4.16 | 18.6 | 10.8 |
| p Value | 8.7E−7 | 1.2E−7 | 1.3E−7 | 1.1E−9 | 2.9E−10 |
| Lower limit of 95% CI | 4.79 | 3.72 | 2.45 | 7.26 | 5.15 |
| Upper limit of 95% CI | 38.1 | 17.5 | 7.07 | 47.6 | 22.6 |

C-C motif chemokine 14 X Cystatin-C/(Weight adjusted urine output)

| | | | | | |
|---|---|---|---|---|---|
| AUC | 0.77 | 0.75 | 0.76 | 0.79 | 0.76 |
| SE | 0.027 | 0.028 | 0.030 | 0.025 | 0.027 |
| p Value | 0 | 0 | 0 | 0 | 0 |
| nCohort Non-persistent | 94 | 109 | 194 | 127 | 135 |
| nCohort Persistent | 211 | 195 | 111 | 178 | 169 |
| Cutoff Quartile 2 | 1580000 | 1580000 | 1580000 | 1580000 | 1580000 |
| Sensitivity | 83% | 83% | 91% | 85% | 85% |
| Specificity | 44% | 39% | 34% | 39% | 37% |
| Cutoff Quartile 3 | 6710000 | 6570000 | 6710000 | 6710000 | 6570000 |
| Sensitivity | 64% | 65% | 76% | 69% | 69% |
| Specificity | 80% | 76% | 64% | 76% | 73% |
| Cutoff Quartile 4 | 3.85E7 | 3.84E7 | 3.85E7 | 3.85E7 | 3.84E7 |
| Sensitivity | 34% | 34% | 41% | 39% | 38% |
| Specificity | 94% | 91% | 84% | 94% | 92% |
| OR Quartile 2 | 3.89 | 3.20 | 5.21 | 3.80 | 3.24 |
| p Value | 1.1E−6 | 2.2E−5 | 6.0E−6 | 1.8E−6 | 2.4E−5 |
| Lower limit of 95% CI | 2.25 | 1.87 | 2.55 | 2.20 | 1.88 |
| Upper limit of 95% CI | 6.71 | 5.47 | 10.6 | 6.56 | 5.58 |
| OR Quartile 3 | 6.87 | 5.83 | 5.64 | 7.23 | 6.02 |
| p Value | 5.5E−11 | 6.7E−11 | 9.8E−11 | 7.4E−14 | 2.2E−12 |
| Lower limit of 95% CI | 3.86 | 3.43 | 3.34 | 4.31 | 3.65 |
| Upper limit of 95% CI | 12.2 | 9.90 | 9.52 | 12.1 | 9.93 |
| OR Quartile 4 | 7.44 | 5.07 | 3.72 | 11.1 | 7.05 |
| p Value | 6.9E−6 | 8.6E−6 | 1.7E−6 | 8.4E−9 | 2.9E−8 |
| Lower limit of 95% CI | 3.10 | 2.48 | 2.17 | 4.90 | 3.53 |
| Upper limit of 95% CI | 17.8 | 10.4 | 6.38 | 25.2 | 14.0 |

| | Persistence duration (hr) | | | |
|---|---|---|---|---|
| | 48 | 72 | | |
| | UO only | sCr or UO | sCr only | UO only |
| C-C motif chemokine 14 | | | | |
| AUC | 0.70 | 0.79 | 0.79 | 0.70 |
| SE | 0.032 | 0.024 | 0.025 | 0.032 |
| p Value | 1.1E−10 | 0 | 0 | 3.3E−10 |
| nCohort Non-persistent | 220 | 157 | 167 | 225 |
| nCohort Persistent | 109 | 174 | 162 | 104 |
| Cutoff Quartile 2 | 0.732 | 0.725 | 0.719 | 0.732 |
| Sensitivity | 89% | 90% | 91% | 89% |
| Specificity | 32% | 42% | 40% | 32% |
| Cutoff Quartile 3 | 1.64 | 1.64 | 1.64 | 1.64 |
| Sensitivity | 74% | 72% | 73% | 74% |
| Specificity | 62% | 74% | 72% | 61% |
| Cutoff Quartile 4 | 5.09 | 5.16 | 5.09 | 5.09 |
| Sensitivity | 37% | 41% | 43% | 38% |
| Specificity | 80% | 92% | 92% | 80% |
| OR Quartile 2 | 3.77 | 6.70 | 6.57 | 3.90 |
| p Value | 8.8E−5 | 3.1E−10 | 2.0E−9 | 1.0E−4 |
| Lower limit of 95% CI | 1.94 | 3.70 | 3.55 | 1.96 |
| Upper limit of 95% CI | 7.32 | 12.1 | 12.1 | 7.73 |
| OR Quartile 3 | 4.68 | 7.22 | 6.85 | 4.44 |
| p Value | 2.7E−9 | 1.6E−15 | 6.2E−15 | 1.3E−8 |
| Lower limit of 95% CI | 2.82 | 4.44 | 4.22 | 2.66 |
| Upper limit of 95% CI | 7.79 | 11.7 | 11.1 | 7.42 |
| OR Quartile 4 | 2.39 | 8.33 | 8.11 | 2.47 |
| p Value | 8.8E−4 | 3.4E−10 | 7.3E−11 | 6.0E−4 |
| Lower limit of 95% CI | 1.43 | 4.30 | 4.32 | 1.47 |
| Upper limit of 95% CI | 3.98 | 16.1 | 15.2 | 4.13 |
| Weight adjusted urine output | | | | |
| AUC | 0.29 | 0.37 | 0.41 | 0.30 |
| SE | 0.031 | 0.030 | 0.031 | 0.032 |
| p Value | 2.0E−11 | 7.2E−6 | 0.0028 | 1.1E−9 |
| nCohort Non-persistent | 224 | 159 | 170 | 229 |
| nCohort Persistent | 113 | 178 | 165 | 108 |
| Cutoff Quartile 2 | 0.189 | 0.189 | 0.192 | 0.189 |
| Sensitivity | 56% | 67% | 70% | 57% |
| Specificity | 15% | 16% | 20% | 17% |
| Cutoff Quartile 3 | 0.366 | 0.366 | 0.368 | 0.366 |
| Sensitivity | 33% | 42% | 44% | 33% |
| Specificity | 41% | 40% | 44% | 42% |
| Cutoff Quartile 4 | 0.857 | 0.857 | 0.862 | 0.857 |
| Sensitivity | 14% | 20% | 21% | 15% |
| Specificity | 69% | 69% | 71% | 70% |
| OR Quartile 2 | 0.225 | 0.376 | 0.592 | 0.268 |
| p Value | 2.0E−8 | 2.9E−4 | 0.041 | 5.9E−7 |
| Lower limit of 95% CI | 0.134 | 0.222 | 0.358 | 0.160 |
| Upper limit of 95% CI | 0.379 | 0.639 | 0.978 | 0.449 |
| OR Quartile 3 | 0.339 | 0.479 | 0.597 | 0.361 |
| p Value | 8.1E−6 | 9.3E−4 | 0.019 | 3.0E−5 |
| Lower limit of 95% CI | 0.211 | 0.310 | 0.387 | 0.224 |
| Upper limit of 95% CI | 0.545 | 0.741 | 0.919 | 0.582 |
| OR Quartile 4 | 0.371 | 0.569 | 0.665 | 0.403 |
| p Value | 0.0012 | 0.026 | 0.11 | 0.0031 |
| Lower limit of 95% CI | 0.203 | 0.346 | 0.403 | 0.221 |
| Upper limit of 95% CI | 0.675 | 0.936 | 1.10 | 0.736 |
| Serum Creatinine | | | | |
| AUC | 0.69 | 0.75 | 0.76 | 0.68 |
| SE | 0.030 | 0.026 | 0.025 | 0.031 |
| p Value | 7.0E−10 | 0 | 0 | 5.6E−9 |
| nCohort Non-persistent | 227 | 158 | 169 | 233 |
| nCohort Persistent | 125 | 196 | 183 | 119 |
| Cutoff Quartile 2 | 1.65 | 1.64 | 1.64 | 1.65 |
| Sensitivity | 88% | 89% | 91% | 88% |
| Specificity | 32% | 42% | 42% | 32% |
| Cutoff Quartile 3 | 2.44 | 2.44 | 2.44 | 2.44 |
| Sensitivity | 67% | 66% | 68% | 67% |
| Specificity | 59% | 70% | 70% | 58% |
| Cutoff Quartile 4 | 3.32 | 3.32 | 3.31 | 3.32 |
| Sensitivity | 41% | 37% | 39% | 40% |
| Specificity | 83% | 89% | 90% | 82% |
| OR Quartile 2 | 3.48 | 5.82 | 7.07 | 3.49 |
| p Value | 5.8E−5 | 2.2E−10 | 5.6E−11 | 8.2E−5 |
| Lower limit of 95% CI | 1.89 | 3.38 | 3.94 | 1.87 |
| Upper limit of 95% CI | 6.38 | 10.0 | 12.7 | 6.50 |
| OR Quartile 3 | 2.95 | 4.51 | 4.99 | 2.88 |
| p Value | 3.5E−6 | 5.3E−11 | 3.5E−12 | 7.7E−6 |
| Lower limit of 95% CI | 1.87 | 2.88 | 3.17 | 1.81 |
| Upper limit of 95% CI | 4.67 | 7.08 | 7.84 | 4.57 |
| OR Quartile 4 | 3.43 | 4.92 | 5.67 | 3.17 |
| p Value | 1.3E−6 | 7.4E−8 | 5.4E−9 | 5.7E−6 |
| Lower limit of 95% CI | 2.08 | 2.75 | 3.16 | 1.92 |
| Upper limit of 95% CI | 5.64 | 8.80 | 10.2 | 5.21 |
| Cystatin-C | | | | |
| AUC | 0.71 | 0.74 | 0.72 | 0.71 |
| SE | 0.033 | 0.028 | 0.029 | 0.033 |
| p Value | 5.8E−11 | 0 | 1.4E−14 | 2.1E−10 |
| nCohort Non-persistent | 205 | 142 | 150 | 209 |
| nCohort Persistent | 101 | 166 | 157 | 97 |
| Cutoff Quartile 2 | 1170000 | 1170000 | 1170000 | 1170000 |
| Sensitivity | 90% | 87% | 86% | 90% |
| Specificity | 33% | 38% | 36% | 32% |
| Cutoff Quartile 3 | 1660000 | 1660000 | 1650000 | 1660000 |
| Sensitivity | 72% | 67% | 67% | 72% |
| Specificity | 61% | 70% | 67% | 60% |
| Cutoff Quartile 4 | 2150000 | 2150000 | 2150000 | 2150000 |
| Sensitivity | 42% | 37% | 38% | 41% |
| Specificity | 83% | 89% | 88% | 82% |
| OR Quartile 2 | 4.42 | 4.02 | 3.45 | 4.10 |
| p Value | 4.7E−5 | 1.3E−6 | 1.5E−5 | 1.1E−4 |
| Lower limit of 95% CI | 2.16 | 2.29 | 1.97 | 2.01 |
| Upper limit of 95% CI | 9.03 | 7.05 | 6.05 | 8.40 |
| OR Quartile 3 | 4.07 | 4.94 | 4.16 | 3.94 |
| p Value | 1.1E−7 | 1.1E−10 | 4.4E−9 | 2.9E−7 |
| Lower limit of 95% CI | 2.43 | 3.04 | 2.58 | 2.33 |
| Upper limit of 95% CI | 6.84 | 8.02 | 6.70 | 6.64 |
| OR Quartile 4 | 3.46 | 4.58 | 4.41 | 3.26 |
| p Value | 6.1E−6 | 9.6E−7 | 7.7E−7 | 1.7E−5 |
| Lower limit of 95% CI | 2.02 | 2.49 | 2.45 | 1.90 |
| Upper limit of 95% CI | 5.92 | 8.41 | 7.96 | 5.59 |
| C-C motif chemokine 14/(Weight adjusted urine output) | | | | |
| AUC | 0.75 | 0.77 | 0.75 | 0.74 |
| SE | 0.030 | 0.026 | 0.027 | 0.031 |
| p Value | 0 | 0 | 0 | 4.2E−15 |
| nCohort Non-persistent | 219 | 155 | 165 | 224 |
| nCohort Persistent | 109 | 173 | 161 | 104 |
| Cutoff Quartile 2 | 1.26 | 1.26 | 1.24 | 1.26 |
| Sensitivity | 91% | 86% | 86% | 90% |
| Specificity | 33% | 37% | 36% | 32% |
| Cutoff Quartile 3 | 4.59 | 4.59 | 4.54 | 4.59 |
| Sensitivity | 74% | 70% | 70% | 74% |
| Specificity | 62% | 72% | 69% | 61% |
| Cutoff Quartile 4 | 24.3 | 24.3 | 22.9 | 24.3 |
| Sensitivity | 45% | 41% | 41% | 45% |
| Specificity | 85% | 93% | 90% | 84% |
| OR Quartile 2 | 4.85 | 3.71 | 3.34 | 4.45 |
| p Value | 1.3E−5 | 1.9E−6 | 1.4E−5 | 3.7E−5 |
| Lower limit of 95% CI | 2.39 | 2.16 | 1.94 | 2.19 |
| Upper limit of 95% CI | 9.85 | 6.37 | 5.76 | 9.05 |
| OR Quartile 3 | 4.74 | 6.06 | 5.11 | 4.49 |
| p Value | 2.1E−9 | 1.6E−13 | 1.1E−11 | 1.0E−8 |
| Lower limit of 95% CI | 2.85 | 3.75 | 3.19 | 2.69 |
| Upper limit of 95% CI | 7.89 | 9.78 | 8.18 | 7.51 |
| OR Quartile 4 | 4.60 | 9.11 | 6.47 | 4.45 |
| p Value | 1.5E−8 | 2.4E−10 | 1.4E−9 | 3.0E−8 |
| Lower limit of 95% CI | 2.71 | 4.60 | 3.54 | 2.63 |
| Upper limit of 95% CI | 7.81 | 18.1 | 11.8 | 7.55 |
| C-C motif chemokine 14 X Serum Creatinine | | | | |
| AUC | 0.72 | 0.81 | 0.81 | 0.72 |
| SE | 0.032 | 0.024 | 0.024 | 0.032 |
| p Value | 1.9E−12 | 0 | 0 | 8.3E−12 |
| nCohort Non-persistent | 216 | 152 | 162 | 221 |
| nCohort Persistent | 106 | 172 | 160 | 101 |
| Cutoff Quartile 2 | 1.30 | 1.29 | 1.29 | 1.30 |
| Sensitivity | 90% | 91% | 91% | 90% |
| Specificity | 32% | 43% | 41% | 32% |
| Cutoff Quartile 3 | 3.97 | 3.97 | 3.91 | 3.97 |
| Sensitivity | 76% | 73% | 74% | 76% |
| Specificity | 63% | 76% | 74% | 62% |
| Cutoff Quartile 4 | 18.0 | 18.3 | 16.8 | 18.0 |
| Sensitivity | 40% | 41% | 44% | 40% |
| Specificity | 82% | 93% | 93% | 81% |
| OR Quartile 2 | 4.14 | 7.28 | 7.35 | 4.31 |
| p Value | 4.9E−5 | 1.4E−10 | 5.8E−10 | 5.7E−5 |
| Lower limit of 95% CI | 2.08 | 3.97 | 3.91 | 2.11 |
| Upper limit of 95% CI | 8.22 | 13.4 | 13.8 | 8.77 |
| OR Quartile 3 | 5.51 | 8.27 | 8.29 | 5.23 |
| p Value | 2.2E−10 | 2.2E−16 | 0 | 1.1E−9 |
| Lower limit of 95% CI | 3.25 | 5.02 | 5.03 | 3.07 |
| Upper limit of 95% CI | 9.33 | 13.6 | 13.7 | 8.91 |
| OR Quartile 4 | 2.98 | 9.98 | 10.7 | 2.88 |
| p Value | 4.1E−5 | 2.1E−10 | 1.4E−11 | 7.5E−5 |
| Lower limit of 95% CI | 1.77 | 4.91 | 5.37 | 1.71 |
| Upper limit of 95% CI | 5.02 | 20.3 | 21.2 | 4.86 |
| C-C motif chemokine 14 X Cystatin-C | | | | |
| AUC | 0.73 | 0.81 | 0.81 | 0.73 |
| SE | 0.032 | 0.024 | 0.025 | 0.033 |
| p Value | 2.8E−12 | 0 | 0 | 2.3E−12 |
| nCohort Non-persistent | 205 | 142 | 150 | 209 |
| nCohort Persistent | 101 | 166 | 157 | 97 |
| Cutoff Quartile 2 | 958000 | 956000 | 955000 | 958000 |
| Sensitivity | 89% | 90% | 90% | 90% |
| Specificity | 32% | 43% | 41% | 32% |
| Cutoff Quartile 3 | 2880000 | 2880000 | 2860000 | 2880000 |
| Sensitivity | 76% | 73% | 73% | 76% |
| Specificity | 63% | 77% | 74% | 62% |
| Cutoff Quartile 4 | 1.03E7 | 1.04E7 | 1.03E7 | 1.03E7 |
| Sensitivity | 43% | 42% | 43% | 44% |
| Specificity | 83% | 94% | 94% | 84% |
| OR Quartile 2 | 3.88 | 7.06 | 6.04 | 4.10 |
| p Value | 1.2E−4 | 4.2E−10 | 8.0E−9 | 1.1E−4 |
| Lower limit of 95% CI | 1.95 | 3.82 | 3.28 | 2.01 |
| Upper limit of 95% CI | 7.75 | 13.0 | 11.1 | 8.40 |
| OR Quartile 3 | 5.45 | 8.88 | 7.79 | 5.29 |
| p Value | 7.0E−10 | 2.2E−16 | 2.2E−15 | 2.1E−9 |
| Lower limit of 95% CI | 3.18 | 5.29 | 4.69 | 3.07 |
| Upper limit of 95% CI | 9.33 | 14.9 | 13.0 | 9.13 |
| OR Quartile 4 | 3.73 | 11.9 | 12.0 | 4.10 |
| p Value | 1.7E−6 | 4.2E−10 | 6.2E−11 | 3.6E−7 |
| Lower limit of 95% CI | 2.17 | 5.48 | 5.69 | 2.38 |
| Upper limit of 95% CI | 6.40 | 25.9 | 25.2 | 7.06 |
| C-C motif chemokine 14 X Serum Creatinine/(Weight adjusted urine output) | | | | |
| AUC | 0.77 | 0.80 | 0.78 | 0.76 |
| SE | 0.030 | 0.024 | 0.026 | 0.031 |
| p Value | 0 | 0 | 0 | 0 |
| nCohort Non-persistent | 215 | 150 | 160 | 220 |
| nCohort Persistent | 106 | 171 | 159 | 101 |
| Cutoff Quartile 2 | 2.17 | 2.17 | 2.15 | 2.17 |
| Sensitivity | 90% | 88% | 88% | 89% |
| Specificity | 32% | 40% | 38% | 31% |
| Cutoff Quartile 3 | 10.2 | 10.2 | 10.1 | 10.2 |
| Sensitivity | 77% | 73% | 72% | 77% |
| Specificity | 63% | 75% | 71% | 62% |
| Cutoff Quartile 4 | 69.8 | 69.8 | 67.6 | 69.8 |
| Sensitivity | 44% | 43% | 43% | 45% |
| Specificity | 84% | 95% | 92% | 84% |
| OR Quartile 2 | 4.08 | 5.03 | 4.54 | 3.74 |
| p Value | 6.0E−5 | 2.7E−8 | 2.6E−7 | 1.7E−4 |
| Lower limit of 95% CI | 2.05 | 2.85 | 2.55 | 1.88 |
| Upper limit of 95% CI | 8.11 | 8.90 | 8.07 | 7.44 |
| OR Quartile 3 | 5.88 | 8.06 | 6.28 | 5.60 |
| p Value | 7.1E−11 | 2.2E−16 | 1.3E−13 | 3.8E−10 |
| Lower limit of 95% CI | 3.45 | 4.88 | 3.86 | 3.27 |
| Upper limit of 95% CI | 10.0 | 13.3 | 10.2 | 9.60 |
| OR Quartile 4 | 4.24 | 13.2 | 9.22 | 4.11 |
| p Value | 9.2E−8 | 6.2E−11 | 6.7E−11 | 1.8E−7 |
| Lower limit of 95% CI | 2.50 | 6.10 | 4.73 | 2.42 |
| Upper limit of 95% CI | 7.21 | 28.7 | 18.0 | 6.98 |
| C-C motif chemokine 14 X Cystatin-C/(Weight adjusted urine output) | | | | |
| AUC | 0.76 | 0.79 | 0.76 | 0.76 |
| SE | 0.031 | 0.026 | 0.027 | 0.032 |
| p Value | 0 | 0 | 0 | 4.4E−16 |
| nCohort Non-persistent | 204 | 140 | 148 | 208 |
| nCohort Persistent | 101 | 165 | 156 | 97 |
| Cutoff Quartile 2 | 1580000 | 1580000 | 1580000 | 1580000 |
| Sensitivity | 91% | 87% | 86% | 91% |
| Specificity | 33% | 39% | 36% | 32% |
| Cutoff Quartile 3 | 6710000 | 6710000 | 6570000 | 6710000 |
| Sensitivity | 77% | 72% | 71% | 77% |
| Specificity | 63% | 75% | 72% | 62% |
| Cutoff Quartile 4 | 3.85E7 | 3.85E7 | 3.84E7 | 3.85E7 |
| Sensitivity | 43% | 41% | 40% | 42% |
| Specificity | 83% | 93% | 91% | 83% |
| OR Quartile 2 | 5.00 | 4.08 | 3.50 | 4.65 |
| p Value | 2.3E−5 | 9.9E−7 | 1.2E−5 | 5.3E−5 |
| Lower limit of 95% CI | 2.37 | 2.32 | 2.00 | 2.21 |
| Upper limit of 95% CI | 10.5 | 7.17 | 6.13 | 9.79 |
| OR Quartile 3 | 5.83 | 7.53 | 6.04 | 5.68 |
| p Value | 2.3E−10 | 9.1E−15 | 1.2E−12 | 6.9E−10 |
| Lower limit of 95% CI | 3.38 | 4.52 | 3.67 | 3.27 |
| Upper limit of 95% CI | 10.1 | 12.5 | 9.91 | 9.87 |
| OR Quartile 4 | 3.71 | 8.89 | 6.31 | 3.50 |
| p Value | 1.9E−6 | 2.0E−9 | 1.4E−8 | 5.4E−6 |
| Lower limit of 95% CI | 2.16 | 4.35 | 3.34 | 2.04 |
| Upper limit of 95% CI | 6.36 | 18.2 | 11.9 | 6.00 |

### Example 9: Use of C-C Motif Chemokine 14 in Combination with Additional Markers for Evaluating Renal Status in Patients Admitted to the ICU: Persistent at KDIGO Stage 3

With the same study and patient cohort as in Examples 2 and 8 above, kidney status was assessed by the KDIGO criteria based on serum creatinine only, based on urine output only, or based on either serum creatinine or urine output. Two cohorts were defined to represent a “persistent” and a “non-persistent” population. “Persistent” indicates those patients whose minimum KDIGO stage during a period of 24, 48 or 72 hours was Stage 3 where the persistence period can start from the time of sample collection to 24, 48, or 72 hours after sample collection. “Non-persistent” indicates those patients who were not persistent at Stage 3 and whose minimum KDIGO stage during a period of 24, 48 or 72 hours was Stage 2 or below where the persistence period can start from the time of sample collection to 24, 48, or 72 hours after sample collection. If a patient reached KDIGO stage 3 and died or was placed on renal replacement therapy (RRT) during the persistence period, the patient was considered “persistent.”

Predictive ability of the biomarkers, including all combinations of two markers for persistent AKI were assessed using the area under the receiver operating characteristic (ROC) curve. Confidence intervals of the area under the ROC curve (AUC) and pair-wise comparisons of AUC were calculated by the Delong method. Occurrence of MAKE₉₀ was compared across CCL14 tertiles using the Cochran-Armitage test. The cumulative incidence curves for MAKE₉₀ the composite of RRT initiation or death were estimated by the Kaplan-Meier method, and a log-rank test was used to compare the groups. Statistical analyses were performed using R 3.5.1. Two-sided p-values <0.05 were considered statistically significant.

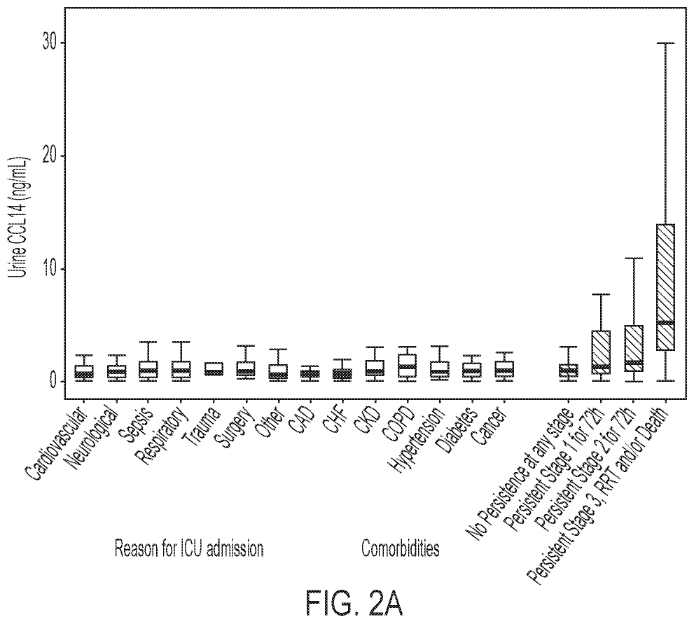

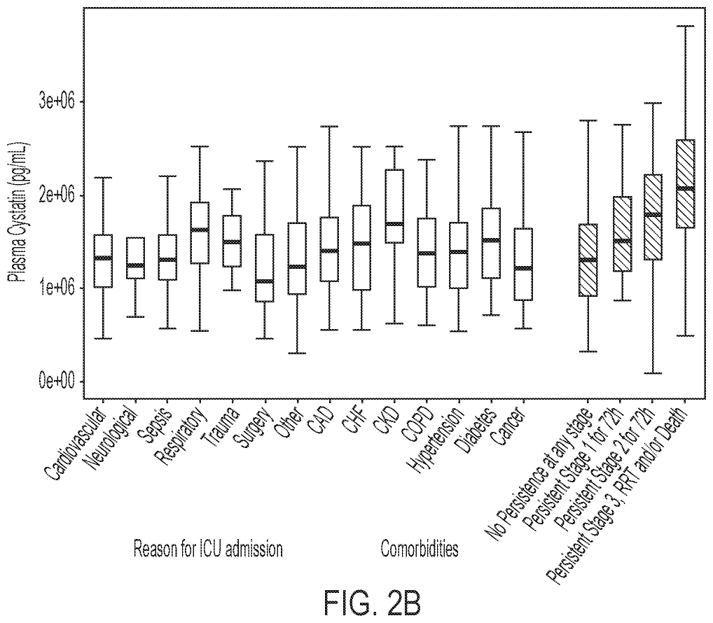

FIGS. 2A and 2B depict the measured concentrations of urinary C-C motif chemokine 14 (FIG. 2A) and plasma cystatin C (FIG. 2B) across various groups of patients. Open boxes indicate marker levels for patients having various acute and chronic conditions who did not persist at any stage of acute kidney injury. Shaded boxes indicate marker levels for patients that maintained a minimum KDIGO stage of 1, 2, or 3 acute level kidney injury over a persistence period of at least 72 hours beginning within a progression window of 48 hours from enrollment. Both CCL14 and cystatin C demonstrated increasing concentrations with increasing stages of acute kidney injury. Both CCL14 and cystatin C were determined to be “positive going” for persistent acute kidney injury. Urinary C-C motif chemokine 14 levels in patients who did not persist at any stage of acute kidney injury were similar among patients experiencing the different comorbid conditions indicated in FIG. 2A.

Urinary C-C motif chemokine ligand 14 demonstrated predictability of persistent stage 3 acute kidney injury lasting at least 72 hours over a persistence period beginning within 48 hours of enrollment with an area under the receiver operating characteristic curve (AUC) (95% CI) of 0.83 (0.78-0.87). A sensitivity analysis on the definition of acute kidney injury persistence demonstrated consistent performance of C-C motif chemokine 14 across persistence periods of 24, 48, and 72 hours duration, wherein the persistence periods begin within progression windows of 24, 48, and 72 hours following enrollment. The ROC AUC (95% CI) ranged from 0.79 (0.74-0.84) for 24-hour persistence and progression within 24 hours to 0.84 (0.79-0.88) for 48-hour persistence and progression within 48 hours. Moreover, a sensitivity analysis in which the 53 patients retrospectively determined not to be at KDIGO stage 2-3 at enrollment (Table 1) were excluded showed little change in the results for persistent KDIGO stage 3 acute kidney injury lasting over a persistence period of at least 72 hours beginning within 48 hours of enrollment. (AUC (95% CI)=0.81 (0.76−0.86)).

TABLE 25
ROC AUC (95% CI) of urinary C-C motif chemokine 14 for
prediction of persistent KDIGO stage 3 acute kidney injury
of different durations and different windows for progression
to persistent KDIGO stage 3 for those patients enrolled
with KDIGO stage 2 acute kidney injury.

| Persistence | Progression | | Urine CCL14 AUC |
|---|---|---|---|
| Duration (h) | Window (h) | % Persistent | (95% CI) |
| 24 | 24 | 37% | 0.791 (0.740-0.843) |
| 24 | 48 | 41% | 0.799 (0.749-0.849) |
| 24 | 72 | 45% | 0.791 (0.741-0.842) |
| 48 | 24 | 33% | 0.829 (0.781-0.877) |
| 48 | 48 | 36% | 0.837 (0.791-0.883) |
| 48 | 72 | 40% | 0.823 (0.776-0.871) |
| 72 | 24 | 30% | 0.808 (0.757-0.859) |
| 72 | 48 | 33% | 0.826 (0.778-0.873) |
| 72 | 72 | 37% | 0.805 (0.755-0.855) |

TABLE 26
Comparison of ROC AUC (95% CI) for urinary CCL14
and plasma cystatin C with respect to prediction
of persistent stage KDIGO stage 3 acute kidney injury
lasting at least 72 hours over a persistence period
beginning within 48 hours of enrollment.

| | | | P-value for AUC |
|---|---|---|---|
| | | | difference between |
| | | | urine CCL14 and |
| Biomarker | N | AUC (95% CI) | biomarker |
| Urine CCL14 | 331 | 0.83 (0.78-0.87) | NA |
| Plasma Cystatin C | 308 | 0.75 (0.69-0.80) | 0.047 |

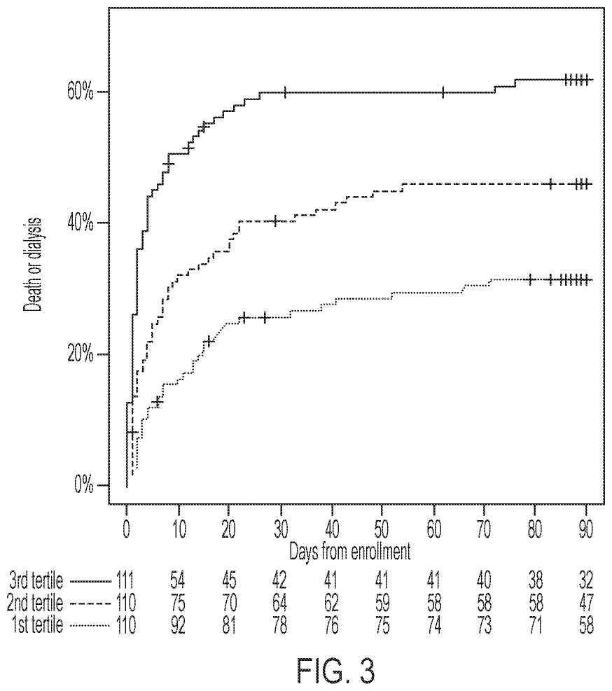

FIG. 3 depicts the composite percentage of patients beginning renal replacement therapy (RRT) or suffering death over 90 days following enrollment for each of the three tertiles defined by urinary C-C motif chemokine 14 measurements in the enrollment samples. Development of the composite endpoint increased across tertiles (log-rank p<0.001) and approximately doubled from about 30% to about 60% between the first tertile and the third tertile. As shown in FIG. 3, most qualifying events occurred within the 30 days following enrollment. Incidence of MAKE₉₀ endpoints, including loss in eGFR greater than or equal to 25%, (not shown) was 29%, 41%, and 46% for the first, second, and third tertiles of C-C motif chemokine 14 measurements, respectively (p=0.01).

The ability of C-C motif chemokine ligand 14 to enhance the predictability of persistent KDIGO stage 3 acute kidney injury over other clinical variables was tested using a multivariable logistic regression model. Urinary C-C motif chemokine ligand 14 significantly improved risk prediction when added to a five-parameter clinical model for persistent KDIGO stage 3 acute kidney injury persisting at least 72 hours over a persistence period beginning within 48 hours of enrollment using any of ROC AUC, integrated discrimination improvement (IDI), and category free net reclassification improvement (cfNRI) analyses.

TABLE 27
Comparison between odds ratios and p-values for clinical parameters
of a 5-parameter reference logistic regression model and the same
model updated to include urinary C-C motif chemokine ligand 14.

| | Reference Model | | New Model with Urine CCL14 | |
|---|---|---|---|---|
| | AUC = 0.85 (0.81-0.89) | | AUC = 0.89 (0.85-0.93) | |
| Variable | Odds Ratio | P-value | Odds Ratio | P-value |
| Body mass index | 0.76 (0.54-1.08) | 0.130 | 0.80 (0.55-1.17) | 0.26 |
| Non-renal APACHE III score | 1.45 (1.08-1.95) | 0.014 | 1.40 (1.02-1.94) | 0.04 |
| Serum creatinine trajectory | 1.53 (1.14-2.07) | 0.005 | 1.37 (0.99-1.89) | 0.06 |
| AKI stage at enrollment | 5.28 (3.39-8.21) | <0.001 | 4.11 (2.56-6.57) | <0.001 |
| Diabetes | 0.62 (0.32-1.19) | 0.15 | 0.49 (0.24-1.00) | 0.05 |
| Urine CCL14 | Not Included | NA | 5.53 (3.03-10.08) | <0.001 |

Difference between AUCs of the reference model and new model were statistically significant (p=0.015). Serum creatinine trajectory was measured as a change in serum creatinine concentration over the prior day as determined using two serum creatinine results with mean (±SD) collection times at 18 (±9) hours and 7 (±4) hours prior to enrollment. Urinary CCL14 concentrations were log-transformed. All numeric variables were standardized by subtracting the mean and dividing by the standard deviation. Out of a total of 308 patients, 34% were classified as persistent.

TABLE 28
Analyses of the new logistic regression model relative to the
reference model of Table 27 using ROC AUC, integrated discrimination
improvement (IDI), and category free net reclassification
improvement (cfNRI) analyses, where an event is persistent
KDIGO stage 3 acute kidney injury lasting at least 72 hours
over a persistence period beginning within 48 hours of enrollment
and a non-event is non-persistent acute kidney injury defined
according to the same parameters.

| | Value | 95% CI | P-value |
|---|---|---|---|
| IDI | 0.108 | 0.071-0.145 | <0.001 |
| IDI_event | 0.071 | 0.038-0.104 | <0.001 |
| IDI_non_event | 0.037 | 0.019-0.055 | <0.001 |
| cfNRI | 0.811 | 0.575-1.046 | <0.001 |
| cfNRI_event | 0.352 | 0.161-0.544 | <0.001 |
| cfNRI_non_event | 0.458 | 0.321-0.596 | <0.001 |
| AUC_ref_model | 0.852 | 0.808-0.893 | <0.001 |
| AUC_new_model | 0.894 | 0.854-0.925 | <0.001 |
| AUC difference | 0.042 | 0.014-0.069 | 0.003 |

Combinations of C-C motif chemokine ligand 14 with plasma cystatin C and with serum creatinine via a logistic regression model significantly improved the AUC for prediction of persistent acute kidney injury lasting at least 72 hours over a persistence period beginning within 48 hours of enrollment (AUC increase=0.028, p=0.04, for plasma cystatin C and AUC increase=0.04, p<0.01, for serum creatinine).

TABLE 29
ROC AUC difference (95% CI) between 2-marker combinations of urinary C-C motif
chemokine ligand 14 and plasma cystatin C or serum creatinine and urinary C-C
motif chemokine ligand 14 alone. Marker concentrations were log-transformed.

| | | AUC (95% CI) | | AUC (95% CI) | | | | |
|---|---|---|---|---|---|---|---|---|
| | | of CCL14 for | | of CCL14 and | | AUC | | P-value for |
| | | Matched | | Biomarker | | Difference | | AUC |
| Biomarker | N | Samples | | Combination* | | (95% C %) | | Difference |
| Plasma Cystatin C | 308 | 0.814 | (0.764-0.865) | 0.843 | (0.796-0.889) | 0.028 | (0.001-0.056) | 0.04 |
| Serum Creatinine | 324 | 0.82 | (0.78-0.87) | 0.87 | (0.83-0.91) | 0.04 | (0.01-0.07) | <0.01 |

TABLE 30
Sensitivity, specificity and odds ratio at probability cutoffs
ranging from 0.1 to 0.9 in 0.1 intervals from the logistic
regression models of urinary C-C motif chemokine ligand
14 with plasma cystatin C or serum creatinine.

| | | | |
|---|---|---|---|
| Cutoff | Sensitivity | Specificity | Odds Ratio |

Urinary C-C motif chemokine 14 with plasma cystatin C

| | | | | |
|---|---|---|---|---|
| 0.1 | 0.95 (0.91-0.99) | 0.37 (0.30-0.43) | 12 | (4.5-30) |
| 0.2 | 0.91 (0.86-0.97) | 0.59 (0.52-0.66) | 15 | (7.2-32) |
| 0.3 | 0.84 (0.77-0.91) | 0.72 (0.65-0.78) | 13 | (7.1-24) |
| 0.4 | 0.73 (0.65-0.82) | 0.80 (0.75-0.86) | 11 | (6.4-19) |
| 0.5 | 0.60 (0.50-0.69) | 0.89 (0.84-0.93) | 12 | (6.5-21) |
| 0.6 | 0.45 (0.36-0.55) | 0.91 (0.87-0.95) | 8.5 | (4.6-16) |
| 0.7 | 0.33 (0.24-0.42) | 0.96 (0.93-0.99) | 12 | (5.3-27) |
| 0.8 | 0.19 (0.12-0.27) | 0.97 (0.94-0.99) | 6.7 | (2.7-16) |
| 0.9 | 0.03 (0.00-0.06) | 1.00 (0.99-1.00) | 6.0 | (0.62-59) |

Urinary C-C motif chemokine 14 with serum creatinine

| | | | | |
|---|---|---|---|---|
| 0.1 | 0.96 (0.93-1.00) | 0.43 (0.36-0.49) | 20 | (7.0-55) |
| 0.2 | 0.89 (0.83-0.95) | 0.65 (0.59-0.71) | 15 | (7.8-29) |
| 0.3 | 0.83 (0.75-0.90) | 0.76 (0.70-0.82) | 15 | (8.3-27) |
| 0.4 | 0.75 (0.67-0.83) | 0.80 (0.75-0.85) | 12 | (7.0-21) |
| 0.5 | 0.65 (0.56-0.74) | 0.87 (0.82-0.91) | 12 | (7.1-22) |
| 0.6 | 0.54 (0.45-0.63) | 0.93 (0.90-0.96) | 16 | (8.2-30) |
| 0.7 | 0.44 (0.35-0.53) | 0.96 (0.93-0.98) | 18 | (8.4-39) |
| 0.8 | 0.26 (0.17-0.34) | 0.99 (0.97-1.00) | 24 | (7.2-83) |
| 0.9 | 0.11 (0.05-0.17) | 1.00 (0.99-1.00) | 26 | (3.4-206) |

Elevated urinary C-C motif chemokine 14 was shown to predict the development of persistent acute kidney injury in a large heterogeneous cohort of critically ill patients with KDIGO stage 2 or 3 acute kidney injury. These finding support the role of chemokine signaling and macrophage trafficking in renal damage and repair and indicate that C-C motif chemokine 14 may be an important mediator of renal tissue damage and/or non-recovery. Acute kidney injury is associated renal inflammation. During renal inflammation, circulating monocytes are recruited, become activated and differentiate into macrophages. Both glomerular and interstitial macrophage infiltration can be observed following injury. Macrophages are believed to play a diverse role in kidney damage and repair (Meng X M, Tang P M, Li J, Lan H Y. Kidney Dis (Basel). 2015; 1(2):138-46). In response to tissue injury, macrophages become activated with the functional state of the macrophage depending on the stage of tissue injury and repair. Therefore, macrophages may contribute both to tissue injury and repair after acute kidney injury (Huen S C, Cantley L G. Annu Rev Physiol. 2017; 79:449-69). Such macrophage polarization is dictated by the immediate microenvironment and appears to play a critical role in the recovery of renal function after acute kidney injury.

### Example 10. Use of C-C Motif Chemokine 14 and One or More of Serum Creatinine, Urine Output, and Cystatin C in a Product Model for Evaluating Renal Status in Patients Admitted to the ICU: RIFLE Stage I or F or KDIGO Stage 2 or 3

Acutely ill patients from the intensive care unit (ICU) who are believed to be at risk of developing acute kidney injury were enrolled in the following study. Patients were excluded if they had a prior kidney transplant, a known moderate to severe AKI prior to enrollment (e.g., RIFLE stage I or RIFLE stageF/AKIN stage II or AKIN stage III/KDIGO stage 2 or KDIGO stage 3), were receiving or were in imminent need of renal replacement therapy, or had known infection with human immunodeficiency virus or active hepatitis. Patients were a subset of the population studied in Kashani K., et al. Crit Care. 2013; 17(1):R25, herein incorporated by reference in its entirety. Blood samples (10 mL for EDTA plasma and 3 mL for serum) and urine samples (50 mL) were collected from each patient at enrollment, and at every 12 hours up to day 4, and then every 24 hours thereafter up to day 7 while the subject was hospitalized. Analyte concentrations and urine output were measured in urine and/or blood sample collections at the times indicated in Tables 31-33 below.

Renal status was assessed by both the RIFLE and KDIGO criteria based on serum creatinine or urine output, serum creatinine only or urine output only. Two cohorts were defined to represent a “diseased” and a “non-diseased” population. “Diseased” indicates those patients who achieved the specified maximum RIFLE or KDIGO stage within the specified time period. The time “prior to AKI stage” represents the time at which a sample was collected, relative to the time a particular patient reached the lowest disease stage as defined for that cohort, binned into three groups which are +/−12 hours. For example, “24 hr prior” which used 0 vs R, I, F as the two cohorts would mean 24 hr (+/−12 hours) prior to reaching stage R (or I if no sample at R, or F if no sample at R or I).

The ability to distinguish between the “diseased” and “non-diseased” cohorts was determined using a ROC analysis. Additionally, sensitivity and specificity were determined at specific values (70%, 80% or 90%) or at cutoffs corresponding to the 25^{th}, 50^{th} and 75^{th} percentiles (or Quartile 2, Quartile 3 and Quartile 4) of C-C motif chemokine, serum creatinine and cystatin C concentrations and weight adjusted urine output.

The individual marker results were combined to provide a single result as indicated herein: C-C motif chemokine 14×Serum creatinine/(Weight adjusted urine output) and C-C motif chemokine 14×Cystatin C/(Weight adjusted urine output). The single result was then treated as an individual biomarker using standard statistical methods. In expressing these combinations, the arithmetic operators such as “X” (multiplication) and “I” (division) are used in their ordinary mathematical sense.

TABLE 31
AUC comparison between the “non-diseased” cohort (patients that did not progress beyond RIFLE stage 0 or R) and
the “diseased” cohort (patients that progressed to stage I or F, at 0, 24 hours, and 48 hours prior to reaching stage I or F).

| 0 hr prior to AKI stage | | | 24 hr prior to AKI stage | | | 48 hr prior to AKI stage | | |
|---|---|---|---|---|---|---|---|---|
| sCr or UO | sCr only | UO only | sCr or UO | sCr only | UO only | sCr or UO | sCr only | UO only |

C-C motif chemokine 14

| | | | | | | | | | |
|---|---|---|---|---|---|---|---|---|---|
| AUC | 0.71 | 0.79 | 0.66 | 0.60 | 0.72 | 0.55 | 0.58 | 0.69 | 0.54 |
| SE | 0.023 | 0.036 | 0.025 | 0.028 | 0.043 | 0.029 | 0.036 | 0.053 | 0.037 |
| p | 0 | 3.3E−15 | 5.7E−11 | 3.6E−4 | 1.4E−7 | 0.066 | 0.018 | 2.9E−4 | 0.33 |
| nCohort 1 | 951 | 1433 | 1089 | 951 | 1433 | 1089 | 951 | 1433 | 1089 |
| nCohort 2 | 179 | 57 | 153 | 128 | 47 | 115 | 74 | 32 | 66 |
| Cutoff 1 | 0.522 | 0.731 | 0.516 | 0.361 | 0.561 | 0.356 | 0.336 | 0.561 | 0.336 |
| Sens 1 | 70% | 70% | 71% | 70% | 70% | 70% | 70% | 72% | 71% |
| Spec 1 | 60% | 71% | 55% | 42% | 59% | 38% | 38% | 59% | 35% |
| Cutoff 2 | 0.398 | 0.522 | 0.403 | 0.275 | 0.490 | 0.275 | 0.303 | 0.527 | 0.297 |
| Sens 2 | 80% | 81% | 80% | 80% | 81% | 80% | 81% | 81% | 80% |
| Spec 2 | 47% | 55% | 44% | 29% | 51% | 27% | 32% | 55% | 29% |
| Cutoff 3 | 0.270 | 0.376 | 0.282 | 0.183 | 0.275 | 0.183 | 0.205 | 0.320 | 0.189 |
| Sens 3 | 91% | 91% | 90% | 91% | 91% | 90% | 91% | 91% | 91% |
| Spec 3 | 28% | 38% | 27% | 17% | 25% | 16% | 20% | 31% | 16% |
| Cutoff 4 | 0.632 | 0.720 | 0.717 | 0.632 | 0.720 | 0.717 | 0.632 | 0.720 | 0.717 |
| Sens 4 | 62% | 70% | 58% | 45% | 55% | 37% | 35% | 47% | 29% |
| Spec 4 | 70% | 70% | 70% | 70% | 70% | 70% | 70% | 70% | 70% |
| Cutoff 5 | 0.885 | 1.01 | 1.11 | 0.885 | 1.01 | 1.11 | 0.885 | 1.01 | 1.11 |
| Sens 5 | 49% | 65% | 42% | 28% | 47% | 25% | 27% | 34% | 23% |
| Spec 5 | 80% | 80% | 80% | 80% | 80% | 80% | 80% | 80% | 80% |
| Cutoff 6 | 1.39 | 1.66 | 2.77 | 1.39 | 1.66 | 2.77 | 1.39 | 1.66 | 2.77 |
| Sens 6 | 37% | 53% | 18% | 21% | 43% | 11% | 16% | 28% | 9% |
| Spec 6 | 90% | 90% | 90% | 90% | 90% | 90% | 90% | 90% | 90% |

C-C motif chemokine 14 with serum creatinine and weight adjusted urine output

| | | | | | | | | | |
|---|---|---|---|---|---|---|---|---|---|
| AUC | 0.83 | 0.81 | 0.79 | 0.68 | 0.75 | 0.62 | 0.61 | 0.68 | 0.55 |
| SE | 0.020 | 0.035 | 0.023 | 0.029 | 0.042 | 0.030 | 0.037 | 0.056 | 0.038 |
| p | 0 | 0 | 0 | 2.9E−10 | 1.3E−9 | 4.4E−5 | 0.0040 | 0.0015 | 0.18 |
| nCohort 1 | 808 | 1253 | 948 | 808 | 1253 | 948 | 808 | 1253 | 948 |
| nCohort 2 | 167 | 56 | 142 | 118 | 46 | 105 | 71 | 29 | 63 |
| Cutoff 1 | 1.16 | 1.98 | 1.29 | 0.535 | 1.12 | 0.549 | 0.228 | 0.631 | 0.226 |
| Sens 1 | 70% | 71% | 70% | 70% | 72% | 70% | 70% | 72% | 71% |
| Spec 1 | 79% | 78% | 74% | 60% | 68% | 55% | 40% | 53% | 36% |
| Cutoff 2 | 0.758 | 0.947 | 0.781 | 0.330 | 0.644 | 0.363 | 0.147 | 0.255 | 0.143 |
| Sens 2 | 80% | 80% | 80% | 81% | 80% | 80% | 80% | 83% | 81% |
| Spec 2 | 67% | 64% | 62% | 48% | 53% | 45% | 30% | 33% | 26% |
| Cutoff 3 | 0.432 | 0.556 | 0.535 | 0.107 | 0.175 | 0.164 | 0.0649 | 0.0715 | 0.0649 |
| Sens 3 | 90% | 91% | 90% | 91% | 91% | 90% | 90% | 93% | 90% |
| Spec 3 | 55% | 49% | 54% | 23% | 26% | 29% | 17% | 14% | 15% |
| Cutoff 4 | 0.824 | 1.23 | 1.09 | 0.824 | 1.23 | 1.09 | 0.824 | 1.23 | 1.09 |
| Sens 4 | 77% | 77% | 73% | 54% | 70% | 37% | 48% | 62% | 37% |
| Spec 4 | 70% | 70% | 70% | 70% | 70% | 70% | 70% | 70% | 70% |
| Cutoff 5 | 1.26 | 2.35 | 2.18 | 1.26 | 2.35 | 2.18 | 1.26 | 2.35 | 2.18 |
| Sens 5 | 69% | 68% | 59% | 40% | 63% | 27% | 32% | 41% | 21% |
| Spec 5 | 80% | 80% | 80% | 80% | 80% | 80% | 80% | 80% | 80% |
| Cutoff 6 | 3.12 | 5.53 | 6.05 | 3.12 | 5.53 | 6.05 | 3.12 | 5.53 | 6.05 |
| Sens 6 | 51% | 54% | 42% | 24% | 46% | 13% | 18% | 34% | 13% |
| Spec 6 | 90% | 90% | 90% | 90% | 90% | 90% | 90% | 90% | 90% |

C-C motif chemokine 14 X Cystatin-C/(Weight adjusted urine output)

| | | | | | | | | | |
|---|---|---|---|---|---|---|---|---|---|
| AUC | 0.85 | 0.82 | 0.77 | 0.63 | 0.73 | 0.53 | 0.54 | 0.59 | 0.47 |
| SE | 0.021 | 0.037 | 0.027 | 0.034 | 0.048 | 0.034 | 0.044 | 0.068 | 0.045 |
| p | 0 | 0 | 0 | 2.2E−4 | 1.2E−6 | 0.43 | 0.35 | 0.20 | 0.45 |
| nCohort 1 | 325 | 640 | 433 | 325 | 640 | 433 | 325 | 640 | 433 |
| nCohort 2 | 140 | 50 | 117 | 94 | 37 | 87 | 52 | 20 | 47 |
| Cutoff 1 | 4970000 | 7990000 | 5400000 | 963000 | 3380000 | 1120000 | 511000 | 1180000 | 511000 |
| Sens 1 | 70% | 70% | 70% | 70% | 70% | 70% | 71% | 70% | 70% |
| Spec 1 | 81% | 77% | 71% | 51% | 63% | 44% | 32% | 40% | 25% |
| Cutoff 2 | 2650000 | 4770000 | 2730000 | 643000 | 1850000 | 643000 | 398000 | 909000 | 398000 |
| Sens 2 | 80% | 80% | 80% | 81% | 81% | 80% | 81% | 80% | 81% |
| Spec 2 | 71% | 68% | 60% | 37% | 51% | 30% | 27% | 36% | 22% |
| Cutoff 3 | 1610000 | 2400000 | 1580000 | 240000 | 648000 | 198000 | 114000 | 362000 | 86700 |
| Sens 3 | 90% | 90% | 91% | 90% | 92% | 91% | 90% | 90% | 91% |
| Spec 3 | 62% | 56% | 51% | 19% | 28% | 14% | 12% | 18% | 8% |
| Cutoff 4 | 2510000 | 5070000 | 5230000 | 2510000 | 5070000 | 5230000 | 2510000 | 5070000 | 5230000 |
| Sens 4 | 83% | 76% | 70% | 43% | 65% | 28% | 33% | 40% | 23% |
| Spec 4 | 70% | 70% | 70% | 70% | 70% | 70% | 70% | 70% | 70% |
| Cutoff 5 | 3940000 | 1.01E7 | 1.20E7 | 3940000 | 1.01E7 | 1.20E7 | 3940000 | 1.01E7 | 1.20E7 |
| Sens 5 | 76% | 64% | 58% | 33% | 51% | 15% | 23% | 35% | 17% |
| Spec 5 | 80% | 80% | 80% | 80% | 80% | 80% | 80% | 80% | 80% |
| Cutoff 6 | 8840000 | 2.79E7 | 6.37E7 | 8840000 | 2.79E7 | 6.37E7 | 8840000 | 2.79E7 | 6.37E7 |
| Sens 6 | 60% | 48% | 34% | 20% | 35% | 3% | 13% | 20% | 4% |
| Spec 6 | 90% | 90% | 90% | 90% | 90% | 90% | 90% | 90% | 90% |

TABLE 32
AUC comparison between the “non-diseased” cohort (patients that did not progress beyond RIFLE stage 0) using
the maximum marker level during the study and the “diseased” cohort (patients that progressed to stage F)
using the maximum marker level from samples collected between enrollment and 0, 24 hours, and 48 hours prior to reaching stage F.

| 0 hr prior to AKI stage | | | 24 hr prior to AKI stage | | | 48 hr prior to AKI stage | | |
|---|---|---|---|---|---|---|---|---|
| sCr or UO | sCr only | UO only | sCr or UO | sCr only | UO only | sCr or UO | sCr only | UO only |

C-C motif chemokine 14

| | | | | | | | | | |
|---|---|---|---|---|---|---|---|---|---|
| AUC | 0.85 | 0.85 | 0.84 | 0.84 | 0.86 | 0.82 | 0.68 | 0.69 | 0.61 |
| SE | 0.037 | 0.047 | 0.045 | 0.042 | 0.049 | 0.051 | 0.066 | 0.073 | 0.083 |
| p | 0 | 7.5E−14 | 2.4E−14 | 2.2E−16 | 2.9E−13 | 4.4E−10 | 0.0057 | 0.0094 | 0.20 |
| nCohort 1 | 137 | 315 | 172 | 137 | 315 | 172 | 137 | 315 | 172 |
| nCohort 2 | 47 | 28 | 32 | 38 | 24 | 27 | 22 | 17 | 14 |
| Cutoff 1 | 2.51 | 2.77 | 2.37 | 1.82 | 2.39 | 1.89 | 0.701 | 0.717 | 0.701 |
| Sens 1 | 70% | 71% | 72% | 71% | 71% | 70% | 73% | 71% | 71% |
| Spec 1 | 90% | 92% | 86% | 88% | 89% | 83% | 58% | 51% | 55% |
| Cutoff 2 | 1.10 | 0.774 | 1.23 | 1.13 | 1.14 | 1.23 | 0.505 | 0.554 | 0.382 |
| Sens 2 | 81% | 82% | 81% | 82% | 83% | 81% | 82% | 82% | 86% |
| Spec 2 | 74% | 53% | 74% | 76% | 72% | 74% | 36% | 35% | 22% |
| Cutoff 3 | 0.579 | 0.579 | 0.740 | 0.548 | 0.717 | 0.653 | 0.382 | 0.482 | 0.248 |
| Sens 3 | 91% | 93% | 91% | 92% | 92% | 93% | 91% | 94% | 93% |
| Spec 3 | 45% | 37% | 57% | 44% | 51% | 52% | 23% | 27% | 8% |
| Cutoff 4 | 1.06 | 1.10 | 1.10 | 1.06 | 1.10 | 1.10 | 1.06 | 1.10 | 1.10 |
| Sens 4 | 81% | 79% | 88% | 82% | 83% | 85% | 50% | 53% | 43% |
| Spec 4 | 70% | 70% | 70% | 70% | 70% | 70% | 70% | 70% | 70% |
| Cutoff 5 | 1.32 | 1.49 | 1.57 | 1.32 | 1.49 | 1.57 | 1.32 | 1.49 | 1.57 |
| Sens 5 | 77% | 79% | 78% | 76% | 75% | 74% | 50% | 53% | 43% |
| Spec 5 | 80% | 80% | 80% | 80% | 80% | 80% | 80% | 80% | 80% |
| Cutoff 6 | 2.60 | 2.51 | 2.77 | 2.60 | 2.51 | 2.77 | 2.60 | 2.51 | 2.77 |
| Sens 6 | 68% | 71% | 59% | 61% | 67% | 44% | 36% | 41% | 29% |
| Spec 6 | 91% | 90% | 90% | 91% | 90% | 90% | 91% | 90% | 90% |

C-C motif chemokine 14 with serum creatinine and weight adjusted urine output

| | | | | | | | | | |
|---|---|---|---|---|---|---|---|---|---|
| AUC | 0.90 | 0.88 | 0.90 | 0.86 | 0.86 | 0.85 | 0.76 | 0.74 | 0.71 |
| SE | 0.031 | 0.043 | 0.037 | 0.040 | 0.049 | 0.048 | 0.062 | 0.071 | 0.080 |
| p | 0 | 0 | 0 | 0 | 1.0E−13 | 7.6E−13 | 3.7E−5 | 8.7E−4 | 0.0096 |
| nCohort 1 | 134 | 310 | 171 | 134 | 310 | 171 | 134 | 310 | 171 |
| nCohort 2 | 47 | 28 | 32 | 38 | 24 | 27 | 22 | 17 | 14 |
| Cutoff 1 | 15.9 | 18.6 | 19.6 | 4.63 | 6.07 | 5.04 | 1.75 | 2.06 | 1.75 |
| Sens 1 | 70% | 71% | 72% | 71% | 71% | 70% | 73% | 71% | 71% |
| Spec 1 | 96% | 92% | 95% | 91% | 84% | 88% | 76% | 65% | 70% |
| Cutoff 2 | 4.05 | 4.71 | 5.04 | 1.99 | 2.78 | 2.57 | 0.636 | 0.966 | 0.538 |
| Sens 2 | 81% | 82% | 81% | 82% | 83% | 81% | 82% | 82% | 86% |
| Spec 2 | 87% | 80% | 88% | 78% | 71% | 75% | 50% | 45% | 39% |
| Cutoff 3 | 1.36 | 1.36 | 3.46 | 0.755 | 1.98 | 0.891 | 0.512 | 0.678 | 0.512 |
| Sens 3 | 91% | 93% | 91% | 92% | 92% | 93% | 91% | 94% | 93% |
| Spec 3 | 72% | 57% | 78% | 52% | 65% | 53% | 43% | 32% | 39% |
| Cutoff 4 | 1.23 | 2.62 | 1.84 | 1.23 | 2.62 | 1.84 | 1.23 | 2.62 | 1.84 |
| Sens 4 | 91% | 86% | 94% | 84% | 83% | 85% | 73% | 59% | 64% |
| Spec 4 | 70% | 70% | 70% | 70% | 70% | 70% | 70% | 70% | 70% |
| Cutoff 5 | 2.55 | 4.64 | 3.76 | 2.55 | 4.64 | 3.76 | 2.55 | 4.64 | 3.76 |
| Sens 5 | 85% | 82% | 88% | 76% | 79% | 78% | 59% | 53% | 43% |
| Spec 5 | 81% | 80% | 80% | 81% | 80% | 80% | 81% | 80% | 80% |
| Cutoff 6 | 4.56 | 13.7 | 7.61 | 4.56 | 13.7 | 7.61 | 4.56 | 13.7 | 7.61 |
| Sens 6 | 79% | 75% | 78% | 71% | 67% | 59% | 45% | 47% | 36% |
| Spec 6 | 90% | 90% | 90% | 90% | 90% | 90% | 90% | 90% | 90% |

C-C motif chemokine 14 X Cystatin-C/(Weight adjusted urine output)

| | | | | | | | | | |
|---|---|---|---|---|---|---|---|---|---|
| AUC | 0.90 | 0.85 | 0.90 | 0.88 | 0.84 | 0.87 | 0.68 | 0.69 | 0.64 |
| SE | 0.031 | 0.046 | 0.037 | 0.038 | 0.051 | 0.046 | 0.067 | 0.073 | 0.083 |
| p | 0 | 1.1E−14 | 0 | 0 | 2.0E−11 | 1.8E−15 | 0.0084 | 0.011 | 0.085 |
| nCohort 1 | 127 | 295 | 162 | 127 | 295 | 162 | 127 | 295 | 162 |
| nCohort 2 | 47 | 28 | 32 | 37 | 24 | 26 | 22 | 17 | 14 |
| Cutoff 1 | 3.16E7 | 4.84E7 | 3.96E7 | 8480000 | 2.43E7 | 8480000 | 934000 | 2500000 | 953000 |
| Sens 1 | 70% | 71% | 72% | 70% | 71% | 73% | 73% | 71% | 71% |
| Spec 1 | 98% | 92% | 97% | 91% | 86% | 86% | 45% | 54% | 44% |
| Cutoff 2 | 8480000 | 7980000 | 9900000 | 5230000 | 6250000 | 6580000 | 511000 | 1440000 | 511000 |
| Sens 2 | 81% | 82% | 81% | 81% | 83% | 81% | 82% | 82% | 86% |
| Spec 2 | 91% | 73% | 86% | 86% | 71% | 84% | 30% | 40% | 27% |
| Cutoff 3 | 1280000 | 1440000 | 3050000 | 934000 | 3050000 | 953000 | 192000 | 221000 | 446000 |
| Sens 3 | 91% | 93% | 91% | 92% | 92% | 92% | 91% | 94% | 93% |
| Spec 3 | 54% | 40% | 71% | 45% | 59% | 44% | 20% | 15% | 26% |
| Cutoff 4 | 2180000 | 6130000 | 2990000 | 2180000 | 6130000 | 2990000 | 2180000 | 6130000 | 2990000 |
| Sens 4 | 87% | 86% | 91% | 86% | 83% | 88% | 55% | 59% | 50% |
| Spec 4 | 70% | 70% | 70% | 70% | 70% | 70% | 70% | 70% | 70% |
| Cutoff 5 | 3560000 | 1.32E7 | 5150000 | 3560000 | 1.32E7 | 5150000 | 3560000 | 1.32E7 | 5150000 |
| Sens 5 | 85% | 75% | 88% | 84% | 71% | 85% | 50% | 47% | 43% |
| Spec 5 | 80% | 80% | 80% | 80% | 80% | 80% | 80% | 80% | 80% |
| Cutoff 6 | 8480000 | 3.31E7 | 1.33E7 | 8480000 | 3.31E7 | 1.33E7 | 8480000 | 3.31E7 | 1.33E7 |
| Sens 6 | 81% | 71% | 78% | 70% | 62% | 65% | 41% | 47% | 21% |
| Spec 6 | 91% | 90% | 90% | 91% | 90% | 90% | 91% | 90% | 90% |

TABLE 33
AUC comparison between the “non-diseased” cohort (patients that did not progress beyond KDIGO stage 0 or 1 within
12, 24, and 36 hours of sample collection) and the “diseased” cohort (patients that progressed to KDIGO stage
2 or 3 within 12, 24, and 36 hours of sample collection) where urine and blood samples were collected within 18 hours of enrollment.

| AKI onset from sample collection (hours) | | | | | | | | |
|---|---|---|---|---|---|---|---|---|
| 12 | | | 24 | | | 36 | | |
| sCr or UO | sCr only | UO only | sCr or UO | sCr only | UO only | sCr or UO | sCr only | UO only |

C-C motif chemokine 14

| | | | | | | | | | |
|---|---|---|---|---|---|---|---|---|---|
| AUC | 0.79 | 0.79 | 0.69 | 0.73 | 0.79 | 0.69 | 0.71 | 0.77 | 0.68 |
| SE | 0.026 | 0.040 | 0.028 | 0.026 | 0.040 | 0.028 | 0.025 | 0.037 | 0.027 |
| p Value | 0 | 0 | 0 | 0 | 0 | 0 | 0 | 0 | 0 |
| nCohort Non-persistent | 401 | 459 | 383 | 359 | 459 | 383 | 338 | 452 | 366 |
| nCohort Persistent | 107 | 48 | 122 | 149 | 48 | 122 | 170 | 55 | 139 |
| Cutoff Quartile 2 | 0.33 | 0.32 | 0.33 | 0.33 | 0.32 | 0.33 | 0.33 | 0.32 | 0.33 |
| Sensitivity | 93% | 92% | 86% | 87% | 92% | 86% | 86% | 91% | 85% |
| Specificity | 30% | 27% | 29% | 30% | 27% | 29% | 30% | 27% | 29% |
| Cutoff Quartile 3 | 0.56 | 0.56 | 0.56 | 0.56 | 0.56 | 0.56 | 0.56 | 0.56 | 0.56 |
| Sensitivity | 81% | 79% | 71% | 71% | 79% | 71% | 69% | 78% | 68% |
| Specificity | 58% | 53% | 57% | 59% | 53% | 57% | 59% | 54% | 57% |
| Cutoff Quartile 4 | 1.14 | 1.13 | 1.14 | 1.14 | 1.13 | 1.14 | 1.14 | 1.13 | 1.14 |
| Sensitivity | 62% | 69% | 48% | 52% | 69% | 48% | 47% | 62% | 45% |
| Specificity | 85% | 80% | 83% | 86% | 80% | 83% | 86% | 79% | 83% |

C-C motif chemokine 14 with serum creatinine and weight adjusted urine output

| | | | | | | | | | |
|---|---|---|---|---|---|---|---|---|---|
| AUC | 0.87 | 0.83 | 0.78 | 0.81 | 0.83 | 0.78 | 0.80 | 0.80 | 0.77 |
| SE | 0.020 | 0.034 | 0.025 | 0.023 | 0.034 | 0.025 | 0.022 | 0.035 | 0.024 |
| p Value | 0 | 0 | 0 | 0 | 0 | 0 | 0 | 0 | 0 |
| nCohort Non-persistent | 378 | 434 | 364 | 338 | 434 | 364 | 321 | 428 | 350 |
| nCohort Persistent | 103 | 46 | 117 | 143 | 46 | 117 | 160 | 52 | 131 |
| Cutoff Quartile 2 | 0.27 | 0.27 | 0.27 | 0.27 | 0.27 | 0.27 | 0.27 | 0.27 | 0.27 |
| Sensitivity | 97% | 96% | 93% | 93% | 96% | 93% | 92% | 94% | 93% |
| Specificity | 31% | 27% | 31% | 33% | 27% | 31% | 34% | 27% | 32% |
| Cutoff Quartile 3 | 0.86 | 0.86 | 0.86 | 0.86 | 0.86 | 0.86 | 0.86 | 0.86 | 0.86 |
| Sensitivity | 92% | 87% | 79% | 80% | 87% | 79% | 78% | 85% | 78% |
| Specificity | 62% | 54% | 60% | 63% | 54% | 60% | 64% | 54% | 61% |
| Cutoff Quartile 4 | 3.92 | 3.93 | 3.92 | 3.92 | 3.93 | 3.92 | 3.92 | 3.93 | 3.92 |
| Sensitivity | 69% | 72% | 56% | 57% | 72% | 56% | 53% | 65% | 53% |
| Specificity | 87% | 80% | 85% | 89% | 80% | 85% | 89% | 80% | 85% |

C-C motif chemokine 14 with Cystatin-C and weight adjusted urine output

| | | | | | | | | | |
|---|---|---|---|---|---|---|---|---|---|
| AUC | 0.88 | 0.84 | 0.77 | 0.80 | 0.84 | 0.77 | 0.79 | 0.80 | 0.76 |
| SE | 0.019 | 0.032 | 0.027 | 0.023 | 0.032 | 0.027 | 0.023 | 0.034 | 0.026 |
| p Value | 0 | 0 | 0 | 0 | 0 | 0 | 0 | 0 | 0 |
| nCohort Non-persistent | 380 | 434 | 367 | 341 | 434 | 367 | 323 | 428 | 352 |
| nCohort Persistent | 101 | 46 | 114 | 140 | 46 | 114 | 158 | 52 | 129 |
| Cutoff Quartile 2 | 6.1e+05 | 6.1e+05 | 6.1e+05 | 6.1e+05 | 6.1e+05 | 6.1e+05 | 6.1e+05 | 6.1e+05 | 6.1e+05 |
| Sensitivity | 98% | 98% | 91% | 92% | 98% | 91% | 91% | 96% | 91% |
| Specificity | 31% | 27% | 30% | 32% | 27% | 30% | 33% | 28% | 31% |
| Cutoff Quartile 3 | 2.1e+06 | 2.1e+06 | 2.1e+06 | 2.1e+06 | 2.1e+06 | 2.1e+06 | 2.1e+06 | 2.1e+06 | 2.1e+06 |
| Sensitivity | 94% | 91% | 79% | 81% | 91% | 79% | 77% | 87% | 77% |
| Specificity | 62% | 54% | 59% | 63% | 54% | 59% | 63% | 54% | 60% |
| Cutoff Quartile 4 | 1.0e+07 | 1.0e+07 | 1.0e+07 | 1.0e+07 | 1.0e+07 | 1.0e+07 | 1.0e+07 | 1.0e+07 | 1.0e+07 |
| Sensitivity | 73% | 70% | 59% | 59% | 70% | 59% | 54% | 63% | 55% |
| Specificity | 88% | 80% | 86% | 89% | 80% | 86% | 89% | 80% | 86% |

The results disclosed herein demonstrate that using CCL14 together with (i.e. in combination with) markers and/or indicia (parameters) disclosed herein which are alone at least somewhat predictive of renal status (particularly one or more of cystatin C, creatinine (e.g., serum creatinine or urine creatinine), and urine output) may provide improved predictability of renal status, particularly acute kidney injury and especially persistent acute kidney injury, over use of the constituent individual markers or indicia alone. In some embodiments, using CCL14 together with (i.e. in combination with) one or more additional kidney injury markers disclosed herein comprises combining a measurement for CCL14 with the one or more measurements of the additional kidney injury markers into a single composite value or assay result. In some embodiments, using CCL14 together with (i.e. in combination with) one or more additional kidney injury markers disclosed herein comprises using a measurement or other assay result for CCL14 with the one or more measurements or assay results of the additional kidney injury markers without combining the measurements/assay results into a single composite value or assay result. For instance, the measurements/assay results may be used together in a decision tree analysis, random forests analysis, and/or n-of-m (number positive) analysis. If the predictive information captured by measuring/assay for one or more additional kidney injury markers is non-redundant with the predictive information captured by measuring/assaying for CCL14 then the use of the markers together may result in improved predictive power over use of the markers alone. The measurements used for evaluating each parameter may be from the same sample or from different samples. The measurements may be made substantially concurrently or may be temporally spaced (e.g., within about 6 hours, 12 hours, 24 hours, 48 hours, 72 hours, or more). The improved predictive power of using the markers together may be assessed, for example, by differences in AUC, differences in probabilities, differences in sensitivity, differences in specificity, differences in outcomes of any other suitable models, including those described elsewhere herein (e.g., differences in scores for predicted events and/or non-events), and/or other suitable measures relative to individual parameters.

In some aspects, the parameters or combinations of parameters disclosed herein may predict development of persistent acute kidney injury. Persistent acute kidney injury may be defined as a subject maintaining a minimum level of kidney injury (e.g., KDIGO stage 1, KDIGO stage 2 or KDIGO stage 3) over a persistence period having a minimum duration of time. In some instances, the duration of time may be at least about 24 hours, 48 hours, 72 hours, 96 hours, 120 hours, 144 hours, or 168 hours. The persistent acute kidney injury may be measured over any persistence period beginning at the time of or after the parameters are ascertained (e.g., the time the subject sample(s) are obtained). For example, the persistence period may begin at the time of sample obtainment or within a 12 hour, 24 hour, 48 hour, 72 hour, 96 hour, 120 hour, 144 hour, 188 hour, or longer progression window beginning from the time of sampling the patient or a related time point. A persistence period beginning at the time of sample obtainment may be considered a diagnosis that the subject is currently experiencing persistent acute kidney injury. In some aspects, the parameters or combinations thereof disclosed herein may correlate to the likelihood of persistent acute kidney injury beginning within a certain time frame or at an approximate time point. In some implementations, the onset of the persistence period may be extrapolated from data related to the likelihood of persistent acute kidney injury developing within certain progression windows. For instance, comparison of the likelihood of persistent acute kidney injury developing within 12 hours and the likelihood of persistent acute kidney injury developing within 24 hours may inform the likelihood of the persistent acute kidney injury developing with an onset between about 12 hours and 24 hours.

The persistent acute kidney injury may be defined according to any stage of severity. For instance, the acute kidney injury may persist at KDIGO stage 1, KDIGO stage 2, or KDIGO stage 3. In some implementations, other classifications of the level of kidney injury may be suitably substituted for KDIGO stages or vice versa (e.g., RIFLE stage R may correspond to KDIGO stage 1, RIFLE stage I may correspond to KDIGO stage 2, and RIFLE stage F may correspond to KDIGO stage 3). In some implementations, the parameters or combinations disclosed herein may be used predict persistent acute kidney injury which is maintained at a certain level of severity or within a certain range of severity. For instance, the parameters may be able to predict persistent acute kidney injury which persists at KDIGO stage 2 but does not reach KDIGO stage 3 during the persistence period, acute kidney injury which persists at KDIGO stage 1 but does not reach KDIGO stage 2 during the persistence period, acute kidney injury which persists at KDIGO stage 1 or 2 but does not reach KDIGO stage 3 during the persistence period, etc. In some instances, the likelihood of persistence at a particular stage of acute kidney injury may be extrapolated from the likelihood of persistence at two or more minimal stages. For examples, the likelihood of persistent acute kidney injury at KDIGO stage 3 and the likelihood of persistent acute kidney injury at KDIGO stage 2 or 3 may inform the likelihood of persistent acute kidney injury at KDIGO stage 2 which does not progress to KDIGO stage 3 during the persistence period.

In some aspects, the subject may be experiencing acute kidney injury when the parameters for predicting likelihood of persistent acute kidney injury are ascertained (e.g., when samples are collected). For instance, the subject may have at least KDIGO stage 1, at least KDIGO stage 2, or at least KDIGO stage 3 when the parameters are ascertained. In some aspects, the subject may not be experiencing acute kidney injury when the parameters are ascertained. In some aspects, whether the subject is experiencing acute kidney injury and/or what stage of acute kidney injury the subject has may not be known or may not be ascertained until after the parameters for assessing renal status (e.g., predicting persistent acute kidney injury are obtained). In some implementations, the subject may be tested based on having a risk for acute kidney injury and/or persistent acute kidney injury. The risk may be a relatively recent exposure to an agent that predisposes the subject for renal injury, such as persistent acute kidney injury (e.g., NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin) and/or a relatively recent acute medical event that predisposes the patient to a risk of renal injury, such as persistent acute kidney injury (shock, sepsis, hemorrhage, an ischemic surgery, increased intra-abdominal pressure with acute decompensate heart failure, ischemia, pulmonary embolism, pancreatitis, a burn, or excess diuresis). In some aspects, the subject may been diagnosed with one or more conditions or comorbidities that increases the risk of renal injury, such as persistent acute kidney injury (e.g., congestive heart failure, preeclampsia, eclampsia, diabetes mellitus, hypertension, coronary artery disease, proteinuria, renal insufficiency, glomerular filtration below the normal range, cirrhosis, serum creatinine above the normal range, sepsis, injury to renal function, reduced renal function, or acute kidney injury). In some instances, the exposure, acute medical event, and/or diagnosis may have occurred within approximately the last 24 hours, the last 48 hours, the last 72 hours, the last 96 hours, the last 120 hours, the last 7 days, or the last month prior to testing the subject.

In some aspects, the subject may have had acute kidney injury or a minimum level of acute kidney injury for certain period prior to testing. For instance, the subject may have been experiencing acute kidney injury for at least 24 hours, at least 24 hours, at least 48 hours, at least 72 hours, at least 96 hours, at least 120 hours, at least 7 days, or longer. In some implementations, predictability of renal status, such as acute kidney injury, may improve if the patient is assessed after experiencing acute kidney injury for a longer period of time or for at least a minimum duration of time prior to testing. In some implementations, the subject may have been experiencing acute kidney injury for no longer than 24 hours, no longer than 24 hours, no longer than 48 hours, no longer than 72 hours, no longer than 96 hours, no longer than 120 hours, or no longer than 7 days. In some implementations, predictability of renal status, such as persistent acute kidney injury, may improve if the patient is assessed after experiencing acute kidney injury for a longer period of time or for at least a minimum duration of time prior to testing. In some implementations, predictability may be optimized by testing the subject after the subject has experienced acute kidney injury for a range of time no longer than a maximum threshold and no shorter than a minimum threshold. In some aspects, the assessment of renal status by one or more of the parameters disclosed herein may predict the likelihood of a subject progressing to persistent acute kidney injury which is more severe than the level of acute kidney injury the subject is currently experiencing (e.g., the likelihood of a subject experiencing KDIGO stage 2 acute kidney injury progressing to persistent KDIGO stage 3 acute kidney injury or the likelihood of a subject experiencing KDIGO stage 1 kidney injury progressing to persistent KDIGO stage 2 or 3 acute kidney injury). In some aspects, the assessment may predict the likelihood of the subject maintaining at least his or her current level of acute kidney injury over a persistence period (e.g., the likelihood of a subject experiencing KDIGO stage 2 acute kidney injury progressing to persistent KDIGO stage 2 or 3 acute kidney injury over a persistence period).

The likelihood of persistent acute kidney injury at any stage or stages may be suggestive of the likelihood of recovery from acute kidney injury. For instance, a relatively low likelihood that a subject experiencing KDIGO stage 3 acute kidney injury progresses to persistent KDIGO stage 3 acute kidney injury may be informative that a subject is more likely to recover from stage 3 acute kidney injury as defined, for example, by the subject maintaining a maximum KDIGO stage of 2 over the persistence period. In some embodiments, persistent acute kidney injury is defined by maintaining a minimum level/stage of AKI over the duration period (i.e. persistence period) and recovery is correspondingly defined by achieving during the duration period (i.e. recovery period) a level/stage of AKI below that minimum level/stage defining persistence. In some embodiments, recovery from acute kidney injury is defined by maintaining a maximum level/stage of AKI over the duration period (i.e. recovery period) and persistence is correspondingly defined by achieving during the duration period (i.e. persistence period) a level/stage of AKI above that maximum level/stage defining recovery.

In some embodiments, the subject may be persistent at RIFLE stage R or KDIGO stage 1 acute kidney injury. In some embodiments, the subject may be persistent at RIFLE stage I or KDIGO stage 2 acute kidney injury. In some embodiments, the subject may be persistent at RIFLE stage F or KDIGO stage 3 acute kidney injury. In some embodiments, the subject may become persistent at RIFLE stage R or KDIGO stage 1 acute kidney injury. In some embodiments, the subject may become persistent at RIFLE stage I or KDIGO stage 2 acute kidney injury. In some embodiments, the subject may become persistent at RIFLE stage F or KDIGO stage 3 acute kidney injury. In some embodiments, the subject may recover from RIFLE stage R or KDIGO stage 1 acute kidney injury. In some embodiments, the subject may recover from RIFLE stage I or KDIGO stage 2 acute kidney injury (to RIFLE/KDIGO stage 0 or to RIFLE stage R/KDIGO stage 1). In some embodiments, the subject may recover from RIFLE stage F or KDIGO stage 3 acute kidney injury (to RIFLE/KDIGO stage 0, RIFLE stage R/KDIGO stage 1, or to RIFLE stage I/KDIGO stage 2). CCL14 in combination with one or more of cystatin C, creatinine, and urine output may be correlated according to any of the methods disclosed herein to predict any of these outcomes (renal statuses).

In some embodiments, the subject may be at RIFLE stage R or KDIGO stage 1 acute kidney injury. In some embodiments, the subject may be at RIFLE stage I or KDIGO stage 2 acute kidney injury. In some embodiments, the subject may be at RIFLE stage F or KDIGO stage 3 acute kidney injury. In some embodiments, the subject may develop RIFLE stage R or KDIGO stage 1 acute kidney injury. In some embodiments, the subject may develop RIFLE stage I or KDIGO stage 2 acute kidney injury. In some embodiments, the subject may develop RIFLE stage F or KDIGO stage 3 acute kidney injury. CCL14 in combination with one or more of cystatin C, creatinine, and urine output may be correlated according to the methods disclosed herein to any of these outcomes (renal statuses).

In some embodiments, the subject may be at RIFLE stage R or KDIGO stage 1 acute kidney injury when the sample is obtained. In some embodiments, the subject may be at RIFLE stage I or KDIGO stage 2 acute kidney injury when the sample is obtained. In some embodiments, the subject may be at RIFLE stage F or KDIGO stage 3 acute kidney injury when the sample is obtained. The current renal status of the subject may be known (diagnosed) or unknown. In some embodiments, a diagnosis of the current renal status may be used to facilitate a correlation to a future renal status. In some embodiments, the correlation to a future renal status (e.g., a change in renal status) may be made concurrently with a diagnosis of the current renal status.

In some embodiments, the subject may have persistent RIFLE stage R or KDIGO stage 1 acute kidney injury when the sample is obtained. In some embodiments, the subject may have persistent RIFLE stage I or KDIGO stage 2 acute kidney injury when the sample is obtained. In some embodiments, the subject may have persistent RIFLE stage F or KDIGO stage 3 acute kidney injury when the sample is obtained. The current renal status of the subject may be known (diagnosed) or unknown. In some embodiments, a diagnosis of the current renal status may be used to facilitate a correlation to a future renal status. In some embodiments, the correlation to a future renal status (e.g., a change in renal status) may be made concurrently with a diagnosis of the current renal status.

In particular, C-C motif chemokine 14 measurements alone or in combination with one or more markers/indicia such as creatinine, urine output, and/or cystatin C may be used to predict renal status, such as persistent acute kidney injury. In some aspects, two or more of these parameters may be combined into a single composite value according to any suitable method, including those described elsewhere herein. For instance, the concentration of CCL14 may be multiplied by serum creatinine and/or may be multiplied by the concentration of cystatin C. The concentration of any of these markers or any composite of these markers may be divided by urine output (e.g., weight-adjusted urine output). Alternatively, all of these markers may be combined using a logistic regression model or other suitable model or some of these markers may be first combined by a “product model” and those composite value(s) may then combined with additional parameters using a logistic regression model or other suitable model. One or more of these parameters may further be combined according to the same methodologies with one or more additional parameters, including those described elsewhere herein, or those known in the art.

C-C motif chemokine 14 measurements alone or in combination with one or more markers/indicia such as creatinine (e.g., serum or plasma), urine output, and/or cystatin C may provide valuable information influencing a patient's prospective treatment regimen. Patients identified as having a relatively greater likelihood for persistent acute kidney injury, particularly persistent KDIGO stage 2 or 3 acute kidney injury, and especially persistent KDIGO stage 3 acute kidney injury may be identified as candidates for initiation of RRT. The predictive information from such an analysis may enable initiation of RRT earlier than it would otherwise begin, allowing more effective treatment of the patient. To the contrary, it may be decided that a patient identified as having a relatively less likelihood of persistent acute kidney injury or a patient identified as being likely only to persist at a less severe stage (e.g., KDIGO stage 1) may benefit from avoiding initiation of RRT, and the risks inherent therein, as such a patient is more likely to recover without such treatment. Patients at relatively higher risk of persistent acute kidney injury may be triaged to a higher level of care (e.g., a referral center) to mitigate potential implications of the persisting acute kidney injury. Patients at relatively higher risk of persistent acute kidney injury may benefit from additional interventions which may prevent or mitigate a transition from acute kidney injury to chronic kidney disease (Chawla L S, Bellomo R, Bihorac A, Goldstein S L, Siew E D, Bagshaw S M, et al. Nat Rev Nephrol. 2017; 13(4):241-57).

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

Other aspects are set forth within the following claims.

We claim:

1. A method for diagnosing an occurrence or nonoccurrence of renal injury, reduced renal function, or acute kidney injury (AKI) in a subject, comprising:
a. performing an analyte binding assay configured to detect C-C motif chemokine 14 in a first body fluid sample obtained from the subject; and
b. determining a measured value of one or more kidney injury markers.

2. The method of claim 1, wherein the one or more kidney injury markers is selected from the group consisting of: a creatinine concentration in the first or a second body fluid sample obtained from the subject, a cystatin C concentration in the first or the second body fluid obtained from the subject, a calculated glomerular filtration rate, and a urine output.

3. A method for risk stratification of a subject, comprising assigning a likelihood of the subject having persistent acute kidney injury, comprising:
a. performing an analyte binding assay configured to detect C-C motif chemokine 14 in a first body fluid sample obtained from the subject; and
b. determining a measured value of one or more kidney injury markers.

4. The method of claim 3, wherein the one or more kidney injury markers is selected from the group consisting of: a creatinine level in the first or a second body fluid sample obtained from the subject, a cystatin C level in the first or the second body fluid obtained from the subject, a calculated glomerular filtration rate, and a urine output.

5. A method for monitoring a renal injury in a subject for future persistence of acute kidney injury comprising:
a. performing an analyte binding assay configured to detect C-C motif chemokine 14 in a first body fluid sample obtained from the subject; and
b. determining a measured value of one or more kidney injury markers.

6. The method of claim 5, wherein the one or more kidney injury markers is selected from the group consisting of: a creatinine level in the first or a second body fluid sample obtained from the subject, a cystatin C level in the first or the second body fluid obtained from the subject, a calculated glomerular filtration rate, and a urine output.

7. The method of any one of the preceding claims, further comprising measuring a volume of urine output, a urine flow rate, a blood creatinine level, or a urine creatinine level within 7 days after the sample is obtained.

8. The method of claim 7, further comprising calculating a glomerular filtration rate.

9. The method of any one of claim 1, 2, 7, or 8 further comprising the step of combining the assay result and the measured value of one or more kidney injury markers into a single value to provide a composite assay result.

10. The method of any one of claim 1, 2, or 7-9, further comprising the step of correlating the assay result together with the measured value, optionally using a composite assay result, to a diagnosis of the renal status of the subject, wherein the diagnosis comprises an occurrence or nonoccurrence of renal injury, reduced renal function, or acute kidney injury (AKI).

11. The method of claim 10, wherein the correlating step comprises assigning the subject to a predetermined subpopulation of individuals having a known predisposition for renal injury, reduced renal function, or acute kidney injury (AKI), optionally the assignment being made by comparing a composite assay result to a threshold selected in a population study, wherein the threshold separates the population into a first subpopulation above the threshold which is at an increased predisposition for having renal injury, reduced renal function, or acute kidney injury (AKI) relative to a second subpopulation at or below the threshold.

12. The method of claim 11, further comprising the step of treating the subject based on the predetermined subpopulation of individuals to which the subject is assigned.

13. The method of claim 12, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration.

14. The method of claim 12, wherein treating the subject comprises treating a subject assigned to the first subpopulation, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration, optionally wherein the renal replacement therapy comprises continuous renal replacement therapy, intermittent hemodialysis, peritoneal dialysis, or renal transplantation.

15. The method of any one of claims 3-8, further comprising the step of combining the assay result and the measured value of one or more kidney injury markers into a single value to provide a composite assay result.

16. The method of any one of claim 3-8 or 15, further comprising the step of correlating the assay result together with the measured value, optionally using a composite assay result, to a likelihood of the subject having persistent acute kidney injury.

17. The method of claim 16, wherein the correlating step comprises assigning the subject to a predetermined subpopulation of individuals having a known predisposition for persistent acute kidney injury (AKI), optionally, the assignment being made by comparing a composite assay result to a threshold selected in a population study, wherein the threshold separates the population into a first subpopulation above the threshold which is at an increased predisposition for having persistent acute kidney injury (AKI) relative to a second subpopulation at or below the threshold.

18. The method of claim 17, further comprising the step of treating the subject based on the predetermined subpopulation of individuals to which the subject is assigned.

19. The method of claim 18, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration.

20. The method of claim 18, wherein treating the subject comprises treating a subject assigned to the first subpopulation, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration, optionally wherein the renal replacement therapy comprises continuous renal replacement therapy, intermittent hemodialysis, peritoneal dialysis, or renal transplantation.

21. The method of any one of claim 3-8 or 15, further comprising the step of correlating the assay result together with the measured value, optionally using a composite assay result to an occurrence or nonoccurrence of a change in renal status in the subject.

22. The method of claim 21, wherein the correlating step comprises assigning the subject to a predetermined subpopulation of individuals having a known predisposition for persistent acute kidney injury (AKI), optionally, the assignment being made by comparing a composite assay result to a threshold selected in a population study, wherein the threshold separates the population into a first subpopulation above the threshold which is at an increased predisposition for having persistent acute kidney injury (AKI) relative to a second subpopulation at or below the threshold.

23. The method of claim 22, further comprising the step of treating the subject based on the predetermined subpopulation of individuals to which the subject is assigned.

24. The method of claim 23, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration.

25. The method of claim 23, wherein treating the subject comprises treating a subject assigned to the first subpopulation, wherein the treatment comprises one or more of initiating renal replacement therapy, modifying administration of compounds known to be damaging to the kidney by adjusting the amount or selection of the compound, delaying or avoiding procedures that are known to be damaging to the kidney, or modifying diuretic administration, optionally wherein the renal replacement therapy comprises continuous renal replacement therapy, intermittent hemodialysis, peritoneal dialysis, or renal transplantation.

26. The method of any one of the preceding claims, wherein the subject has RIFLE stage I or F or KDIGO stage 2 or 3 when the first and second body fluid samples are obtained.

27. The method of any one of the preceding claims, wherein the binding reagent is an antibody.

28. The method of any one of the preceding claims, wherein the first body fluid sample is a urine sample.

29. The method of any one of the preceding claims, wherein the second body fluid sample is a blood sample.

30. The method of any one of the preceding claims, wherein the first body fluid sample and the second body fluid sample are the same sample.

31. The method of any one of claims 1-29, wherein the first body fluid sample and the second body fluid sample are different samples.

32. The method of any one of claim 9-14 or 26-31, wherein a composite assay result is generated via a function obtained using logistic regression.

33. The method of any one of claim 9-14 or 26-31, wherein a composite assay result is generated via a function obtained using linear discriminant analysis.

34. The method of claim 32 or 33, wherein the composite assay result is derived from a logistic regression model or a linear discriminant analysis comprising two or more independent variables selected from the group consisting of CCL14, cystatin C, creatinine, urine output, CCL14×cystatin C, CCL14×cystatin C×creatinine, CCL14×cystatin C/urine output, CCL14×creatinine, CCL14×creatinine/urine output, and CCL14/urine output.

35. The method of any one of claim 9-14 or 26-31, wherein the composite assay result is selected from the group consisting of CCL14×cystatin C, CCL14×cystatin C×creatinine, CCL14×cystatin C/urine output, CCL14×creatinine, CCL14×creatinine/urine output, CCL14/urine output, and CCL14×cystatin C×creatinine/urine output.

36. The method of any one of claim 10-14 or 26-31, wherein correlating the assay result together with the measured value comprises the use of a decision tree analysis or a random forests analysis.

37. The method of any one of claim 10-14 or 26-31, wherein correlating the assay result together with the measured value comprises the use of a number positive analysis.

38. The method of any one of claims 15-31, wherein the composite assay result is generated via a function obtained using logistic regression.

39. The method of any one of claims 15-31, wherein the composite assay result is generated via a function obtained using linear discriminant analysis.

40. The method of claim 38 or 39, wherein the composite assay result is derived from a logistic regression model or a linear discriminant analysis comprising two or more independent variables selected from the group consisting of CCL14, cystatin C, creatinine, urine output, CCL14×cystatin C, CCL14×cystatin C×creatinine, CCL14×cystatin C/urine output, CCL14×creatinine, CCL14×creatinine/urine output, and CCL14/urine output.

41. The method of any one of claims 15-31, wherein the single composite value is selected from the group consisting of CCL14×cystatin C, CCL14×cystatin C×creatinine, CCL14×cystatin C/urine output, CCL14×creatinine, CCL14×creatinine/urine output, CCL14/urine output and CCL14×cystatin C×creatinine/urine output.

42. The method of any one of claims 15-31, wherein correlating the assay result together with the measurement comprises the use of a decision tree analysis or a random forests analysis.

43. The method of any one of claims 15-31, wherein correlating the assay result together with the measurement comprises the use of a number positive analysis.

44. The method of any one of the preceding claims, wherein the subject has been diagnosed with one or more of congestive heart failure, preeclampsia, eclampsia, diabetes mellitus, hypertension, coronary artery disease, proteinuria, renal insufficiency, glomerular filtration below the normal range, cirrhosis, serum creatinine above the normal range, sepsis, injury to renal function, reduced renal function, and acute kidney injury.

45. The method of any one of the preceding claims, wherein the first and second body fluid samples were obtained within 7 days after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis.

46. The method of any one of the preceding claims, wherein the first and second body fluid samples were obtained within 72 hours after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis.

47. The method of any one of the preceding claims, wherein the first and second body fluid samples were obtained within 48 hours after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis.

48. The method of any one of the preceding claims, wherein the first and second body fluid samples were obtained within 24 hours after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis.

49. The method of any one of the preceding claims, wherein the first and second body fluid samples were obtained within 12 hours after an acute medical event which predisposes the patient for developing acute renal failure, wherein the acute medical event comprises shock, sepsis, hemorrhage, an ischemic surgery, increased intra-abdominal pressure, acute decompensated heart failure, ischemia, pulmonary embolism, pancreatitis, a burn, or excess diuresis.

50. The method of any one of the preceding claims, wherein the first and second body fluid samples were obtained within 7 days after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

51. The method of any one of the preceding claims, wherein the first and second body fluid samples were obtained within 72 hours after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

52. The method of any one of the preceding claims, wherein the first and second body fluid samples were obtained within 48 hours after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

53. The method of any one of the preceding claims, wherein the first and second body fluid samples were obtained within 24 hours after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

54. The method of any one of the preceding claims, wherein the first and second body fluid samples were obtained within 12 hours after an acute medical event which predisposes the patient for developing acute renal failure, wherein the acute medical event comprises exposure to NSAIDs, cyclosporines, tacrolimus, aminoglycosides, foscarnet, ethylene glycol, hemoglobin, myoglobin, ifosfamid, heavy metals, methotrexate, radiopaque contrast media, or streptozotocin.

55. The method of any one of the preceding claims, wherein the analyte binding assay configured to detect C-C motif chemokine 14 is performed by introducing a first body fluid sample obtained from the subject into an assay instrument which (i) contacts all or a portion of the first body fluid sample with a binding reagent which specifically binds to C-C motif chemokine 14 for detection of C-C motif chemokine 14 and (ii) generates an assay result indicative of binding of C-C motif chemokine 14 to the binding reagent.

56. The method of any one of the preceding claims, wherein the subject has RIFLE stage R or KDIGO stage 1 when the first and second body fluid samples are obtained.

57. The method of any one of the preceding claims, wherein the subject has RIFLE stage I or KDIGO stage 2 when the first and second body fluid samples are obtained.

58. The method of any one of the preceding claims, wherein the subject has RIFLE stage F or KDIGO stage 3 when the first and second body fluid samples are obtained.
